(19) United States
(12) Patent Application Publication
(10) Pub. No.: US 2026/0167650 A1
(43) Pub. Date: Jun. 18, 2026

(54) KRAS MODULATING COMPOUNDS

(71) Applicant: Gilead Sciences, Inc., Foster City, CA (US)

(72) Inventors: Eda Y. Canales, San Mateo, CA (US); Zhimin Du, Belmont, CA (US); Yassin M.A. Elbatrawi, Burlingame, CA (US); Quinn B. Greven, San Francisco, CA (US); Tezcan Guney, Foster City, CA (US); Darryl Kato, San Francisco, CA (US); Henok H. Kinfe, Foster City, CA (US); Scott E. Lazerwith, San Francisco, CA (US); Christopher J. Swank, San Mateo, CA (US); Rhiannon Thomas-Tran, San Jose, CA (US); Guanghu Tong, Foster City, CA (US); William J. Watkins, Mountain View, CA (US)

(21) Appl. No.: 19/354,567

(22) Filed: Oct. 9, 2025

Related U.S. Application Data
(60) Provisional application No. 63/706,467, filed on Oct. 11, 2024.
(60) Provisional application No. 63/789,114, filed on Apr. 15, 2025.

(51) Int. Cl. C07D 498/22; A61K 31/553; A61P 35/00; C07D 519/00

(52) U.S. Cl. CPC C07D 498/22; A61K 31/553; A61P 35/00; C07D 519/00

(57) ABSTRACT

Provided herein are compounds, and pharmaceutically acceptable salts thereof, useful as KRAS inhibitors and/or degraders, methods of making and using the same (singly or in combination with additional agents), and pharmaceutical compositions thereof.

### CROSS-REFERENCE TO RELATED APPLICATIONS

This application claims priority to U.S. Provisional Application No. 63/789,114 filed Apr. 15, 2025, and U.S. Provisional Application No. 63/706,467 filed Oct. 11, 2024, each of which is incorporated by reference herein in its entirety for all purposes.

### BACKGROUND

The KRAS protein, Kirsten Rat Sarcoma 2 Viral Oncogene Homolog (“KRAS”), is a GTPase. KRAS gene mutations have been observed in a number of conditions including, for instance, pancreatic cancer, endometrial cancer, lung adenocarcinoma, colorectal cancer, rectal carcinoma, gall bladder cancer, thyroid cancer, bile duct cancer, small cell lung cancer, and non-small cell lung cancer (NSCLC). Accordingly, there is a need for compounds, pharmaceutical compositions, and methods for inhibiting KRAS (e.g., wild type, KRAS G12C, KRAS G12D, and/or KRAS G12V) and treating associated cancers.

### SUMMARY

In one embodiment, the present disclosure provides a compound of Formula (I):

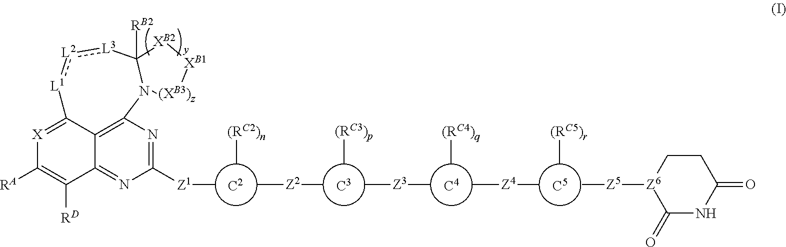

or a pharmaceutically acceptable salt thereof,
wherein
Z¹ is O, 4- to 16-membered heterocyclyl having at least one ring nitrogen, or

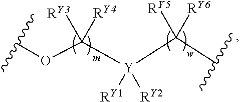

wherein the heterocyclyl is substituted with 0, 1, 2, 3, or 4 Z^{1A};
each Z^{1A} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
R^{Z1A} and R^{Z1B} are each independently C₁-C₆ alkyl, C₁-C₆ haloalkyl, or C₃-C₈ cycloalkyl;
Y is C or Si;
m is 0, 1, 2, or 3;
w is 0, 1, 2, or 3;
R^{Y1} is H, C₁-C₆ alkyl, C₁-C₆ haloalkyl or C₁-C₆ haloalkoxy;
R^{Y2} is H, C₁-C₆ alkyl, C₁-C₆ haloalkyl or C₁-C₆ haloalkoxy;
alternatively, R^{Y1} and R^{Y2} can combine to form ═CH₂, ═CHF, or ═CF₂;
alternatively, R^{Y1} and R^{Y2} can combine with the atom to which they are attached to form C₃-C₆ cycloalkyl, or a 3- to 10-membered heterocyclyl, wherein the cycloalkyl or heterocyclyl is substituted with 0, 1, 2, 3, or 4 R^{Y1C};
each R^{Y1C} is independently halo, C₁-C₆ alkyl, or C₁-C₆ haloalkyl;
each R^{Y3}, R^{Y4}, R^{Y5}, and R^{Y6} is independently H, halo, C₁-C₃ alkyl, C₁-C₃ haloalkyl or C₁-C₃ haloalkoxy;
ring C2 is 4- to 16-membered heterocyclyl having at least one ring nitrogen;
each R^{C2} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
n is 0, 1, 2, 3, or 4;
Z² is C₁-C₄ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z2a}—, —C(O)—C₁-C₃ alkyl, —C(O)NR^{Z2a}—, —C(O)—, —S(O)—, S(O)₂—, S(O)(═NR^{Z2})—, 4- to 10-membered heterocyclyl, or a bond, wherein the alkyl, cycloalkyl, and heterocyclyl is substituted with 0, 1, 2, 3, or 4 Z^{2a}, wherein the alkenyl is substituted with 0, 1, or 2 Z^{2c};
each Z^{2a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
each Z^{2c} is independently C₁-C₆ alkyl, halo, C₁-C₆ haloalkyl, or C₃-C₆ cycloalkyl;
R^{Z2} is H, C₁-C₆ alkyl, C₁-C₆ haloalkyl, or C₃-C₆ cycloalkyl;
R^{Z2a} is H, C₁-C₆ alkyl, C₁-C₆ haloalkyl, or C₃-C₆ cycloalkyl;
ring C3 is C₃-C₈ cycloalkyl, 4- to 16-membered heterocyclyl, C₆-C₁₀ aryl, 5- to 10-membered heteroaryl, or absent;
each R^{C3} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
p is 0, 1, 2, 3, or 4;
Z³ is C₁-C₄ alkyl, C₂-C₃ alkenyl, C₂-C₃ alkynyl, —O—C₁-C₃ alkyl, —O—C₂-C₃ alkenyl, —O—C₂-C₃ alkynyl, —O—, —S—, —NR^{Z3a}—, —C(O)—C₁-C₃ alkyl, —C(O)NR^{Z3a}—, —C(O)—, —S(O)—, —S(O)₂—, —S(O)(═NR^{Z3})—, C₃-C₄ cycloalkyl, 4- to 10-membered heterocyclyl, or a bond, wherein the alkyl and cycloalkyl is substituted with 0, 1, 2, 3, or 4 Z^{3a};
each Z^{3a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z3} is H, C₁-C₆ alkyl, C₁-C₆ haloalkyl or C₃-C₆ cycloalkyl;
R^{Z3a} is H, C₁-C₆ alkyl, C₁-C₆ haloalkyl, or C₃-C₆ cycloalkyl;
ring C4 is C₃-C₈ cycloalkyl, 4- to 16-membered heterocyclyl, C₆-C₁₀ aryl, 5- to 10-membered heteroaryl, or absent;
each R^{C4} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
q is 0, 1, 2, 3, or 4;
Z⁴ is C₁-C₄ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z4a}—, —C(O)—C₁-C₃ alkyl, —C(O)NR^{Z4a}—, —C(O)—, —S(O)—, S(O)₂—, S(O)(═NR^{Z4})—, 4- to 10-membered heterocyclyl, or a bond, wherein the alkyl and cycloalkyl is substituted with 0, 1, 2, 3, or 4 Z^{4a};
each Z^{4a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z4} is H, C₁-C₆ alkyl, C₁-C₆ haloalkyl or C₃-C₆ cycloalkyl;
R^{Z4a} is H, C₁-C₆ alkyl, C₁-C₆ haloalkyl, or C₃-C₆ cycloalkyl;
ring C5 is 5 to 14-membered heterocyclyl, C₆-C₁₀ aryl, or 5- to 10-membered heteroaryl;
each R^{C5} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₃ cyanoalkyl, C₃-C₆ cycloalkyl, or 4- to 10-membered heterocyclyl;
r is 0, 1, 2, 3, or 4;
Z⁵ is —NH—, —C(O)NH—, or a bond, and Z⁶ is —CH—;
alternatively, Z⁵ is a bond and Z⁶ is N;
X is N, CH, or CR^{x};
R^{x} is halo, C₁-C₃ haloalkyl, C₁-C₃ cyanoalkyl, or C₃-C₆ cycloalkyl;
L¹ is O, CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6-membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl; alternatively, R^{1e} and R^{2e} can combine with the atoms to which they are attached to form a C5-C6 cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
such that when L² is ═C(R^{2c})— then L¹ is —C(R^{1e})═ or L³ is ═C(R^{3e})—, and when L³ is ═C(R^{3e})— then L² is ═C(R^{2e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆haloalkyl, —CN, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SF₅, C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, SR^{A3}a —N(R^{A3a})(R^{A3b}) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ thiohaloalkyl, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} attached to two different atoms can combine to form a C₃-C₁₀ cycloalkyl or 4- to 10-membered heterocyclyl, wherein the cycloalkyl and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{D} is halo;
wherein, unless otherwise indicated, each heterocyclyl has 1, 2, 3, or 4 heteroatoms selected from N, O, S, and Si; and
each heteroaryl has 1, 2, 3, or 4 heteroatoms selected from N, O, and S. In some embodiments, L¹ is CR^{1a}R^{1b}, C(═CR^{1c}R^{1d}), C(═O), or —C(R^{1e})═.

In another embodiment, the present disclosure provides a pharmaceutical composition comprising a compound of the present disclosure, and a pharmaceutically acceptable excipient.

In another embodiment, the present disclosure provides a method of inhibiting KRAS protein, e.g., KRAS wild type, G12C, G12D, or G12V protein, in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of degrading KRAS protein, e.g., KRAS wild type, G12C, G12D, or G12V protein, in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of treating cancer in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

Also disclosed herein are compounds and pharmaceutically acceptable salts thereof of sub-formulas of Formula (I).

### DETAILED DESCRIPTION

### I. General

The disclosure relates generally to methods and compounds, and pharmaceutically acceptable salts thereof, for inhibiting and/or degrading KRAS protein, e.g., KRAS wild type, KRAS^{G12D}, KRAS^{G12C} and/or KRAS^{G12V}. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

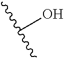 [SMILES: *O]

indicates a point of attachment, i.e., it shows the broken bond by which the group is connected to another described group.

As used herein, “a compound of the disclosure” can mean a compound of any of the Formula (I), (Ia), (Ib), (Ic), and/or (Id), or a pharmaceutically acceptable salt thereof. Similarly, the phrase “a compound of Formula (number)” means a compound of that formula and pharmaceutically acceptable salts thereof.

The prefix “Cᵤ-Cᵥ” indicates that the following group has from u to v carbon atoms. For example, “C₁-C₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadcyl, hexadecyl, heptadecyl and octadecyl. The alkyl group can be monovalent or multivalent, e.g., divalent.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkenyl group can be monovalent or multivalent, e.g., divalent.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl. The alkynyl group can be monovalent or multivalent, e.g., divalent.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, hexoxy, and the like. The alkoxy group can be monovalent or multivalent, e.g., divalent.

“Alkoxyalkyl” refers an alkoxy group linked to an alkyl group which is linked to the remainder of the compound. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). Alkoxy and alkyl are as defined above. Examples of “alkoxyalkyl” include, but are not limited to, methoxymethyl (CH₃OCH₂—), and methoxyethyl (CH₃OCH₂CH₂). The alkoxyalkyl group can be monovalent or multivalent, e.g., divalent.

“Bridged” means a ring system in which non-adjacent atoms on a ring are connected by a divalent substituent, such as an alkylenyl or heteroalkylenyl group or a single heteroatom.

“Hydroxyalkyl” refers to a hydroxy group, —OH, linked to an alkyl group which is linked to the remainder of the compound such that the alkyl group is divalent. Hydroxyalkyl can have any suitable number of carbons, such as from 1 to 8 (C₁₋₈ hydroxyalkyl), 1 to 6 (C₁₋₆ hydroxyalkyl), 2 to 6 (C₂₋₆ hydroxyalkyl), 2 to 4 (C₂₋₄ hydroxyalkyl), or 2 to 3 (C₂₋₃ hydroxyalkyl). Alkyl is as defined above where the alkyl is divalent.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Thioalkyl” refers to a thio group, —SH, linked to an alkyl group which is linked to the remainder of the compound such that the alkyl group is divalent. Thioalkyl can have any suitable number of carbons, such as from 1 to 8 (C₁₋₈ thioalkyl), 1 to 6 (C₁₋₆ thioalkyl), 2 to 6 (C₂₋₆ thioalkyl), 2 to 4 (C₂₋₄ thioalkyl), or 2 to 3 (C₂₋₃ thioalkyl). Alkyl is as defined above where the alkyl is divalent.

“Thiohaloalkyl” is a thioalkyl group, as defined above, in which one or more hydrogen atoms of the alkyl group is replaced with a halogen atom. The alkyl portion of a thiohaloalkyl group can have 1 to 20 carbon atoms (i.e., C₁-C₂₀ thiohaloalkyl), 1 to 12 carbon atoms (i.e., C₁-C₁₂ thiohaloalkyl), 1 to 8 carbon atoms (i.e., C₁-C₈ thiohaloalkyl), 1 to 6 carbon atoms (i.e., C₁-C₆ thiohaloalkyl) or 1 to 3 carbon atoms (i.e., C₁-C₃ thiohaloalkyl). The thiohaloalkyl groups can be substituted with 1, 2, 3, 4, 5, 6, 7, 8, 9 or more halogens. Alkyl is as defined above where the alkyl is divalent.

“Cyanoalkyl” refers to a cyano group, —CN, linked to an alkyl group which is linked to the remainder of the compound such that the alkyl group is divalent. Cyanoalkyl can have any suitable number of carbons, such as from 1 to 8 (C₁₋₈ cyanoalkyl), 1 to 6 (C₁₋₆ cyanoalkyl), 2 to 6 (C2-6 cyanoalkyl), 2 to 4 (C₂₋₄ cyanoalkyl), or 2 to 3 (C₂₋₃ cyanoalkyl). Alkyl is as defined above where the alkyl is divalent.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

The term “fused” refers to a ring system in which two or more rings in the system share a pair of adjacent ring atoms.

“Spiro” refers to at least two rings are linked together by one common atom. “Spiro” also refers to a ring substituent which is joined by two bonds at the same carbon atom. Examples of spiro groups include, but are not limited to, 1,1-diethylcyclopentane, dimethyl-dioxolane, and 4-benzyl-4-methylpiperidine, wherein the cyclopentane and piperidine, respectively, are the spiro substituents.

“Alkyl-cycloalkyl” refers to a radical having an alkyl component and a cycloalkyl component, where the alkyl component links the cycloalkyl component to the point of attachment.

The alkyl component is as defined above, except that the alkyl component is at least divalent, e.g., an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycle” or “heterocyclyl” or “heterocycloalkyl” refer to a saturated or partially saturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. For instance, heterocyclyl includes, without limitation, fused rings indoline, isoindoline, 4,5,6,7-tetrahydro-2H-pyrazolo[3,4-c]pyridine, and 2,3-dihydro-1H-benzo[d]imidazole; and spiro rings 2-azaspiro[3.3]heptane, 2-azaspiro[3.5]nonane, and 7-azaspiro[3.5]nonane. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, piperazinyl, oxetanyl, dioxolanyl, azetidinyl, and morpholinyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, e.g., an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkyl-aryl” refers to a radical having an alkyl component and an aryl component, where the alkyl component links the aryl component to the point of attachment. The alkyl component is as defined above, except that the alkyl component is at least divalent, e.g., an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, e.g., an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent.

The heteroaryl component is as defined within.

“KRAS G12C” refers to the G12C mutation of the KRAS protein, where cysteine replaces glycine at amino acid position 12.

“KRAS G12C inhibitor” refers to compounds of the present disclosure, including compounds of Formula (I). The compounds modulate or inhibit some or all of the activity of KRAS G12C.

“KRAS G12C-associated disease or disorder” refers to diseases or disorders associated with or mediated by or having a KRAS G12C mutation. Representative diseases or disorders include, but are not limited to, KRAS G12C-associated cancer.

“KRAS G12D” refers to the G12D mutation of the KRAS protein, where aspartic acid replaces glycine at amino acid position 12.

“KRAS G12D inhibitor” refers to compounds of the present disclosure, including compounds of Formula (I). The compounds modulate or inhibit some or all of the activity of KRAS G12D.

“KRAS G12D-associated disease or disorder” refers to diseases or disorders associated with or mediated by or having a KRAS G12D mutation. Representative diseases or disorders include, but are not limited to, KRAS G12D-associated cancer.

“KRAS G12V” refers to the G12V mutation of the KRAS protein, where aspartic acid replaces valine at amino acid position 12.

“KRAS G12V inhibitor” refers to compounds of the present disclosure, including compounds of Formula (I). The compounds modulate or inhibit some or all of the activity of KRAS G12V.

“KRAS G12V-associated disease or disorder” refers to diseases or disorders associated with or mediated by or having a KRAS G12V mutation. Representative diseases or disorders include, but are not limited to, KRAS G12V-associated cancer.

A “KRAS degrader” refers to any agent that is capable of binding to and inducing the degradation of KRAS protein. Generally, protein degraders are believed to induce targeted protein degradation through recruitment of the cellular ubiquitinylation and proteasomal protein degradation machinery.

“KRAS inhibitor” and “KRAS modulator” are used interchangeably to refer to a compound disclosed herein capable of inhibiting, modulating, and/or degrading a KRAS protein (mutant or wild-type) in an assay indicative of KRAS inhibition efficacy. “KRAS inhibition” or “KRAS modulation” includes modulation or promotion of degradation of a KRAS protein (mutant or wild-type).

“Oxo” refers to the group (═O) or (O).

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), (Ia), (Ib), (Ic), and/or (Id) can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. Similarly, an “arylalkyl” group, for example, can be attached to the remainder of the molecule at either an aryl or an alkyl portion of the group. A prefix such as “Cᵤ-Cᵥ” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compounds of Formula (I) are intended to have a valence of four. If, in some chemical structure representations, carbon atoms do not have a sufficient number of variables attached to produce a valence of four, the remaining carbon substituents needed to provide a valence of four should be assumed to be hydrogen.

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein. The term “therapeutically effective amount” or “effective amount” also means amounts that eliminate or reduce the subject's viral burden and/or viral reservoir.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Subject” refers to animals such as mammals, including, but not limited to, primates (e.g., humans), cows, sheep, goats, horses, dogs, cats, rabbits, rats, mice and the like. In some embodiments, the subject is a human.

“Disease” or “condition” refer to a state of being or health status of a patient or subject capable of being treated with a compound, pharmaceutical composition, or method provided herein. The disease may be an autoimmune, inflammatory, cancer, infectious (e.g., a viral infection), metabolic, developmental, cardiovascular, liver, intestinal, endocrine, neurological, or other disease. In some embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

“Cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma.

Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

“Leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

“Melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

“Carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basal oid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

“Metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

The term “adjacent carbons” as used herein refers to consecutive carbons atoms that are directly attached to each other. For example, in

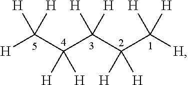

C₁ and C₂ are adjacent carbons, C₂ and C₃ are adjacent carbons, C₃ and C₄ are adjacent carbons, and C₄ and C₅ are adjacent carbons. Similarly,

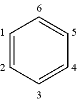

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

Disclosed herein are, among other things, compounds of Formula (I), (Ia), (Ib), (Ic) and/or (Id). In some embodiments, the present disclosure provides a compound of Formula (I):

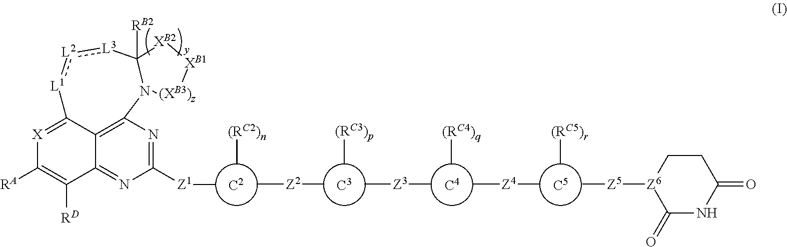

or a pharmaceutically acceptable salt thereof,
wherein
Z¹ is O, 4- to 16-membered heterocyclyl having at least one ring nitrogen, or

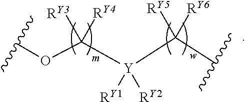

wherein the heterocyclyl is substituted with 0, 1, 2, 3, or 4 Z^{1A};
each Z^{1A} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
R^{Z1A} and R^{Z1B} are each independently C₁-C₆ alkyl, C₁-C₆ haloalkyl, or C₃-C₈ cycloalkyl;
Y is C or Si;
m is 0, 1, 2, or 3;
w is 0, 1, 2, or 3;
R^{Y1} is H, C₁-C₆ alkyl, C₁-C₆ haloalkyl or C₁-C₆ haloalkoxy;
R^{Y2} is H, C₁-C₆ alkyl, C₁-C₆ haloalkyl or C₁-C₆ haloalkoxy;
alternatively, R^{Y1} and R^{Y2} can combine to form ═CH₂, ═CHF, or ═CF₂;
alternatively, R^{Y1} and R^{Y2} can combine with the atom to which they are attached to form C₃-C₆ cycloalkyl, or a 3- to 10-membered heterocyclyl, wherein the cycloalkyl or heterocyclyl is substituted with 0, 1, 2, 3, or 4 R^{Y1C};
each R^{Y1C} is independently halo, C₁-C₆ alkyl, or C₁-C₆ haloalkyl;
each R^{Y3}, R^{Y4}, R^{Y5}, and R^{Y6} is independently H, halo, C₁-C₃ alkyl, C₁-C₃ haloalkyl or C₁-C₃ haloalkoxy;
ring C2 is 4- to 16-membered heterocyclyl having at least one ring nitrogen;
each R^{C2} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
n is 0, 1, 2, 3, or 4;
Z² is C₁-C₄ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z2a}—, —C(O)—C₁-C₃ alkyl, —C(O)NR^{Z2a}—, —C(O)—, —S(O)—, S(O)₂—, S(O)(═NR^{Z2})—, 4- to 10-membered heterocyclyl, or a bond, wherein the alkyl, cycloalkyl, and heterocyclyl is substituted with 0, 1, 2, 3, or 4 Z^{2a}, wherein the alkenyl is substituted with 0, 1, or 2 Z^{2c};
each Z^{2a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
each Z^{2c} is independently C₁-C₆ alkyl, halo, C₁-C₆ haloalkyl, or C₃-C₆ cycloalkyl;
R^{Z2} is H, C₁-C₆ alkyl, C₁-C₆ haloalkyl, or C₃-C₆ cycloalkyl;
R^{Z2a} is H, C₁-C₆ alkyl, C₁-C₆ haloalkyl, or C₃-C₆ cycloalkyl;
ring C3 is C₃-C₈ cycloalkyl, 4- to 16-membered heterocyclyl, C₆-C₁₀ aryl, 5- to 10-membered heteroaryl, or absent;
each R^{C3} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
p is 0, 1, 2, 3, or 4;
Z³ is C₁-C₄ alkyl, C₂-C₃ alkenyl, C₂-C₃ alkynyl, —O—C₁-C₃ alkyl, —O—C₂-C₃ alkenyl, —O—C₂-C₃ alkynyl, —O—, —S—, —NR^{Z3a}—, —C(O)—C₁-C₃ alkyl, —C(O)NR^{Z3a}—, —C(O)—, —S(O)—, —S(O)₂—, —S(O)(═NR^{Z3})—, C₃-C₄ cycloalkyl, 4- to 10-membered heterocyclyl, or a bond, wherein the alkyl and cycloalkyl is substituted with 0, 1, 2, 3, or 4 Z^{3a};
each Z^{3a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z3} is H, C₁-C₆ alkyl, C₁-C₆ haloalkyl or C₃-C₆ cycloalkyl;
R^{Z3a} is H, C₁-C₆ alkyl, C₁-C₆ haloalkyl, or C₃-C₆ cycloalkyl;
ring C4 is C₃-C₈ cycloalkyl, 4- to 16-membered heterocyclyl, C₆-C₁₀ aryl, 5- to 10-membered heteroaryl, or absent;
each R^{C4} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
q is 0, 1, 2, 3, or 4;
Z⁴ is C₁-C₄ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z4a}—, —C(O)—C₁-C₃ alkyl, —C(O)NR^{Z4a}—, —C(O)—, —S(O)—, S(O)₂—, S(O)(═NR⁴)—, 4- to 10-membered heterocyclyl, or a bond, wherein the alkyl and cycloalkyl is substituted with 0, 1, 2, 3, or 4 Z^{4a};
each Z^{4a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z4} is H, C₁-C₆ alkyl, C₁-C₆ haloalkyl or C₃-C₆ cycloalkyl;
R^{Z4a} is H, C₁-C₆ alkyl, C₁-C₆ haloalkyl, or C₃-C₆ cycloalkyl;
ring C5 is 5 to 14-membered heterocyclyl, C₆-C₁₀ aryl, or 5- to 10-membered heteroaryl;
each R^{C5} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₃ cyanoalkyl, C₃-C₆ cycloalkyl, or 4- to 10-membered heterocyclyl;
r is 0, 1, 2, 3, or 4;
Z⁵ is —NH—, —C(O)NH—, or a bond, and Z⁶ is —CH—;
alternatively, Z⁵ is a bond and Z⁶ is N;
X is N, CH, or CR^{x};
R^{x} is halo, C₁-C₃ haloalkyl, C₁-C₃ cyanoalkyl, or C₃-C₆ cycloalkyl;
L¹ is O, CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6-membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1c} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl; such that when L² is ═C(R^{2e})— then L¹ is —C(R^{1e})═ or L³ is ═C(R^{3e})—, and when L³ is ═C(R^{3e})— then L² is ═C(R^{2e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, S(O)₂OR^{A2a}, —N(R^{A2a})(R^{A2b}), —(C₀-C₃ alkyl)-SF₅, C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, —SR^{A3a}, —N(R^{A3a})(R^{A3b}) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ thiohaloalkyl, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} attached to two different atoms can combine to form a C₃-C₁₀ cycloalkyl or 4- to 10-membered heterocyclyl, wherein the cycloalkyl and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{D} is halo;
wherein, unless otherwise indicated, each heterocyclyl has 1, 2, 3, or 4 heteroatoms selected from N, O, S, and Si; and each heteroaryl has 1, 2, 3, or 4 heteroatoms selected from N, O, and S. In some embodiments, L¹ is CR^{1a}R^{1b}, C(═CR^{1c}R^{1d}), C(═O), or —C(R^{1e})═.

In some embodiments, the present disclosure provides the compound of Formula (I), or a pharmaceutically acceptable salt thereof, wherein X^{B1} is 0. In some embodiments, the present disclosure provides the compound of Formula (I), or a pharmaceutically acceptable salt thereof, wherein X^{B1} is CH₂.

In some embodiments, the present disclosure provides the compound of Formula (I), or a pharmaceutically acceptable salt thereof, wherein y is 1. In some embodiments, the present disclosure provides the compound of Formula (I), or a pharmaceutically acceptable salt thereof, wherein y is 2.

In some embodiments, the present disclosure provides the compound of Formula (I), or a pharmaceutically acceptable salt thereof, wherein X^{B2} is CH₂ or CF₂.

In some embodiments, the present disclosure provides the compound of Formula (I), or a pharmaceutically acceptable salt thereof, wherein X^{B3} is CH₂ or CF₂.

In some embodiments, the present disclosure provides the compound of Formula (I), or a pharmaceutically acceptable salt thereof, wherein z is 2. In some embodiments, the present disclosure provides the compound of Formula (I), or a pharmaceutically acceptable salt thereof, wherein z is 3.

In some embodiments, the present disclosure provides the compound of Formula (I), or a pharmaceutically acceptable salt thereof, wherein X^{B1} is CH₂ or O; X^{B2} and X^{B3} are each CH₂; y is 1 or 2; and z is 2 or 3.

In some embodiments, the present disclosure provides the compound of Formula (I), or a pharmaceutically acceptable salt thereof, having the structure of Formula (Ia):

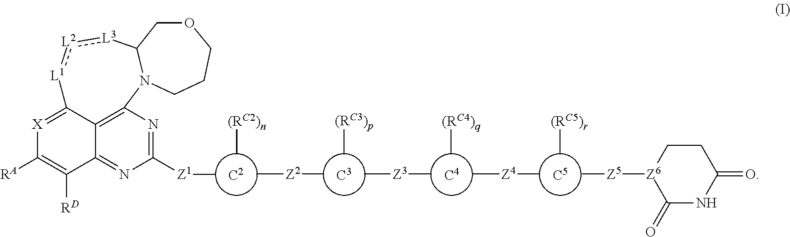

In some embodiments, the present disclosure provides the compound of Formula (I) and/or (Ia), or a pharmaceutically acceptable salt thereof, having the structure of Formula (Ib):

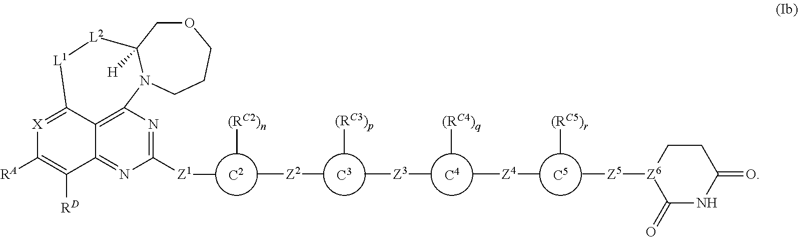

In some embodiments, the present disclosure provides the compound of Formula (I), (Ia), and/or (Ib), or a pharmaceutically acceptable salt thereof, having the structure of Formula (Ic):

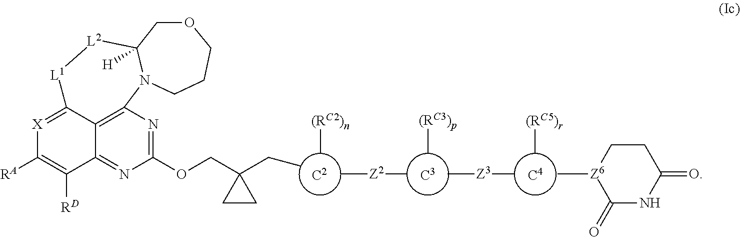

In some embodiments, the present disclosure provides the compound of Formula (I), (Ia), (Ib), and/or (Ic), or a pharmaceutically acceptable salt thereof, having the structure of Formula (Id):

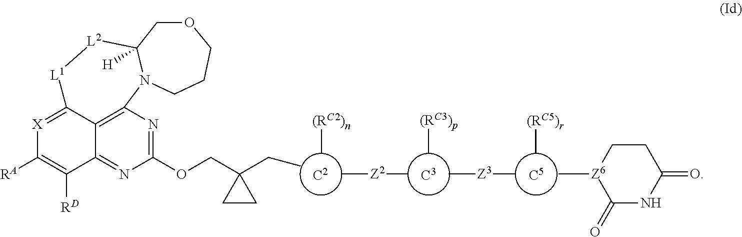

In some embodiments, the present disclosure provides the compound of Formula (I), (Ia), (Ib), (Ic), and/or (Id), or a pharmaceutically acceptable salt thereof, wherein X is N. In some embodiments, the present disclosure provides the compound of Formula (I), (Ia), (Ib), (Ic), and/or (Id), or a pharmaceutically acceptable salt thereof, wherein X is CH. In some embodiments, the present disclosure provides the compound of Formula (I), (Ia), (Ib), (Ic), and/or (Id), or a pharmaceutically acceptable salt thereof, wherein X is C—F. In some embodiments, the present disclosure provides the compound of Formula (I), (Ia), (Ib), (Ic), and/or (Id), or a pharmaceutically acceptable salt thereof, wherein X is C—Cl.

In some embodiments, the present disclosure provides the compound of Formula (I), (Ia), (Ib), (Ic), and/or (Id), or a pharmaceutically acceptable salt thereof, wherein L¹ is O.

In some embodiments, the present disclosure provides the compound of Formula (I), (Ia), (Ib), (Ic), and/or (Id), or a pharmaceutically acceptable salt thereof, wherein L¹ is CR^{1a}R^{1l}, C(═CR^{1c}R^{1d}), C(═O), or —C(R^{1e})═.

In some embodiments, the present disclosure provides the compound of Formula (I), (Ia), (Ib), (Ic), and/or (Id), or a pharmaceutically acceptable salt thereof, wherein L¹ is CR^{1a}R^{1b} or —C(R^{1e})═. In some embodiments, the present disclosure provides the compound of Formula (I), (Ia), (Ib), (Ic), and/or (Id), or a pharmaceutically acceptable salt thereof, wherein L¹ is O, S, or CR^{1a}R^{1b}.

In some embodiments, the present disclosure provides the compound of Formula (I), (Ia), (Ib), (Ic), and/or (Id), or a pharmaceutically acceptable salt thereof, wherein L¹ is CR^{1a}R^{1l}. In some embodiments, the present disclosure provides the compound of Formula (I), (Ia), (Ib), (Ic), and/or (Id), or a pharmaceutically acceptable salt thereof, wherein L¹ is CHR^{1b}.

In some embodiments, the present disclosure provides the compound of Formula (I), (Ia), (Ib), (Ic), and/or (Id), or a pharmaceutically acceptable salt thereof, wherein R^{1a} and R^{1b} are each independently H, C₁-C₃ alkyl, or halo.

In some embodiments, the present disclosure provides the compound of Formula (I), (Ia), (Ib), (Ic), and/or (Id), or a pharmaceutically acceptable salt thereof, wherein R^{1b} is C₁-C₃ alkyl or halo. In some embodiments, the present disclosure provides the compound of Formula (I), (Ia), (Ib), (Ic), and/or (Id), or a pharmaceutically acceptable salt thereof, wherein R^{1b} is methyl. In some embodiments, the present disclosure provides the compound of Formula (I), (Ia), (Ib), (Ic), and/or (Id), or a pharmaceutically acceptable salt thereof, wherein R^{1b} is H.

In some embodiments, the present disclosure provides the compound of Formula (I), (Ia), (Ib), (Ic), and/or (Id), or a pharmaceutically acceptable salt thereof, wherein L¹ is CH₂, CHCH₃, CHCH₂CH₃, CHF, or CF₂.

In some embodiments, the present disclosure provides the compound of (I), (Ia), (Ib), (Ic), and/or (Id), or a pharmaceutically acceptable salt thereof, wherein L¹ is CH₂.

In some embodiments, the present disclosure provides the compound of Formula (I), (Ia), (Ib), (Ic), and/or (Id), or a pharmaceutically acceptable salt thereof, wherein R^{2a} and R^{2b} are each independently H, C₁-C₃ alkyl, halo, or C₁-C₆ haloalkyl.

In some embodiments, the present disclosure provides the compound of Formula (I), (Ia), (Ib), (Ic), and/or (Id), or a pharmaceutically acceptable salt thereof, wherein R^{2a} is H.

In some embodiments, the present disclosure provides the compound of Formula (I), (Ia), (Ib), (Ic), and/or (Id), or a pharmaceutically acceptable salt thereof, wherein L² is CHR²b.

In some embodiments, the present disclosure provides the compound of Formula (I), (Ia), (Ib), (Ic), and/or (Id), or a pharmaceutically acceptable salt thereof, wherein R^{2b} is H or C₁-C₃ alkyl. In some embodiments, the present disclosure provides the compound of Formula (I), (Ia), (Ib), (Ic), and/or (Id), or a pharmaceutically acceptable salt thereof, wherein R^{2b} is H. In some embodiments, the present disclosure provides the compound of Formula (I), (Ia), (Ib), (Ic), and/or (Id), or a pharmaceutically acceptable salt thereof, wherein R^{2b} is C₁-C₃ alkyl. In some embodiments, the present disclosure provides the compound of Formula (I), (Ia), (Ib), (Ic), and/or (Id), or a pharmaceutically acceptable salt thereof, wherein R^{2b} is methyl.

In some embodiments, the present disclosure provides the compound of Formula (I), (Ia), (Ib), (Ic), and/or (Id), or a pharmaceutically acceptable salt thereof, wherein L¹ is —C(R^{1e})═; and L² is ═C(R^{2e})—. In some embodiments, the present disclosure provides the compound of Formula (I), (Ia), (Ib), (Ic), and/or (Id), or a pharmaceutically acceptable salt thereof, wherein L¹ is —C(CH₃)═; and L² is ═CH—.

In some embodiments, the present disclosure provides the compound of Formula (I) and/or (Ia), or a pharmaceutically acceptable salt thereof, wherein L¹ is CR^{1a}R^{1l}, L² is CR^{2a}R^{2b}; and Ria R^{1b}, R^{2a}, and R^{2b} are each independently H or C₁-C₃ alkyl; or L¹ is —C(R^{1e})═, and L² is —C(R^{2e})═; and R^{1e} and R^{2e} are each independently H or C₁-C₃ alkyl.

In some embodiments, the present disclosure provides the compound of Formula (I), (Ia), and/or (Ib), or a pharmaceutically acceptable salt thereof, wherein L³ is a bond. In some embodiments, the present disclosure provides the compound of Formula (I), (Ia), and/or (Ib), or a pharmaceutically acceptable salt thereof, wherein L³ is CR^{3a}R^{3b}.

In some embodiments, the present disclosure provides the compound of Formula (I), (Ia), and/or (Ib), or a pharmaceutically acceptable salt thereof, wherein R^{3a} and R^{3b} are H.

In some embodiments, the present disclosure provides the compound of Formula (I), (Ia), and/or (Ib), or a pharmaceutically acceptable salt thereof, wherein L³ is CH₂ or CHCH₃.

In some embodiments, the present disclosure provides the compound of Formula (I), (Ia), and/or (Ib), or a pharmaceutically acceptable salt thereof, wherein L¹ is CR^{1a}R^{1b}; L² is a bond, CR^{2a}R^{2b}, O, or S; and L³ is a bond or CR^{3a}R^{3b}. In some embodiments, the present disclosure provides the compound of Formula (I), (Ia), and/or (Ib), or a pharmaceutically acceptable salt thereof, wherein Ria and R^{1b} are each independently H, C₁-C₃ alkyl, or halo. In some embodiments, the present disclosure provides the compound of Formula (I), (Ia), and/or (Ib), or a pharmaceutically acceptable salt thereof, wherein L¹ is CHR^{1b}. In some embodiments, the present disclosure provides the compound of Formula (I), (Ia), and/or (Ib), or a pharmaceutically acceptable salt thereof, wherein R^{1b} is C₁-C₃ alkyl, halo, or —OH. In some embodiments, the present disclosure provides the compound of Formula (I), (Ia), and/or (Ib), or a pharmaceutically acceptable salt thereof, wherein R^{1b} is C₁-C₃ alkyl or halo. In some embodiments, the present disclosure provides the compound of Formula (I), (Ia), and/or (Ib), or a pharmaceutically acceptable salt thereof, wherein R^{1b} is methyl, F, or —OH. In some embodiments, the present disclosure provides the compound of Formula (I), (Ia), and/or (Ib), or a pharmaceutically acceptable salt thereof, wherein R^{1b} is methyl. In some embodiments, the present disclosure provides the compound of Formula (I), (Ia), and/or (Ib), or a pharmaceutically acceptable salt thereof, wherein L¹ is CH₂.

In some embodiments, the present disclosure provides the compound of Formula (I), (Ia), (Ib), (Ic), and/or (Id), or a pharmaceutically acceptable salt thereof, wherein R^{A} is phenyl substituted with 0, 1, or 2 R^{A2}. In some embodiments, the present disclosure provides the compound of Formula (I), (Ta), (Tb), (Ic), and/or (Id), or a pharmaceutically acceptable salt thereof, wherein R^{A} is phenyl substituted with 3 R^{A2}. In some embodiments, the present disclosure provides the compound of Formula (I), (Ia), (Tb), (Ic), and/or (Id), or a pharmaceutically acceptable salt thereof, wherein R^{A} is naphthyl substituted with 0, 1, 2, 3, 4, or 5 R^{A2}. In some embodiments, the present disclosure provides the compound of Formula (I), (Ia), (Tb), (Ic), and/or (Id), or a pharmaceutically acceptable salt thereof, wherein R^{A} is naphthyl substituted with 2, 3, or 4 R^{A2}. In some embodiments, the present disclosure provides the compound of Formula (I), (Ia), (Tb), (Ic), and/or (Id), or a pharmaceutically acceptable salt thereof, wherein R^{A} is 5- to 14-membered heteroaryl, wherein R^{A} is substituted with 0, 1, 2, 3, 4, or 5 R^{A2}. In some embodiments, the present disclosure provides the compound of Formula (I), (Ia), (Tb), (Ic), and/or (Id), or a pharmaceutically acceptable salt thereof, wherein R^{A} is 5- to 6-membered heteroaryl, wherein R^{A} is substituted with 0, 1, or 2 R^{A2}. In some embodiments, the present disclosure provides the compound of Formula (I), (Ta), (Tb), (Ic), and/or (Id), or a pharmaceutically acceptable salt thereof, wherein R^{A} is pyridyl, wherein R^{A} is substituted with 0, 1, or 2 R^{A2}.

In some embodiments, the present disclosure provides the compound of Formula (I), (Ia), (Tb), (Ic), and/or (Id), or a pharmaceutically acceptable salt thereof, wherein R^{A} is

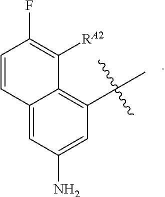

In some embodiments, the present disclosure provides the compound of Formula (I), (Ia), (Ib), (Ic), and/or (Id), or a pharmaceutically acceptable salt thereof, wherein each R^{A2} is independently C₁-C₆ alkyl, —OH, C₂-C₆ alkenyl, C₂-C₆ alkynyl, halo, C₁-C₆ haloalkyl, —CN, oxo, —OR^{A2a}, —SR^{A2a}, —N(R^{A2a})(R^{A2b}) or —(C₁-C₆ alkyl)-(C₃-C₈ cycloalkyl), wherein each alkenyl is substituted with 0, 1, 2, or 3 R^{A3}; each R^{A2a} and R^{A2b} is independently H, C₁-C₆ haloalkyl, or C₃-C₈ cycloalkyl; and each R^{A3} is independently halo.

In some embodiments, the present disclosure provides the compound of Formula (I), (Ia), (Ib), (Ic), and/or (Id), or a pharmaceutically acceptable salt thereof, wherein each R^{A2} is independently Me, Et, —OH, —CH═CHMe, —C(Cl)═CH₂, —CH═CHF, —C≡CH, F, Cl, CF₃, —CH₂CF₃, —CN, oxo, —OCF₃, —O-cyclopropyl, —SCF₃, —NH₂, or —CH₂-cyclopropyl. In some embodiments, the present disclosure provides the compound of Formula (I), (Ia), (Ib), (Ic), and/or (Id), or a pharmaceutically acceptable salt thereof, wherein each R^{A2} is independently Et, —OH, —C≡CH, F, Cl, CF₃, —CN, or —NH₂.

In some embodiments, the present disclosure provides the compound of Formula (I), (Ia), (Ib), (Ic), and/or (Id), or a pharmaceutically acceptable salt thereof, wherein each R^{A2} is independently C₁-C₆ alkyl, —OH, C₂-C₆ alkenyl, C₂-C₆ alkynyl, halo, C₁-C₆ haloalkyl, —OR^{A2a}, —SR^{A2a}, or —(C₁-C₆ alkyl)-(C₃-C₈ cycloalkyl), wherein each alkenyl is substituted with 0, 1, 2, or 3 R^{A3}; each R^{A2a} and R^{A2b} is independently H, C₁-C₆ haloalkyl, or C₃-C₈ cycloalkyl; and each R^{A3} is independently halo. In some embodiments, each R^{A2} is independently Me, —OH, —C(Cl)═CH₂, —CH═CHF, —C≡CH, F, Cl, —CH₂CF₃, —OCF₃, —O-cyclopropyl, —SCF₃, or —CH₂-cyclopropyl. In some embodiments, each R^{A2} is independently Et, OH, —C≡CH, F, Cl, CF₃, —CN, or —NH₂.

In some embodiments, the present disclosure provides the compound of Formula (I), (Ia), (Ib), (Ic), and/or (Id), or a pharmaceutically acceptable salt thereof, wherein R^{A} is

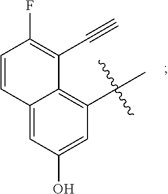 [SMILES: *c1cc(O)cc2ccc(F)c(C#C)c12]

### and Q is H, OH, or NH₂.

In some embodiments, the present disclosure provides the compound of Formula (I), (Ia), (Ib), (Ic), and/or (Id), or a pharmaceutically acceptable salt thereof, wherein R^{A} is

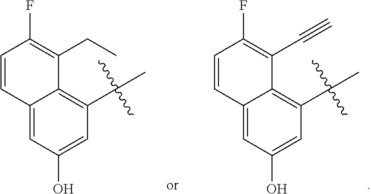 [SMILES: *c1cc(O)cc2ccc(F)c(CC)c12 ; *c1cc(O)cc2ccc(F)c(C#C)c12]

In some embodiments, the present disclosure provides the compound of (I), (Ia), (Ib), (Ic), and/or (Id), or a pharmaceutically acceptable salt thereof, wherein R^{D} is F.

In some embodiments, the present disclosure provides the compound of Formula (I), (Ia), and/or (Ib), or a pharmaceutically acceptable salt thereof, wherein L¹ is O or CR^{1a}R^{1b}; Ria and R^{1b} are each independently H, C₁-C₃ alkyl, or halo; L² is CR^{2a}R^{2b}; R^{2a} and R^{2b} are each independently H, C₁-C₃ alkyl, C₁-C₆ haloalkyl, or C₃-C₆ cycloalkyl; L³ is a bond or CR^{3a}R^{3b}; R^{3a} and R^{3b} are each independently H or C₁-C₃ alkyl; R^{A} is naphthyl, wherein R^{A} is substituted with 0, 1, 2, 3, 4, or 5 R^{A2}; each R^{A2} is independently —OH, C₁-C₃ alkyl, C₂-C₆ alkenyl, C₂-C₆ alkynyl, C₁-C₆ alkoxy, C₁-C₆ thioalkyl, halo, C₁-C₆ haloalkyl, —OR^{A2a}, —SR^{A2a}, —N(R^{A2a})(R^{A2b}), or —(C₁-C₆ alkyl)-(C₃-C₈ cycloalkyl), wherein each alkyl, alkenyl, alkynyl, alkoxy, and haloalkyl is substituted with 0, 1, 2, or 3 R^{A3}; each R^{A2a} and R^{A2b} is independently H, C₁-C₆ haloalkyl, or C₃-C₈ cycloalkyl; and each R^{A3} is independently halo.

In some embodiments, the present disclosure provides the compound of Formula (I), (Ia), and/or (Ib), or a pharmaceutically acceptable salt thereof, wherein X is N, CH, or CR^{x}; R^{x} is halo; L¹ is O or CR^{1a}R^{1b}; Ria and Rib are each independently H, C₁-C₃ alkyl, or halo; L² is CR^{2a}R^{2b}; R^{2a} and R^{2b} are each independently H, C₁-C₃ alkyl, C₁-C₆ haloalkyl, or C₃-C₆ cycloalkyl; L³ is a bond or CR^{3a}R^{3b}; R^{3a} and R^{3b} are each independently H or C₁-C₃ alkyl; R¹ is H or F; R² is F; R³, R⁴, and R⁵ are each H; R^{A} is naphthyl, wherein R^{A} is substituted with 0, 1, 2, 3, 4, or 5 R^{A2}; each R^{A2} is independently —OH, C₁-C₃ alkyl, C₂-C₆ alkenyl, C₂-C₆ alkynyl, C₁-C₆ alkoxy, C₁-C₆ thioalkyl, halo, C₁-C₆ haloalkyl, —OR^{A2a}, —SR^{A2a}, —N(R^{A2a})(R^{A2b}), or —(C₁-C₆ alkyl)-(C₃-C₈ cycloalkyl), wherein each alkyl, alkenyl, alkynyl, alkoxy, and haloalkyl is substituted with 0, 1, 2, or 3 R^{A3}; each R^{A2a} and R^{A2b} is independently H, C₁-C₆ haloalkyl, or C₃-C₈ cycloalkyl; each R^{A3} is independently halo; and R^{D} is F.

In some embodiments, the present disclosure provides the compound of Formula (I), (Ia), and/or (Ib), or a pharmaceutically acceptable salt thereof, wherein X is N, CH, or C—Cl; L¹ is O, CH₂, CHCH₃, CHCH₂CH₃, CHF, or CF₂; L² is CH₂, CHCH₃, CHCH₂CH₃, CHCHF₂, or

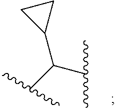 [SMILES: *C(*)C1CC1]

L³ is a bond, CH₂, or CHCH₃; R¹ is H or F; R² is F; R³, R⁴, and R⁵ are each H; R^{A} is

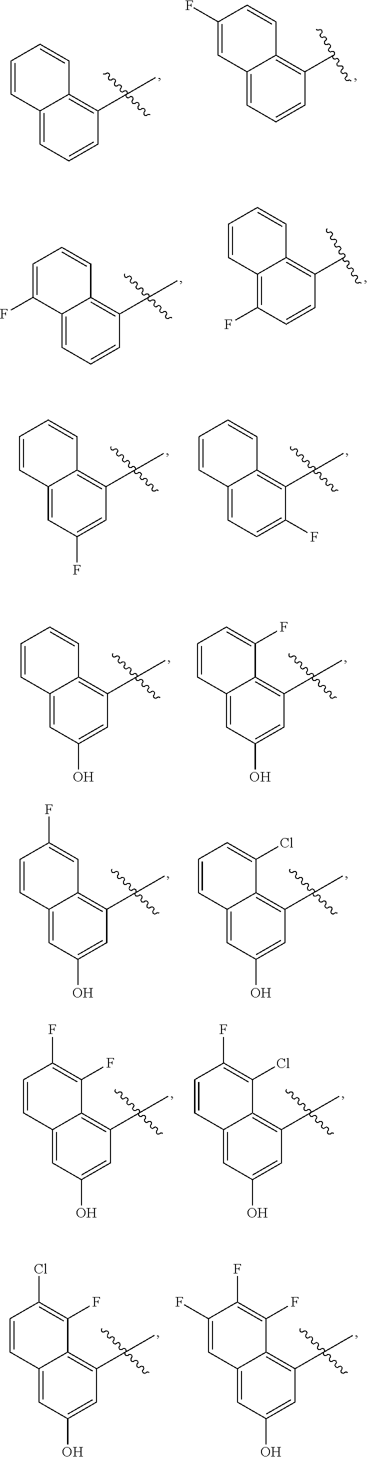

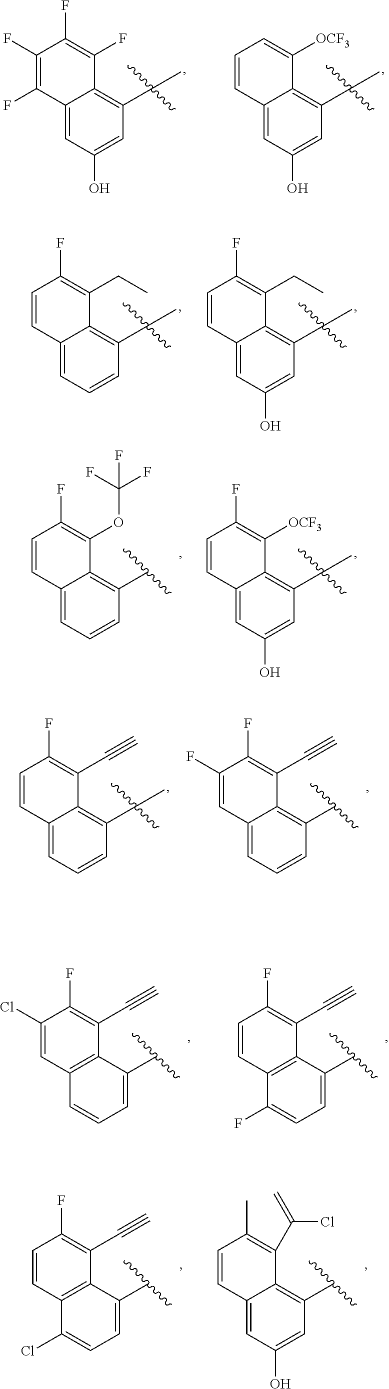

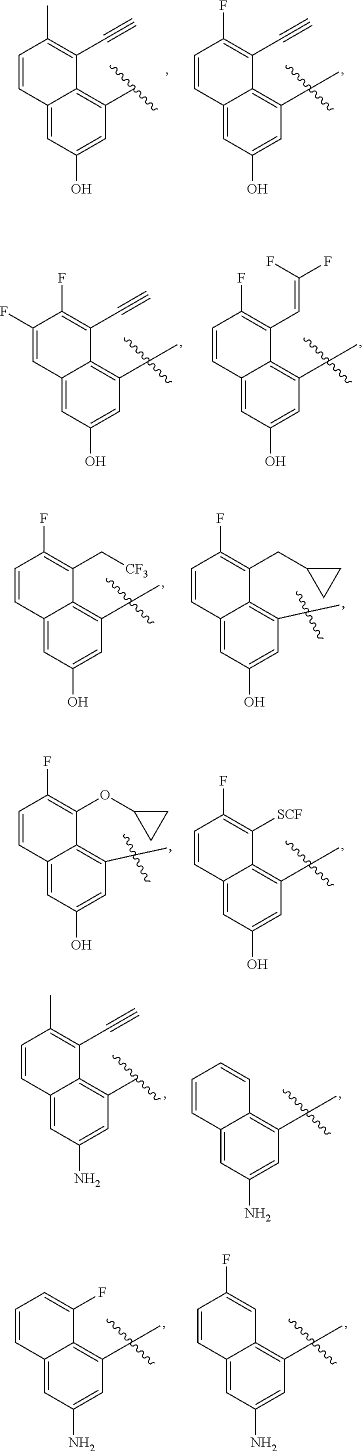

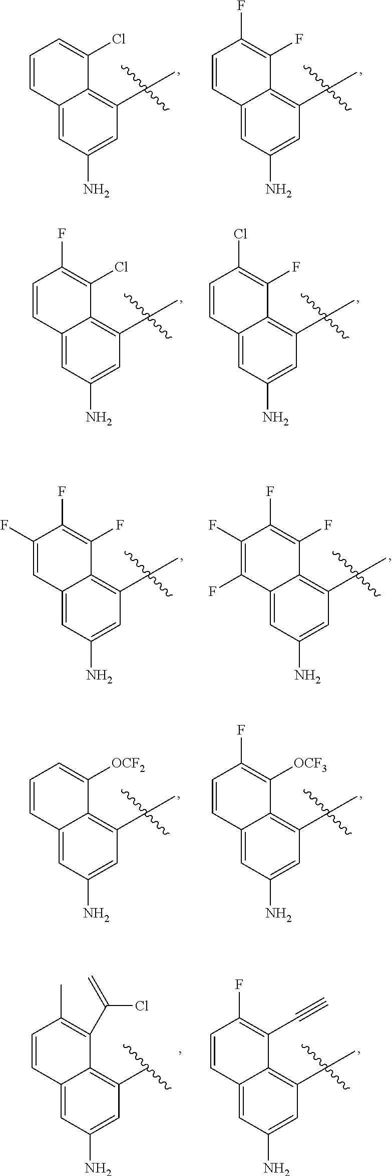

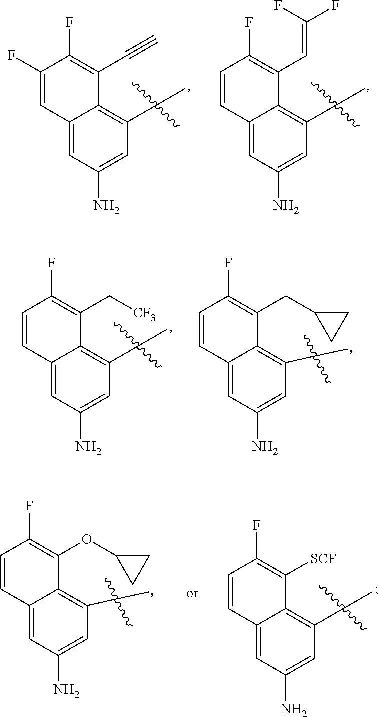

and R^{D} is F.

In some embodiments, the present disclosure provides the compound of Formula (I), (Ia), (Ib), and/or (Ic), or a pharmaceutically acceptable salt thereof, wherein Z¹ is

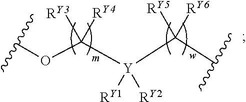

m is 0 or 1; and w is 0 or 1.

In some embodiments, the present disclosure provides the compound of Formula (I), (Ia), (Ib), and/or (Ic), or a pharmaceutically acceptable salt thereof, wherein Z¹ is

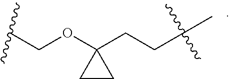 [SMILES: *CCC1(OC*)CC1]

In some embodiments, the present disclosure provides the compound of Formula (I), (Ia), (Ib), and/or (Ic), or a pharmaceutically acceptable salt thereof, wherein Z¹ is

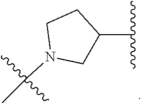 [SMILES: *C1CCN(*)C1]

In some embodiments, the present disclosure provides the compound of Formula (I), (Ia), (Ib), and/or (Ic), or a pharmaceutically acceptable salt thereof, wherein Z¹ is

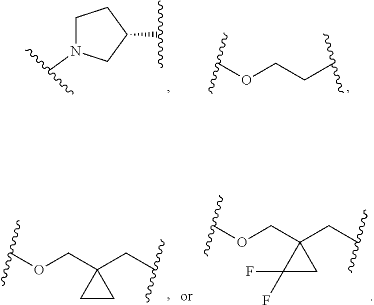 [SMILES: *[C@H]1CCN(*)C1 ; *CCO* ; *CC1(CO*)CC1 ; *CC1(CO*)CC1(F)F]

As described herein, the ring designated as

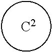

in the compound of Formula (I), (Ta), (Tb), (Ic), and/or (Id), or a pharmaceutically acceptable salt thereof, is also referred to herein interchangeably as ring C2.

In some embodiments, the present disclosure provides the compound of Formula (I), (Ia), (Ib), (Ic), and/or (Id), or a pharmaceutically acceptable salt thereof, wherein ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl, halo, C₁-C₆ haloalkyl, or —OH; and n is 0 or 1.

In some embodiments, the present disclosure provides the compound of Formula (I), (Ia), (Ib), (Ic), and/or (Id), or a pharmaceutically acceptable salt thereof, wherein ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl or —OH; and n is 0 or 1.

In some embodiments, the present disclosure provides the compound of Formula (I), (Ia), (Ib), (Ic), and/or (Id), or a pharmaceutically acceptable salt thereof, wherein
ring C2 is 6- to 14-membered heterocyclyl having one or two ring nitrogen, wherein the heterocyclyl is monocyclic or spiro bicyclic; each R^{C2} is independently C₁-C₆ alkyl, halo, C₁-C₆ haloalkyl, or —OH; and n is 0 or 1.

In some embodiments, the present disclosure provides the compound of Formula (I), (Ia), (Tb), (Ic), and/or (Id), or a pharmaceutically acceptable salt thereof, wherein ring C2 is 6- to 14-membered heterocyclyl having at least one ring nitrogen, wherein the heterocyclyl has 1 or 2 additional heteroatoms selected from N and O; each R^{C2} is independently C₁-C₆ alkyl or —OH; and n is 0 or 1.

In some embodiments, the present disclosure provides the compound of Formula (I), (Ia), (Ib), (Ic), and/or (Id), or a pharmaceutically acceptable salt thereof, wherein
ring C2 is 6- to 14-membered heterocyclyl having one or two ring nitrogen, wherein the heterocyclyl is monocyclic or spiro bicyclic; each R^{C2} is independently C₁-C₆ alkyl or —OH; and n is 0 or 1.

In some embodiments, the present disclosure provides the compound of Formula (I), (Ia), (Ib), (Ic), and/or (Id), or a pharmaceutically acceptable salt thereof, wherein ring C2 is

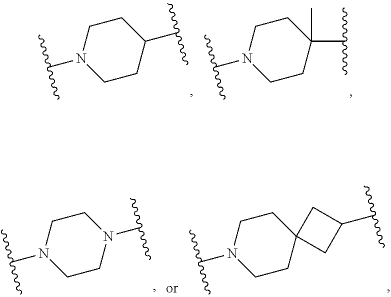

In some embodiments, the present disclosure provides the compound of Formula (I), (Ia), (Ib), (Ic), and/or (Id), or a pharmaceutically acceptable salt thereof, wherein ring C2 is

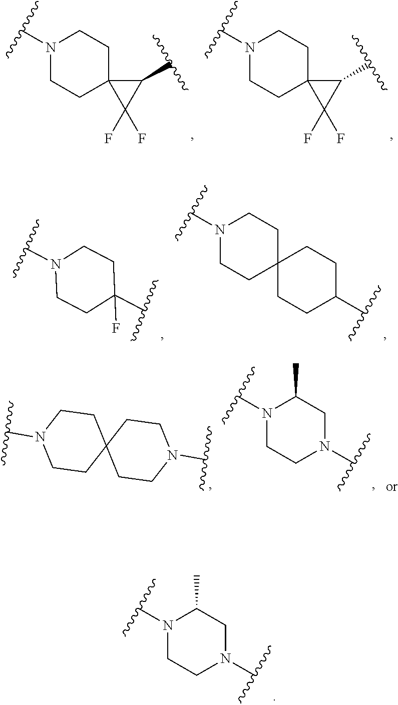

In some embodiments, the present disclosure provides the compound of Formula (I), (Ia), (Ib), (Ic), and/or (Id), or a pharmaceutically acceptable salt thereof, wherein Z² is C₁-C₃ alkyl, C₂-C₃ alkynyl, —O—C₁-C₃ alkyl, —O—C₂-C₃ alkynyl, —O—, or a bond. In some embodiments, the present disclosure provides the compound of Formula (I), (Ia), (Ib), (Ic), and/or (Id), or a pharmaceutically acceptable salt thereof, wherein Z² is C₁-C₃ haloalkyl, or —NR^{Z2a}—; and R^{Z2a} is C₁-C₃ alkyl. In some embodiments, the present disclosure provides the compound of Formula (I), (Ia), (Ib), (Ic), and/or (Id), or a pharmaceutically acceptable salt thereof, wherein Z² is CH₂, —CH₂CH₂—, —C≡C—, —OCH₂—, —CH₂O—, —OCH₂—C≡C—, —O—, or a bond. In some embodiments, the present disclosure provides the compound of Formula (I), (Ia), (Ib), (Ic), and/or (Id), or a pharmaceutically acceptable salt thereof, wherein Z² is CH₂ or a bond. In some embodiments, the present disclosure provides the compound of Formula (I), (Ia), (Ib), (Ic), and/or (Id), or a pharmaceutically acceptable salt thereof, wherein Z² is CF₂, —OCH₂C≡C—, —O—, or —N(CH₃)—.

As described herein, the ring designated as

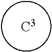

in the compound of Formula (I), (Ia), (Ib), (Ic), and/or (Id), or a pharmaceutically acceptable salt thereof, is also referred to herein interchangeably as ring C3.

In some embodiments, the present disclosure provides the compound of Formula (I), (Ia), (Ib), (Ic), and/or (Id), or a pharmaceutically acceptable salt thereof, wherein ring C3 is 4- to 16-membered heterocyclyl or absent, wherein the heterocyclyl has 1, 2, 3, or 4 heteroatoms selected from N, O, and S; each R^{C3} is independently C₁-C₆ alkyl; and p is 0 or 1.

In some embodiments, the present disclosure provides the compound of Formula (I), (Ia), (Ib), (Ic), and/or (Id), or a pharmaceutically acceptable salt thereof, wherein ring C3 is 4- to 16-membered heterocyclyl or absent, wherein the heterocyclyl has 1 or 2 heteroatoms that is N; each R^{C3} is independently C₁-C₆ alkyl; and p is 0 or 1. In some embodiments, each R^{C3} is independently C₁-C₆ alkyl or halo.

In some embodiments, the present disclosure provides the compound of Formula (I), (Ia), (Ib), (Ic), and/or (Id), or a pharmaceutically acceptable salt thereof, wherein
ring C3 is 5- to 14-membered heterocyclyl having one or two ring nitrogen, wherein the heterocyclyl is monocyclic or spiro bicyclic; each R^{C3} is independently C₁-C₆ alkyl; and n is 0 or 1. In some embodiments, each R^{C3} is independently C₁-C₆ alkyl or halo.

In some embodiments, the present disclosure provides the compound of Formula (I), (Ia), (Ib), (Ic), and/or (Id), or a pharmaceutically acceptable salt thereof, wherein
ring C3 is 6- to 9-membered heterocyclyl or absent, wherein the heterocyclyl has 1 or 2 heteroatoms that is N; each R^{C3} is independently C₁-C₆ alkyl; and p is 0 or 1. In some embodiments, each R^{C3} is independently C₁-C₆ alkyl or halo.

In some embodiments, the present disclosure provides the compound of Formula (I), (Ia), (Ib), (Ic), and/or (Id), or a pharmaceutically acceptable salt thereof, wherein
ring C3 is:

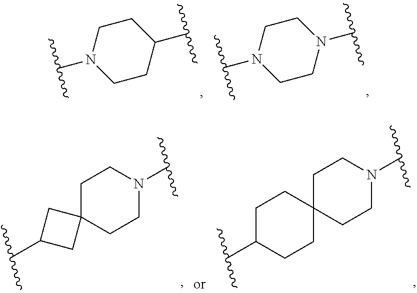 [SMILES: *C1CCN(*)CC1 ; *N1CCN(*)CC1 ; *C1CC2(CCN(*)CC2)C1 ; *C1CCC2(CC1)CCN(*)CC2]

In some embodiments, the present disclosure provides the compound of Formula (I), (Ia), (Tb), (Ic), and/or (Id), or a pharmaceutically acceptable salt thereof, wherein
ring C3 is

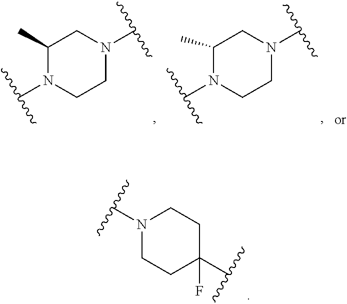

or absent.

In some embodiments, the present disclosure provides the compound of Formula (I), (Ia), (Ib), (Ic), and/or (Id), or a pharmaceutically acceptable salt thereof, wherein Z³ is C₁-C₃ alkyl, C2-C₃ alkynyl, —O—C₁-C₃ alkyl, —O—C₂-C₃ alkynyl, —O—, —C(O)—, or a bond. In some embodiments, the present disclosure provides the compound of Formula (I), (Ia), (Ib), (Ic), and/or (Id), or a pharmaceutically acceptable salt thereof, wherein Z³ is CH₂, —CH₂CH₂—, —C≡C—, —OCH₂—, —CH₂O—, —OCH₂—C≡C—, —O—, —C(O)—, or a bond. In some embodiments, the present disclosure provides the compound of Formula (I), (Ia), (Ib), (Ic), and/or (Id), or a pharmaceutically acceptable salt thereof, wherein Z³ is —C(O)— or a bond.

As described herein, the ring designated as

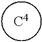

in the compound of Formula (I), (Ia), and/or (Ib), or a pharmaceutically acceptable salt thereof, is also referred to herein interchangeably as ring C4.

In some embodiments, the present disclosure provides the compound of Formula (I), (Ia), and/or (Tb), or a pharmaceutically acceptable salt thereof, wherein
ring C4 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S; each R^{C4} is independently C₁-C₆ alkyl, halo, or oxo; and q is 0, 1 or 2.

In some embodiments, the present disclosure provides the compound of Formula (I), (Ia), and/or (Ib), or a pharmaceutically acceptable salt thereof, wherein
ring C4 is 5 to 14-membered heterocyclyl, C₆-C₁₀ aryl or 5- to 10-membered heteroaryl, wherein the heterocyclyl and the heteroaryl each has 1 or 2 heteroatoms that is N; each R^{C}4 is independently C₁-C₆ alkyl, halo, or oxo; and q is 0, 1 or 2.

In some embodiments, the present disclosure provides the compound of Formula (I), (Ia), and/or (Ib), or a pharmaceutically acceptable salt thereof, wherein
ring C4 is 9-membered heterocyclyl, phenyl, or 6- to 9-membered heteroaryl, wherein the heterocyclyl and the heteroaryl each has 1 or 2 heteroatoms that is N; each R^{C4} is independently C₁-C₆ alkyl, halo, or oxo; and q is 0, 1 or 2.

In some embodiments, the present disclosure provides the compound of Formula (I), (Ia), and/or (Ib), or a pharmaceutically acceptable salt thereof, wherein ring C4 is absent.

In some embodiments, the present disclosure provides the compound of Formula (I), (Ia), and/or (Ib), or a pharmaceutically acceptable salt thereof, wherein Z⁴ is a bond.

As described herein, the ring designated as

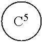

in the compound of Formula (I), (Ia), (Ib), (Ic), and/or (Id), or a pharmaceutically acceptable salt thereof, is also referred to herein interchangeably as ring C5.

In some embodiments, the present disclosure provides the compound of Formula (I), (Ia), (Tb), (Ic), and/or (Id), or a pharmaceutically acceptable salt thereof, wherein ring C5 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S; each R^{C5} is independently C₁-C₆ alkyl, C₁-C₆ alkoxy, halo, or oxo; and r is 0, 1 or 2.

In some embodiments, the present disclosure provides the compound of Formula (I), (Ia), (Tb), (Ic), and/or (Id), or a pharmaceutically acceptable salt thereof, wherein ring C5 is C₆-C₁₀ aryl or 5- to 10-membered heteroaryl, wherein the heterocyclyl and heteroaryl each has 1 or 2 heteroatoms selected from N and O; each R^{C5} is independently C₁-C₃ alkyl, C₁-C₃ alkoxy, halo, or oxo; and r is 0, 1 or 2.

In some embodiments, the present disclosure provides the compound of Formula (I), (Ia), (Ib), (Ic), and/or (Id), or a pharmaceutically acceptable salt thereof, wherein
ring C5 is

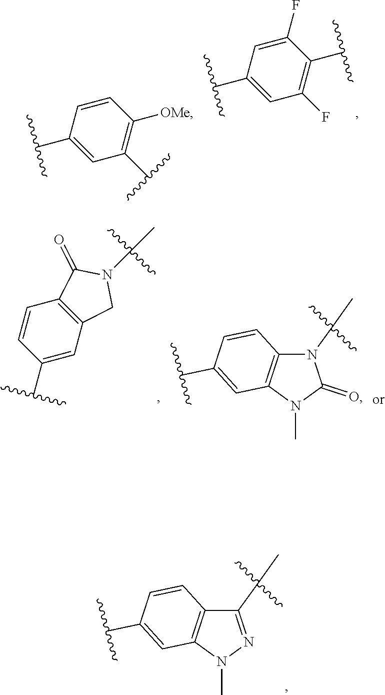 [SMILES: *c1ccc(OC)c(*)c1 ; *c1cc(F)c(*)c(F)c1 ; *c1ccc2c(c1)CN(*)C2=O ; *c1ccc2c(c1)n(C)c(=O)n2* ; *c1ccc2c(*)nn(C)c2c1]

In some embodiments, the present disclosure provides the compound of Formula (I), (Ia), (Ib), (Ic), and/or (Id), or a pharmaceutically acceptable salt thereof, wherein
ring C5 is

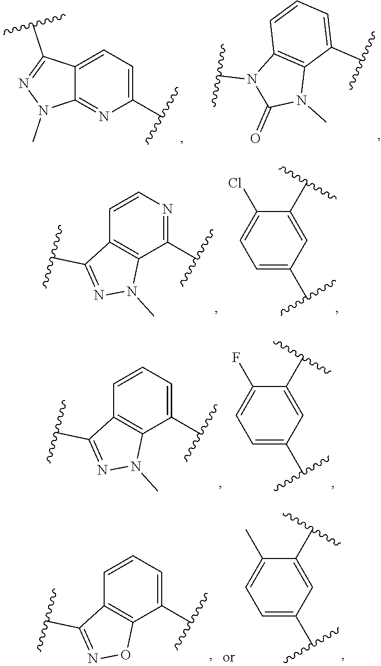

In some embodiments, the present disclosure provides the compound of Formula (I), (Ia), and/or (Ib), or a pharmaceutically acceptable salt thereof, wherein Z⁵ is a bond.

In some embodiments, the present disclosure provides the compound of Formula (I), (Ia), (Ib), (Ic), and/or (Id), or a pharmaceutically acceptable salt thereof, wherein Z⁶ is —CH—.

In some embodiments, the present disclosure provides the compound of Formula (I), (Ia), (Ib), (Ic), and/or (Id), or a pharmaceutically acceptable salt thereof, wherein Z⁶ is N.

In some embodiments, the present disclosure provides the compound Formula (I), (Ia), (Ib), (Ic), and/or (Id), or a pharmaceutically acceptable salt thereof, wherein
ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl or —OH; n is 0 or 1; Z² is C₁-C₃ alkyl, C₂-C₃ alkynyl, —O—C₁-C₃ alkyl, —O—C₂-C₃ alkynyl, —O—, or a bond;
ring C3 is 4- to 16-membered heterocyclyl or absent, wherein the heterocyclyl has 1 or 2 heteroatoms that is N; each R^{C3} is independently C₁-C₆ alkyl; p is 0 or 1; Z⁵ is a bond;
ring C5 is 5 to 14-membered heterocyclyl, C₆-C₁₀ aryl or 5- to 10-membered heteroaryl, wherein the heterocyclyl and the heteroaryl each has 1 or 2 heteroatoms selected from N and O; each R^{C}4 is independently C₁-C₆ alkyl, halo, or oxo; q is 0, 1 or 2; Z⁵ is a bond; and Z⁶ is —CH— or N.

In some embodiments, the present disclosure provides the compound of Formula (I), (Ia), and/or (Ib), or a pharmaceutically acceptable salt thereof, wherein the moiety

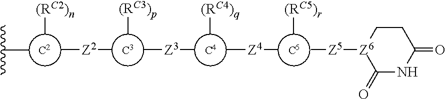

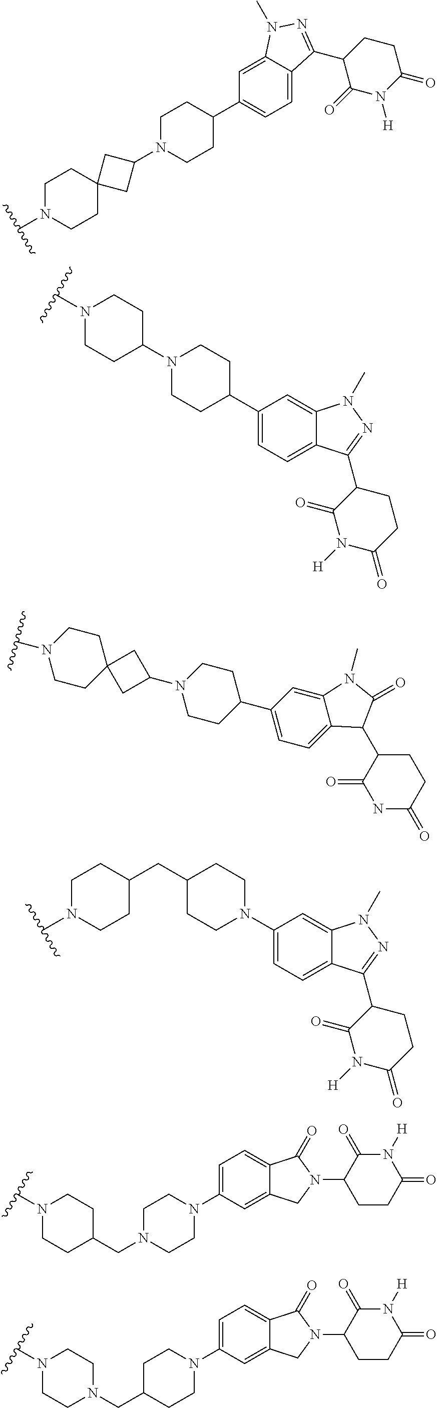

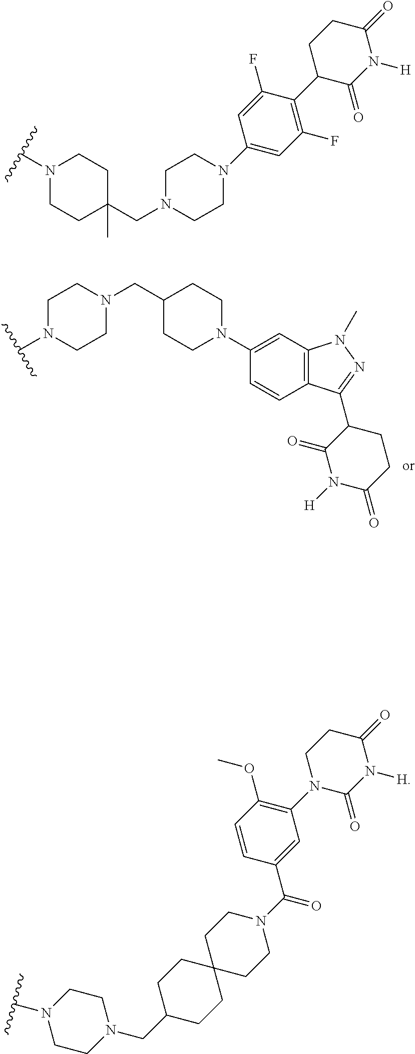

In some embodiments, the present disclosure provides the compound of Formula (I), (Ia), and/or (Ib), or a pharmaceutically acceptable salt thereof, wherein the moiety

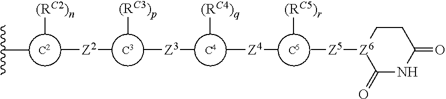

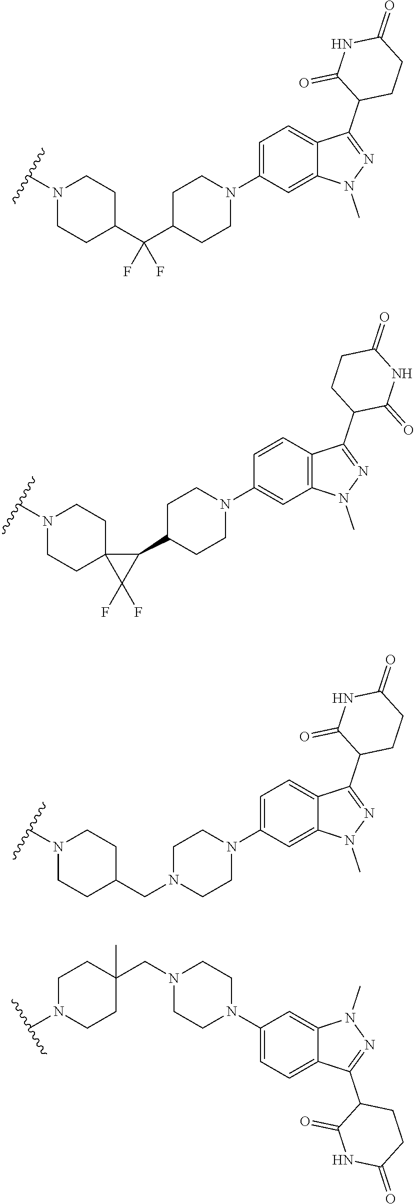 [SMILES: *N1CCC(C(F)(F)C2CCN(c3ccc4c(C5CCC(=O)NC5=O)nn(C)c4c3)CC2)CC1 ; *N1CCC2(CC1)[C@@H](C1CCN(c3ccc4c(C5CCC(=O)NC5=O)nn(C)c4c3)CC1)C2(F)F ; *N1CCC(CN2CCN(c3ccc4c(C5CCC(=O)NC5=O)nn(C)c4c3)CC2)CC1 ; *N1CCC(C)(CN2CCN(c3ccc4c(C5CCC(=O)NC5=O)nn(C)c4c3)CC2)CC1]

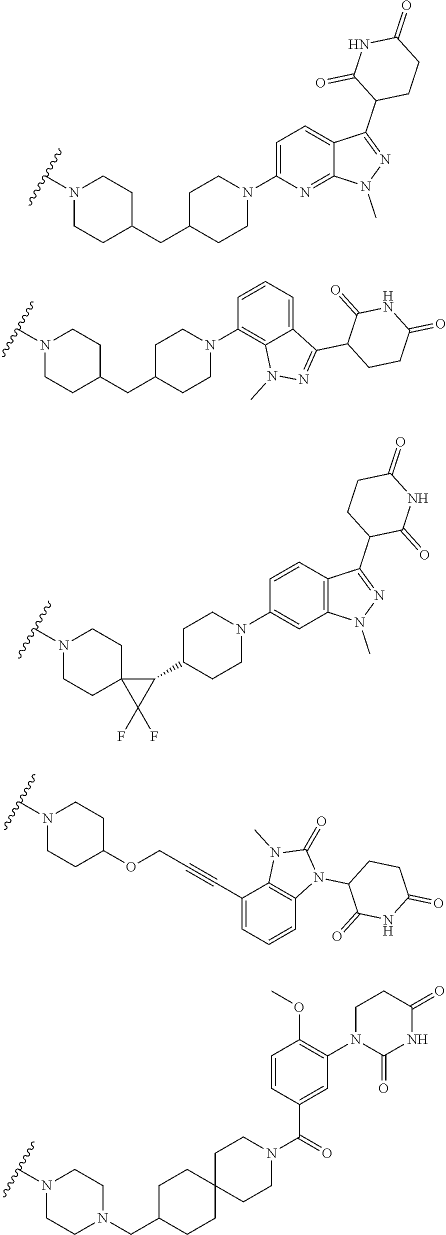 [SMILES: *N1CCC(CC2CCN(c3ccc4c(C5CCC(=O)NC5=O)nn(C)c4n3)CC2)CC1 ; *N1CCC(CC2CCN(c3cccc4c(C5CCC(=O)NC5=O)nn(C)c34)CC2)CC1 ; *N1CCC2(CC1)[C@H](C1CCN(c3ccc4c(C5CCC(=O)NC5=O)nn(C)c4c3)CC1)C2(F)F ; *N1CCC(OCC#Cc2cccc3c2n(C)c(=O)n3C2CCC(=O)NC2=O)CC1 ; *N1CCN(CC2CCC3(CC2)CCN(C(=O)c2ccc(OC)c(N4CCC(=O)NC4=O)c2)CC3)CC1]

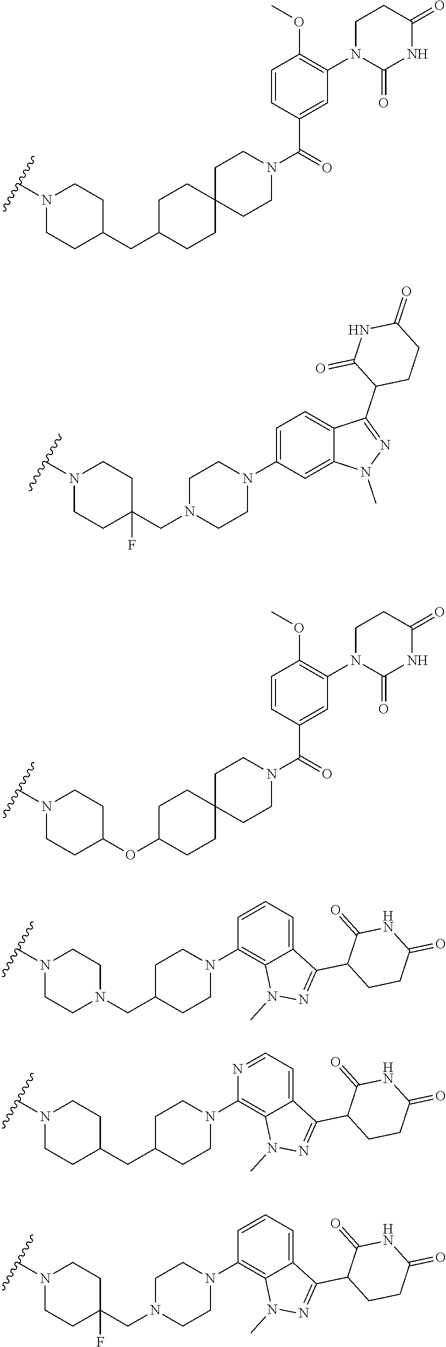

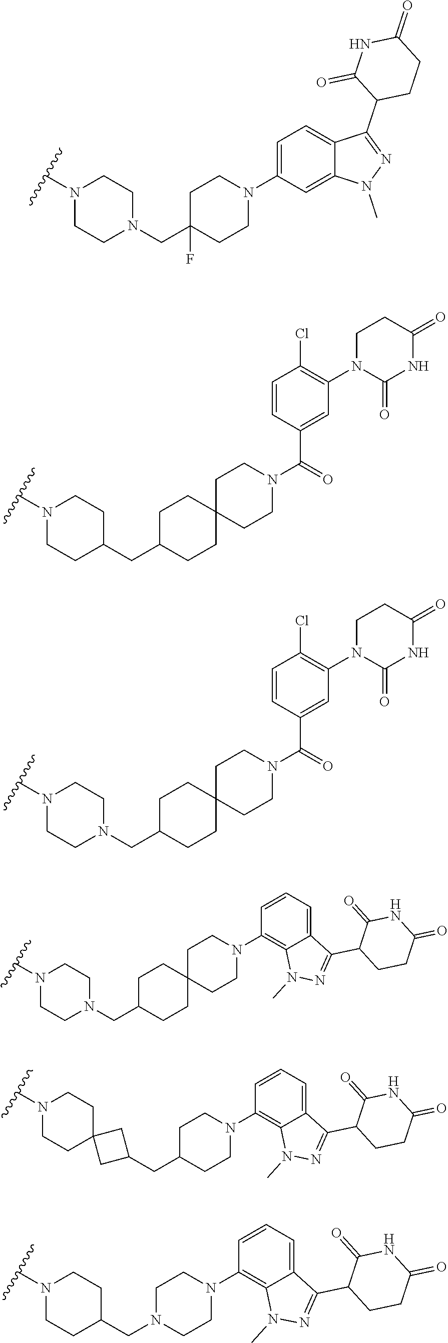 [SMILES: *N1CCN(CC2(F)CCN(c3ccc4c(C5CCC(=O)NC5=O)nn(C)c4c3)CC2)CC1 ; *N1CCC(CC2CCC3(CC2)CCN(C(=O)c2ccc(Cl)c(N4CCC(=O)NC4=O)c2)CC3)CC1 ; *N1CCN(CC2CCC3(CC2)CCN(C(=O)c2ccc(Cl)c(N4CCC(=O)NC4=O)c2)CC3)CC1 ; *N1CCN(CC2CCC3(CC2)CCN(c2cccc4c(C5CCC(=O)NC5=O)nn(C)c24)CC3)CC1 ; *N1CCC2(CC1)CC(CC1CCN(c3cccc4c(C5CCC(=O)NC5=O)nn(C)c34)CC1)C2 ; *N1CCC(CN2CCN(c3cccc4c(C5CCC(=O)NC5=O)nn(C)c34)CC2)CC1]

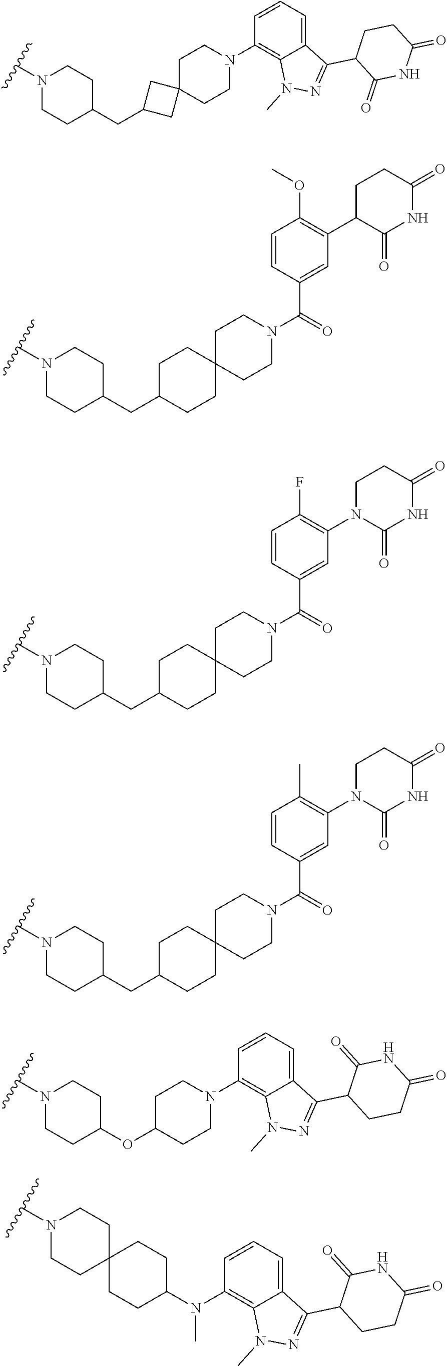 [SMILES: *N1CCC(CC2CC3(CCN(c4cccc5c(C6CCC(=O)NC6=O)nn(C)c45)CC3)C2)CC1 ; *N1CCC(CC2CCC3(CC2)CCN(C(=O)c2ccc(OC)c(C4CCC(=O)NC4=O)c2)CC3)CC1 ; *N1CCC(CC2CCC3(CC2)CCN(C(=O)c2ccc(F)c(N4CCC(=O)NC4=O)c2)CC3)CC1 ; *N1CCC(CC2CCC3(CC2)CCN(C(=O)c2ccc(C)c(N4CCC(=O)NC4=O)c2)CC3)CC1 ; *N1CCC(OC2CCN(c3cccc4c(C5CCC(=O)NC5=O)nn(C)c34)CC2)CC1 ; *N1CCC2(CCC(N(C)c3cccc4c(C5CCC(=O)NC5=O)nn(C)c34)CC2)CC1]

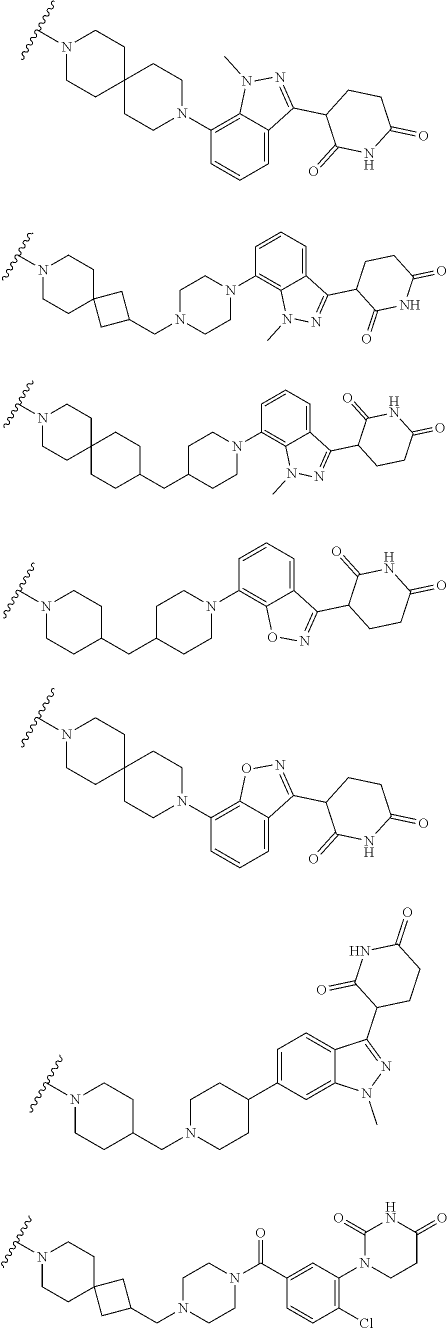

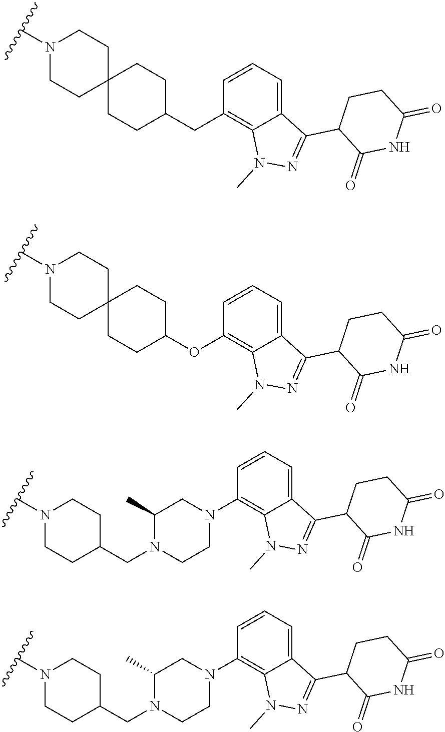

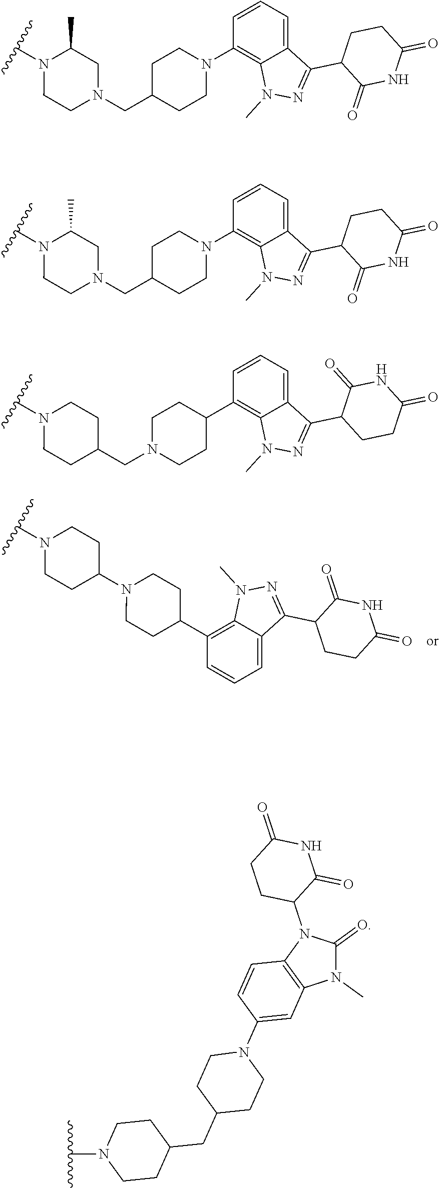 [SMILES: *N1CCN(CC2CCN(c3cccc4c(C5CCC(=O)NC5=O)nn(C)c34)CC2)C[C@@H]1C ; *N1CCN(CC2CCN(c3cccc4c(C5CCC(=O)NC5=O)nn(C)c34)CC2)C[C@H]1C ; *N1CCC(CN2CCC(c3cccc4c(C5CCC(=O)NC5=O)nn(C)c34)CC2)CC1 ; *N1CCC(N2CCC(c3cccc4c(C5CCC(=O)NC5=O)nn(C)c34)CC2)CC1 ; *N1CCC(CC2CCN(c3ccc4c(c3)n(C)c(=O)n4C3CCC(=O)NC3=O)CC2)CC1]

In some embodiments, the present disclosure provides the compound of Formula (I), (Ia), (Ib), (Ic), and/or (Id), or a pharmaceutically acceptable salt thereof, wherein the compound has the structure of a compound of Example 1-1 to Example 1-10, Example 2-1 to Example 2-5, Example 3-1 to 3-40, Example 4-1, or Example 4-2.

In some embodiments, the present disclosure provides the compound having the structure:

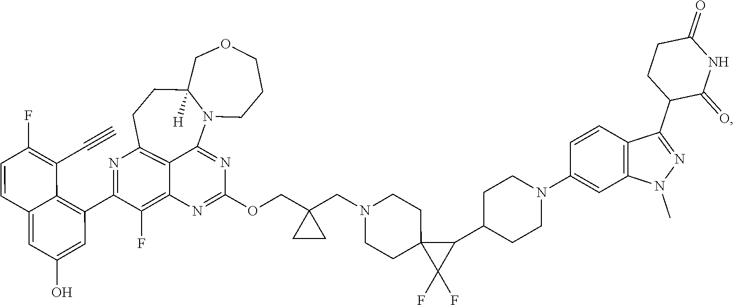

or a pharmaceutically acceptable salt thereof.

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug after overcoming biological barriers to oral delivery.

### IV. Pharmaceutical Compositions

Also disclosed herein are pharmaceutical compositions comprising a pharmaceutically effective amount of a compound of the present disclosure (e.g., a compound of Formula (I), (Ia), (Ib), (Ic), and/or (Id)), or a pharmaceutically acceptable salt thereof), and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s). When formulated in an ointment, the active ingredients can be employed in some embodiments with either a paraffinic or a water-miscible ointment base. Alternatively, the active ingredients can be formulated in a cream with an oil-in-water cream base.

In some embodiments, the cream base can include, for instance, a compound that enhances absorption or penetration of the active ingredient through the skin or other affected areas. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

The choice of suitable oils or fats for the formulation can be based on achieving the desired cosmetic properties.

In some embodiments, the compounds disclosed herein are administered alone. In some embodiments, the compounds disclosed herein are administered in pharmaceutical compositions. In some embodiments, the pharmaceutical compositions are for veterinary use. In some embodiments, the pharmaceutical compositions are for human use. In some embodiments, the pharmaceutical compositions disclosed herein include at least one additional therapeutic agent. In some embodiments, the pharmaceutical compositions disclosed herein include one or more additional therapeutic agent. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

Pharmaceutical compositions disclosed herein can be in any form suitable for the intended method of administration. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical compositions and such formulations can contain at least agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. Tablets can be uncoated or can be coated by known techniques including microencapsulation to delay disintegration and adsorption in the gastrointestinal tract and thereby provide a sustained action over a longer period.

Formulations for oral use can be also presented as hard gelatin capsules where the active ingredient is mixed with an inert solid diluent, or as soft gelatin capsules wherein the active ingredient is mixed with water or an oil medium.

Aqueous suspensions can contain the active materials in admixture with excipients suitable for the manufacture of aqueous suspensions. The aqueous suspension can also contain, for example, at least preservatives, one or more coloring agents, one or more flavoring agents, one or more sweetening agents, or combinations thereof.

Oil suspensions can be formulated by suspending the active ingredient in a vegetable oil, a mineral oil, or a combination thereof. The oral suspensions can contain, for instance, a thickening agent. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Additional excipients, for example sweetening, flavoring and coloring agents, can also be present.

The pharmaceutical compositions can also be in the form of oil-in-water emulsions. The oily phase can be a vegetable oil, a mineral oil, or a mixture of these. The emulsion can also contain sweetening and flavoring agents. Syrups and elixirs can be formulated with sweetening agents. Such formulations can also contain, for instance, a demulcent, a preservative, a flavoring, a coloring agent, or a combination thereof.

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents. The sterile injectable or intravenous preparation can also be a sterile injectable solution or suspension in a non-toxic parenterally acceptable diluent or solvent, or prepared as a lyophilized powder.

The amount of active ingredient that can be combined with the carrier material to produce a single dosage form will vary depending upon the host treated and the particular mode of administration. The pharmaceutical composition can be prepared to provide easily measurable amounts for administration.

Formulations suitable for topical administration to the eye also include eye drops wherein the active ingredient is dissolved or suspended in a suitable carrier, especially an aqueous solvent for the active ingredient.

Formulations suitable for topical administration in the mouth include lozenges can comprise an active ingredient (i.e., a compound disclosed herein and/or additional therapeutic agents) in a flavored basis; pastilles comprising the active ingredient in an inert basis; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical composition, and will be determined by the clinician using conventional dose escalation studies.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is viral infection.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of the compounds of Formula (I), (Ia), (Ib), (Ic), and/or (Id), or a pharmaceutically acceptable salt thereof (herein referred to as the active ingredients), are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the route may vary with for example the condition of the recipient. An advantage of the compounds herein is that they are orally bioavailable and can be dosed orally.

The compounds of the present disclosure (also referred to herein as the active ingredients), can be administered by any route appropriate to the condition to be treated.

Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the route may vary with for example the condition of the recipient. An advantage of certain compounds disclosed herein is that they are orally bioavailable and can be dosed orally.

A compound of the present disclosure may be administered to an individual in accordance with an effective dosing regimen for a desired period of time or duration.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration.

A compound of the present disclosure may be combined with one or more additional therapeutic agents in any dosage amount of the compound of the present disclosure (e.g., from about 1 mg to about 1000 mg of compound). A single dose can be administered hourly, daily, or weekly.

The frequency of dosage of the compound of the present disclosure can be determined by the needs of the individual patient and can be, for example, once per day or twice, or more times, per day. Administration of the compound continues for as long as necessary to treat the disease or condition.

Administration can be intermittent, with a period of several or more days during which a patient receives a daily dose of the compound of the present disclosure followed by a period of several or more days during which a patient does not receive a daily dose of the compound. Alternating periods of administration of the compound, followed by non-administration of the compound, can be repeated as clinically required to treat the patient.

In some embodiments, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits comprising a compound of the present disclosure, or a pharmaceutically acceptable salt thereof, in combination with one or more (e.g., one, two, three, four, one or two, one to three, or one to four) additional therapeutic agents are provided.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure is combined with one or more additional therapeutic agents as described herein, the components of the composition are administered as a simultaneous or sequential regimen. When administered sequentially, the combination may be administered in two or more administrations.

In some embodiments, a compound of the present disclosure is combined with one or more additional therapeutic agents in a unitary dosage form for simultaneous administration to a patient, for example as a solid dosage form for oral administration.

In some embodiments, a compound of the present disclosure is co-administered with one or more additional therapeutic agents.

In order to prolong the effect of a compound of the present disclosure, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers. Depending upon the ratio of compound to polymer and the nature of the particular polymer employed, the rate of compound release can be controlled. Depot injectable formulations are also prepared by entrapping a compound in liposomes or microemulsions that are compatible with body tissues.

### VII. Methods of Use

The disclosure further relates to the use of compounds disclosed herein for the treatment and/or prophylaxis of diseases and/or conditions through inhibition of KRAS protein, e.g., KRAS wild type, G12C, G12D and/or G12V protein. Further, the present disclosure relates to the use of said compounds for the preparation of a medicament for the treatment and/or prophylaxis of cancer.

The disclosure further relates to the use of compounds disclosed herein for the treatment and/or prophylaxis of diseases and/or conditions through degradation of KRAS protein, e.g., KRAS wild type, G12C, G12D and/or G12V protein. In some embodiments, the compounds disclosed herein are degraders of KRAS wild type, G12C, G12D and/or G12V.

Medicaments as referred to herein can be prepared by conventional processes, including the combination of a compound according to the present disclosure and a pharmaceutically acceptable carrier.

In some embodiments, provided herein is a method of inhibiting KRAS protein, e.g., KRAS wild type, G12C, G12D and/or G12V protein, in a subject in need thereof, the method comprising administering to the subject a therapeutically effective amount of a compound of Formula (I), (Ia), (Ib), (Ic), and/or (Id), or a pharmaceutically acceptable salt thereof, or a pharmaceutical composition comprising a compound of Formula (I), (Ia), (Ib), (Ic), and/or (Id).

In some embodiments, provided herein is a method of inhibiting and/or degrading KRAS protein, e.g., KRAS wild type, G12C, G12D and/or G12V protein, in a subject in need thereof, the method comprising administering to the subject a therapeutically effective amount of a compound of Formula (I), (Ia), (Ib), (Ic), and/or (Id), or a pharmaceutically acceptable salt thereof, or a pharmaceutical composition comprising a compound of Formula (I), (Ia), (Ib), (Ic), and/or (Id).

In some embodiments, provided herein is treating cancer in a subject in need thereof, the method comprising administering to the subject a therapeutically effective amount of a compound of Formula (I), (Ia), (Ib), (Ic), and/or (Id), or a pharmaceutically acceptable salt thereof, or a pharmaceutical composition comprising a compound of Formula (I), (Ia), (Ib), (Ic), and/or (Id).

In some embodiments, provided herein is a method of treating and/or preventing a cancer.

In some embodiments, provided herein is a method of treating and/or preventing a KRAS G12D-associated cancer.

In some embodiments, provided herein is a method of reducing the proliferation of a cell comprising contacting the cell with a compound of Formula (I), (Ia), (Ib), (Ic), and/or (Id), or a pharmaceutically acceptable salt thereof.

In some embodiments, the KRAS G12C, G12D and/or G12V associated disease or condition includes cancer. In some embodiments, the cancer is a hematological cancer. In some embodiments, the cancer includes a solid tumor. In some embodiments, the cancer includes a malignant tumor. In some embodiments the cancer includes a metastatic cancer. In some embodiments, the cancer is resistant or refractory to one or more anticancer therapies. In some embodiments, greater than about 50% of the cancer cells detectably express one or more cell surface immune checkpoint receptors (e.g., so-called “hot” cancer or tumor). In some embodiments, greater than about 1% and less than about 50% of the cancer cells detectably express one or more cell surface immune checkpoint receptors (e.g., so called “warm” cancer or tumor). In some embodiments, less than about 1% of the cancer cells detectably express one or more cell surface immune checkpoint receptors (e.g., so called “cold” cancer or tumor).

In some embodiments, the KRAS G12C, G12D and/or G12V associated disease or condition is a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CML), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)).

In some embodiments, the KRAS G12C, G12D and/or G12V associated disease or condition is an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), or a lymphoma.

In some embodiments, the KRAS G12C, G12D and/or G12V associated disease or condition includes a solid tumor in or arising from a tissue or organ, such as bone, lips and oral cavity, esophagus, gastrointestinal tract, pancreas, gall bladder, neuro-endocrine, thyroid, liver, kidney, breast, peritoneum, female sex organ tissues, male sex organ tissues, bladder, brain, eye, head and neck, thymus, heart, lung, lymph, central nervous system (CNS), neuroendocrine tissues, skin, and soft tissues.

In some embodiments, the KRAS G12C, G12D and/or G12V associated disease or condition is a cancer selected from a lung cancer, a colorectal cancer, a breast cancer, a prostate cancer, a cervical cancer, a pancreatic cancer and a head and neck cancer. In some embodiments, the cancer is metastatic.

In some embodiments, the KRAS G12C, G12D and/or G12V associated disease or condition is a cancer selected from non-small lung cancer (NSCLC), melanoma, triple-negative breast cancer (TNBC), nasopharyngeal cancer (NPC), microsatellite stable colorectal cancer (mssCRC), thymoma, and gastrointestinal stromal tumor (GIST). In some embodiments, the cancer is metastatic.

In some embodiments, the KRAS G12C, G12D and/or G12V associated disease or condition is a cancer of pancreatic cancer, bladder cancer, colorectal cancer, breast cancer, prostate cancer, renal cancer, hepatocellular cancer, lung cancer, ovarian cancer, cervical cancer, uterine cancer, gastric cancer, bile duct cancer, testicular cancer, esophageal cancer, head and neck cancer, melanoma, neuroendocrine cancer, CNS cancer, brain cancer, bone cancer, soft tissue sarcoma, non-small cell lung cancer, small-cell lung cancer, myelodysplastic syndrome, thyroid cancer, or colon cancer.

In some embodiments, the KRAS G12C, G12D and/or G12V associated disease or condition is a cancer of pancreatic cancer, colorectal cancer, non-small cell lung cancer, endometrial cancer, uterine endometrical carcinoma, cholangio carcinoma, testicular germ cell cancer, cervical squamous carcinoma, or myelodysplastic syndrome.

In some embodiments, the cancer is or myelodysplastic syndrome. In some embodiments, the cancer is high risk myelodysplastic syndrome or low risk myelodysplastic syndrome. In some embodiments, the cancer is high risk myelodysplastic syndrome. In some embodiments, the cancer is high risk myelodysplastic syndrome.

In some embodiments, the cancer is colorectal cancer. In some embodiments, the cancer is non-small cell lung cancer. In some embodiments, the cancer is pancreatic cancer. In some embodiments, the cancer is endometrial cancer. In some embodiments, the cancer is uterine endometrical carcinoma. In some embodiments, the cancer is testicular germ cell cancer. In some embodiments, the cancer is cervical squamous carcinoma. In some embodiments, the cancer is cholangio carcinoma.

The effective dosage of active ingredient employed may vary depending on the particular compound employed, the mode of administration, the condition being treated and the severity of the condition being treated. Such dosage may be ascertained readily by a person skilled in the art.

When treating or preventing a KRAS G12C, G12D and/or G12V associated disease or condition for which compounds of the present disclosure are indicated, generally satisfactory results are obtained when the compounds of the present disclosure are administered at a daily dosage of from about 0.1 milligram to about 300 milligram per kilogram of animal body weight. For most large mammals, the total daily dosage is from about 1 milligram to about 1000 milligrams.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are frequently alternated with resting periods of about 1 to 28 days, commonly about 7 days or about 14 days, between cycles. The treatment cycles, in other embodiments, may also be continuous.

In some embodiments, the compound or pharmaceutically acceptable salt thereof of the present disclosure is administered in combination with one or more additional therapeutic agent or therapeutic modality.

In some embodiments, the present disclosure provides the pharmaceutical composition or the method wherein the one or more additional therapeutic agent or additional therapeutic modality comprises one, two, three, or four additional therapeutic agents and/or therapeutic modalities.

In some embodiments, the present disclosure provides the pharmaceutical composition or the method wherein the additional therapeutic agent or therapeutic modalities are selected from an immune checkpoint modulator, an antibody-drug conjugate (ADC), an antiapoptotic agent, a targeted anticancer therapeutic, a chemotherapeutic agent, surgery, or radiation therapy.

In some embodiments, the present disclosure provides the pharmaceutical composition or the method wherein the immune checkpoint modulator is selected from an anti-PD-(L)₁ antibody, an anti-TIGIT antibody, an anti-CTLA4 antibody, an anti-CCR8 antibody, an anti-TREM1 antibody, an anti-TREM2 antibody, a CD47 inhibitor, a DGKX inhibitor, an HPK1 inhibitor, a FLT3 agonist, an adenosine receptor antagonist, a CD39 inhibitor, a CD73 inhibitor, an IL-2 variant (IL-2v), and a CAR-T cell therapy.

In some embodiments, the present disclosure provides the pharmaceutical composition or the method wherein the anti-PD-(L)1 antibody is selected from pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab.

In some embodiments, the present disclosure provides the pharmaceutical composition or the method wherein the anti-TIGIT antibody is selected from tiragolumab, vibostolimab, domvanalimab, AB308, AK127, BMS-986207, and etigilimab.

In some embodiments, the present disclosure provides the pharmaceutical composition or the method wherein the anti-CTLA4 antibody is selected from ipilimumab, tremelimumab, and zalifrelimab.

In some embodiments, the present disclosure provides the pharmaceutical composition or the method wherein the CD47 inhibitor is selected from magrolimab, letaplimab, lemzoparlimab, AL-008, RRx-001, CTX-5861, FSI-189 (GS-0189), ES-004, BI-765063, ADU1805, CC-95251, and Q-1801.

In some embodiments, the present disclosure provides the pharmaceutical composition or the method wherein the adenosine receptor antagonist is etrumadenant (AB928), taminadenant, TT-10, TT-4, or M1069.

In some embodiments, the present disclosure provides the pharmaceutical composition or the method wherein the CD39 inhibitor is TTX-030.

In some embodiments, the present disclosure provides the pharmaceutical composition or the method wherein the CD73 inhibitor is quemliclustat (AB680), uliledlimab, mupadolimab, ORIC-533, ATG-037, PT-199, AK131, NZV930, BMS-986179, or oleclumab.

In some embodiments, the present disclosure provides the pharmaceutical composition or the method wherein the IL-2v is aldesleukin (Proleukin), bempegaldesleukin (NKTR-214), nemvaleukin alfa (ALKS-4230), THOR-202 (SAR-444245), BNT-151, ANV-419, XTX-202, RG-6279 (RO-7284755), NL-201, STK-012, SHR-1916, or GS-4528.

In some embodiments, the present disclosure provides the pharmaceutical composition or the method wherein the ADC is selected from sacituzumab govitecan, datopotamab deruxtecan, enfortumab vedotin, and trastuzumab deruxtecan.

In some embodiments, the present disclosure provides the pharmaceutical composition or the method wherein the additional therapeutic agent is selected from idealisib, sacituzumab govitecan, magrolimab, GS-0189, GS-3583, zimberelimab, GS-4224, GS-9716, GS-6451, GS-1811 (JTX-1811), quemliclustat (AB680), etrumadenant (AB928), domvanalimab, AB308, PY159, PY314, AGEN-1223, AGEN-2373, axicabtagene ciloleucel and brexucabtagene autoleucel.

In some embodiments, the method includes administering one or more additional therapeutic agents. The one or more additional therapeutic agents can be one or more therapeutic agents as described below. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

In some embodiments, the one or more additional therapeutic agents includes therapeutic agents used to treat high risk myelodysplastic syndrome (HR MDS), low risk myelodyplastic syndrome (LR MDS), colorectal cancer, non-small cell lung cancer (NSCLC), pancreatic cancer, or endometrial cancer. In some embodiments, the one or more additional therapeutic agents includes therapeutic agents used to treat high risk myelodysplastic syndrome (HR MDS).

In some embodiments, the one or more additional therapeutic agents includes therapeutic agents used to treat low risk myelodyplastic syndrome (LR MDS).

In some embodiments, the one or more additional therapeutic agents includes therapeutic agents used to treat colorectal cancer.

In some embodiments, the one or more additional therapeutic agents includes therapeutic agents used to treat non-small cell lung cancer (NSCLC).

In some embodiments, the one or more additional therapeutic agents includes therapeutic agents used to treat pancreatic cancer.

In some embodiments, the one or more additional therapeutic agents includes therapeutic agents used to treat endometrial cancer.

In some embodiments, the one or more additional therapeutic agents is independently SNS-301, 5-FU+leucovorin+oxaliplatin+irinotecan, 5-FU+nanoliposomal irinotecan, 5-FU, afatinib (Gilotrif©), aflibercept (Zaltrap®), aflibercept+FOLFIRI, albumin-bound paclitaxel, alectinib (Alecensa®), anastrozole (Arimidex®), atezolizumab, avelumab, azacitidine (Vidaza®), bevacizumab (Avastin®), bevacizumab+carboplatin+nab-paclitaxel, bevacizumab+carboplatin+pemetrexed, bevacizumab+FOLFIRI, bevacizumab+FOLFOX, bevacizumab+FOLFOXIRI, bevacizumab+leucovorin+5-FU+oxaliplatin (FOLFOX), bevacizumab+XELOX, bevacizumab, BGB324, binimetinib+encorafenib+cetuximab, brigatinib, cabozantinib, canakinumab, capecitabine, carboplatin+nab-paclitaxel, carboplatin+pemetrexed, carboplatin, cemiplimab, cetuximab (Erbitux®), cetuximab+FOLFIRI, cisplatin+gemcitabine, cisplatin+pemetrexed, cisplatin, crizotinib (Xalkori®), cytarabine+daunorubicin, cytarabine+idarubicin, cytarabine, dabrafenib (Tafinlar®), dabrafenib+trametinib, datopotamab deruxtecan (DS-1062), datopotamab deruxtecan+durvalumab, datopotamab deruxtecan+pembrolizumab, daunorubicin, decitabine (Dacogen®), docetaxel, domvanalimab, dostarlimab (Jemperli©), doxorubicin, DSP-7888, durvalumab+tremelimumab, durvalumab, enasidenib, enfortumab vedotin (Padcev©), entrectinib (Tarceva®), erlotinib, etoposide, exemestane (Aromasin®), fluorouracil, FOLFIRI, FOLFIRINOX, FOLFOXIRI, gefitinib (Iressa®), gemcitabine+nab-paclitaxel, gemcitabine, guadecitabine, idarubicin, ifosfamide, imetelstat, irinotecan, ivosidenib, LB-100, lenalidomide (Revlimid®), lenalidomide, lenvatinib (Lenvima®), letrozole (Femara®), leucovorin+nanoliposomal irinotecan, leucovorin, Lonsurf (Orcantas®), luspatercept, nab-paclitaxel (Abraxane®), napabucasin+FOLFIRI+bevacizumab, nivolumab (Opdivo®), nivolumab+docetaxel, nivolumab+ipilimumab, nogapendekin alfa (N-803)+pembrolizumab, nogapendekin alfa, ociperlimab+tislelizumab, ociperlimab, osimertinib (Tagrisso®), oxaliplatin (FOLFOX), paclitaxel, panitumumab, pembrolizumab (Keytruda®), pembrolizumab+carboplatin+nab-paclitaxel, pembrolizumab+carboplatin+pemetrexed, pembrolizumab+lenvatinib+pemetrexed, pembrolizumab+olaparib, pembrolizumab+pemetrexed+carboplatin, pemetrexed (Alimta®), pemetrexed+cisplatin+carboplatin, pevonedistat, progesterone, ramucirumab (Cyramza®), ramucirumab+docetaxel, regorafenib (Stivarga®), rigosertib, roxadustat, sabatolimab, selinexor, tiragolumab+atezolizumab, tiragolumab, trametinib (Mekinist®), trametinib+dabrafenib+panitumumab, trastuzumab+pertuzumab, trastuzumab deruxtecan (Enhertu®), trastuzumab, vandetanib, vemurafenib, venetoclax, vibostolimab+pembrolizumab, vibostolimab, vinblastine, vinorelbine, XELOX, or ziv-aflibercept.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

### VIII. Combination Therapy

In some embodiments, a compound of Formula (I), (Ia), (Ib), (Ic), and/or (Id), provided herein, or pharmaceutically acceptable salt thereof, is administered in combination with one or more additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the pharmaceutical compositions provided herein have a compound of Formula (I), (Ia), (Ib), (Ic), and/or (Id), provided herein, or pharmaceutically acceptable salt thereof, and one or more additional therapeutic agents. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments the additional therapeutic agent includes, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (e.g., a mono- and multi-specific antibody, or fragment thereof, in any format, such as DART®, Duobody®, BiTE®, BiKE, TriKE, XmAb®, TandAb®, scFv, Fab, Fab derivative), a bi-specific antibody, a non-immunoglobulin antibody mimetic (e.g., including adnectin, affibody, affilin, affimer, affitin, alphabody, anticalin, peptide aptamer, armadillo repeat protein (ARM), atrimer, avimer, designed ankyrin repeat protein (DARPin®), fynomer, knottin, Kunitz domain peptide, monobody, and nanoCLAMPs), an antibody-drug conjugate (ADC), antibody-peptide conjugate), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

### Illustrative Targets

In some embodiments, the one or more additional therapeutic agents include, e.g., an inhibitor, agonist, antagonist, ligand, modulator, stimulator, blocker, activator or suppressor of a target (e.g., polypeptide or polynucleotide), such as: 2′-5′-oligoadenylate synthetase (OAS1; NCBI Gene ID: 4938); 5′-3′ exoribonuclease 1 (XRN1; NCBI Gene ID: 54464); 5′-nucleotidase ecto (NT5E, CD73; NCBI Gene ID: 4907); ABL proto-oncogene 1, non-receptor tyrosine kinase (ABL1, BCR-ABL, c-ABL, v-ABL; NCBI Gene ID: 25); absent in melanoma 2 (AIM2; NCBI Gene ID: 9447); acetyl-CoA acyltransferase 2 (ACAA2; NCBI Gene ID: 10499); acid phosphatase 3 (ACP3; NCBI Gene ID: 55); adenosine deaminase (ADA, ADA1; NCBI Gene ID: 100); adenosine receptors (e.g., ADORA1 (A1), ADORA2A (A2a, A2AR), ADORA2B (A2b, A2BR), ADORA3 (A3); NCBI Gene IDs: 134, 135, 136, 137); AKT serine/threonine kinase 1 (AKT1, AKT, PKB; NCBI Gene ID: 207); alanyl aminopeptidase, membrane (ANPEP, CD13; NCBI Gene ID: 290); ALK receptor tyrosine kinase (ALK, CD242; NCBI Gene ID: 238); alpha fetoprotein (AFP; NCBI Gene ID: 174); amine oxidase copper containing (e.g., AOC1 (DA01), AOC2, AOC3 (VAP1); NCBI Gene IDs: 26, 314, 8639); androgen receptor (AR; NCBI Gene ID: 367); angiopoietins (ANGPT1, ANGPT2; NCBI Gene IDs: 284, 285); angiotensin II receptor type 1 (AGTR1; NCBI Gene ID: 185); angiotensinogen (AGT; NCBI Gene ID: 183); apolipoprotein A1 (APOA1; NCBI Gene ID: 335); apoptosis inducing factor mitochondria associated 1 (AIFM1, AIF; NCBI Gene ID: 9131); arachidonate 5-lipoxygenase (ALOX5; NCBI Gene ID: 240); asparaginase (ASPG; NCBI Gene ID: 374569); asteroid homolog 1 (ASTE1; NCBI Gene ID: 28990); ATM serine/threonine kinase (ATM; NCBI Gene ID: 472); ATP binding cassette subfamily B member 1 (ABCB1, CD243, GP170; NCBI Gene ID: 5243); ATP-dependent Clp-protease (CLPP; NCBI Gene ID: 8192); ATR serine/threonine kinase (ATR; NCBI Gene ID: 545); AXL receptor tyrosine kinase (AXL; NCBI Gene ID: 558); B and T lymphocyte associated (BTLA, CD272; NCBI Gene ID: 151888); baculoviral IAP repeat containing proteins (BIRC2 (cIAP1), BIRC3 (cIAP2), XIAP (BIRC4, IAP3), BIRC5 (survivin); NCBI Gene IDs: 329, 330, 331, 332); basigin (Ok blood group) (BSG, CD147; NCBI Gene ID: 682); B-cell lymphoma 2 (BCL2; NCBI Gene ID: 596); BCL2 binding component 3 (BBC3, PUMA; NCBI Gene ID: 27113); BCL2 like (e.g., BCL2L1 (Bcl-x), BCL2L2 (BIM); Bcl-x; NCBI Gene IDs: 598, 10018); beta 3-adrenergic receptor (ADRB3; NCBI Gene ID: 155); bone gamma-carboxyglutamate protein (BGLAP; NCBI Gene ID: 632); bone morphogenetic protein-10 ligand (BMP10; NCBI Gene ID: 27302); bradykinin receptors (e.g., BDKRB1, BDKRB2; NCBI Gene IDs: 623, 624); B-RAF (BRAF; NCBI Gene ID: 273); breakpoint cluster region (BCR; NCBI Gene ID: 613); bromodomain and external domain (BET) bromodomain containing proteins (e.g., BRD2, BRD3, BRD4, BRDT; NCBI Gene IDs: 6046, 8019, 23476, 676); Bruton's tyrosine kinase (BTK; NCBI Gene ID: 695); cadherins (e.g., CDH3 (p-cadherin), CDH6 (k-cadherin); NCBI Gene IDs: 1001, 1004); cancer/testis antigens (e.g., CTAG1A, CTAG1B, CTAG2; NCBI Gene IDs: 1485, 30848, 246100); cannabinoid receptors (e.g., CNR1 (CB1), CNR2 (CB2); NCBI Gene IDs: 1268, 1269); carbohydrate sulfotransferase 15 (CHST15; NCBI Gene ID: 51363); carbonic anhydrases (e.g., CA1, CA2, CA3, CA4, CASA, CASB, CA6, CA7, CA8, CA9, CA10, CA1l, CA12, CA13, CA14; NCBI Gene IDs: 759, 760, 761, 762, 763, 765, 766, 767, 768, 770, 771, 11238, 23632, 56934, 377677); carcinoembryonic antigen related cell adhesion molecules (e.g., CEACAM3 (CD66d), CEACAM5 (CD66e), CEACAM6 (CD66c); NCBI Gene IDs: 1048, 1084, 4680); casein kinases (e.g., CSNK1A1 (CK1), CSNK2A1 (CK2); NCBI Gene IDs: 1452, 1457); caspases (e.g., CASP3, CASP7, CASP8; NCBI Gene IDs: 836, 840, 841, 864); catenin beta 1 (CTNNB1; NCBI Gene ID: 1499); cathepsin G (CTSG; NCBI Gene ID: 1511); Cbl proto-oncogene B (CBLB, Cbl-b; NCBI Gene ID: 868); C—C motif chemokine ligand 21 (CCL21; NCBI Gene ID: 6366); C—C motif chemokine receptor 2 (CCR2; NCBI Gene ID: 729230); C—C motif chemokine receptors (e.g., CCR3 (CD193), CCR4 (CD194), CCR5 (CD195), CCR8 (CDwl98); NCBI Gene IDs: 1232, 1233, 1234, 1237); CCAAT enhancer binding protein alpha (CEBPA, CEBP; NCBI Gene ID: 1050); cell adhesion molecule 1 (CADM1; NCBI Gene ID: 23705); cell division cycle 7 (CDC7; NCBI Gene ID: 8317); cellular communication network factor 2 (CCN2; NCBI Gene ID: 1490); cereblon (CRBN; NCBI Gene ID: 51185); checkpoint kinases (e.g., CHEK1 (CHK1), CHEK2 (CHK2); NCBI Gene IDs: 1111, 11200); cholecystokinin B receptor (CCKBR; NCBI Gene ID: 887); chorionic somatomammotropin hormone 1 (CSH1; NCBI Gene ID: 1442); claudins (e.g., CLDN6, CLDN18; NCBI Gene IDs: 9074, 51208); cluster of differentiation markers (e.g., CD1A, CD1C, CD1D, CD1E, CD2, CD3 alpha (TRA), CD beta (TRB), CD gamma (TRG), CD delta (TRD), CD4, CD8A, CD8B, CD19, CD20 (MS4A1), CD22, CD24, CD25 (IL2RA, TCGFR), CD28, CD33 (SIGLEC3), CD37, CD38, CD39 (ENTPD1), CD40 (TNFRSF5), CD44 (MIC4, PGP1), CD47 (IAP), CD48 (BLASTI), CD52, CD55 (DAF), CD58 (LFA3), CD74, CD79a, CD79b, CD80 (B7-1), CD84, CD86 (B7-2), CD96 (TACTILE), CD99 (MIC2), CD115 (CSF1R), CD116 (GMCSFR, CSF2RA), CD122 (IL2RB), CD123 (IL3RA), CD128 (IL8R1), CD132 (IL2RG), CD135 (FLT3), CD137 (TNFRSF9, 4-1BB), CD142 (TF, TFA), CD152 (CTLA4), CD160, CD182 (IL8R2), CD193 (CCR3), CD194 (CCR4), CD195 (CCR5), CD207, CD221 (IGF1R), CD222 (IGF2R), CD223 (LAG3), CD226 (DNAM1), CD244, CD247, CD248, CD276 (B7-H3), CD331 (FGFR1), CD332 (FGFR2), CD333 (FGFR3), CD334 (FGFR4); NCBI Gene IDs: 909, 911, 912, 913, 914, 919, 920, 923, 925, 926, 930, 931, 933, 940, 941, 942, 945, 951, 952, 953, 958,960, 961, 962, 965, 972, 973, 974, 1043, 1232, 1233, 1234, 1237, 1436, 1438, 1493, 1604, 2152, 2260, 2261, 2263, 2322, 3480, 3482, 3559, 3560, 3561, 3563, 3577, 3579, 3604, 3902, 4267, 6955, 6957, 6964, 6965, 8832, 10666, 11126, 50489, 51744, 80381, 100133941); clusterin (CLU; NCBI Gene ID: 1191); coagulation factors (e.g., F7, FXA, NCBI Gene IDs: 2155, 2159); collagen type IV alpha chains (e.g., COL4A1, COL4A2, COL4A3, COL4A4, COL4A5; NCBI Gene IDs: 1282, 1284, 1285, 1286, 1287); collectin subfamily member 10 (COLEC10; NCBI Gene ID: 10584); colony stimulating factors (e.g., CSF1 (MCSF), CSF2 (GMCSF), CSF3 (GCSF); NCBI Gene IDs: 1435, 1437, 1440); complement factors (e.g., C3, C5; NCBI Gene IDs: 718, 727); COP9 signalosome subunit 5 (COPS5; NCBI Gene ID: 10987); C-type lectin domain family member (e.g., CLEC4C (CD303), CLEC9A (CD370), CLEC12A (CD371); CD371; NCBI Gene ID: 160364, 170482, 283420); C—X—C motif chemokine ligand 12 (CXCL12; NCBI Gene ID: 6387); C—X—C motif chemokine receptors (CXCR1 (IL8R1, CD128), CXCR2 (IL8R2, CD182), CXCR3 (CD182, CD183, IP-10R), CXCR4 (CD184); NCBI Gene ID: 2833, 3577, 3579, 7852); cyclin D1 (CCND1, BCL1; NCBI Gene ID: 595); cyclin dependent kinases (e.g., CDK1, CDK2, CDK3, CDK4, CDK5, CDK6, CDK7, CDK8, CDK9, CDK10, CDK12; NCBI Gene ID: 983, 1017, 1018, 1019, 1020, 1021, 1022, 1024, 1025, 8558, 51755); cyclin G1 (CCNG1; NCBI Gene ID: 900); cytochrome P450 family members (e.g., CYP2D6, CYP3A4, CYP11A1, CYP11B2, CYP17A1, CYP19A1, CYP51A1; NCBI Gene IDs: 1565, 1576, 1583, 1585, 1586, 1588, 1595); cytochrome P450 oxidoreductase (POR; NCBI Gene ID: 5447); cytokine inducible SH2 containing protein (CISH; NCBI Gene ID: 1154); cytotoxic T-lymphocyte associated protein 4 (CTLA4, CD152; NCBI Gene ID: 1493); DEAD-box helicases (e.g., DDX5, DDX6, DDX58; NCBI Gene IDs: 1655, 1656, 23586); delta like canonical Notch ligands (e.g., DLL3, DLL4; NCBI Gene IDs: 10683, 54567); diablo IAP-binding mitochondrial protein (DIABLO, SMAC; NCBI Gene ID: 56616); diacylglycerol kinases (e.g., DGKA, DGKZ; NCBI Gene IDs: 1606, 8525); dickkopf WNT signaling pathway inhibitors (e.g., DKK1, DKK3; NCBI Gene ID: 22943, 27122); dihydrofolate reductase (DHFR; NCBI Gene ID: 1719); dihydropyrimidine dehydrogenase (DPYD; NCBI Gene ID: 1806); dipeptidyl peptidase 4 (DPP4; NCBI Gene ID: 1803); discoidin domain receptor tyrosine kinases (e.g., DDR1 (CD167), DDR2; CD167; NCBI Gene ID: 780, 4921); DNA dependent protein kinase (PRKDC; NCBI Gene ID: 5591); DNA topoisomerases (e.g., TOP1, TOP2A, TOP2B, TOP3A, TOP3B; NCBI Gene ID: 7150, 7153, 7155, 7156, 8940); dopachrome tautomerase (DCT; NCBI Gene ID: 1638); dopamine receptor D2 (DRD2; NCBI Gene ID: 1318); DOT1 like histone lysine methyltransferase (DOTIL; NCBI Gene ID: 84444); ectonucleotide pyrophosphatase/phosphodiesterase 3 (ENPP3, CD203c; NCBI Gene ID: 5169); EMAP like 4 (EML4; NCBI Gene ID: 27436); endoglin (ENG; NCBI Gene ID: 2022); endoplasmic reticulum aminopeptidases (e.g., ERAP1, ERAP2; NCBI Gene ID: 51752, 64167); enhancer of zeste 2 polycomb repressive complex 2 subunit (EZH2; NCBI Gene ID: 2146); ephrin receptors (e.g., EPHA1, EPHA2EPHA3, EPHA4, EPHA5, EPHA7, EPHB4; NCBIGene ID:1969, 2041, 2042, 2043, 2044, 2045, 2050); ephrins (e.g., EFNA1, EFNA4, EFNB2; NCBI Gene ID: 1942, 1945, 1948); epidermal growth factor receptors (e.g., ERBB1 (HER1, EGFR), ERBB1 variant III (EGFRvIII), ERBB2 (HER2, NEU, CD340), ERBB3 (HER3), ERBB4 (HER4); NCBI Gene ID: 1956, 2064, 2065, 2066); epithelial cell adhesion molecule (EPCAM; NCBI Gene ID: 4072); epithelial mitogen (EPGN; NCBI Gene ID: 255324); eukaryotic translation elongation factors (e.g., EEF1A2, EEF2; NCBI Gene ID: 1917, 1938); eukaryotic translation initiation factors (e.g., EIF4A1, EIFSA; NCBI Gene ID: 1973, 1984); exportin-1 (XPO1; NCBI Gene ID: 7514); farnesoid X receptor (NR1H4, FXR; NCBI Gene ID: 9971); Fas ligand (FASLG, FASL, CD95L, CD178, TNFSF6; NCBI Gene ID: 356); fatty acid amide hydrolase (FAAH; NCBI Gene ID: 2166); fatty acid synthase (FASN; FAS; NCBI Gene ID: 2194); Fc fragment of Ig receptors (e.g., FCER1A, FCGRT, FCGR3A (CD16); NCBI Gene IDs: 2205, 2214, 2217); Fc receptor like 5 (FCRL5, CD307; NCBI Gene ID: 83416); fibroblast activation protein alpha (FAP; NCBI Gene ID: 2191); fibroblast growth factor receptors (e.g., FGFR1 (CD331), FGFR2 (CD332), FGFR3 (CD333), FGFR4 (CD334); NCBI Gene IDs: 2260, 2261, 2263, 2264); fibroblast growth factors (e.g., FGF1 (FGF alpha), FGF2 (FGF beta), FGF4, FGF5; NCBI Gene IDs: 2246, 2247, 2249, 2250); fibronectin 1 (FN1, MSF; NCBI Gene ID: 2335); fms related receptor tyrosine kinases (e.g., FLT1 (VEGFR1), FLT3 (STK1, CD135), FLT4 (VEGFR2); NCBI Gene IDs: 2321, 2322, 2324); fms related receptor tyrosine kinase 3 ligand (FLT3LG; NCBI Gene ID: 2323); focal adhesion kinase 2 (PTK2, FAK1; NCBI Gene ID: 5747); folate hydrolase 1 (FOLH1, PSMA; NCBI Gene ID: 2346); folate receptor 1 (FOLR1; NCBI Gene ID: 2348); forkhead box protein M1 (FOXM1; NCBI Gene ID: 2305); FURIN (FURIN, PACE; NCBI Gene ID: 5045); FYN tyrosine kinase (FYN, SYN; NCBI Gene ID: 2534); galectins (e.g., LGALS3, LGALS8 (PCTA1), LGALS9; NCBI Gene ID: 3958, 3964, 3965); glucocorticoid receptor (NR3C1, GR; NCBI Gene ID: 2908); glucuronidase beta (GUSB; NCBI Gene ID: 2990); glutamate metabotropic receptor 1 (GRM1; NCBI Gene ID: 2911); glutaminase (GLS; NCBI Gene ID: 2744); glutathione S-transferase Pi (GSTP1; NCBI Gene ID: 2950); glycogen synthase kinase 3 beta (GSK3B; NCBI Gene ID: 2932); glypican 3 (GPC3; NCBI Gene ID: 2719); gonadotropin releasing hormone 1 (GNRH1; NCBI Gene ID: 2796); gonadotropin releasing hormone receptor (GNRHR; NCBI Gene ID: 2798); GPNMB glycoprotein nmb (GPNMB, osteoactivin; NCBI Gene ID: 10457); growth differentiation factor 2 (GDF2, BMP9; NCBI Gene ID: 2658); growth factor receptor-bound protein 2 (GRB2, ASH; NCBI Gene ID: 2885); guanylate cyclase 2C (GUCY2C, STAR, MECIL, MUCIL, NCBI Gene ID: 2984); H19 imprinted maternally expressed transcript (H19; NCBI Gene ID: 283120); HCK proto-oncogene, Src family tyrosine kinase (HCK; NCBI Gene ID: 3055); heat shock proteins (e.g., HSPA5 (HSP70, BIP, GRP78), HSPB1 (HSP27), HSP90B1 (GP96); NCBI Gene IDs: 3309, 3315, 7184); heme oxygenases (e.g., HMOX1 (HO1), HMOX2 (HO1); NCBI Gene ID: 3162, 3163); heparanase (HPSE; NCBI Gene ID: 10855); hepatitis A virus cellular receptor 2 (HAVCR2, TIM3, CD366; NCBI Gene ID: 84868); hepatocyte growth factor (HGF; NCBI Gene ID: 3082); HERV-H LTR-associating 2 (HHLA2, B7-H7; NCBI Gene ID: 11148); histamine receptor H2 (HRH2; NCBI Gene ID: 3274); histone deacetylases (e.g., HDAC1, HDAC7, HDAC9; NCBI Gene ID: 3065, 9734, 51564); HRas proto-oncogene, GTPase (HRAS; NCBI Gene ID: 3265); hypoxia-inducible factors (e.g., HIF1A, HIF2A (EPAS1); NCBI Gene IDs: 2034, 3091); I-Kappa-B kinase (IKK beta; NCBI Gene IDs: 3551, 3553); IKAROS family zinc fingers (IKZF1 (LYF1), IKZF3; NCBI Gene ID: 10320, 22806); immunoglobulin superfamily member 11 (IGSF11; NCBI Gene ID: 152404); indoleamine 2,3-dioxygenases (e.g., IDO1, ID02; NCBI Gene IDs: 3620, 169355); inducible T cell costimulator (ICOS, CD278; NCBI Gene ID: 29851); inducible T cell costimulator ligand (ICOSLG, B7-H2; NCBI Gene ID: 23308); insulin like growth factor receptors (e.g., IGF1R, IGF2R; NCBI Gene ID: 3480, 3482); insulin like growth factors (e.g., IGF1, IGF2; NCBI Gene IDs: 3479, 3481); insulin receptor (INSR, CD220; NCBI Gene ID: 3643); integrin subunits (e.g., ITGA5 (CD49e), ITGAV (CD51), ITGB1 (CD29), ITGB2 (CD18, LFA1, MAC1), ITGB7; NCBI Gene IDs: 3678, 3685, 3688, 3695, 3698); intercellular adhesion molecule 1 (ICAM1, CD54; NCBI Gene ID: 3383); interleukin 1 receptor associated kinase 4 (IRAK4; NCBI Gene ID: 51135); interleukin receptors (e.g., IL2RA (TCGFR, CD25), IL2RB (CD122), IL2RG (CD132), IL3RA, IL6R, IL13RA2 (CD213A2), IL22RA1; NCBI Gene IDs: 3598, 3559, 3560, 3561, 3563, 3570, 58985); interleukins (e.g., IL1A, IL1B, IL2, IL3, IL6 (HGF), IL7, IL8 (CXCL8), IL10 (TGIF), IL12A, IL12B, IL15, IL17A (CTLA8), IL18, IL23A, IL24, IL-29 (IFNL1); NCBI Gene IDs: 3552, 3553, 3558, 3562, 3565, 3569, 3574, 3586, 3592, 3593, 3600, 3605, 3606, 11009, 51561, 282618); isocitrate dehydrogenases (NADP(+)1) (e.g., IDH1, IDH2; NCBI Gene IDs: 3417, 3418); Janus kinases (e.g., JAK1, JAK2, JAK3; NCBI Gene IDs: 3716, 3717, 3718); kallikrein related peptidase 3 (KLK3; NCBI Gene ID: 354); killer cell immunoglobulin like receptor, Ig domains and long cytoplasmic tails (e.g., KIR2DL1 (CD158A), KIR2DL2 (CD158B1), KIR2DL3 (CD158B), KIR2DL4 (CD158D), KIR2DL5A (CD158F), KIR2DL5B, KIR3DL1 (CD158E1), KIR3DL2 (CD158K), KIR3DP1 (CD158c), KIR2DS2 (CD158J); NCBI Gene IDs: 3802, 3803, 3804, 3805, 3811, 3812, 57292, 553128, 548594, 100132285); killer cell lectin like receptors (e.g., KLRC1 (CD159A), KLRC2 (CD159c), KLRC3, KLRRC4, KLRD1 (CD94), KLRG1, KLRK1 (NKG2D, CD314); NCBI Gene IDs: 3821, 3822, 3823, 3824, 8302, 10219, 22914); kinase insert domain receptor (KDR, CD309, VEGFR2; NCBI Gene ID: 3791); kinesin family member 11 (KIF11; NCBI Gene ID: 3832); KiSS-1 metastasis suppressor (KISS1; NCBI Gene ID: 3814); KIT proto-oncogene, receptor tyrosine kinase (KIT, C-KIT, CD117; NCBI Gene ID: 3815); KRAS proto-oncogene, GTPase (KRAS; NCBI Gene ID: 3845); lactotransferrin (LTF; NCBI Gene ID: 4057); LCK proto-oncogene, Src family tyrosine kinase (LCK; NCBI Gene ID: 3932); LDL receptor related protein 1 (LRP1, CD91, IGFBP3R; NCBI Gene ID: 4035); leucine rich repeat containing 15 (LRRC15; NCBI Gene ID: 131578); leukocyte immunoglobulin like receptors (e.g., LILRB1 (ILT2, CD85J), LILRB2 (ILT4, CD85D); NCBI Gene ID: 10288, 10859); leukotriene A4 hydrolase (LTA4H; NCBI Gene ID: 4048); linker for activation of T-cells (LAT; NCBI Gene ID: 27040); luteinizing hormone/choriogonadotropin receptor (LHCGR; NCBI Gene ID: 3973); LY6/PLAUR domain containing 3 (LYPD3; NCBI Gene ID: 27076); lymphocyte activating 3 (LAG3; CD223; NCBI Gene ID: 3902); lymphocyte antigens (e.g., LY9 (CD229), LY75 (CD205); NCBI Gene IDs: 4063, 17076); LYN proto-oncogene, Src family tyrosine kinase (LYN; NCBI Gene ID: 4067); lypmphocyte cytosolic protein 2 (LCP2; NCBI Gene ID: 3937); lysine demethylase 1A (KDM1A; NCBI Gene ID: 23028); lysophosphatidic acid receptor 1 (LPAR1, EDG2, LPA1, GPR26; NCBI Gene ID: 1902); lysyl oxidase (LOX; NCBI Gene ID: 4015); lysyl oxidase like 2 (LOXL2; NCBI Gene ID: 4017); macrophage migration inhibitory factor (MIF, GIF; NCBI Gene ID: 4282); macrophage stimulating 1 receptor (MST1R, CD136; NCBI Gene ID: 4486); MAGE family members (e.g., MAGEA1, MAGEA2, MAGEA2B, MAGEA3, MAGEA4, MAGEA5, MAGEA6, MAGEA10, MAGEA11, MAGEC1, MAGEC2, MAGED1, MAGED2; NCBI Gene IDs: 4100, 4101, 4102, 4103, 4104, 4105, 4109, 4110, 9500, 9947, 10916, 51438, 266740); major histocompatibility complexes (e.g., HLA-A, HLA-E, HLA-F, HLA-G; NCBI Gene IDs: 3105, 3133, 3134, 3135); major vault protein (MVP, VAULTI; NCBI Gene ID: 9961); MALT1 paracaspase (MALT1; NCBI Gene ID: 10892); MAPK activated protein kinase 2 (MAPKAPK2; NCBI Gene ID: 9261); MAPK interacting serine/threonine kinases (e.g., MKNK1, MKNK2; NCBI Gene IDs: 2872, 8569); matrix metallopeptidases (e.g., MMP1, MMP2, MMP3, MMP7, MMP8, MMP9, MMP10, MMP11, MMP12, MMP13, MMP14, MMP15, MMP16, MMP17, MMP19, MMP20, MMP21, MMP24, MMP25, MMP26, MMP27, MMP28; NCBI Gene IDs: 4312, 4313, 4314, 4316, 4317, 4318, 4319, 4320, 4321, 4322, 4323, 4324, 4325, 4326, 4327, 9313, 10893, 56547, 64066, 64386, 79148, 118856); MCL1 apoptosis regulator, BCL2 family member (MCL1; NCBI Gene ID: 4170); MDM2 proto-oncogene (MDM2; NCBI Gene ID: 4193); MDM4 regulator of p53 (MDM4; BMFS6; NCBI Gene ID: 4194); mechanistic target of rapamycin kinase (MTOR, FRAP1; NCBI Gene ID: 2475); melan-A (MLANA; NCBI Gene ID: 2315); melanocortin receptors (MC1R, MC2R; NCBI Gene IDs: 4157, 4148); MER proto-oncogene, tyrosine kinase (MERTK; NCBI Gene ID: 10461); mesothelin (MSLN; NCBI Gene ID: 10232); MET proto-oncogene, receptor tyrosine kinase (MET, c-Met, HGFR; NCBI Gene ID: 4233); methionyl aminopeptidase 2 (METAP2, MAP2; NCBI Gene ID: 10988); MHC class I polypeptide-related sequences (e.g., MICA, MICB; NCBI Gene IDs: 4277, 100507436); mitogen activated protein kinases (e.g., MAPK1 (ERK2), MAPK3 (ERK1), MAPK8 (JNK1), MAPK9 (JNK2), MAPK10 (JNK3), MAPK11 (p38 beta), MAPK12; NCBI Gene IDs: 5594, 5595, 5599, 5600, 5601, 5602, 819251); mitogen-activated protein kinase kinase kinases (e.g., MAP3K5 (ASK1), MAP3K8 (TPL2, AURA2); NCBI Gene IDs: 4217, 1326); mitogen-activated protein kinase kinase kinase kinase 1 (MAP4K1, HPK1; NCBI Gene ID: 11184); mitogen-activated protein kinase kinases (e.g., MAP2K1 (MEK1), MAP2K2 (MEK2), MAP2K7 (MEK7); NCBI Gene IDs: 5604, 5605, 5609); MPL proto-oncogene, thrombopoietin receptor (MPL; NCBI Gene ID: 4352); mucins (e.g., MUC1 (including splice variants thereof (e.g., including MUC1/A, C, D, X, Y, Z and REP)), MUCSAC, MUC16 (CA125); NCBI Gene IDs: 4582, 4586, 94025); MYC proto-oncogene, bHLH transcription factor (MYC; NCBI Gene ID: 4609); myostatin (MSTN, GDF8; NCBI Gene ID: 2660); myristoylated alanine rich protein kinase C substrate (MARCKS; NCBI Gene ID: 4082); natriuretic peptide receptor 3 (NPR3; NCBI Gene ID: 4883); natural killer cell cytotoxicity receptor 3 ligand 1 (NCR3LG1, B7-H6; NCBI Gene ID: 374383); necdin, MAGE family member (NDN; NCBI Gene ID: 4692); nectin cell adhesion molecules (e.g., NECTIN2 (CD112, PVRL2), NECTIN4 (PVRL4); NCBI Gene IDs: 5819, 81607); neural cell adhesion molecule 1 (NCAM1, CD56; NCBI Gene ID: 4684); neuropilins (e.g., NRP1 (CD304, VEGF165R), NRP2 (VEGF165R2); NCBI Gene IDs: 8828, 8829); neurotrophic receptor tyrosine kinases (e.g., NTRK1 (TRKA), NTRK2 (TRKB), NTRK3 (TRKC); NCBI Gene IDs: 4914, 4915, 4916); NFKB activating protein (NKAP; NCBI Gene ID: 79576); NIMA related kinase 9 (NEK9; NCBI Gene ID: 91754); NLR family pyrin domain containing 3 (NLRP3, NALP3; NCBI Gene ID: 114548); notch receptors (e.g., NOTCH1, NOTCH2, NOTCH3, NOTCH4; NCBI Gene IDs: 4851, 4853, 4854, 4855); NRAS proto-oncogene, GTPase (NRAS; NCBI Gene ID: 4893); nuclear factor kappa B (NFKB1, NFKB2; NCBI Gene IDs: 4790, 4791); nuclear factor, erythroid 2 like 2 (NFE2L2; NRF2; NCBI Gene ID: 4780); nuclear receptor subfamily 4 group A member 1 (NR4A1; NCBI Gene ID: 3164); nucleolin (NCL; NCBI Gene ID: 4691); nucleophosmin 1 (NPM1; NCBI Gene ID: 4869); nucleotide binding oligomerization domain containing 2 (NOD2; NCBI Gene ID: 64127); nudix hydrolase 1 (NUDT1; NCBI Gene ID: 4521); 0-6-methylguanine-DNA methyltransferase (MGMT; NCBI Gene ID: 4255); opioid receptor delta 1 (OPRD1; NCBI Gene ID: 4985); ornithine decarboxylase 1 (ODC1; NCBI Gene ID: 4953); oxoglutarate dehydrogenase (OGDH; NCBI Gene ID: 4967); parathyroid hormone (PTH; NCBI Gene ID: 5741); PD-L1 (CD274; NCBI Gene ID: 29126); periostin (POSTN; NCBI Gene ID: 10631); peroxisome proliferator activated receptors (e.g., PPARA (PPAR alpha), PPARD (PPAR delta), PPARG (PPAR gamma); NCBI Gene IDs: 5465, 5467, 5468); phosphatase and tensin homolog (PTEN; NCBI Gene ID: 5728); phosphatidylinositol-4,5-bisphosphate 3-kinases (PIK3CA (PI3K alpha), PIK3CB (PI3K beta), PIK3CD (PI3K delta), PIK3CG (PI3K gamma); NCBI Gene IDs: 5290, 5291, 5293, 5294); phospholipases (e.g., PLA2G1B, PLA2G2A, PLA2G2D, PLA2G3, PLA2G4A, PLA2G5, PLA2G7, PLA2G10, PLA2G12A, PLA2G12B, PLA2G15; NCBI Gene IDs: 5319, 5320, 5321, 5322, 7941, 8399, 50487, 23659, 26279, 81579, 84647); Pim proto-oncogene, serine/threonine kinases (e.g., PIM1, PIM2, PIM3; NCBI Gene IDs: 5292, 11040, 415116); placenta growth factor (PGF; NCBI Gene ID: 5228); plasminogen activator, urokinase (PLAU, u-PA, ATF; NCBI Gene ID: 5328); platelet derived growth factor receptors (e.g., PDGFRA (CD140A, PDGFR2), FDGFRB (CD140B, PDGFR1); NCBI Gene IDs: 5156, 5159); plexin BI (PLXNB1; NCBI Gene ID: 5364); poliovirus receptor (PVR) cell adhesion molecule (PVR, CD155; NCBI Gene ID: 5817); polo like kinase 1 (PLK1; NCBI Gene ID: 5347); poly(ADP-ribose) polymerases (e.g., PARP1, PARP2, PARP3; NCBI Gene IDs: 142, 10038, 10039); polycomb protein EED (EED; NCBI Gene ID: 8726); porcupine O-acyltransferase (PORCN; NCBI Gene ID: 64840); PRAME nuclear receptor transcriptional regulator (PRAME; NCBI Gene ID: 23532); premelanosome protein (PMEL; NCBI Gene ID: 6490); progesterone receptor (PGR; NCBI Gene ID: 5241); programmed cell death 1 (PDCD1, PD-1, CD279; NCBI Gene ID: 5133); programmed cell death 1 ligand 2 (PDCD1LG2, CD273, PD-L2; NCBI Gene ID: 80380); prominin 1 (PROM1, CD133; NCBI Gene ID: 8842); promyelocytic leukemia (PML; NCBI Gene ID: 5371); prosaposin (PSAP; NCBI Gene ID: 5660); prostaglandin E receptor 4 (PTGER4; NCBI Gene ID: 5734); prostaglandin E synthase (PTGES; NCBI Gene ID: 9536); prostaglandin-endoperoxide synthases (PTGS1 (COX1), PTGS2 (COX2); NCBI Gene ID: 5742, 5743); proteasome 20S subunit beta 9 (PSMB9; NCBI Gene ID: 5698); protein arginine methyltransferases (e.g., PRMT1, PRMT5; NCBI Gene ID: 3276, 10419); protein kinase N3 (PKN3; NCBI Gene ID: 29941); protein phosphatase 2A (PPP2CA; NCBI Gene ID: 5515); protein tyrosine kinase 7 (inactive) (PTK7; NCBI Gene ID: 5754); protein tyrosine phosphatase receptors (PTPRB (PTPB), PTPRC (CD45R); NCBI Gene ID: 5787, 5788); prothymosin alpha (PTMA; NCBI Gene ID: 5757); purine nucleoside phosphorylase (PNP; NCBI Gene ID: 4860); purinergic receptor P2X 7 (P2RX7; NCBI Gene ID: 5027); PVR related immunoglobulin domain containing (PVRIG, CD112R; NCBI Gene ID: 79037); Raf-1 proto-oncogene, serine/threonine kinase (RAF1, c-Raf; NCBI Gene ID: 5894); RAR-related orphan receptor gamma (RORC; NCBI Gene ID: 6097); ras homolog family member C (RHOC); NCBI Gene ID: 389); Ras homolog, mTORC1 binding (RHEB; NCBI Gene ID: 6009); RB transcriptional corepressor 1 (RB1; NCBI Gene ID: 5925); receptor-interacting serine/threonine protein kinase 1 (RIPK1; NCBI Gene ID: 8737); ret proto-oncogene (RET; NCBI Gene ID: 5979); retinoic acid early transcripts (e.g., RAETIE, RAETIG, RAETIL; NCBI Gene IDs: 135250, 154064, 353091); retinoic acid receptors alpha (e.g., RARA, RARG; NCBI Gene IDs: 5914, 5916); retinoid X receptors (e.g., RXRA, RXRB, RXRG; NCBI Gene IDs: 6256, 6257, 6258); Rho associated coiled-coil containing protein kinases (e.g., ROCK1, ROCK2; NCBI Gene IDs: 6093, 9475); ribosomal protein S6 kinase B1 (RPS6KB1, S6K-beta 1; NCBI Gene ID: 6198); ring finger protein 128 (RNF128, GRAIL; NCBI Gene ID: 79589); ROS proto-oncogene 1, receptor tyrosine kinase (ROS1; NCBI Gene ID: 6098); roundabout guidance receptor 4 (ROBO4; NCBI Gene ID: 54538); RUNX family transcription factor 3 (RUNX3; NCBI Gene ID: 864); S100 calcium binding protein A9 (S100A9; NCBI Gene ID: 6280); secreted frizzled related protein 2 (SFRP2; NCBI Gene ID: 6423); secreted phosphoprotein 1 (SPP1; NCBI Gene ID: 6696); secretoglobin family 1A member 1 (SCGB1A1; NCBI Gene ID: 7356); selectins (e.g., SELE, SELL (CD62L), SELP (CD62); NCBI Gene IDs: 6401, 6402, 6403); semaphorin 4D (SEMA4D; CD100; NCBI Gene ID: 10507); sialic acid binding Ig like lectins (SIGLEC7 (CD328), SIGLEC9 (CD329), SIGLEC10; NCBI Gene ID: 27036, 27180, 89790); signal regulatory protein alpha (SIRPA, CD172A; NCBI Gene ID: 140885); signal transducer and activator of transcription (e.g., STAT1, STAT3, STAT5A, STAT5B; NCBI Gene IDs: 6772, 6774, 6776, 6777); sirtuin-3 (SIRT3; NCBI Gene ID: 23410); signaling lymphocytic activation molecule (SLAM) family members (e.g., SLAMF1 (CD150), SLAMF6 (CD352), SLAMF7 (CD319), SLAMF8 (CD353), SLAMF9; NCBI Gene IDs: 56833, 57823, 89886, 114836); SLIT and NTRK like family member 6 (SLITRK6; NCBI Gene ID: 84189); smoothened, frizzled class receptor (SMO; NCBI Gene ID: 6608); soluble epoxide hydrolase 2 (EPHX2; NCBI Gene ID: 2053); solute carrier family members (e.g., SLC3A2 (CD98), SLC5A5, SLC6A2, SLC10A3, SLC34A2, SLC39A6, SLC43A2 (LAT4), SLC44A4; NCBI Gene IDs: 6520, 6528, 6530, 8273, 10568, 25800, 80736, 124935); somatostatin receptors (e.g., SSTR1, SSTR2, SSTR3, SSTR4, SSTR5; NCBI Gene IDs: 6751, 6752, 6753, 6754, 6755); sonic hedgehog signaling molecule (SHH; NCBI Gene ID: 6469); Spl transcription factor (SP1; NCBI Gene ID: 6667); sphingosine kinases (e.g., SPHK1, SPHK2; NCBI Gene IDs: 8877, 56848); sphingosine-1-phosphate receptor 1 (S1PR1, CD363; NCBI Gene ID: 1901); spleen associated tyrosine kinase (SYK; NCBI Gene ID: 6850); splicing factor 3B factor 1 (SF3B1; NCBI Gene ID: 23451); SRC proto-oncogene, non-receptor tyrosine kinase (SRC; NCBI Gene ID: 6714); stabilin 1 (STAB1, CLEVER-1; NCBI Gene ID: 23166); STEAP family member 1 (STEAP1; NCBI Gene ID: 26872); steroid sulfatase (STS; NCBI Gene ID: 412); stimulator of interferon response cGAMP interactor 1 (STINGI; NCBI Gene ID: 340061); superoxide dismutase 1 (SOD1, ALS1; NCBI Gene ID: 6647); suppressors of cytokine signaling (SOCS1 (CISH1), SOCS3 (CISH3); NCBI Gene ID: 8651, 9021); synapsin 3 (SYN3; NCBI Gene ID: 8224); syndecan 1 (SDC1, CD138, syndecan; NCBI Gene ID: 6382); synuclein alpha (SNCA, PARK1; NCBI Gene ID: 6622); T cell immunoglobulin and mucin domain containing 4 (TIMD4, SMUCKLER; NCBI Gene ID: 91937); T cell immunoreceptor with Ig and ITIM domains (TIGIT; NCBI Gene ID: 201633); tachykinin receptors (e.g., TACR1, TACR3; NCBI Gene ID: 6869, 6870); TANK binding kinase 1 (TBK1; NCBI Gene ID: 29110); tankyrase (TNKS; NCBI Gene ID: 8658); TATA-box binding protein associated factor, RNA polymerase I subunit B (TAF1B; NCBI Gene ID: 9014); T-box transcription factor T (TBXT; NCBI Gene ID: 6862); TCDD inducible poly(ADP-ribose) polymerase (TIPARP, PAPR7; NCBI Gene ID: 25976); tec protein tyrosine kinase (TEC; NCBI Gene ID: 7006); TEK receptor tyrosine kinase (TEK, CD202B, TIE2; NCBI Gene ID: 7010); telomerase reverse transcriptase (TERT; NCBI Gene ID: 7015); tenascin C (TNC; NCBI Gene ID: 3371); three prime repair exonucleases (e.g., TREX1, TREX2; NCBI Gene ID: 11277, 11219); thrombomodulin (THBD, CD141; NCBI Gene ID: 7056); thymidine kinases (e.g., TK1, TK2; NCBI Gene IDs: 7083, 7084); thymidine phosphorylase (TYMP; NCBI Gene ID: 1890); thymidylate synthase (TYMS; NCBI Gene ID: 7298); thyroid hormone receptor (THRA, THRB; NCBI Gene IDs: 7606, 7608); thyroid stimulating hormone receptor (TSHR; NCBI Gene ID: 7253); TNF superfamily members (e.g., TNFSF4 (OX40L, CD252), TNFSF5 (CD40L), TNFSF7 (CD70), TNFSF8 (CD153, CD30L), TNFSF9 (4-1BB-L, CD137L), TNFSF10 (TRAIL, CD253, APO2L), TNFSF11 (CD254, RANKL2, TRANCE), TNFSF13 (APRIL, CD256, TRAIL2), TNFSF13b (BAFF, BLYS, CD257), TNFSF14 (CD258, LIGHT), TNFSF18 (GITRL); NCBI Gene IDs: 944, 959, 970, 7292, 8600, 8740, 8741, 8743, 8744, 8995); toll like receptors (e.g., TLR1 (CD281), TLR2 (CD282), TLR3 (CD283), TLR4 (CD284), TLR5, TLR6 (CD286), TLR7, TLR8 (CD288), TLR9 (CD289), TLR10 (CD290); NCBI Gene IDs: 7096, 7097, 7098, 7099, 10333, 51284, 51311, 54106, 81793); transferrin (TF; NCBI Gene ID: 7018); transferrin receptor (TFRC, CD71; NCBI Gene ID: 7037); transforming growth factors (e.g., TGFA, TGFB1; NCBI Gene ID: 7039, 7040); transforming growth factor receptors (e.g., TGFBR1, TGFBR2, TGFBR3; NCBI Gene ID: 7046, 7048, 7049); transforming protein E7 (E7; NCBI Gene ID: 1489079); transglutaminase 5 (TGM5; NCBI Gene ID: 9333); transient receptor potential cation channel subfamily V member 1 (TRPV1, VR1; NCBI Gene ID: 7442); transmembrane and immunoglobulin domain containing 2 (TMIGD2, CD28H, IGPR1; NCBI Gene ID: 126259); triggering receptors expressed on myeloid cells (e.g., TREM1 (CD354), TREM2; NCBI Gene ID: 54209, 54210); trophinin (TRO, MAGED3; NCBI Gene ID: 7216); trophoblast glycoprotein (TPBG; NCBI Gene ID: 7162); tryptophan 2,3-dioxygenase (TDO2; NCBI Gene ID: 6999); tryptophan hydroxylases (e.g., TPH1, TPH2; NCBI Gene ID: 7166, 121278); tumor associated calcium signal transducer 2 (TACSTD2, TROP2, EGP1; NCBI Gene ID: 4070); tumor necrosis factor (TNF; NCBI Gene ID: 7124); tumor necrosis factor (TNF) receptor superfamily members (e.g., TNFRSF1A (CD120a), TNFRSF1B (CD120b), TNFRSF4 (OX40), TNFRSF5 (CD40), TNFRSF6 (CD95, FAS receptor), TNFRSF7 (CD27), TNFRSF8 (CD30), TNFRSF9 (CD137, 4-1BB), TNFRSF10A (CD261), TNFRSF10B (TRAIL, DR5, CD262), TNFRSF10C, TNFRSF10D, TNFRSF11A, TNFRSF11B (OPG), TNFRSF12A, TNFRSF13B, TNFR13C (CD268, BAFFR), TNFRSF14 (CD270, LIGHTR), TNFRSF16, TNFRSF17 (CD269, BCMA), TNFRSF18 (GITR, CD357), TNFRSF19, TNFRSF21, TNFRSF25, NCBI Gene IDs: 355, 608, 939, 943, 958, 3604, 4804, 4982, 7132, 7133, 7293, 8718, 8764, 8784, 8792, 8793, 8794, 8795, 8797, 23495, 27242, 51330, 55504); tumor protein p53 (TP53; NCBI Gene ID: 7157); tumor suppressor 2, mitochondrial calcium regulator (TUSC2; NCBI Gene ID: 11334); TYRO3 protein tyrosine kinase (TYRO3; BYK; NCBI Gene ID: 7301); tyrosinase (TYR; NCBI Gene ID: 7299); tyrosine hydroxylase (TH; NCBI Gene ID: 7054); tyrosine kinase with immunoglobulin like and EGF like domains 1 (e.g., TIE1, TIE1; NCBI Gene ID: 7075); tyrosine-protein phosphatase non-receptor type 11 (PTPN11, SHP2; NCBI Gene ID: 5781); ubiquitin conjugating enzyme E2 I (UBE2I, UBC9; NCBI Gene ID: 7329); ubiquitin C-terminal hydrolase L5 (UCHL5; NCBI Gene ID: 51377); ubiquitin specific peptidase 7 (USP7; NCBI Gene ID: 7874); ubiquitin-like modifier activating enzyme 1 (UBA1; NCBI Gene ID: 7317); UL16 binding proteins (e.g., ULBP1, ULBP2, ULBP3; NCBI Gene ID: 79465, 80328, 80328); valosin-containing protein (VCP, CDC48; NCBI Gene ID: 7415); vascular cell adhesion molecule 1 (VCAM1, CD106; NCBI Gene ID: 7412); vascular endothelial growth factors (e.g., VEGFA, VEGFB; NCBI Gene ID: 7422, 7423); vimentin (VIM; NCBI Gene ID: 7431); vitamin D receptor (VDR; NCBI Gene ID: 7421); V-set domain containing T cell activation inhibitor 1 (VTCN1, B7-H4; NCBI Gene ID: 79679); V-set immunoregulatory receptor (VSIR, VISTA, B7-H5; NCBI Gene ID: 64115); WEE1 G2 checkpoint kinase (WEE1; NCBI Gene ID: 7465); WRN RecQ like helicase (WRN; RECQ3; NCBI Gene ID: 7486); WT1 transcription factor (WT1; NCBI Gene ID: 7490); WW domain containing transcription regulator 1 (WWTR1; TAZ; NCBI Gene ID: 25937); X—C motif chemokine ligand 1 (XCL1, ATAC; NCBI Gene ID: 6375); X—C motif chemokine receptor 1 (XCR1, GPR5, CCXCR1; NCBI Gene ID: 2829); Yesl associated transcriptional regulator (YAPi; NCBI Gene ID: 10413); zeta chain associated protein kinase 70 (ZAP70; NCBI Gene ID: 7535).

In some embodiments, the one or more additional therapeutic agents include, e.g., an agent targeting 5′-nucleotidase ecto (NT5E or CD73; NCBI Gene ID: 4907); adenosine A_{2A} receptor (ADORA2A; NCBI Gene ID: 135); adenosine A_{2B} receptor (ADORA2B; NCBI Gene ID: 136); C—C motif chemokine receptor 8 (CCR8, CDwl98; NCBI Gene ID: 1237); cytokine inducible SH2 containing protein (CISH; NCBI Gene ID: 1154); diacylglycerol kinase alpha (DGKA, DAGK, DAGK1 or DGK-alpha; NCBI Gene ID: 1606); fms like tyrosine kinase 3 (FLT3, CD135; NCBI Gene ID: 2322); integrin associated protein (IAP, CD47; NCBI Gene ID: 961); interleukine-2 (IL2; NCBI Gene ID:3558); interleukine 2 receptor (IL2RA, IL2RB, IL2RG; NCBI Gene IDs: 3559, 3560, 3561); Kirsten rat sarcoma virus (KRAS; NCBI Gene ID: 3845; including mutations, such as KRAS G12C or G12D); mitogen-activated protein kinase kinase kinase kinase 1 (MAP4K1) (also called Hematopoietic Progenitor Kinase 1 (HPK1), NCBI Gene ID: 11184); myeloid cell leukemia sequence 1 apoptosis regulator (MCL1; NCBI Gene ID: 4170); phosphatidylinositol-4,5-bisphosphate 3-kinase, catalytic subunit delta (PIK3CD; NCBI Gene ID: 5293); programmed death-ligand 1 (PD-L1, CD274; NCBI Gene ID 29126); programmed cell death protein 1 (PD-1, CD279; NCBI Gene ID: 5133); proto-oncogen c-KIT (KIT, CD117; NCBI Gene ID: 3815); signal-regulatory protein alpha (SIRPA, CD172A; NCBI Gene ID: 140885); TCDD inducible poly(ADP-ribose) polymerase (TIPARP, PARP7; NCBI Gene ID: 25976); T cell immunoreceptor with Ig and ITIM domains (TIGIT; NCBI Gene ID: 201633); triggering receptor expressed on myeloid cells 1 (TREM1; NCBI Gene ID: 54210); triggering receptor expressed on myeloid cells 2 (TREM2; NCBI Gene ID: 54209); tumor-associated calcium signal transducer 2 (TACSTD2, TROP2, EGP1; NCBI Gene ID: 4070); tumor necrosis factor receptor superfamily, member 4 (TNFRSF4, CD134, OX40; NCBI Gene ID:7293); tumor necrosis factor receptor superfamily, member 9 (TNFRSF9, 4-1BB, CD137; NCBI Gene ID: 3604); tumor necrosis factor receptor superfamily, member 18 (TNFRSF18, CD357, GITR; NCBI Gene ID: 8784); WRN RecQ like helicase (WRN; NCBI Gene ID: 7486); zinc finger protein Helios (IKZF2; NCBI Gene ID: 22807).

### Illustrative Mechanisms of Action

### Immune Checkpoint Modulators

In some embodiments a compound provided herein is administered with one or more blockers or inhibitors of inhibitory immune checkpoint proteins or receptors and/or with one or more stimulators, activators or agonists of one or more stimulatory immune checkpoint proteins or receptors. In some embodiments, the immune checkpoint proteins or receptors regulate T cell responses (e.g., reviewed in Xu, et al., J Exp Clin Cancer Res. (2018) 37:110). In some embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688). Inhibition of regulatory T-cells (Treg) or Treg depletion can alleviate their suppression of antitumor immune responses and have anticancer effects (e.g., reviewed in Plitas and Rudensky, Annu. Rev. Cancer Biol. (2020) 4:459-77; Tanaka and Sakaguchi, Eur. J. Immunol. (2019) 49:1140-1146).

### Cluster of Differentiation Agonists or Activators

In some embodiments, the compound provided herein is administered with agents targeting a cluster of differentiation (CD) marker.

### SIRPa Targeting Agents

In some embodiments the compound provided herein is administered with a SIRPa targeting agent (NCBI Gene ID: 140885; UniProt P78324).

### FLT3R Agonists

In some embodiments the compound provided herein is administered with a FLT3R agonist. In some embodiments, the compound provided herein is administered with a FLT3 ligand. In some embodiments, the compound provided herein is administered with a FLT3L-Fc fusion protein.

### TNF Receptor Superfamily (TNFRSF) Member Agonists or Activators

In some embodiments, the compound provided herein is administered with an agonist of one or more TNF receptor superfamily (TNFRSF) members.

### Bi-Specific T-Cell Engagers

In some embodiments, the compound provided herein is administered with a bi-specific T-cell engager (e.g., not having an Fc) or an anti-CD3 bi-specific antibody (e.g., having an Fc).

### Bi- and Tri-Specific Natural Killer (NK)-Cell Engagers

In some embodiments, the compound provided herein is administered with a bi-specific NK-cell engager (BiKE) or a tri-specific NK-cell engager (TriKE) (e.g., not having an Fc) or bi-specific antibody (e.g., having an Fc) against an NK cell activating receptor.

### MCL1 Apoptosis Regulator, BCL2 Family Member (MCL1) Inhibitors

In some embodiments, the compound provided herein is administered with an inhibitor of MCL1 apoptosis regulator, BCL2 family member (MCL1, TM; EAT; MCL1L; MCL1S; Mel-1; BCL2L3; MCL1-ES; bcl2-L-3; mcl1/EAT; NCBI Gene ID: 4170).

### SHP2 Inhibitors

In some embodiments, compound provided herein is administered with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781).

### Hematopoietic Progenitor Kinase 1 (HPK1) Inhibitors and Degraders

In some embodiments, the compound provided herein is administered with an inhibitor of mitogen-activated protein kinase kinase kinase kinase 1 (MAP4K1, HPK1; NCBI Gene ID: 11184).

### Apoptosis Signal-Regulating Kinase (ASK) Inhibitors

In some embodiments, the compound provided herein is administered with an ASK inhibitor, e.g., mitogen-activated protein kinase kinase kinase 5 (MAP3K5; ASK1, MAPKKK5, MEKK5; NCBI Gene ID: 4217).

### Bruton Tyrosine Kinase (BTK) Inhibitors

In some embodiments, the compound provided herein is administered with an inhibitor of Bruton tyrosine kinase (BTK, AGMX1, AT, ATK, BPK, IGHD3, IMD1, PSCTK1, XLA; NCBI Gene ID: 695).

### Cyclin-Dependent Kinase (CDK) Inhibitors

In some embodiments, the compound provided herein is administered with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3, NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022), or cyclin dependent kinase 9 (CDK9, TAK; C-2k; CTK1; CDC2L4; PITALRE; NCBI Gene ID: 1025).

### Discoidin Domain Receptor (DDR) Inhibitors

In some embodiments, the compound provided herein is combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). WO2013/034933 (Imperial Innovations).

### Targeted E3 Ligase Ligand Conjugates

In some embodiments, the compound provided herein is administered with a targeted E3 ligase ligand conjugate. Such conjugates have a target protein binding moiety and an E3 ligase binding moiety (e.g., an inhibitor of apoptosis protein (IAP) (e.g., XIAP, c-IAP1, c-IAP2, NIL-IAP, Bruce, and surviving) E3 ubiquitin ligase binding moiety, Von Hippel-Lindau E3 ubiquitin ligase (VHL) binding moiety, a cereblon E3 ubiquitin ligase binding moiety, mouse double minute 2 homolog (MDM2) E3 ubiquitin ligase binding moiety), and can be used to promote or increase the degradation of targeted proteins, e.g., via the ubiquitin pathway.

### Histone Deacetylase (HDAC) Inhibitors

In some embodiments, the compound provided herein is administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734).

### Indoleamine-Pyrrole-2,3-Dioxygenase (IDO1) Inhibitors

In some embodiments the compound provided herein is administered with an inhibitor of indoleamine 2,3-dioxygenase 1 (IDO1; NCBI Gene ID: 3620).

### Janus Kinase (JAK) Inhibitors

In some embodiments, the compound provided herein is administered with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718).

### Lysyl Oxidase-Like Protein (LOXL) Inhibitors

In some embodiments the compound provided herein is administered with an inhibitor of a LOXL protein, e.g., LOXL1 (NCBI Gene ID: 4016), LOXL2 (NCBI Gene ID: 4017), LOXL3 (NCBI Gene ID: 84695), LOXL4 (NCBI Gene ID: 84171), and/or LOX (NCBI Gene ID: 4015).

### Matrix Metalloprotease (MMP) Inhibitors

In some embodiments the compound provided herein is administered with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148).

### RAS and RAS Pathway Inhibitors

In some embodiments the compound provided herein is administered with an inhibitor of KRAS proto-oncogene, GTPase (KRAS; a.k.a., NS; NS3; CFC2; RALD; K-Ras; KRAS1; KRAS2; RASK2; KI-RAS; C—K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893) or HRAS proto-oncogene, GTPase (HRAS; a.k.a., CTLO; KRAS; HAMSV; HRAS1; KRAS2; RASH1; RASK2; Ki-Ras; p21ras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR.

### Mitogen-Activated Protein Kinase (MEK) Inhibitors

In some embodiments the compound provided herein is administered with an inhibitor of mitogen-activated protein kinase kinase 7 (MAP2K7, JNKK2, MAPKK7, MEK, MEK 7, MKK7, PRKMK7, SAPKK-4, SAPKK4; NCBI Gene ID: 5609).

### Phosphatidylinositol 3-Kinase (PI3K) Inhibitors

In some embodiments compounds provided herein is administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293).

### Spleen Tyrosine Kinase (SYK) Inhibitors

In some embodiments the compound provided herein is administered with an inhibitor of spleen associated tyrosine kinase (SYK, p72-Syk, NCBI Gene ID: 6850).

### Toll-Like Receptor (TLR) Agonists

In some embodiments compound provided herein is administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793).

### Tyrosine-Kinase Inhibitors (TKIs)

In some embodiments the compound provided herein is administered with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF).

### Chemotherapeutic Agents

In some embodiments the compound provided herein is administered with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

### Anti-Angiogenic Agents

In some embodiments the compound provided herein is administered with an anti-angiogenic agent.

### Anti-Fibrotic Agents

In some embodiments the compound provided herein is administered with an anti-fibrotic agent.

### Anti-Inflammatory Agents

In some embodiments the compound provided herein is administered with an anti-inflammatory agent.

### Tumor Oxygenation Agents

In some embodiments the compound provided herein is administered with an agent that promotes or increases tumor oxygenation or reoxygenation, or prevents or reduces tumor hypoxia.

### Immunotherapeutic Agents

In some embodiments the compound provided herein is administered with an immunotherapeutic agent. In some embodiments the immunotherapeutic agent is an antibody.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein.

### Cancer Gene Therapy and Cell Therapy

In some embodiments the compound provided herein is administered with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In some embodiments the compound provided herein is administered with one or more cellular therapies. As appropriate, a cellular therapy can entail the co-administration of cells that are autologous, syngeneic or allogeneic to the subject.

In some embodiments the cellular therapy entails co-administering cells comprising chimeric antigen receptors (CARs). In such therapies, a population of immune effector cells engineered to express a CAR, wherein the CAR comprises a tumor antigen-binding domain. In T cell therapies, the T cell receptors (TCRs) are engineered to target tumor derived peptides presented on the surface of tumor cells.

### Additional Exemplified Combination Therapies

In some embodiments the compound provided herein is administered with one or more therapeutic agents selected from a PI3K inhibitor, a Trop-2 binding agent, CD47 antagonist, a SIRPα antagonist, a FLT3R agonist, a PD-1 antagonist, a PD-L1 antagonist, an MCL1 inhibitor, a CCR8 binding agent, an HPK1 antagonist, a DGKa inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, ion exchange chromatography, and supercritical fluid chromatography (SFC). Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4th ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or disatereomerically pure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### EXAMPLES

### I. Abbreviations

Certain abbreviations and acronyms are used in describing the experimental details. Although most of these would be understood by one skilled in the art, Table 1 contains a list of many of these abbreviations and acronyms.

TABLE 1
Abbreviations

| Abbreviation | Meaning |
|---|---|
| ° C. | degree(s) Celsius |
| aq | aqueous |
| Boc | tert-butoxycarbonyl |
| Bn | benzyl |
| br s | broad singlet |
| Cbz | benzyloxycarbonyl |
| d | doublet |
| Dₘₐₓ | maximal degradation |
| DC₅₀ | half-maximal degradation concentration |
| DCM | dichloromethane |
| dd | doublet of doublets |
| ddd | doublet of doublet of doublets |
| ddt | doublet of doublet of triplets |
| Dess-Martin | 3-oxo-1λ⁵,2-benziodoxole-1,1,1(3H)-triyl triacetate |
| Periodinane | |
| DMB | 2,4-dimethoxybenzyl |
| DMSO | dimethyl sulfoxide |
| dt | doublet of triplets |
| Et | ethyl |
| EtOAc | ethyl acetate |
| g | gram(s) |
| h | hour(s) |
| HPLC | high-performance liquid chromatography |
| Hz | Hertz |
| ⁱPr | isopropyl |
| IPA or iPrOH | isopropyl alcohol |
| J | coupling constant |
| LCMS | liquid chromatography mass spectrometry |
| m | multiplet |
| M | molarity |
| Me | methyl |
| MeCN | acetonitrile |
| mg | milligram(s) |
| MHz | megahertz |
| min | minute(s) |
| mL | milliliter(s) |
| mm | millimeter(s) |
| mmol | millimole(s) |
| MOMO or OMOM | methoxymethoxy |
| MPa | megapascals |
| NMR | nuclear magnetic resonance |
| OSEM | 2-(trimethylsilyl)ethoxymethoxy |
| OTf | trifluoromethanesulfonate |
| Ph | phenyl |
| prep-HPLC | preparative high performance liquid chromatography |
| qd | quartet of doublets |
| RP-HPLC | reversed phase high performance liquid chromatography |
| rt or RT | room temperature |
| s | singlet |
| sat. | saturated |
| SEM | 2-(trimethylsilyl)ethoxymethyl |
| SFC | supercritical fluid chromatography |
| t | triplet |
| TBAF | tetra-n-butylammonium fluoride |
| TBS | tert-butyldimethylsilyl |
| ^{t}Bu | tert-butyl |
| td | triplet of doublets |
| TFA | trifluoroacetic acid |
| THF | tetrahydrofuran |
| TIPS | triisopropylsilyl |
| TMS | trimethylsilyl |
| tt | triplet of triplets |
| v/v | volume/volume |
| wt | weight |
| δ | parts per million referenced to residual non-deuterated solvent peak |
| * | * next to a chiral center indicates that the stereochemistry at the chiral |
| | center is assigned arbitrarily, and the specific configuration (R or S) is not |
| | known. |

### II. Intermediates

### Intermediate 1-1.

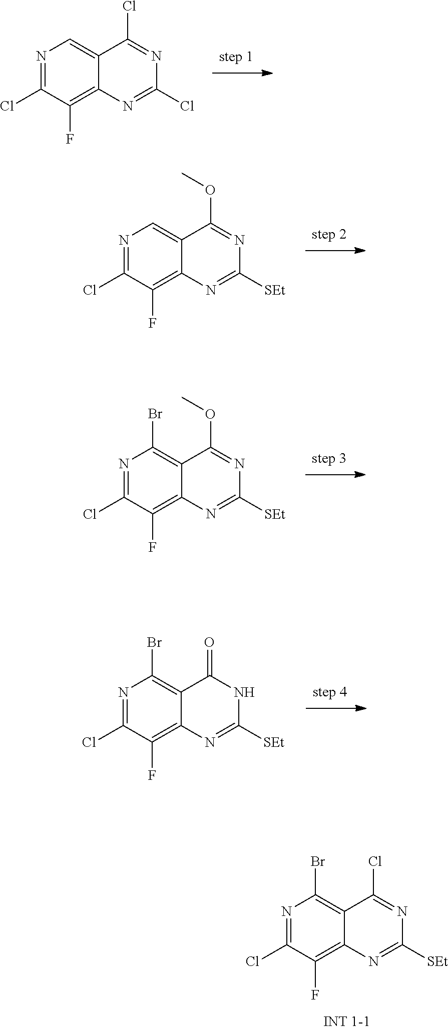

Step 1: 7-chloro-2-(ethylthio)-8-fluoro-4-methoxypyrido[4,3-d]pyrimidine. Sodium methoxide solution (25% wt in methanol, 20 mmol) was added over 15 min via syringe pump to a vigorously stirred solution of 2,4,7-trichloro-8-fluoropyrido[4,3-d]pyrimidine (19.8 mmol) in 2-methyltetrahydrofuran (70 mL) at −20° C. After 11 min, ethanethiol (59.5 mmol) was added over 1 min via syringe. After 1 min, N,N-diisopropylethylamine (63.5 mmol) was added over 2 min via syringe. After 11 min, the resulting mixture was warmed to room temperature. After 20 min, the resulting mixture was heated to 70° C. After 22 h, the resulting mixture was cooled to room temperature, and citric acid (3.0 g), diethyl ether (200 mL), and ethyl acetate (25 mL) were added sequentially. The organic layer was washed with water (200 mL), was dried over anhydrous magnesium sulfate, was filtered, and was concentrated under reduced pressure. The residue was purified by flash column chromatography on silica gel (0% to 11% ethyl acetate in hexanes) to afford the title compound. LCMS: 274.0.

Step 2: 5-bromo-7-chloro-2-(ethylthio)-8-fluoro-4-methoxypyrido[4,3-d]pyrimidine. 2,2,6,6-Tetramethylpiperidinylmagnesium chloride lithium chloride complex solution (1.0 M in tetrahydrofuran, 14 mmol) was added over 20 min via syringe pump to a vigorously stirred solution of 7-chloro-2-(ethylthio)-8-fluoro-4-methoxypyrido[4,3-d]pyrimidine (3.65 mmol) in tetrahydrofuran (3.0 mL) at 0° C. After 60 min, a solution of 1,2-dibromo-1,1,2,2-tetrachloroethane (14.6 mmol) in tetrahydrofuran (8.0 mL) was added via syringe. After 120 min, citric acid (5.0 g), diethyl ether (200 mL), and ethyl acetate (25 mL) were added sequentially. The organic layer was washed with water (2×150 mL), was dried over anhydrous magnesium sulfate, was filtered, and was concentrated under reduced pressure. The residue was purified by flash column chromatography on silica gel (0% to 5% ethyl acetate in hexanes) to give the title compound. LCMS: 351.9.

Step 3: 5-bromo-7-chloro-2-(ethylthio)-8-fluoropyrido[4,3-d]pyrimidin-4(3H)-one. Sodium iodide (1.90 g, 12.7 mmol) was added to a vigorously stirred solution of 5-bromo-7-chloro-2-(ethylthio)-8-fluoro-4-methoxypyrido[4,3-d]pyrimidine (895 mg, 2.54 mmol) in acetic acid (12.0 mL) at room temperature, and the resulting mixture was heated to 80° C. After 2.5 h, the resulting mixture was cooled to room temperature, and ethyl acetate (100 mL) and aqueous sodium thiosulfate solution (1.0 M, 2.0 mL) were added sequentially. The organic layer was washed with water (100 mL), was dried over anhydrous magnesium sulfate, was filtered, and was concentrated under reduced pressure to give the title compound. LCMS: 337.9.

Step 4: 5-bromo-4,7-dichloro-2-(ethylthio)-8-fluoropyrido[4,3-d]pyrimidine (INT 1-1). N,N-Diisopropylethylamine (5.08 mmol) was added via syringe to a mixture of 5-bromo-7-chloro-2-(ethylthio)-8-fluoropyrido[4,3-d]pyrimidin-4(3H)-one (2.48 mmol) and phosphorous(V) oxychloride (10 mL) at room temperature, and the resulting mixture was stirred at rt for 15 min before it was concentrated under reduced pressure. The residue was purified by flash column chromatography on silica gel (0 to 13% ethyl acetate in hexanes) to give the title compound. LCMS: 357.9.

### Intermediate 2-1.

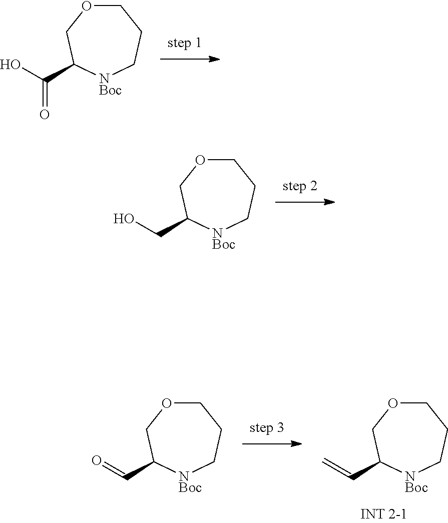 [SMILES: CC(C)(C)OC(=O)N1CCCOC[C@@H]1C(=O)O ; CC(C)(C)OC(=O)N1CCCOC[C@@H]1CO ; CC(C)(C)OC(=O)N1CCCOC[C@@H]1C=O ; C=C[C@H]1COCCCN1C(=O)OC(C)(C)C]

Step 1: tert-butyl (S)-3-(hydroxymethyl)-1,4-oxazepane-4-carboxylate. To a solution of (R)-4-(tert-butoxycarbonyl)-1,4-oxazepane-3-carboxylic acid (10.2 mmol) in anhydrous tetrahydrofuran (25 mL) at −78° C. was added lithium aluminium hydride (10.2 mL, 2M) in tetrahydrofuran. The reaction mixture was stirred at 0° C. for 1 hour. The mixture was diluted with diethyl ether (20 ml), followed by water (0.78 mL), and an aqueous sodium hydroxide solution (2.4 mL, 15 wt. %). The mixture was then allowed to warm up to room temperature and left stirring for 15 minutes. Anhydrous magnesium sulfate was then added with continued stirring for 15 minutes. Filtration was performed to remove the salts, then the filtrate was concentrated under reduced pressure and the crude title compound was taken forward without further purification.

Step 2: tert-butyl (R)-3-formyl-1,4-oxazepane-4-carboxylate. To the solution of tert-butyl (S)-3-(hydroxymethyl)-1,4-oxazepane-4-carboxylate (8.6 mmol) in dichloromethane (10 ml) at 0° C. was added Dess-Martin Periodinane (10 mmol). The mixture was stirred at room temperature for 3 hours, then saturated sodium bicarbonate solution (15 ml) was added to the mixture. The mixture was extracted with ethyl acetate (2×25 ml), the combined organic layers were dried over anhydrous sodium sulfate, and filtered and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 9.68-9.54 (m, 1H), 4.61-4.24 (m, 1H), 4.13 (ddd, J=32.9, 13.4, 6.6 Hz, 1H), 4.04-3.85 (m, 2H), 3.82-3.50 (m, 3H), 1.96-1.78 (m, 2H), 1.48 (d, J=24.7 Hz, 9H).

Step 3: tert-butyl (S)-3-vinyl-1,4-oxazepane-4-carboxylate (INT 2-1). To a suspension of methyltriphenylphosphonium bromide (6.4 mmol) in tetrahydrofuran (10 ml) at room temperature was added a 1.0 M solution of potassium bis(trimethylsilyl)amide (6.1 ml) dropwise to afford a suspension. The mixture was stirred for 1 hour at room temperature and was cooled to −78° C. whereupon a solution of tert-butyl (R)-3-formyl-1,4-oxazepane-4-carboxylate (3.1 mmol) in tetrahydrofuran (2 ml) was added dropwise. The resulting mixture was allowed to gradually warm to room temperature and stir for 3 hours. The mixture was quenched with methanol and stirred for 15 minutes. Saturated aqueous ammonium chloride solution (10 ml) was added, and the mixture was extracted with ethyl acetate (2×25 ml). The combined organic phase was washed with brine and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 5.84-5.57 (m, 1H), 5.16 (q, J=9.4, 8.4 Hz, 2H), 4.75 (d, J=70.6 Hz, 1H), 4.08-3.79 (m, 2H), 3.65-3.31 (m, 2H), 3.07 (t, J=12.9 Hz, 1H), 2.01-1.53 (m, 3H), 1.48 (d, J=10.2 Hz, 9H).

The following intermediates were made in a similar fashion to INT 2-1 and are shown below in Table 2A. To prepare the below intermediates, different reagents/starting materials were used than some of those described in the steps toward INT 2-1 and are noted in the last column of Table 2A—“Changes to Procedure: Different Reagents/Starting Materials”. A person of ordinary skill in the art will readily recognize which reagents/starting materials used for the synthesis of INT 2-1 were replaced with the different reagents/starting materials noted below.

TABLE 2A
Intermediates

| | | | Changes to |
|---|---|---|---|
| | | | Procedure: |
| | | | Different |
| Inter- | | | Reagents/Starting |
| mediate | Structure | NMR | Materials |
| INT 2-2 | 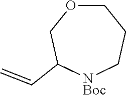 [SMILES: C=CC1COCCCN1C(=O)OC(C)(C)C] | ¹H NMR (400 MHz, Chloroform-d) δ 5.68 (d, J = 14.9 Hz, 1H), 5.16 (d, J = 10.0 Hz, 2H), 4.75 (d, J = 70.3 Hz, 1H), 4.30-3.76 (m, 2H), 3.74-3.24 (m, 2H), 3.07 (t, J = 13.1 Hz, 1H), 1.93 (s, 1H), 1.84-1.51 (m, 2H), 1.55-1.45 (m, 9H). | 4-(tert- butoxycarbonyl)- 1,4-oxazepane-3- carboxylic acid in step 1 |
| INT 2-3 | 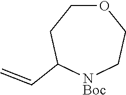 [SMILES: C=CC1CCOCCN1C(=O)OC(C)(C)C] | ¹H NMR (400 MHz, Chloroform-d) δ 5.79 (td, J = 16.5, 8.4 Hz, 1H), 5.15- 5.02 (m, 2H), 4.62 (d, J = 90.5 Hz, 1H), 4.14-3.70 (m, 3H), 3.48 (p, J = 9.9, 9.3 Hz, 2H), 3.06 (ddd, J = 15.2, 10.3, 1.5 Hz, 1H), 2.18 (ddd, J = 22.8, 15.1, 7.3 Hz, | 4-(tert-butyl) 5- methyl 1,4- oxazepane-4,5- dicarboxylate in step 1 |
| | | 1H), 1.86 (dt, J = 15.6, 10.4 Hz, 1H), 1.49 | |
| | | (d, J = 6.8 Hz, 9H). | |
| INT 2-4 | 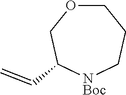 [SMILES: C=C[C@@H]1COCCCN1C(=O)OC(C)(C)C] | ¹H NMR (400 MHz, Chloroform-d) δ 5.65 (d, J = 18.8 Hz, 1H), 5.17 (q, J = 10.5, 7.4 Hz, 2H), 4.76 (d, J = 70.5 Hz, 1H), 4.28-3.80 (m, 2H), 3.67-3.30 (m, 2H), 3.07 (t, J = 13.1 Hz, 1H), 1.93 (s, 1H), 1.79-1.65 (m, 2H), 1.48 (d, J = | (S)-4-(tert- butoxycarbonyl)- 1,4-oxazepane-3- carboxylic acid in step 1 |
| | | 11.6 Hz, 9H). | |
| INT 2-5 | 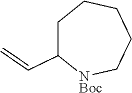 [SMILES: C=CC1CCCCCN1C(=O)OC(C)(C)C] | ¹H NMR (400 MHz, Chloroform-d) δ 5.84-5.63 (m, 1H), 5.09-4.91 (m, 2H), 4.66-4.32 (m, 1H), 3.91-3.61 (m, 1H), 2.68 (ddd, J = 14.5, 11.8, 1.6 Hz, 1H), 2.19-1.94 (m, 1H), 1.88-1.73 (m, 2H), 1.73-1.12 (m, 14H). | Starting with step 2 using tert- butyl 2- (hydroxymethyl) azepane-1- carboxylate |

### Intermediates 2-6-2-9.

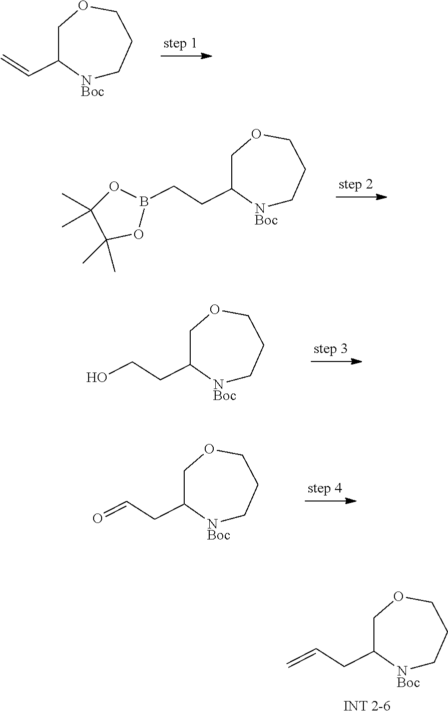 [SMILES: C=CC1COCCCN1C(=O)OC(C)(C)C ; CC(C)(C)OC(=O)N1CCCOCC1CCB1OC(C)(C)C(C)(C)O1 ; CC(C)(C)OC(=O)N1CCCOCC1CCO ; CC(C)(C)OC(=O)N1CCCOCC1CC=O ; C=CCC1COCCCN1C(=O)OC(C)(C)C]

Step 1: tert-butyl 3-(2-(4,4,5,5-tetramethyl-1,3,2-dioxaborolan-2-yl) ethyl)-1,4-oxazepane-4-carboxylate. Tert-butyl 3-vinyl-1,4-oxazepane-4-carboxylate (INT 2-1) (1.9 mmol) was dissolved in 1,2-dichloroethane (10 mL) and the stirred solution was evacuated and refilled with argon (3×). To this solution chloro-1,5-cyclooctadiene iridium(I) dimer (0.29 mmol) was added, followed by 1,2-bis(diphenylphosphino)ethane (0.57 mmol), after which the resulting mixture was evacuated and refilled with argon again (3×). After stirring for 30 minutes at room temperature, the reaction mixture was cooled to 0° C. and a solution of pinacolborane (3.8 mmol) in dichloromethane (2 mL) was added dropwise over 15 minutes. After the addition, the ice bath was removed, and the mixture was stirred for an additional 2 hours at room temperature. The reaction mixture was then quenched with saturated aqueous ammonium chloride solution (5 mL) and the aqueous phase was extracted with dichloromethane (3×20 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 4.32-4.03 (m, 1H), 4.03-3.62 (m, 3H), 3.59-3.20 (m, 2H), 3.06-2.88 (m, 1H), 2.04-1.38 (m, 13H), 1.26 (s, 12H), 0.87-0.70 (m, 2H).

Step 2: tert-butyl 3-(2-hydroxyethyl)-1,4-oxazepane-4-carboxylate. A premixed solution of sodium hydroxide (2 M aqueous)/hydrogen peroxide (30% aqueous) (2:1, 15 mL) was added dropwise to a solution of tert-butyl 3-(2-(4,4,5,5-tetramethyl-1,3,2-dioxaborolan-2-yl) ethyl)-1,4-oxazepane-4-carboxylate (1.9 mmol) in tetrahydrofuran (7.5 mL) at 0° C. After addition the reaction mixture was vigorously stirred at room temperature for 2 hours. The reaction was quenched with aqueous ammonium chloride solution (5 mL). The reaction mixture was extracted with ethyl acetate (2×15 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 4.47 (tdd, J=10.8, 5.8, 2.7 Hz, 1H), 4.09 (ddd, J=12.4, 7.2, 5.0 Hz, 2H), 3.89-3.76 (m, 1H), 3.77-3.57 (m, 1H), 3.57-3.45 (m, 1H), 3.45-3.35 (m, 1H), 3.27 (dd, J=13.4, 10.5 Hz, 1H), 2.94 (ddd, J=14.9, 11.7, 1.4 Hz, 1H), 1.94 (dtdd, J=14.5, 12.0, 5.6, 2.5 Hz, 1H), 1.73-1.43 (m, 11H), 1.35-1.19 (m, 1H).

Step 3: tert-butyl 3-(2-oxoethyl)-1,4-oxazepane-4-carboxylate. To a solution of tert-butyl 3-(2-hydroxyethyl)-1,4-oxazepane-4-carboxylate (1.4 mmol) in dichloromethane (10 mL) at 0° C. was added Dess-Martin Periodinane (1.7 mmol). The mixture was stirred at room temperature for 3 hours. Saturated sodium bicarbonate solution (15 mL) was added to the mixture, the mixture was extracted with ethyl acetate (2×15 mL), the combined organic layers were dried over anhydrous sodium sulfate, and the filtrate was concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 9.77 (d, J=2.4 Hz, 1H), 4.90-4.53 (m, 1H), 4.08-3.69 (m, 3H), 3.64-3.39 (m, 2H), 3.14 (ddd, J=15.1, 10.9, 2.5 Hz, 1H), 2.61-2.48 (m, 2H), 2.02-1.52 (m, 2H), 1.48 (s, 9H).

Step 4: tert-butyl 3-allyl-1,4-oxazepane-4-carboxylate (INT 2-6). To a suspension of methyltriphenylphosphonium bromide (0.94 mmol) in tetrahydrofuran (3 mL) at room temperature was added a 1.0 M solution of potassium bis(trimethylsilyl)amide (0.90 mL) dropwise to afford a suspension. The mixture was stirred for 1 hour at room temperature and was cooled to −78° C. whereupon a solution of tert-butyl 3-(2-oxoethyl)-1,4-oxazepane-4-carboxylate (0.37 mmol) in tetrahydrofuran (1 mL) was added dropwise. The resulting mixture was allowed to gradually warm to room temperature and stirred for 3 hours. The mixture was quenched with methanol and stirred for 15 minutes. Saturated aqueous ammonium chloride solution (5 mL) was added, and the mixture was extracted with ethyl acetate (2×10 mL). The combined organic phase was washed with brine and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 5.78 (tdd, J=14.4, 9.6, 7.3 Hz, 1H), 5.17-4.93 (m, 2H), 4.47-4.12 (m, 1H), 4.10-3.74 (m, 2H), 3.68-3.30 (m, 2H), 3.12-2.96 (m, 1H), 2.23 (dq, J=18.4, 7.4, 6.7 Hz, 2H), 1.91 (dddd, J=14.5, 9.1, 6.1, 3.4 Hz, 1H), 1.78-1.55 (m, 2H), 1.49 (s, 9H).

The following intermediates were made in a similar fashion to INT 2-6 and are shown below in Table 2B. To prepare the below intermediates, different reagents/starting materials were used than some of those described in the steps toward INT 2-6 and are noted in the last column of Table 2B—“Changes to Procedure: Different Reagents/Starting Materials”. A person of ordinary skill in the art will readily recognize which reagents/starting materials used for the synthesis of INT 2-6 were replaced with the different reagents/starting materials noted below.

TABLE 2B
Intermediates

| | | | Changes to |
|---|---|---|---|
| | | | Procedure: |
| | | | Different |
| Inter- | | | Reagents/Starting |
| mediate | Structure | NMR | Materials |
| INT 2-7 | 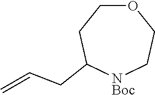 [SMILES: C=CCC1CCOCCN1C(=O)OC(C)(C)C] | ¹H NMR (400 MHz, Chloroform-d) δ 5.79 (dtt, J = 17.2, 10.0, 7.2 Hz, 1H), 5.14- 4.98 (m, 2H), 4.26 (dq, J = 11.2, 6.9 Hz, 0.53H), 4.05 (dt, J = 11.6, 6.6 Hz, 0.44H), 4.00-3.69 (m, 3H), 3.54-3.34 (m, 2H), 3.03 (dddd, J = 15.0, 9.8, 8.1, 1.5 Hz, | INT 2-3 in step 1 |
| | | 1H), 2.36-2.08 (m, 3H), 1.80-1.62 (m, | |
| | | 1H), 1.49 (d, J = 6.3 Hz, 9H). | |
| INT 2-8 | 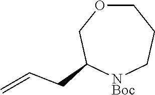 [SMILES: C=CC[C@H]1COCCCN1C(=O)OC(C)(C)C] | ¹H NMR (400 MHz, Chloroform-d) δ 5.94-5.65 (m, 1H), 5.14-4.95 (m, 2H), 4.45-3.79 (m, 3H), 3.67-3.29 (m, 2H), 3.03 (t, J = 12.8 Hz, 1H), 2.23 (dh, J = 20.3, 6.9 Hz, 2H), 1.49 (s, 12H). | INT 2-1 in step 1 |
| INT 2-9 | 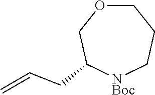 [SMILES: C=CC[C@@H]1COCCCN1C(=O)OC(C)(C)C] | ¹H NMR (400 MHz, Chloroform-d) δ 5.93-5.61 (m, 1H), 5.16-4.92 (m, 2H), 4.43-4.30 (m, 0.42H), 4.26-4.10 (m, 0.66H), 4.11-3.73 (m, 2H), 3.63-3.20 (m, 2H), 3.11-2.96 (m, 1H), 2.35-2.12 (m, 2H), 2.04-1.78 (m, 1H), 1.78-1.54 (m, | INT 2-4 in step 1 |
| | | 2H), 1.49 (s, 9H). | |

### Intermediate 3-1.

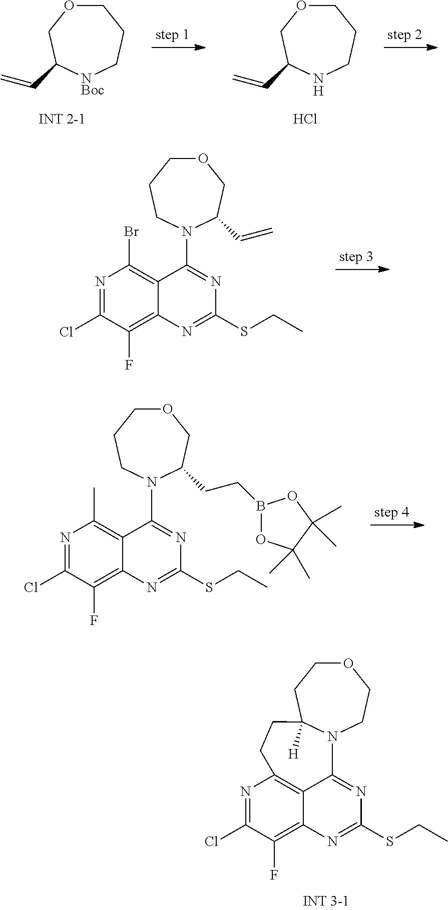

Step 1: (S)-3-vinyl-1,4-oxazepane hydrogen chloride. To a stirred solution of INT 2-1 (1.9 mmol) in dichloromethane (3 ml) was added 4 M hydrochloric acid in 1, 4-dioxane (2.5 ml). Upon completion of the reaction, the mixture was concentrated under reduced pressure and the crude was taken forward without further purification. LCMS: 128.0.

Step 2: ((S)-4-(5-bromo-7-chloro-2-(ethylthio)-8-fluoropyrido[4,3-d]pyrimidin-4-yl)-3-vinyl-1,4-oxazepane. To a stirred mixture of 5-bromo-4,7-dichloro-2-(ethylthio)-8-fluoropyrido[4,3-d]pyrimidine (2.5 mmol) and (S)-3-vinyl-1,4-oxazepane hydrogen chloride (1.9 mmol) in dichloromethane (6 ml) at 0° C. was slowly added N,N-diisopropylethylamine (1.5 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 447.0.

Step 3: (S)-4-(5-bromo-7-chloro-2-(ethylthio)-8-fluoroquinazolin-4-yl)-3-(2-(4,4,5,5-tetramethyl-1,3,2-dioxaborolan-2-yl) ethyl)-1,4-oxazepane. (S)-4-(5-bromo-7-chloro-2-(ethylthio)-8-fluoropyrido[4,3-d]pyrimidin-4-yl)-3-vinyl-1,4-oxazepane (1.9 mmol) was dissolved in 1,2-dichloroethane (10 ml) and the stirred solution was evacuated and refilled with argon (3×). To this solution chloro-1,5-cyclooctadiene iridium(I) dimer (0.28 mmol) was added, followed by 1,2-bis(diphenylphosphino)ethane (0.56 mmol), after which the resulting mixture was evacuated and refilled with argon again (3×). After stirring for 30 minutes at room temperature, the reaction mixture was cooled to 0° C. and a solution of pinacolborane (3.8 mmol) in dichloromethane (2 ml) was added dropwise over 15 minutes. After the addition, the ice bath was removed, and the mixture was stirred for an additional 2 hours at room temperature while monitored by LCMS. The reaction mixture was then quenched with saturated aqueous ammonium chloride solution (5 mL) and the aqueous phase was extracted with dichloromethane (3×20 mL).

The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 574.3.

Step 4: (S)-2-chloro-12-(ethylthio)-1-fluoro-4,5,5a,6,9,10-hexahydro-8H-7-oxa-3,10a,11,13-tetraazanaphtho[1,8-ab]heptalene (INT 3-1). (S)-4-(5-bromo-7-chloro-2-(ethylthio)-8-fluoroquinazolin-4-yl)-3-(2-(4,4,5,5-tetramethyl-1,3,2-dioxaborolan-2-yl) ethyl)-1,4-oxazepane (1.0 mmol) was dissolved in 1,4-dioxane (6 ml) and water (1.2 ml). Sodium carbonate (4.0 mmol) and (1,1′-bis(diphenylphosphino)ferrocene)-dichloropalladium (II) (0.15 mmol) were added and the mixture was degassed and heated at 140° C. After 15 minutes the reaction was complete. The mixture was cooled to room temperature, diluted with ethyl acetate (10 ml) and washed with aqueous ammonium chloride (10 ml). The organic layer was concentrated under reduced pressure. The residue was purified by silica gel chromatography eluting with ethyl acetate/hexanes. Fractions containing the product were pooled and concentrated under reduced pressure to yield the title compound. LCMS: 369.2.

### Intermediate 4-1.

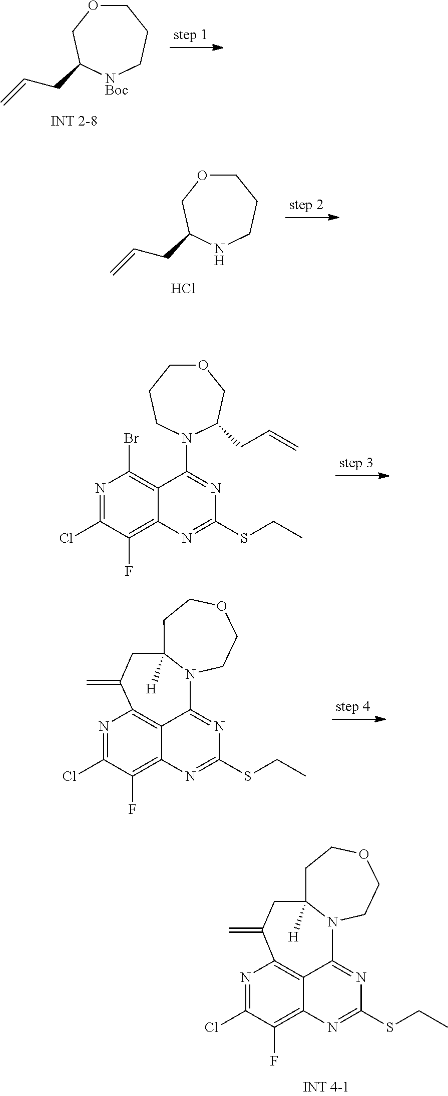

Step 1: (S)-3-allyl-1,4-oxazepane hydrogen chloride. To a stirred solution of tert-butyl (S)-3-allyl-1,4-oxazepane-4-carboxylate (INT 2-8) (7.5 mmol) in dichloromethane (20 ml) was added 4 M HCl in 1, 4-dioxane (5 ml). Upon completion of the reaction, the mixture was concentrated under reduced pressure and the crude was taken forward without further purification. LCMS: 142.1.

Step 2: (S)-3-allyl-4-(5-bromo-7-chloro-2-(ethylthio)-8-fluoropyrido[4,3-d]pyrimidin-4-yl)-1,4-oxazepane. To a stirred mixture of 5-bromo-4,7-dichloro-2-(ethylthio)-8-fluoropyrido[4,3-d]pyrimidine (7.3 mmol) and (S)-3-allyl-1,4-oxazepane hydrogen chloride (7.3 mmol) in dichloromethane (15 ml) at 0° C. was slowly added N,N-diisopropylethylamine (5.7 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 461.2.

Step 3: (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene. (S)-3-allyl-4-(5-bromo-7-chloro-2-(ethylthio)-8-fluoropyrido[4,3-d]pyrimidin-4-yl)-1,4-oxazepane (1.5 mmol) was dissolved in N,N-dimethylformamide (5 ml), and tetrabutylammonium bromide (3.0 mmol), bis(tri-tert-butylphosphine)palladium(0) (0.3 mmol) and triethylamine (0.47 ml) were sequentially added. The mixture was purged with argon and heated at 80° C. LCMS showed complete conversion after 45 min and the reaction was cooled to room temperature. Ethyl acetate and water were added to the mixture. The organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 381.3.

Step 4: (S)-2-chloro-12-(ethylthio)-1-fluoro-4-methyl-5a,6,9,10-tetrahydro-8H-7-oxa-3,10a,11,13-tetraazanaphtho[1,8-ab]heptalene (INT 4-1). To the solution of (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene (1.1 mmol) in toluene (5 ml) was added bis(tri-tert-butylphosphine)palladium (0.1 mmol) and isobutyryl chloride (0.1 mmol). The mixture was purged with argon and heated at 80° C. LCMS showed complete clean conversion after 1 hour. The mixture was cooled to room temperature. Ethyl acetate and water were added to the mixture. The organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 381.3.

### Intermediates 5-1-5-3.

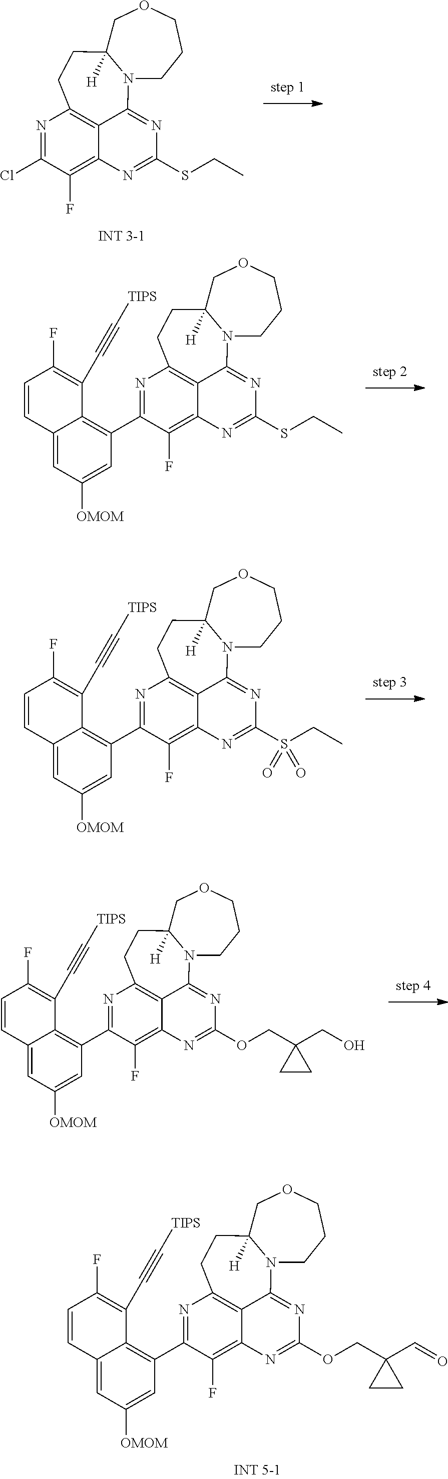

Step 1: (S)-12-(ethylthio)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a solution of (S)-2-chloro-12-(ethylthio)-1-fluoro-4,5,5a,6,9,10-hexahydro-8H-7-oxa-3,10a,11,13-tetraazanaphtho[1,8-ab]heptalene (3.6 mmol) in tetrahydrofuran (55 mL) and water (11 mL) was added 2-[2-fluoro-6-(methoxymethoxy)-8-(4,4,5,5-tetramethyl-1,3,2-dioxaborolan-2-yl)-1-naphthyl]ethynyl-triisopropyl-silane (5.5 mmol), tripotassium phosphate (11.1 mmol) and mesylate[(di(1-adamantyl)-n-butylphosphine)-2-(2′-amino-1,1′-biphenyl)]palladium(II) (0.74 mmol). After the reaction mixture was evacuated and refilled with argon (3×), it was heated to 65° C. for 1 hour. The reaction mixture was cooled to room temperature, diluted with ethyl acetate, then washed with water and a saturated aqueous ammonium chloride solution. Combined organic extracts were washed with brine, dried, filtered and concentrated. The crude product was purified by silica gel column chromatography (0% to 100% ethyl acetate in hexanes) to give the title compound. LCMS: 719.4.

Step 2: (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a stirred solution of (S)-12-(ethylthio)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.97 mmol) in dichloromethane (13 mL) at 0° C. was added 3-chloroperoxybenzoic acid (2.14 mmol) in one portion. After stirring for 30 minutes at room temperature, the reaction mixture was diluted with dichloromethane (30 mL) and washed with a saturated aqueous solution of sodium bicarbonate (10 mL), dried and concentrated to afford the crude product, which was used without purification. LCMS: 751.4.

Step 3: (S)-(1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol. Lithium bis(trimethylsilyl)amide (1.0 M solution in tetrahydrofuran, 3.9 mL) was added dropwise over 5 minutes via syringe to a stirred solution of (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.9 mmol) and cyclopropane-1,1-diyldimethanol (9 mmol) in 2-methyltetrahydrofuran (30 mL) at 0° C. under an argon atmosphere.

After 10 minutes, the resulting mixture was allowed to warm to room temperature. The resulting mixture was stirred at room temperature for 30 minutes. The mixture was then diluted with water (20 mL) and extracted with ethyl acetate (3×25 mL). The organic layer was washed with brine (20 mL), dried over anhydrous sodium sulfate, filtered, and concentrated under reduced pressure to yield the title compound which was used without further purification. LCMS: 759.5.

Step 4: (S)-1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropane-1-carbaldehyde (INT 5-1). To a stirred solution of (S)-(1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol from step 3 in dichloromethane (15 mL) was added Dess-Martin periodinane (1.9 mmol) in one portion. The reaction mixture was stirred for 3 hours. The reaction mixture was diluted with dichloromethane (20 mL), followed by saturated aqueous sodium thiosulfate (10 mL) and saturated aqueous sodium bicarbonate (10 mL). The aqueous layer was extracted with dichloromethane (3×20 mL). The combined organic layers were washed with brine (20 mL), dried over anhydrous sodium sulfate, filtered, and concentrated under reduced pressure. The residue was purified by flash column chromatography on silica (0% to 100% ethyl acetate in hexanes) to yield the title compound. LCMS: 757.4.

The following intermediates were made in a similar fashion to INT 5-1 and are shown below in Table 2C. To prepare the below intermediates, different reagents/starting materials were used than previously described in the steps toward INT 5-1 and are noted in the last column of Table 2C—“Changes to Procedure: Different Reagents/Starting Materials”. A person of ordinary skill in the art will readily recognize which reagents/starting materials used for the synthesis of INT 5-1 were replaced with the different reagents/starting materials noted below.

TABLE 2C
Intermediates

| Inter- | | | Changes to Procedure: Different |
|---|---|---|---|
| mediate | Structure | LCMS | Reagents/Starting Materials |
| INT 5-2 | 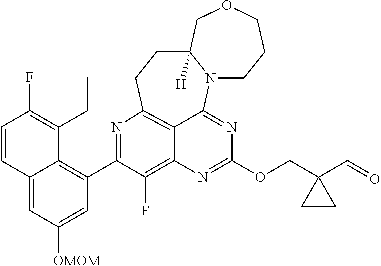 | 605.3 | 2-(8-ethyl-7-fluoro-3- (methoxymethoxy)naphthalen-1-yl)- 4,4,5,5-tetramethyl-1,3,2- dioxaborolane in step 1. |
| INT 5-3 | 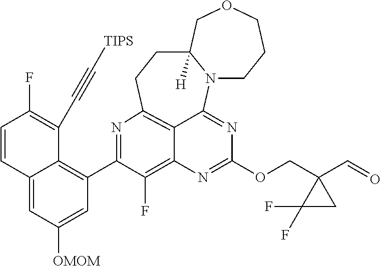 | 793.2 | (2,2-Difluorocyclopropane-1,1- diyl)dimethanol in step 3. |

### Intermediate 5-4.

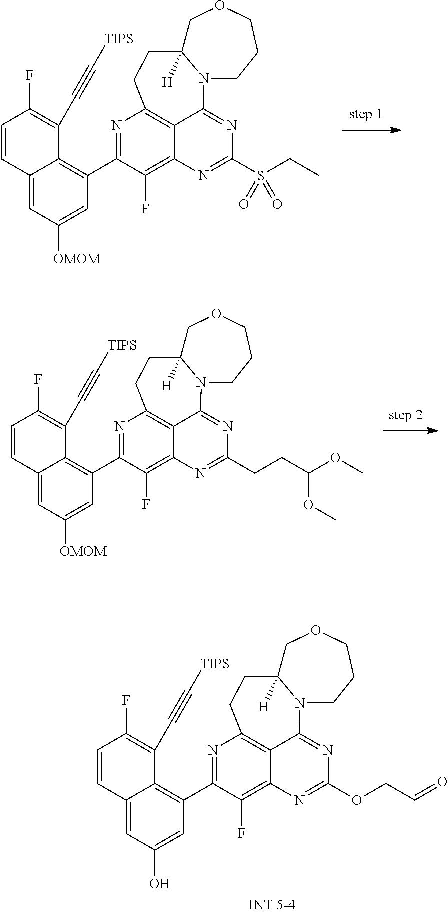

Step 1: (S)-12-(2,2-dimethoxyethoxy)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. Lithium bis(trimethylsilyl)amide (1.0 M solution in tetrahydrofuran, 1.2 mL) was added dropwise over 5 minutes via syringe to a stirred solution of (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.4 mmol) and 2,2-dimethoxyethanol (4 mmol) in 2-methyltetrahydrofuran (4.1 mL) at 0° C. under an argon atmosphere. The resulting mixture was stirred for 15 minutes and then diluted with water and extracted with ethyl acetate. The organic layer was washed with brine, dried over anhydrous sodium sulfate, filtered, and concentrated under reduced pressure to yield the title compound which was used without further purification. LCMS: 763.5.

Step 2: (S)-2-((1-fluoro-2-(7-fluoro-3-hydroxy-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)acetaldehyde (INT 5-4). To a solution of (S)-12-(2,2-dimethoxyethoxy)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.3 mmol) in acetone (1.0 mL) was added HCl (8.3 mmol, 12 M) and the reaction mixture was stirred at room temperature for 30 minutes. The reaction mixture was then diluted with water and ethyl acetate and the aqueous layer was extracted with ethyl acetate. The combined organic layers were washed with brine, dried over anhydrous sodium sulfate, filtered, and concentrated. The residue was purified by silica gel chromatography eluting with ethyl acetate/hexane to yield the title compound. LCMS: 673.4.

Intermediate 5-5a-5-5b.

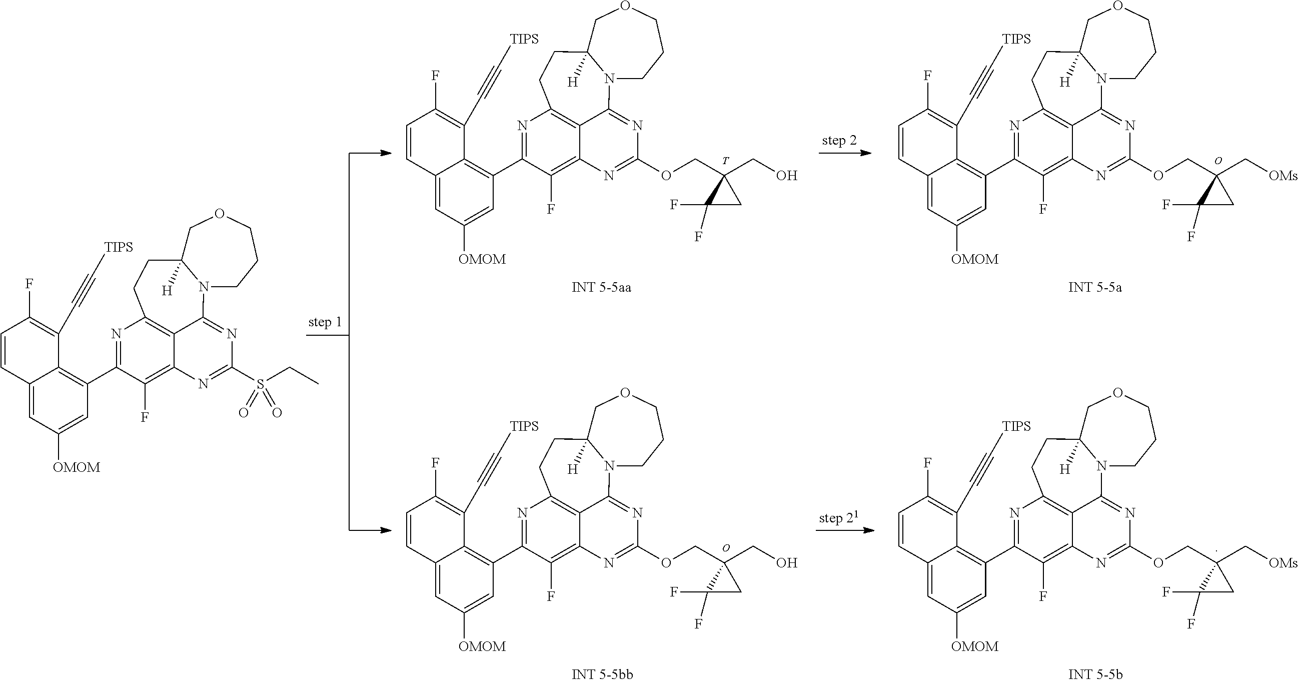

Step 1: (2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol. Lithium bis(trimethylsilyl)amide (1.0 M solution in tetrahydrofuran, 1.9 mL) was added dropwise over 5 minutes via syringe to a stirred solution of (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (1.3 mmol) and [2,2-difluoro-1-(hydroxymethyl)cyclopropyl]methanol (1.6 mmol) in 2-methyltetrahydrofuran (5 mL) at 0° C. under an argon atmosphere. After 10 minutes, the resulting mixture was allowed to warm to room temperature. The resulting mixture was stirred at room temperature for 30 minutes. The mixture was then diluted with water (20 mL) and extracted with ethyl acetate (3×25 mL). The organic fractions were washed with brine (20 mL), dried over anhydrous sodium sulfate, filtered, and concentrated under reduced pressure. The residue was purified by flash column chromatography on silica gel eluting with ethyl acetate in hexanes to afford the title compound as a mixture of two enantiomers which were separated by chiral SFC (IA 4.6×100 mm 5 mic column and IPA-NH₃ as a co-solvent) to give two enantiomers, which were arbitrarily assigned as INT 5-5aa and INT 5-5bb.

INT 5-5aa: ((R)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol. LCMS: 795.2.

INT 5-5bb: ((S)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol. LCMS: 795.2.

Step 2: ((S)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl methanesulfonate (INT 5-5a) To a stirred solution of ((R)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methanol (0.08 mmol) and triethylamine (0.2 mmol) in DCM (1 mL) at 0° C., methanesulfonyl chloride (0.09 mmol) was added. The resulting solution was stirred at room temperature for 2 hours. The reaction mixture was then quenched with water (10 mL) and subsequently extracted with ethyl acetate (3×10 mL). The organic fractions were combined, washed with brine, dried over anhydrous sodium sulfate, filtered and concentrated to dryness under reduced pressure to provide the title compound which was used without further purification. LCMS: 873.2.

((R)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl methanesulfonate (INT 5-5b) prepared analogously to INT 5-5a, substituting INT 5-5aa in step 2 with INT 5-5bb. LCMS: 873.1

### Intermediates 6-1-6-5.

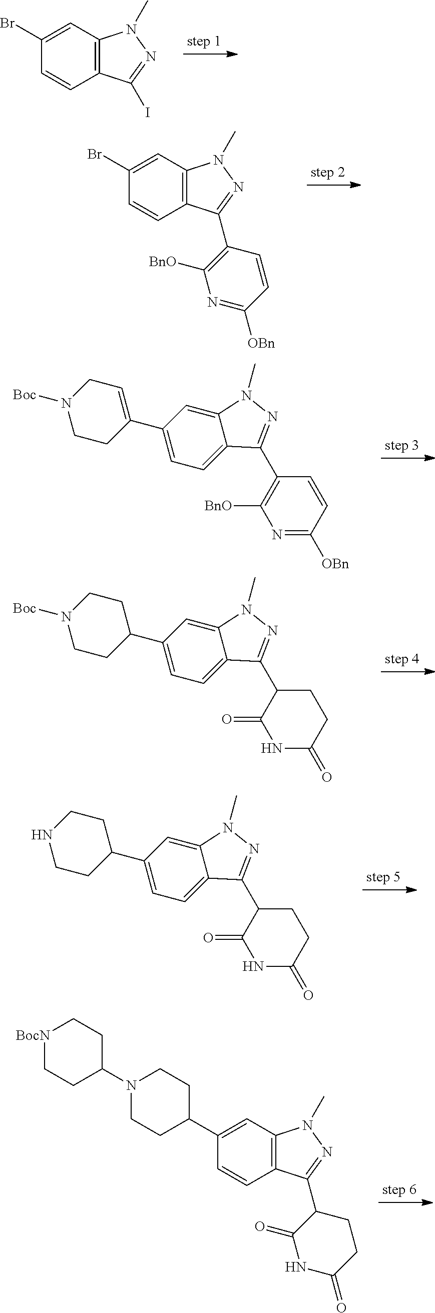

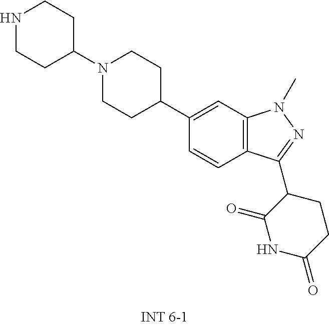 [SMILES: Cn1nc(C2CCC(=O)NC2=O)c2ccc(C3CCN(C4CCNCC4)CC3)cc21]

Step 1: 3-(2,6-bis(benzyloxy)pyridin-3-yl)-6-bromo-1-methyl-1H-indazole. A mixture of 6-bromo-3-iodo-1-methyl-1H-indazole (3.0 mmol), 2,6-bis(benzyloxy)-3-(4,4,5,5-tetramethyl-1,3,2-dioxaborolan-2-yl)pyridine (3.3 mmol), cesium carbonate (8.9 mmol), and [1,1′-bis(diphenylphosphino)ferrocene]dichloropalladium(II) (0.2 mmol) in 1,4-dioxane (12 mL) and water (1.5 mL) was degassed with argon for 1 minute. The reaction mixture was stirred at 80° C. for 12 hours under an argon atmosphere. The reaction mixture was then filtered through Celite and the filtrate was concentrated under reduced pressure. The residue was purified by flash column chromatography on silica gel eluting with ethyl acetate/hexanes to afford the title compound. LCMS: 347.8.

Step 2. tert-butyl 4-(3-(2,6-bis(benzyloxy)pyridin-3-yl)-1-methyl-1H-indazol-6-yl)-3,6-dihydropyridine-1(2H)-carboxylate. To a stirred solution of 3-(2,6-bis(benzyloxy)pyridin-3-yl)-6-bromo-1-methyl-1H-indazole (20 mmol) and tert-butyl 4-(4,4,5,5-tetramethyl-1,3,2-dioxaborolan-2-yl)-3,6-dihydropyridine-1(2H)-carboxylate (30 mmol) in a mixture of 1,4-dioxane and water (100 mL, 3:1) was added potassium carbonate (40 mmol) at room temperature. The reaction was degassed with nitrogen for 30 minutes. [1,1′-bis(diphenylphosphino)ferrocene]dichloropalladium(II) (2.0 mmol) was added to the reaction mixture and it was degassed with nitrogen for 10 minutes and heated to 100° C. for 12 hours. After completion, the reaction mixture was cooled to room temperature and filtered through a pad of Celite and washed with ethyl acetate. The filtrate was diluted with water and extracted with ethyl acetate. The combined organic fractions were washed with brine, dried over sodium sulfate, filtered, and concentrated under reduced pressure to yield the title compound, which was used in the next step without purification. LCMS: 603.3.

Step 3: tert-butyl 4-(3-(2,6-dioxopiperidin-3-yl)-1-methyl-1H-indazol-6-yl)piperidine-1-carboxylate. To a stirred solution of tert-butyl 4-(3-(2,6-bis(benzyloxy)pyridin-3-yl)-1-methyl-1H-indazol-6-yl)-3,6-dihydropyridine-1(2H)-carboxylate (17.4 mmol) in ethyl acetate (105 mL) was added palladium on carbon (12 mmol, 10 wt % Pd) at room temperature. The reaction mixture was stirred in an autoclave under a hydrogen atmosphere (90 psi) for 12 hours at room temperature. After completion, the reaction mixture was cooled to room temperature and filtered through a pad of Celite and washed with ethyl acetate. The combined organic fractions were concentrated under reduced pressure to yield the title compound, which was triturated with diethyl ether and used in the next step.

Step 4: 3-(1-methyl-6-(piperidin-4-yl)-1H-indazol-3-yl)piperidine-2,6-dione. To a stirred solution of tert-butyl 4-(3-(2,6-dioxopiperidin-3-yl)-1-methyl-1H-indazol-6-yl)piperidine-1-carboxylate (11.7 mmol) in dichloromethane (36 mL) was added hydrochloric acid in 1,4-dioxane (11 mL, 4M) at 0° C. for 12 hours. After completion, the reaction mixture was concentrated under reduced pressure to yield the title compound, which was used in the next step without purification. LCMS: 327.3.

Step 5: tert-Butyl 4-(3-(2,6-dioxopiperidin-3-yl)-1-methyl-1H-indazol-6-yl)-[1,4′-bipiperidine]-1′-carboxylate. To a stirring suspension of 3-(1-methyl-6-(piperidin-4-yl)-1H-indazol-3-yl)piperidine-2,6-dione hydrochloride (0.77 mmol), tert-butyl 4-oxopiperidine-1-carboxylate (7.7 mmol), sodium acetate (3.8 mmol) in methanol (4 mL), and N,N-dimethylformamide (1 mL) was added acetic acid (3.8 mmol) followed by sodium cyanoborohydride (7.7 mmol). The resulting mixture was heated to 50° C. After 3 hours, the reaction was cooled to room temperature and concentrated under reduced pressure. The residue was purified by flash column chromatography on silica gel (eluting with methanol in dichloromethane) to afford the title compound. LCMS: 510.0.

Step 6: 3-(6-([1,4′-bipiperidin]-4-yl)-1-methyl-1H-indazol-3-yl)piperidine-2,6-dione (INT 6-1). Trifluoracetic acid (9.1 mmol) was added to a stirring solution of tert-butyl 4-(3-(2,6-dioxopiperidin-3-yl)-1-methyl-1H-indazol-6-yl)-[1,4′-bipiperidine]-1′-carboxylate (0.46 mmol) in dichloromethane (4.0 mL) and the mixture was stirred at room temperature. After 30 minutes, a 4:1 mixture of dichloromethane:isopropanol and aqueous saturated sodium bicarbonate solution were added sequentially. The organic phase was collected and the aqueous phase was washed with 4:1 dichloromethane:isopropanol (2×). The combined organic layers were dried over anhydrous magnesium sulfate, filtered, and concentrated under reduced pressure to give the title compound without further purification. LCMS: 410.0.

The following intermediates were made in a similar fashion to INT 6-1 and are shown below in Table 2D. To prepare the below intermediates, different reagents/starting materials were used than previously described in the steps toward INT 6-1 and are noted in the last column of Table 2D—“Changes to Procedure: Different Reagents/Starting Materials”. A person of ordinary skill in the art will readily recognize which reagents/starting materials used for the synthesis of INT 6-1 were replaced with the different reagents/starting materials noted below.

TABLE 2D
Intermediates

| Inter- | | | Changes to Procedure: Different |
|---|---|---|---|
| mediate | Structure | LCMS | Reagents/Starting Materials |
| INT 6-2 | 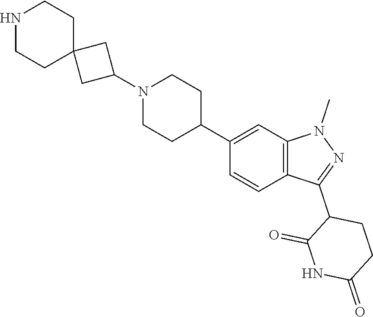 [SMILES: Cn1nc(C2CCC(=O)NC2=O)c2ccc(C3CCN(C4CC5(CCNCC5)C4)CC3)cc21] | 450.3 | tert-butyl 2-oxo-7- azaspiro[3.5]nonane-7-carboxylate in step 5. Hydrochloric acid in 1,4- dioxane in step 6. |
| INT 6-3 | 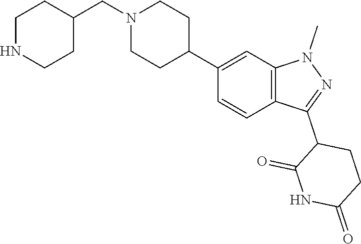 [SMILES: Cn1nc(C2CCC(=O)NC2=O)c2ccc(C3CCN(CC4CCNCC4)CC3)cc21] | 424.3 | tert-butyl 4-formylpiperidine-1- carboxylate in step 5. Hydrochloric acid in 1,4-dioxane in step 6. |
| INT 6-4 | 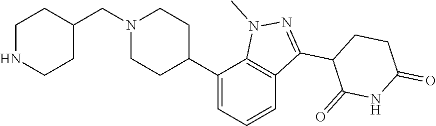 [SMILES: Cn1nc(C2CCC(=O)NC2=O)c2cccc(C3CCN(CC4CCNCC4)CC3)c21] | 424.2 | 7-bromo-3-iodo-1-methyl-indazole in step 1. Cesium carbonate in step 2. H₂ (15 Psi), EtOAc/DMF (2:1) in step 3. tert-butyl 4- formylpiperidine-1-carboxylate, triethylamine, acetic acid, room temperature in step 5. Hydrochloric acid in 1,4-dioxane in step 6. |
| INT 6-5 | 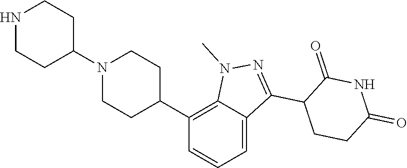 [SMILES: Cn1nc(C2CCC(=O)NC2=O)c2cccc(C3CCN(C4CCNCC4)CC3)c21] | 410.2 | 7-bromo-3-iodo-1-methyl-indazole in step 1. Cesium carbonate in step 2. H₂ (15 Psi), EtOAc/DMF (2:1) in step 3. tert-butyl 4-oxopiperidine-1- carboxylate, diisopropylethylamine, dichloroethane, 60° C. in step 5. Hydrochloric acid in 1,4-dioxane in step 6. |

### Intermediate 7-1.

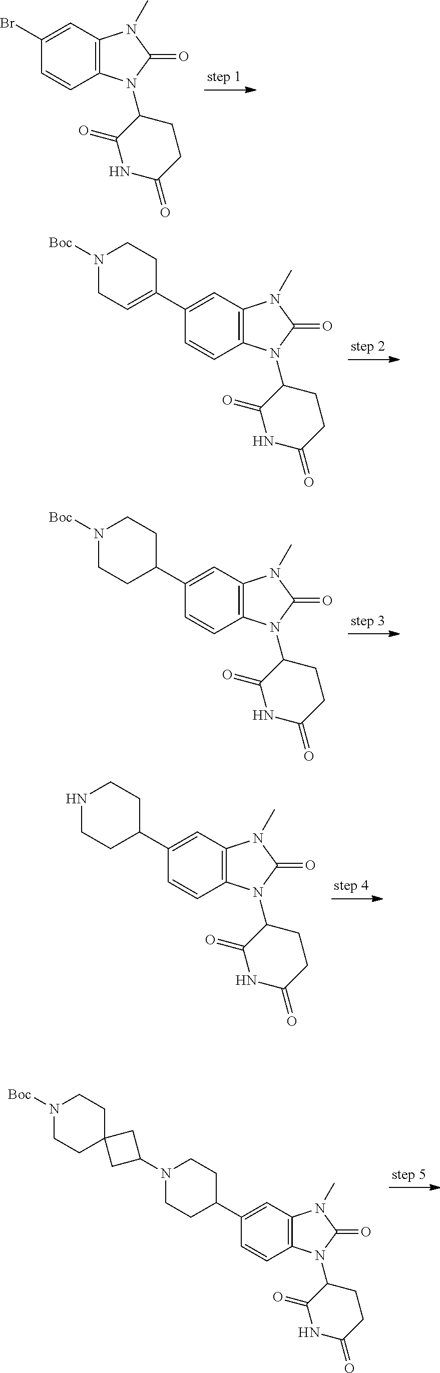 [SMILES: Cn1c(=O)n(C2CCC(=O)NC2=O)c2ccc(Br)cc21 ; Cn1c(=O)n(C2CCC(=O)NC2=O)c2ccc(C3=CCN(C(=O)OC(C)(C)C)CC3)cc21 ; Cn1c(=O)n(C2CCC(=O)NC2=O)c2ccc(C3CCN(C(=O)OC(C)(C)C)CC3)cc21 ; Cn1c(=O)n(C2CCC(=O)NC2=O)c2ccc(C3CCNCC3)cc21 ; Cn1c(=O)n(C2CCC(=O)NC2=O)c2ccc(C3CCN(C4CC5(CCN(C(=O)OC(C)(C)C)CC5)C4)CC3)cc21]

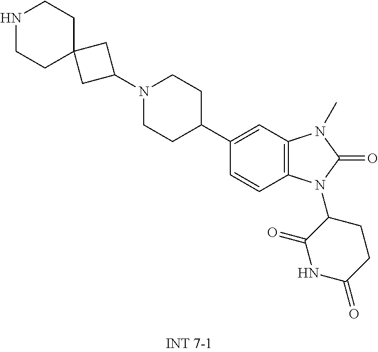 [SMILES: Cn1c(=O)n(C2CCC(=O)NC2=O)c2ccc(C3CCN(C4CC5(CCNCC5)C4)CC3)cc21]

Step 1. tert-butyl 4-(1-(2,6-dioxopiperidin-3-yl)-3-methyl-2-oxo-2,3-dihydro-1H-benzo[d]imidazol-5-yl)-3,6-dihydropyridine-1(2H)-carboxylate. To a stirred solution of 3-(5-bromo-3-methyl-2-oxo-2,3-dihydro-1H-benzo[d]imidazol-1-yl)piperidine-2,6-dione (9 mmol) and tert-butyl 4-(4,4,5,5-tetramethyl-1,3,2-dioxaborolan-2-yl)-3,6-dihydropyridine-1(2H)-carboxylate (13 mmol) in a mixture of 1,4-dioxane and water (35 mL, 6:1) was added potassium phosphate tribasic (17 mmol) at room temperature. The reaction was degassed with nitrogen for 30 minutes. Chloro(2-dicyclohexylphosphino-2′,4′,6′-triisopropyl-1,1′-biphenyl)[2-(2′-amino-1,1′-biphenyl)]palladium (II) (4.4 mmol) was added to the reaction mixture and it was degassed with nitrogen for 10 minutes and heated to 80° C. for 2 hours. After completion, the reaction mixture was cooled to room temperature and filtered through a pad of Celite and washed with ethyl acetate. The filtrate was diluted with water and extracted with ethyl acetate. The combined organic fractions were washed with brine, dried over sodium sulfate, filtered, and concentrated under reduced pressure to yield the title compound, which was used in the next step without purification. LCMS: 441.2.

Step 2: tert-butyl 4-(1-(2,6-dioxopiperidin-3-yl)-3-methyl-2-oxo-2,3-dihydro-1H-benzo[d]imidazol-5-yl)piperidine-1-carboxylate. To a stirred solution of tert-butyl 4-(1-(2,6-dioxopiperidin-3-yl)-3-methyl-2-oxo-2,3-dihydro-1H-benzo[d]imidazol-5-yl)-3,6-dihydropyridine-1(2H)-carboxylate (4.5 mmol) in tetrahydrofuran (30 mL) was added palladium on carbon (2 g, 10 wt. % Pd) at room temperature. The reaction mixture was stirred under a hydrogen atmosphere (15 psi) for 1 hour at room temperature. After completion, the reaction mixture was cooled to room temperature, filtered through a pad of Celite, and washed with ethyl acetate. The combined organic fractions were concentrated under reduced pressure to yield the title compound, which was directly used in the next step.

Step 3: 3-(3-methyl-2-oxo-5-(piperidin-4-yl)-2,3-dihydro-1H-benzo[d]imidazol-1-yl)piperidine-2,6-dione. To a solution of tert-butyl 4-(1-(2,6-dioxopiperidin-3-yl)-3-methyl-2-oxo-2,3-dihydro-1H-benzo[d]imidazol-5-yl)piperidine-1-carboxylate (2.2 mmol) in dichloromethane (6 mL) was added trifluoroacetic acid (2 mL) and the mixture was stirred for 30 minutes. After completion, the reaction mixture was concentrated and purified by RP-HPLC (1 to 20%, 0.1% trifluoroacetic acid in acetonitrile/0.1% trifluoroacetic acid in water). Fractions containing the product were pooled and lyophilized to afford the title compound. LCMS: 343.1.

Step 4: tert-butyl 2-(4-(1-(2,6-dioxopiperidin-3-yl)-3-methyl-2-oxo-2,3-dihydro-1H-benzo[d]imidazol-5-yl)piperidin-1-yl)-7-azaspiro[3.5]nonane-7-carboxylate. To a mixture of 3-(3-methyl-2-oxo-5-(piperidin-4-yl)-2,3-dihydro-1H-benzo[d]imidazol-1-yl)piperidine-2,6-dione trifluoroacetate (0.7 mmol), and tert-butyl 4-oxopiperidine-1-carboxylate (1.2 mmol) in dichloromethane (5 mL) was added N,N-diisopropylethylamine. Acetic acid was added to adjust the pH to 5, followed by sodium triacetoxyborohydride (1.2 mmol) and the resulting mixture was stirred at room temperature for 12 hours. The reaction mixture was quenched by the addition of sodium bicarbonate solution (10 mL, 1M) and extracted with dichloromethane. The combined filtrate was concentrated under reduced pressure to afford the crude material, which was purified by triturating with ethyl acetate to afford the title compound. LCMS: 566.4.

Step 5: 3-(5-(1-(7-azaspiro[3.5]nonan-2-yl)piperidin-4-yl)-3-methyl-2-oxo-2,3-dihydro-1H-benzo[d]imidazol-1-yl)piperidine-2,6-dione (INT 7-1). Trifluoracetic acid (9.1 mmol) was added to a stirring solution of tert-butyl 2-(4-(1-(2,6-dioxopiperidin-3-yl)-3-methyl-2-oxo-2,3-dihydro-1H-benzo[d]imidazol-5-yl)piperidin-1-yl)-7-azaspiro[3.5]nonane-7-carboxylate (0.7 mmol) in dichloromethane (3.0 mL) and the mixture was stirred at room temperature. After completion, the reaction mixture was concentrated and purified by RP-HPLC (1 to 20%, 0.1% trifluoroacetic acid in acetonitrile/0.1% trifluoroacetic acid in water). Fractions containing the product were pooled and lyophilized to afford the title compound. LCMS: 466.2.

### Intermediates 8-1-8-6.

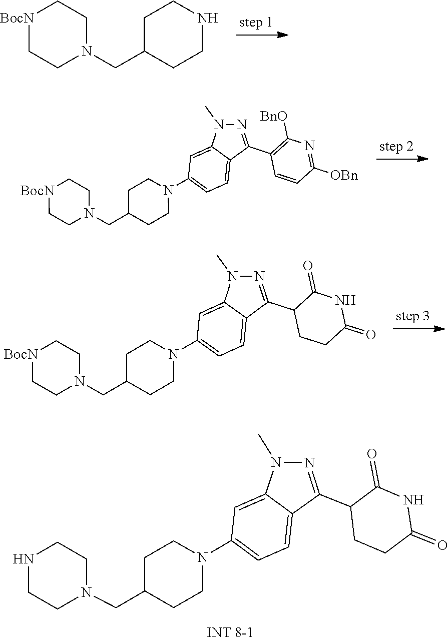 [SMILES: CC(C)(C)OC(=O)N1CCN(CC2CCNCC2)CC1 ; Cn1nc(-c2ccc(OCc3ccccc3)nc2OCc2ccccc2)c2ccc(N3CCC(CN4CCN(C(=O)OC(C)(C)C)CC4)CC3)cc21 ; Cn1nc(C2CCC(=O)NC2=O)c2ccc(N3CCC(CN4CCN(C(=O)OC(C)(C)C)CC4)CC3)cc21 ; Cn1nc(C2CCC(=O)NC2=O)c2ccc(N3CCC(CN4CCNCC4)CC3)cc21]

Step 1: tert-butyl 4-((1-(3-(2,6-bis(benzyloxy)pyridin-3-yl)-1-methyl-1H-indazol-6-yl)piperidin-4-yl)methyl)piperazine-1-carboxylate. A vigorously stirred solution of tert-butyl 4-(piperidin-4-ylmethyl)piperidine-1-carboxylate (0.8 mmol), 6-bromo-3-(2,6-dibenzyloxy-3-pyridyl)-1-methyl-indazole (0.6 mmol), tris(dibenzylideneacetone)dipalladium(0) (0.012 mmol), 2-dicyclohexylphosphino-2′,4′,6′-triisopropylbiphenyl (0.018 mmol), and cesium carbonate (1.8 mmol) in 1,4-dioxane (6 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (100% ethyl acetate) to afford the title compound. LCMS: 702.2.

Step 2: tert-butyl 4-((1-(3-(2,6-dioxopiperidin-3-yl)-1-methyl-1H-indazol-6-yl)piperidin-4-yl)methyl)piperidine-1-carboxylate. A mixture of palladium hydroxide on carbon (2.8 mmol, 20 wt. % Pd) and tert-butyl 4-((1-(3-(2,6-bis(benzyloxy)pyridin-3-yl)-1-methyl-1H-indazol-6-yl)piperidin-4-yl)methyl)piperazine-1-carboxylate (0.55 mmol) in tetrahydrofuran (5 mL) was evacuated and backfilled with hydrogen three times. The reaction mixture was vigorously stirred at room temperature for 12 hours. Upon completion, the solution was filtered through a short pad of Celite, washed with dichloromethane (3×10 mL), then concentrated under reduced pressure. The residue was purified by flash column chromatography on silica gel (0% to 50% ethyl acetate in hexanes) to afford the title compound. LCMS: 524.1.

Step 3: 3-(1-methyl-6-(4-(piperazin-1-ylmethyl)piperidin-1-yl)-1H-indazol-3-yl)piperidine-2,6-dione (INT 8-1). Trifluoroacetic acid (0.5 mL) was added into a solution of tert-butyl 4-((1-(3-(2,6-dioxopiperidin-3-yl)-1-methyl-1H-indazol-6-yl)piperidin-4-yl)methyl)piperidine-1-carboxylate (0.39 mmol) in dichloromethane (5 mL) at room temperature. The resulting solution was stirred at room temperature for 2 hours. Upon completion, the solution was concentrated under reduced pressure and used for the next step without further purification. LCMS: 424.3.

The following intermediates were made in a similar fashion to INT 8-1 and are shown below in Table 2E. To prepare the below intermediates, different reagents/starting materials were used than previously described in the steps toward INT 8-1 and are noted in the last column of Table 2E—“Changes to Procedure: Different Reagents/Starting Materials”. A person of ordinary skill in the art will readily recognize which reagents/starting materials used for the synthesis of INT 8-1 were replaced with the different reagents/starting materials noted below.

TABLE 2E
Intermediates

| Inter- | | | Changes to Procedure: Different |
|---|---|---|---|
| mediate | Structure | LCMS | Reagents/Starting Materials |
| INT 8-2 | 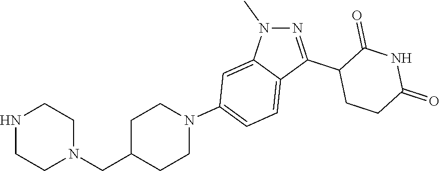 [SMILES: Cn1nc(C2CCC(=O)NC2=O)c2ccc(N3CCC(CN4CCNCC4)CC3)cc21] | 425.3 | tert-butyl 4-(piperidin-4- ylmethyl)piperazine-1-carboxylate in step 1 |
| INT 8-3 | 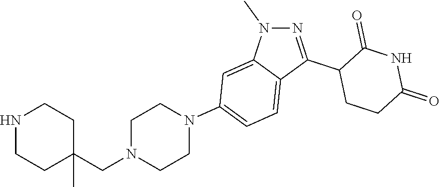 [SMILES: Cn1nc(C2CCC(=O)NC2=O)c2ccc(N3CCN(CC4(C)CCNCC4)CC3)cc21] | 439.3 | tert-butyl 4-methyl-4-(piperazin-1- ylmethyl)piperidine-1-carboxylate in step 1. The final product was treated with Amberlyst A21 ion exchange resin resin after step 3 and isolated as free base. |
| INT 8-4 | 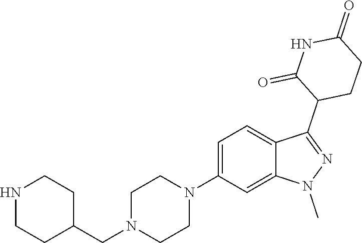 [SMILES: Cn1nc(C2CCC(=O)NC2=O)c2ccc(N3CCN(CC4CCNCC4)CC3)cc21] | 425.3 | tert-butyl 4-((4-methylpiperazin-1- yl)methyl)piperidine-1-carboxylate in step 1. The final product was treated with Amberlyst A21 ion exchange resin resin after step 3 and isolated as free base. |
| INT 8-5 | 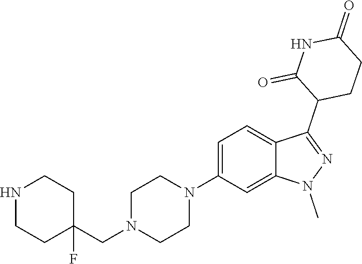 [SMILES: Cn1nc(C2CCC(=O)NC2=O)c2ccc(N3CCN(CC4(F)CCNCC4)CC3)cc21] | 443.4 | INT-10-1.1, sodium tert-butoxide, Pd(OAc)₂, and 2- dicyclohexylphosphino-2′,6′- diisopropoxybiphenyl in step 1. The final product was treated with Amberlyst A21 ion exchange resin resin after step 3 and isolated as free base. |
| INT 8-6 | 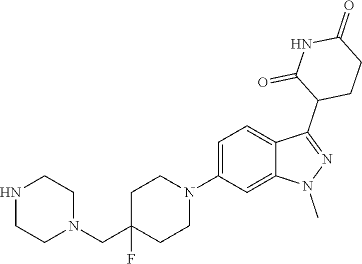 [SMILES: Cn1nc(C2CCC(=O)NC2=O)c2ccc(N3CCC(F)(CN4CCNCC4)CC3)cc21] | 443.3 | INT-10-1.2 and Pd-PEPPSI-IHept- Cl (10 mol %) in step 2. H₂ (15 psi) at 30° C. for 12 hours in step 3. The final product was treated with Amberlyst A21 ion exchange resin resin after step 3 and isolated as free base. |

### Intermediates 9-1-9-2.

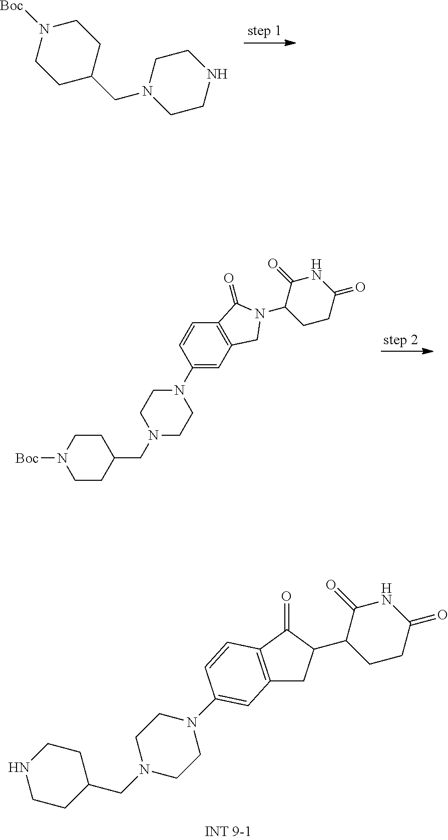 [SMILES: CC(C)(C)OC(=O)N1CCC(CN2CCNCC2)CC1 ; CC(C)(C)OC(=O)N1CCC(CN2CCN(c3ccc4c(c3)CN(C3CCC(=O)NC3=O)C4=O)CC2)CC1 ; O=C1CCC(C2Cc3cc(N4CCN(CC5CCNCC5)CC4)ccc3C2=O)C(=O)N1]

Step 1: tert-butyl 4-(4-(2-(2,6-dioxopiperidin-3-yl)-1-oxoisoindolin-5-yl)piperazin-1-yl)piperidine-1-carboxylate. A vigorously stirred solution of tert-butyl 4-(piperazin-1-ylmethyl)piperidine-1-carboxylate (0.8 mmol), 3-(5-bromo-1-oxo-isoindolin-2-yl)piperidine-2,6-dione (0.6 mmol), dichloro[1,3-bis(2,6-di-4-heptylphenyl)imidazol-2-ylidene](3-chloropyridyl)palladium(II) (0.06 mmol), and cesium carbonate (1.9 mmol) in 1,4-dioxane (6 mL) was heated under argon at 100° C. for 3 hours. The reaction mixture was diluted with dichloromethane and aqueous acetic acid solution (20% by volume) and extracted with dichloromethane. The organic layers were combined, washed with brine (30 mL), dried over anhydrous sodium sulfate, filtered, and concentrated under reduced pressure to afford the title compound which was used in the next step. LCMS: 526.4.

Step 2: 3-[1-oxo-5-[4-(4-piperidylmethyl)piperazin-1-yl]isoindolin-2-yl]piperidine-2,6-dione (INT 9-1). Trifluoroacetic acid (2.5 mL) was added into a solution of tert-butyl 4-(4-(2-(2,6-dioxopiperidin-3-yl)-1-oxoisoindolin-5-yl)piperazin-1-yl)piperidine-1-carboxylate (0.7 mmol) in dichloromethane (2.5 mL) at room temperature. The resulting solution was stirred for 2 hours. Upon completion, the solution was concentrated under reduced pressure and used in the next step without further purification. LCMS: 426.3.

The following intermediates were made in a similar fashion to INT 9-1 and are shown below in Table 2F. To prepare the below intermediates, different reagents/starting materials were used than previously described in the steps toward INT 9-1 and are noted in the last column of Table 2F—“Changes to Procedure: Different Reagents/Starting Materials”. A person of ordinary skill in the art will readily recognize which reagents/starting materials used for the synthesis of INT 9-1 were replaced with the different reagents/starting materials noted below.

TABLE 2F
Intermediates

| Inter- | | | Changes to Procedure: Different |
|---|---|---|---|
| mediate | Structure | LCMS | Reagents/Starting Materials |
| INT 9-2 | 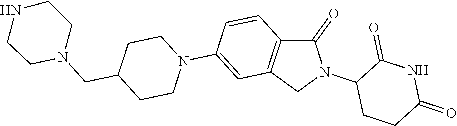 [SMILES: O=C1CCC(N2Cc3cc(N4CCC(CN5CCNCC5)CC4)ccc3C2=O)C(=O)N1] | 426.3 | tert-butyl 4-(piperidin-4- ylmethyl)piperazine-1-carboxylate in step 1 |

### Intermediate 10-1.

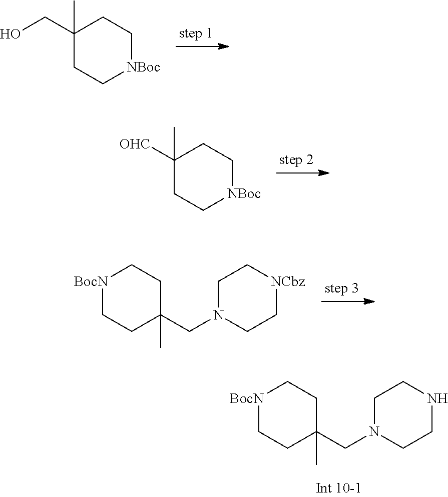 [SMILES: CC1(CO)CCN(C(=O)OC(C)(C)C)CC1 ; CC1(C=O)CCN(C(=O)OC(C)(C)C)CC1 ; CC1(CN2CCN(C(=O)OCc3ccccc3)CC2)CCN(C(=O)OC(C)(C)C)CC1 ; CC1(CN2CCNCC2)CCN(C(=O)OC(C)(C)C)CC1]

Step 1: tert-butyl 4-formyl-4-methylpiperidine-1-carboxylate. To a solution of tert-butyl 4-(hydroxymethyl)-4-methylpiperidine-1-carboxylate (8.7 mmol) in dichloromethane (25 mL) was slowly added pyridinium chlorochromate (26 mmol) at 0° C. The reaction mixture was allowed to warm to room temperature and stirred for 2 hours, then diluted with 50 mL of water. The mixture was extracted with 3×50 mL dichloromethane, then concentrated under reduced pressure. The residue was purified by flash column chromatography on silica gel (0% to 10% ethyl acetate in hexanes) to afford the title compound. ¹H NMR (400 MHz, Chloroform-d) δ 9.46 (s, 1H), 3.70-3.58 (m, 2H), 3.11 (ddd, J=13.5, 9.8, 3.5 Hz, 2H), 1.90 (dddd, J=13.6, 5.2, 3.5, 1.1 Hz, 2H), 1.45 (s, 9H), 1.40 (ddd, J=13.9, 9.8, 4.2 Hz, 2H), 1.08 (s, 3H).

Step 2: benzyl 4-((1-(tert-butoxycarbonyl)-4-methylpiperidin-4-yl)methyl)piperazine-1-carboxylate. To a mixture of tert-butyl 4-formyl-4-methyl-piperidine-1-carboxylate (4.4 mmol), benzyl piperazine-1-carboxylate (4.4 mmol) and acetic acid (4.4 mmol) in 1,2-dichloroethane (10 mL) was slowly added sodium triacetoxyborohydride (8.8 mmol). The reaction mixture was vigorously stirred at room temperature for 48 hours. Upon completion, water (10 mL) and saturated NaHCO₃ solution (10 mL) were added sequentially. The organic layer was separated, dried over anhydrous magnesium sulfate, filtered, and concentrated under reduced pressure. The residue was purified by flash column chromatography on silica gel (0% to 30% ethyl acetate in hexanes) to give the title compound. LCMS: 432.3.

Step 3: tert-butyl 4-methyl-4-(piperazin-1-ylmethyl)piperidine-1-carboxylate (INT 10-1). A mixture of palladium hydroxide on carbon (5.82 mmol, 20 wt % Pd) and benzyl 4-[(1-tert-butoxycarbonyl-4-methyl-4-piperidyl)methyl]piperazine-1-carboxylate (1.9 mmol) in tetrahydrofuran (5 mL) was evacuated and backfilled with hydrogen three times. The reaction mixture was vigorously stirred at room temperature for 12 hours. Upon completion, the solution was filtered through a short pad of Celite, washed with dichloromethane (3×10 mL), concentrated under reduced pressure and used in the next step. LCMS: 298.3.

The following intermediates were made in a similar fashion to INT 10-1 and are shown below in Table 2G. To prepare the below intermediates, different reagents/starting materials were used than previously described in the steps toward INT 10-1 and are noted in the last column of Table 2G—“Changes to Procedure: Different Reagents/Starting Materials”. A person of ordinary skill in the art will readily recognize which reagents/starting materials used for the synthesis of INT 8-1 were replaced with the different reagents/starting materials noted below.

TABLE 2G
Intermediates

| | | | Changes to Procedure: |
|---|---|---|---|
| Inter- | | | Different Reagents/Starting |
| mediate | Structure | LCMS | Materials |
| INT 10-1.1 | 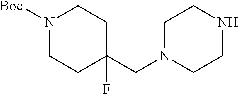 [SMILES: CC(C)(C)OC(=O)N1CCC(F)(CN2CCNCC2)CC1] | 302.1 | tert-butyl 4-fluoro-4- (hydroxymethyl)piperidine-1- carboxylate in step 1 |
| INT 10-1.2 | 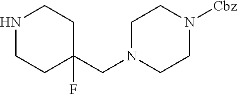 [SMILES: O=C(OCc1ccccc1)N1CCN(CC2(F)CCNCC2)CC1] | 336.1 | Step 3 was omitted. The product from step 2 was dissolved in EtOAc, treated with HCl (4M in dioxane) for 1 hour, concentrated under reduced pressure, and purified |
| | | | by RP-HPLC (0-100%, 0.1% |
| | | | TFA in ACN/H₂O) to afford the |
| | | | title compound. |

### Intermediate 10-2.

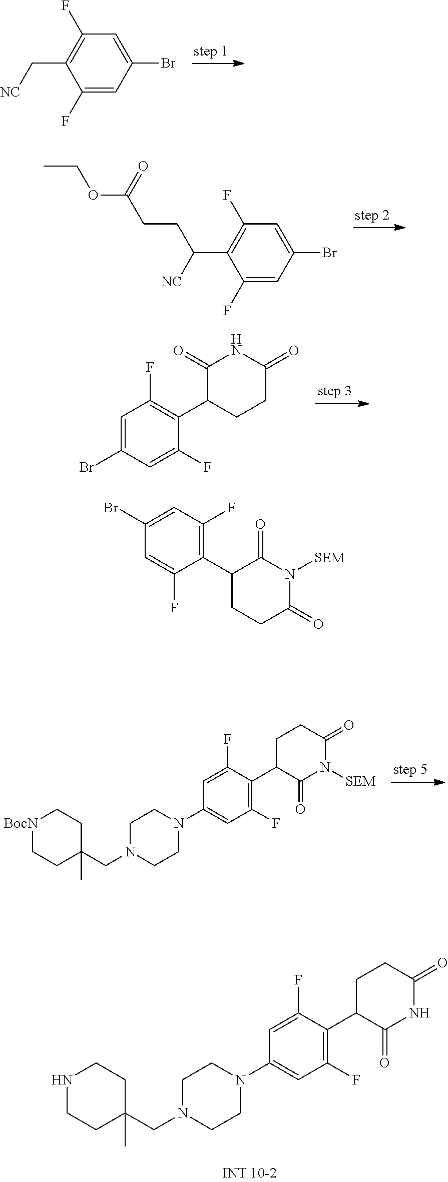

Step 1: Ethyl 4-(4-bromo-2,6-difluorophenyl)-4-cyanobutanoate. To a solution of 2-(4-bromo-2,6-difluorophenyl)acetonitrile (25.8 mmol) and methyl acrylate (25.8 mmol) in tetrahydrofuran (60 mL) was added sodium methoxide (2.5 mmol) at 0° C., and the reaction mixture was stirred at room temperature for 2 hours. The reaction mixture was then diluted with 50 mL of water and extracted with ethyl acetate. The organic layers were combined, washed with brine, dried over anhydrous sodium sulfate, filtered, and concentrated under reduced pressure. The residue was purified by flash column chromatography on silica gel (2% ethyl acetate in hexanes) to afford the title compound. ¹H NMR (400 MHz, DMSO-d₆): δ 7.64-7.61 (m, 1H), 7.60-7.57 (m, 1H), 4.51 (t, 1H), 3.55 (s, 3H), 2.47-2.40 (m, 2H), 2.28-2.16 (m, 1H), 2.14-2.02 (m, 1H).

Step 2: 3-(4-bromo-2,6-difluorophenyl)piperidine-2,6-dione. To a solution of ethyl 4-(4-bromo-2,6-difluorophenyl)-4-cyanobutanoate (18 mmol) in acetic acid (60 mL) was added sulfuric acid (109 mmol) and the reaction mixture was stirred at 90° C. temperature for 2 hours.

The reaction mixture was poured into ice water. The residue was isolated after filtration to afford the title compound. ¹H NMR (400 MHz, DMSO-d₆): δ 11.00 (s, 1H), 7.52 (s, 1H), 7.50 (s, 1H), 4.25 (dd, J=5.1 Hz, J=12.7 Hz, 1H), 2.85-2.74 (m, 1H), 2.55 (t, J=3.2 Hz, 1H), 2.21-2.08 (m, 1H), 2.06-1.95 (m, 1H).

Step 3: 3-(4-bromo-2,6-difluorophenyl)-1-((2-(trimethylsilyl)ethoxy)methyl)piperidine-2,6-dione. To a solution of compound 3-(4-bromo-2,6-difluorophenyl)piperidine-2,6-dione (38 mmol) in tetrahydrofuran (116 mL) was added 1,8-diazabicyclo[5.4.0]undec-7-ene (114.3 mmol) and 2-(trimethylsilyl)ethoxymethyl chloride (107 mmol) at 0° C., and the solution was stirred at room temperature for 3 hours. Upon completion, the reaction mixture was quenched with saturated aqueous sodium bicarbonate solution. The aqueous layer was extracted with ethyl acetate. The organic layers were combined, washed with brine, dried over anhydrous sodium sulfate, filtered, and concentrated under reduced pressure. The residue was purified by flash column chromatography on silica gel (2% ethyl acetate in hexanes) to afford the title compound. ¹H NMR (400 MHz, DMSO-d₆): δ 7.54-7.52 (m, 1H), 7.52-7.49 (m, 1H), 5.09 (s, 2H), 4.41 (dd, J=5.2 Hz, J=12.9 Hz, 1H), 3.55-3.48 (m, 2H), 3.03-2.90 (m, 1H), 2.78-2.69 (m, 1H), 2.21-2.09 (m, 1H), 2.08-1.97 (m, 1H), 0.83 (t, J=8.1 Hz, 2H), −0.01-0.05 (m, 9H).

Step 4: tert-butyl 4-((4-(4-(2,6-dioxo-1-((2-(trimethylsilyl)ethoxy)methyl)piperidin-3-yl)-3,5-difluorophenyl)piperazin-1-yl)methyl)-4-methylpiperidine-1-carboxylate. To a solution of 3-(4-bromo-2,6-difluorophenyl)-1-((2-(trimethylsilyl)ethoxy)methyl)piperidine-2,6-dione (2.3 mmol) and tert-butyl 4-methyl-4-(piperazin-1-ylmethyl)piperidine-1-carboxylate (2.5 mmol) in 1,4-dioxane (19 mL) was added dicyclohexyl[2′,4′,6′-tris(1-methylethyl)[1,1′-biphenyl]-2-yl]phosphine (0.69 mmol) and cesium carbonate (6.9 mmol). The reaction mixture was degassed with nitrogen, then palladium acetate (0.46 mmol) was added and stirred vigorously at 100° C. for 18 hours. The reaction mixture was cooled to room temperature, filtered over Celite and concentrated under reduced pressure. The residue was purified by flash column chromatography on silica gel (50% to 100% ethyl acetate in hexanes) to afford the title compound. LCMS 651.4.

Step 5: 3-(2,6-difluoro-4-(4-((4-methylpiperidin-4-yl)methyl)piperazin-1-yl)phenyl)piperidine-2,6-dione (INT 10-2). To a solution of tert-butyl 4-((4-(4-(2,6-dioxo-1-((2-(trimethylsilyl)ethoxy)methyl)piperidin-3-yl)-3,5-difluorophenyl)piperazin-1-yl)methyl)-4-methylpiperidine-1-carboxylate (1.7 mmol) in dichloromethane (3 mL) was added trifluoroacetic acid (17 mmol) and the mixture was stirred at room temperature for 1 hour. The reaction mixture was then diluted with ethyl acetate and concentrated under reduced pressure. The cycle was repeated until the residual trifluoroacetic acid was removed. The residue was dissolved in dichloromethane (3 mL) and treated with triethylamine (6.9 mmol) and N,N′-dimethylethylenediamine (1.7 mmol). The reaction mixture was stirred at room temperature for 30 minutes, then concentrated under reduced pressure and purified by RP-HPLC (0 to 100%, 0.1% trifluoroacetic acid in acetonitrile/0.1% trifluoroacetic acid in water). Fractions containing the product were pooled and lyophilized. The residue was then neutralized by treating it with a saturated aqueous sodium bicarbonate solution. The aqueous mixture was extracted with a dichloromethane/isopropanol mixture (4:1). The organic fractions were combined, dried over anhydrous sodium sulfate, filtered, and concentrated under reduced pressure to afford the title compound. LCMS 421.3.

### Intermediate 10-3.

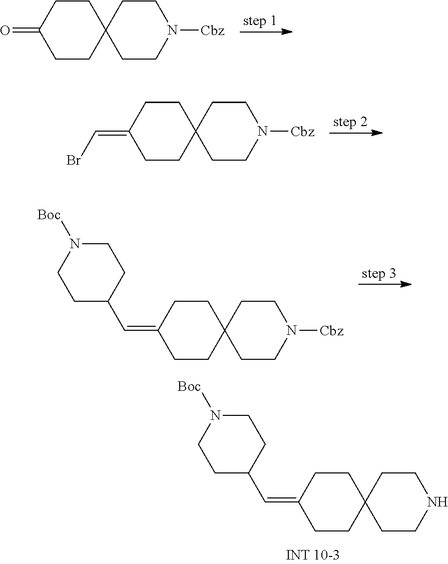

Step 1: Benzyl 9-(bromomethylene)-3-azaspiro[5.5]undecane-3-carboxylate: To a stirred solution of (bromomethyl)triphenyl phosphonium bromide (31.9 mmol) in tetrahydrofuran (160 mL) at 0° C. was added potassium tert-butoxide (79.6 mmol). The reaction mixture was warmed to room temperature and stirred for 30 minutes. Then benzyl 9-oxo-3-azaspiro[5.5]undecane-3-carboxylate (26.5 mmol) was added at room temperature and stirred for 12 hours. The reaction mixture was diluted with water and the aqueous layer was extracted with ethyl acetate. The organic layer was separated, dried over sodium sulfate, filtered, and concentrated under reduced pressure. The residue was purified by silica gel chromatography eluting with 10% ethyl acetate in petroleum ether. Fractions containing the product were pooled and concentrated under reduced pressure to yield the title compound. LCMS: 378.1.

Step 2: Benzyl 9-((1-(tert-butoxycarbonyl)piperidin-4-yl)methylene)-3-azaspiro[5.5]undecane-3-carboxylate: To a stirred solution of benzyl 9-(bromomethylene)-3-azaspiro[5.5]undecane-3-carboxylate (13.2 mmol) in dimethoxyethane (25.0 mL) was added tert-butyl 4-bromopiperidine-1-carboxylate (13.2 mmol) followed by tris(trimethylsilyl)silane (13.2 mmol) and sodium carbonate (26.4 mmol). The mixture was degassed with argon for 15 min. Then, Ir[(ppy]₂(dtbbpy)PF₆ (0.13 mmol), [4,4′-bis(1,1-dimethylethyl)-2,2′-bipyridine]nickel(II) dichloride (0.13 mmol) were added. The reaction mixture was stirred at room temperature under 450 nm LED light for 48 hours. The reaction mixture was diluted with water, and the aqueous layer was extracted with ethyl acetate. The organic layer was washed with brine solution, dried over anhydrous sodium sulfate and concentrated under reduced pressure. The residue was purified by silica gel chromatography eluting with 15% ethyl acetate in petroleum ether to yield the title compound. LCMS: 483.4.

Step 3: tert-butyl 4-((3-azaspiro[5.5]undecan-9-yl)methyl)piperidine-1-carboxylate (INT 10-3): To a stirred solution of benzyl 9-((1-(tert-butoxycarbonyl)piperidin-4-yl)methylene)-3-azaspiro[5.5]undecane-3-carboxylate (6.2 mmol) in tetrahydrofuran (30 mL) was palladium on carbon (2 g, 10 wt. %, wetted with ca. 55% water). The reaction mixture was stirred at room temperature under a H₂ balloon for 48 hours. The reaction mixture was filtered over Celite, and the filter cake was washed with tetrahydrofuran. The filtrate was concentrated under reduced pressure to yield the title compound which was used without further purification. LCMS: 351.4.

### Intermediate 10-4.

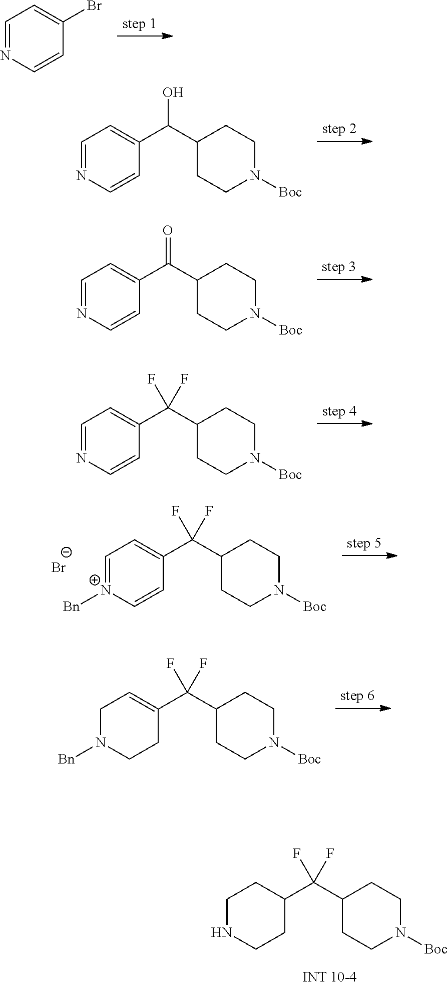

Step 1: tert-butyl 4-(hydroxy(pyridin-4-yl)methyl)piperidine-1-carboxylate. To a solution of 4-bromopyridine (3.2 mmol) in tetrahydrofuran (7.5 mL) was added isopropylmagnesium chloride (4.8 mmol) dropwise at 0° C. The mixture was then stirred at 0° C. for one hour under a nitrogen atmosphere. To the cooled mixture was then added dropwise a solution of tert-butyl 4-formylpiperidine-1-carboxylate (4.8 mmol) in tetrahydrofuran (1.5 mL). The mixture was then gradually warmed to room temperature and stirred for 5 hours under a nitrogen atmosphere. The mixture was then cooled to 0° C., quenched with saturated aqueous ammonium chloride solution (5 mL), and extracted with ethyl acetate (3×5 mL). The combined organic layers was then dried over sodium sulfate, filtered, and concentrated under reduced pressure. The resulting residue was then purified by flash column chromatography on silica gel eluting with ethyl acetate and petroleum ether to afford the title compound. LCMS: 293.1.

Step 2: tert-butyl 4-isonicotinoylpiperidine-1-carboxylate. To a solution of tert-butyl 4-(hydroxy(pyridin-4-yl)methyl)piperidine-1-carboxylate (17 mmol) in dichloromethane (40 mL) was added Dess-Martin Periodinane (26 mmol) at 0° C. The mixture was then warmed to room temperature and stirred for three hours. The mixture was then quenched with saturated aqueous sodium sulfite (15 mL) and sodium bicarbonate (15 mL) solutions and extracted with ethyl acetate (3×30 mL). The combined organic layers were dried over sodium sulfate, filtered, and concentrated under reduced pressure. The resulting residue was then purified by flash column chromatography on silica gel eluting with ethyl acetate and petroleum ether to afford the title compound. LCMS: 291.1.

Step 3: tert-butyl 4-(difluoro(pyridin-4-yl)methyl)piperidine-1-carboxylate. To tert-butyl 4-isonicotinoylpiperidine-1-carboxylate (0.2 mmol) was added diethylaminosulfur trifluoride (3.1 mmol), neat. The reaction mixture was stirred at room temperature for 24 hours under a nitrogen atmosphere. The mixture was then cooled to 0° C., quenched with saturated aqueous sodium bicarbonate solution (1 mL), and extracted with dichloromethane (3×1 mL). The combined organic layers were dried over sodium sulfate, filtered, and concentrated under reduced pressure. The resulting residue was then purified by flash column chromatography on silica gel eluting with ethyl acetate and petroleum ether to afford the title compound. LCMS: 313.1.

Step 4: 1-benzyl-4-((1-(tert-butoxycarbonyl)piperidin-4-yl)difluoromethyl)pyridin-1-ium bromide. To a solution of tert-butyl 4-(difluoro(pyridin-4-yl)methyl)piperidine-1-carboxylate (0.3 mmol) in acetonitrile (0.8 mL) was added (bromomethyl)benzene (0.4 mmol) and the mixture was stirred for one hour at 80° C. under a nitrogen atmosphere. The mixture was then concentrated under reduced pressure to afford the title compound without the need for further purification. LCMS: 403.2.

Step 5: tert-butyl 4-((1-benzyl-1,2,3,6-tetrahydropyridin-4-yl)difluoromethyl)piperidine-1-carboxylate. To a solution of 1-benzyl-4-((1-(tert-butoxycarbonyl)piperidin-4-yl)difluoromethyl)pyridin-1-ium bromide (0.3 mmol) in ethanol (1.2 mL) at 0° C. was added sodium borohydride (1.2 mmol). The mixture was then stirred for five hours at 50° C. under a nitrogen atmosphere. The reaction mixture was then quenched with saturated aqueous sodium bicarbonate solution (2 mL) and extracted with ethyl acetate (3×2 mL). The combined organic layers were dried over sodium sulfate, filtered, and concentrated under reduced pressure to afford the title compound without the need for further purification. LCMS: 407.2.

Step 6: tert-butyl 4-(difluoro(piperidin-4-yl)methyl)piperidine-1-carboxylate (INT 10-4): To a solution of tert-butyl 4-((1-benzyl-1,2,3,6-tetrahydropyridin-4-yl)difluoromethyl)piperidine-1-carboxylate (0.07 mmol) in ethanol (0.3 mL) was added rhodium on alumina (0.14 mmol, 5 wt. % Rh) under an argon atmosphere. The mixture was then evacuated and backfilled with hydrogen three times and stirred at room temperature under a hydrogen atmosphere for 24 hours. The mixture was then filtered, concentrated under reduced pressure, and purified by prep-HPLC (15% to 45%, acetonitrile/0.04% hydrochloric acid in water). Fractions containing the product were pooled and lyophilized to afford the title compound. LCMS: 319.1.

Intermediates 10-5a-10-5b.

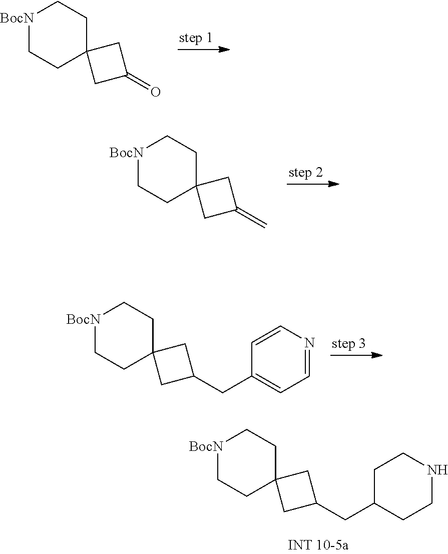

Step 1: tert-butyl 2-methylene-7-azaspiro[3.5]nonane-7-carboxylate. To a solution of methyltriphenylphosphonium bromide (31 mmol) in tetrahydrofuran (50 mL) at 0° C. was added potassium tert-butoxide (42 mmol) and the reaction mixture was stirred for 30 minutes at room temperature. Tert-butyl 2-oxo-7-azaspiro[3.5]nonane-7-carboxylate (21 mmol) was then added in one portion and the resulting mixture was stirred for 12 hours. Upon completion, the mixture was then diluted with water and extracted with ethyl acetate. dried over magnesium sulfate, and concentrated under reduced pressure to give a crude residue. The residue was purified by flash chromatography eluting with a mixture of ethyl acetate and petroleum ether to afford the title compound.

Step 2: tert-butyl 2-(pyridin-4-ylmethyl)-7-azaspiro[3.5]nonane-7-carboxylate. A solution of tert-butyl 2-methylene-7-azaspiro[3.5]nonane-7-carboxylate (21 mmol) and 9-borabicyclo[3.3.1]nonane (50 mL, 0.5 M in THF) was stirred at 65° C. under argon for 2 hours. After cooling the reaction mixture to room temperature, 4-bromopyridine (21 mmol), potassium carbonate (27 mmol) and Pd(dppf)Cl₂·CH₂Cl₂ (1 mmol) were added. The reaction mixture was suspended in DMF (40 mL) and water (10 mL) and then stirred at 70° C. for 12 hours. Upon completion, the mixture was then cooled to room temperature, and filtered through Celite. The filtrate was diluted with water and extracted with ethyl acetate. The combined organic layers were dried over sodium sulfate, filtered, and concentrated under reduced pressure. The residue was purified by flash column chromatography on silica gel (ethyl acetate in hexanes) to afford the title compound. LCMS: 317.3.

Step 3: tert-butyl 2-(piperidin-4-ylmethyl)-7-azaspiro[3.5]nonane-7-carboxylate (INT 10-5a): To a stirred solution of tert-butyl 2-(pyridin-4-ylmethyl)-7-azaspiro[3.5]nonane-7-carboxylate (5 mmol) in ethanol (15 mL) was added acetic acid (0.2 mL) followed by platinum(IV) oxide (0.9 mmol). The reaction mixture was stirred under a hydrogen atmosphere for 12 h at room temperature. The mixture was then filtered through Celite and concentrated under reduced pressure to afford the title compound which was used without further purification. LCMS: 323.0.

The following intermediates were made in a similar fashion to INT 10-5a and are shown below in Table 2H. To prepare the below intermediates, different reagents/starting materials were used than previously described in the steps toward INT 10-5a and are noted in the last column of Table 2H—“Changes to Procedure: Different Reagents/Starting Materials”. A person of ordinary skill in the art will readily recognize which reagents/starting materials used for the synthesis of INT 10-5a were replaced with the different reagents/starting materials noted below.

TABLE 2H
Intermediates

| Inter- | | | Changes to Procedure: Different |
|---|---|---|---|
| mediate | Structure | LCMS | Reagents/Starting Materials |
| INT 10-5b | 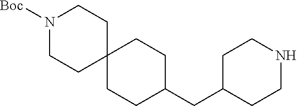 [SMILES: CC(C)(C)OC(=O)N1CCC2(CCC(CC3CCNCC3)CC2)CC1] | 351.4 | tert-butyl 9-oxo-3-azaspiro [5.5]undecane-3-carboxylate in step 1. |

Intermediate 10-6a-10-6b.

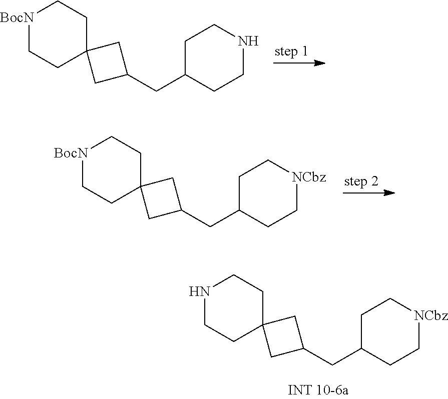 [SMILES: CC(C)(C)OC(=O)N1CCC2(CC1)CC(CC1CCNCC1)C2 ; CC(C)(C)OC(=O)N1CCC2(CC1)CC(CC1CCN(C(=O)OCc3ccccc3)CC1)C2 ; O=C(OCc1ccccc1)N1CCC(CC2CC3(CCNCC3)C2)CC1]

Step 1: tert-butyl 2-((1-((benzyloxy)carbonyl)piperidin-4-yl)methyl)-7-azaspiro[3.5]nonane-7-carboxylate. To a solution of tert-butyl 2-(piperidin-4-ylmethyl)-7-azaspiro[3.5]nonane-7-carboxylate (6 mmol) in THE (17 mL) and water (3 mL) at 0° C. were added sodium bicarbonate (15 mmol) and benzyl chloroformate (6 mmol) and the resulting reaction mixture was stirred at room temperature for 2 hours. The reaction mixture was then quenched with water and extracted with ethyl acetate. The combined organic layers were dried over sodium sulfate, filtered, and concentrated under reduced pressure. The resulting residue product was then purified by flash chromatography eluting with a mixture of ethyl acetate and petroleum ether to afford the title compound.

Step 2: Benzyl 4-((7-azaspiro[3.5]nonan-2-yl)methyl)piperidine-1-carboxylate (INT 10-6a). To a solution of tert-butyl 2-((1-((benzyloxy)carbonyl)piperidin-4-yl)methyl)-7-azaspiro[3.5]nonane-7-carboxylate (4 mmol) in dichloromethane (20 mL) at 0° C. was added trifluoroacetic acid (8 mL) and the resulting reaction mixture was stirred at room temperature for 12 hours. The reaction mixture was then concentrated under reduced pressure and used in the next step without further purification.

The following intermediates were made in a similar fashion to INT 10-6a and are shown below in Table 21. To prepare the below intermediates, different reagents/starting materials were used than previously described in the steps toward INT 10-6a and are noted in the last column of Table 2I—“Changes to Procedure: Different Reagents/Starting Materials”. A person of ordinary skill in the art will readily recognize which reagents/starting materials used for the synthesis of INT 10-6a were replaced with the different reagents/starting materials noted below.

TABLE 2I
Intermediates

| Inter- | | | Changes to Procedure: Different |
|---|---|---|---|
| mediate | Structure | LCMS | Reagents/Starting Materials |
| INT 10-6b | 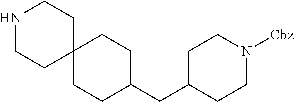 [SMILES: O=C(OCc1ccccc1)N1CCC(CC2CCC3(CCNCC3)CC2)CC1] | 385.4 | tert-butyl 4-((3- azaspiro[5.5]undecan-9- yl)methyl)piperidine-1-carboxylate used in step 1. |

Intermediates 10-7a-10-7b.

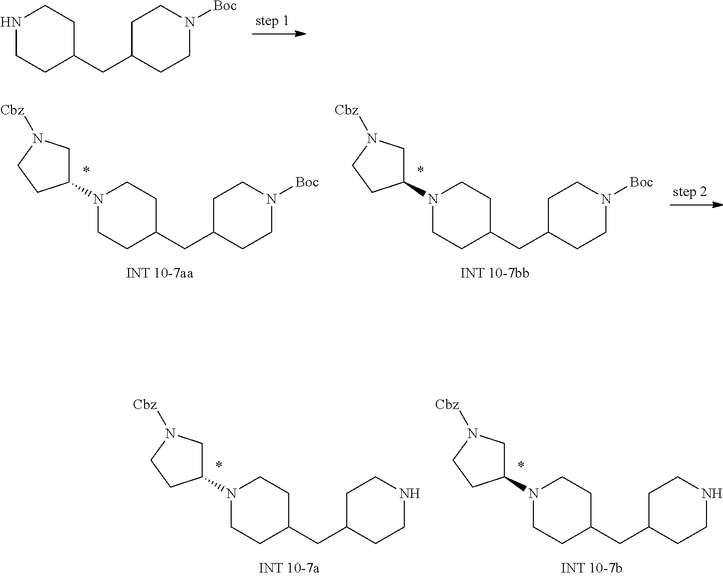

Step-1: Tert-butyl 4-((1-(1-((benzyloxy)carbonyl)pyrrolidin-3-yl)piperidin-4-yl) methyl) piperidine-1-carboxylate: To a solution of benzyl 3-oxopyrrolidine-1-carboxylate (23 mmol) in MeOH (50 mL) was added tert-butyl 4-(piperidin-4-ylmethyl) piperidine-1-carboxylate (25 mmol) and sodium triacetoxy borohydride (46 mmol). The reaction mixture was stirred at room temperature for 16 h. Upon completion, the reaction mixture was quenched with saturated NaHCO₃ solution and extracted with DCM (3×30 mL). The combined organic layers were washed with brine (30 mL), dried over anhydrous sodium sulfate, filtered, and concentrated under reduced pressure. The residue was purified by flash column chromatography on silica (0% to 55% ethyl acetate in hexanes) to afford the title compound as a mixture of two enantiomers which were separated by chiral SFC using CHIRALPAK-AD-H (250×4.6×5p) column with 40% ammonia in methanol as co-solvent to provide two enantiomers which were arbitrarily assigned as INT 10-7aa and INT 10-7bb. INT 10-7a: tert-butyl (R)-4-((1-(1-((benzyloxy)carbonyl)pyrrolidin-3-yl)piperidin-4-yl)methyl)piperidine-1-carboxylate. LCMS: 486.9. INT 10-7b: tert-butyl (S)-4-((1-(1-((benzyloxy)carbonyl)pyrrolidin-3-yl)piperidin-4-yl)methyl)piperidine-1-carboxylate. LCMS: 486.9.

Step 2: Benzyl (R)-3-(4-(piperidin-4-ylmethyl)piperidin-1-yl)pyrrolidine-1-carboxylate (INT 10-7a). 4M HCl in 1,4-dioxane (5 mL) was added into a solution of tert-butyl (R)-4-((1-(1-((benzyloxy)carbonyl)pyrrolidin-3-yl)piperidin-4-yl)methyl)piperidine-1-carboxylate (3.5 mmol) in dichloromethane (10 mL) at 0° C. and the resulting reaction mixture was stirred for 4 hours. Upon completion, the solution was concentrated under reduced pressure. The residue was dissolved in THE and subsequently neutralized with Amberlyst A21 ion exchange resin to provide the title compound. LCMS: 386.3.

Benzyl (R)-3-(4-(piperidin-4-ylmethyl)piperidin-1-yl)pyrrolidine-1-carboxylate (INT 10-7b) was prepared analogously to INT 10-1a, substituting INT 17-1aa in step 2 with INT 17-1bb. LCMS: 386.3.

### Intermediate 11-1.

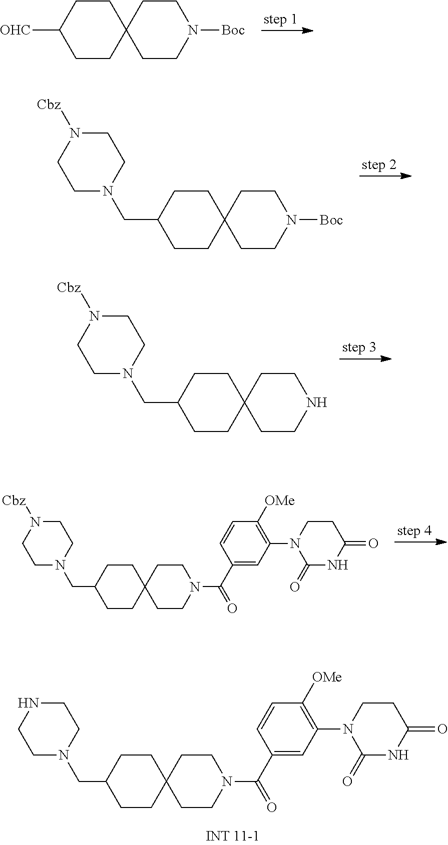 [SMILES: CC(C)(C)OC(=O)N1CCC2(CCC(C=O)CC2)CC1 ; CC(C)(C)OC(=O)N1CCC2(CCC(CN3CCN(C(=O)OCc4ccccc4)CC3)CC2)CC1 ; O=C(OCc1ccccc1)N1CCN(CC2CCC3(CCNCC3)CC2)CC1 ; COc1ccc(C(=O)N2CCC3(CCC(CN4CCN(C(=O)OCc5ccccc5)CC4)CC3)CC2)cc1N1CCC(=O)NC1=O ; COc1ccc(C(=O)N2CCC3(CCC(CN4CCNCC4)CC3)CC2)cc1N1CCC(=O)NC1=O]

Step 1: tert-butyl 9-((4-((benzyloxy)carbonyl)piperazin-1-yl)methyl)-3-azaspiro[5.5]undecane-3-carboxylate. To a mixture of tert-butyl 9-formyl-3-azaspiro[5.5]undecane-3-carboxylate (16 mmol), benzyl piperazine-1-carboxylate (29 mmol) in dichloromethane (54 mL) and methanol (18 mL) was added acetic acid (0.72 mL). The mixture was stirred at room temperature for 30 minutes, cooled to 0° C. and sodium triacetoxyborohydride (37 mmol) was added. The reaction mixture was stirred at 0° C. for 30 minutes then concentrated under reduced pressure. The residue was purified by flash column chromatography on silica gel eluting with 5% methanol in dichloromethane to afford the title compound. LCMS: 486.1.

Step 2: Benzyl 4-((3-azaspiro[5.5]undecan-9-yl)methyl)piperazine-1-carboxylate. To a stirred solution of tert-butyl 9-((4-((benzyloxy)carbonyl)piperazin-1-yl)methyl)-3-azaspiro[5.5]undecane-3-carboxylate (8.2 mmol) in 1,4-dioxane (30 ml) was added 4M hydrochloric acid in 1,4-dioxane (30 mL) and the mixture was stirred for 1 hour. Upon completion of the reaction, the residue was isolated after filtration to afford the title compound. LCMS: 386.4.

Step 3: Benzyl 4-((3-(3-(2,4-dioxotetrahydropyrimidin-1(2H)-yl)-4-methoxybenzoyl)-3-azaspiro[5.5]undecan-9-yl)methyl)piperazine-1-carboxylate. To a mixture of 3-(2,4-dioxotetrahydropyrimidin-1(2H)-yl)-4-methoxybenzoic acid (4.4 mmol) in acetonitrile (80 mL) was added chloro-N,N,N′,N′-tetramethylformamidinium hexafluorophosphate (5.2 mmol), 1-methylimidazole (17.6 mmol), followed by benzyl 4-((3-azaspiro[5.5]undecan-9-yl)methyl)piperazine-1-carboxylate (1.9 g, 4.8 mmol). The mixture was stirred at room temperature for 2 hours then concentrated under reduced pressure. The mixture was purified by flash column chromatography on silica gel eluting with 1% methanol in dichloromethane to afford the title compound. LCMS: 632.1.

Step 4: 1-(2-methoxy-5-(9-(piperazin-1-ylmethyl)-3-azaspiro[5.5]undecane-3-carbonyl)phenyl)dihydropyrimidine-2,4(1H,3H)-dione (INT 11-1). To a stirred solution of benzyl 4-((3-(3-(2,4-dioxotetrahydropyrimidin-1(2H)-yl)-4-methoxybenzoyl)-3-azaspiro[5.5]undecan-9-yl)methyl)piperazine-1-carboxylate (0.08 mmol) in methanol (15 mL) was added palladium on carbon (15 mg) and palladium hydroxide on carbon (15 mg) at room temperature. The reaction mixture was stirred at 50° C. under a hydrogen atmosphere (1.5 MPa) for 16 hours. After completion, the reaction mixture was cooled to room temperature, filtered through a pad of Celite, and concentrated under reduced pressure. The residue was then neutralized by treating it with a saturated aqueous sodium bicarbonate solution. The aqueous mixture was extracted with a dichloromethane/isopropanol mixture (4:1). The organic fractions were combined, dried over anhydrous sodium sulfate, filtered, and concentrated under reduced pressure to afford the title compound. LCMS: 498.4.

### Intermediates 12-1-12-9.

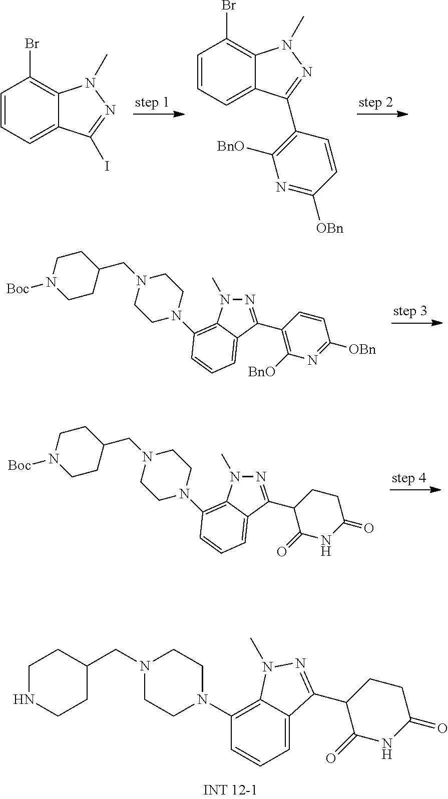 [SMILES: Cn1nc(I)c2cccc(Br)c21 ; Cn1nc(-c2ccc(OCc3ccccc3)nc2OCc2ccccc2)c2cccc(Br)c21 ; Cn1nc(-c2ccc(OCc3ccccc3)nc2OCc2ccccc2)c2cccc(N3CCN(CC4CCN(C(=O)OC(C)(C)C)CC4)CC3)c21 ; Cn1nc(C2CCC(=O)NC2=O)c2cccc(N3CCN(CC4CCN(C(=O)OC(C)(C)C)CC4)CC3)c21 ; Cn1nc(C2CCC(=O)NC2=O)c2cccc(N3CCN(CC4CCNCC4)CC3)c21]

Step 1: 3-(2,6-bis(benzyloxy)pyridin-3-yl)-7-bromo-1-methyl-1H-indazole. To a mixture of 7-bromo-3-iodo-1-methyl-1H-indazole (23.7 mmol) and 2,6-bis(benzyloxy)-3-(4,4,5,5-tetramethyl-1,3,2-dioxaborolan-2-yl)pyridine (23.7 mmol) was added 1,4-dioxane (70.0 mL) and water (10.0 mL). The reaction mixture was degassed with nitrogen for 5 minutes. Cesium carbonate (71 mmol) and [1,1′-bis(diphenylphosphino)ferrocene]dichloropalladium(II) (1.19 mmol) were added under nitrogen. The reaction mixture was then stirred at 95° C. for 16 hours. Water was then added, and the mixture was extracted with ethyl acetate. The organic layers were combined, washed with brine, dried over anhydrous sodium sulfate, filtered, and concentrated under reduced pressure. The residue was purified by silica gel chromatography eluting with 10% ethyl acetate/petroleum ether. Fractions containing the product were pooled and concentrated under reduced pressure to yield the title compound. LCMS: 500.1.

Step 2: tert-butyl 4-((4-(3-(2,6-bis(benzyloxy)pyridin-3-yl)-1-methyl-1H-indazol-7-yl)piperazin-1-yl)methyl)piperidine-1-carboxylate. To a mixture of 3-(2,6-bis(benzyloxy)pyridin-3-yl)-7-bromo-1-methyl-1H-indazole (5.0 mmol) and tert-butyl 4-(piperazin-1-ylmethyl)piperidine-1-carboxylate (5.0 mmol) was added 1,4-dioxane (25.0 mL). The reaction mixture was degassed with nitrogen for 5 minutes. Sodium tert-butoxide (10.5 mmol), 2-dicyclohexylphosphino-2′,6′-diisopropoxybiphenyl (0.35 mmol), and chloro(2-dicyclohexylphosphino-2′,6′-diisopropoxy-1,1′-biphenyl) [2-(2′-amino-1,1′-biphenyl)]palladium(II) (0.17 mmol) were added sequentially under nitrogen gas. The reaction mixture was degassed with nitrogen for 2 minutes, stirred at 100° C. for 16 hours, and then water was added. The mixture was extracted with ethyl acetate. The organic layers were combined, washed with brine, dried over anhydrous sodium sulfate, filtered, and concentrated under reduced pressure. The residue was purified by silica gel chromatography eluting with 40-50% ethyl acetate/petroleum ether. Fractions containing the product were pooled and concentrated under reduced pressure to yield the title compound LCMS: 703.5.

Step 3: tert-butyl 4-((4-(3-(2,6-dioxopiperidin-3-yl)-1-methyl-1H-indazol-7-yl)piperazin-1-yl)methyl)piperidine-1-carboxylate. To a stirred mixture of tert-butyl 4-((4-(3-(2,6-bis(benzyloxy)pyridin-3-yl)-1-methyl-1H-indazol-7-yl)piperazin-1-yl)methyl)piperidine-1-carboxylate (2.4 mmol) in tetrahydrofuran (10 mL) was added palladium hydroxide on carbon, unreduced, 20% palladium, moisture 60% (1.6 g). The reaction vessel was evacuated and backfilled with hydrogen (3×), and the mixture was stirred under a hydrogen atmosphere at room temperature for 16 hours. Upon completion, the mixture was filtered over Celite, rinsed with ethyl acetate, and the filtrate was concentrated under reduced pressure to afford the title compound which was used without further purification. LCMS: 525.5.

Step 4: 3-(1-methyl-7-(4-(piperidin-4-ylmethyl)piperazin-1-yl)-1H-indazol-3-yl)piperidine-2,6-dione. To a stirred solution of tert-butyl 4-((4-(3-(2,6-dioxopiperidin-3-yl)-1-methyl-1H-indazol-7-yl) piperazin-1-yl)methyl)piperidine-1-carboxylate (1.9 mmol) in dichloromethane (10 mL) was added trifluoroacetic acid (19.1 mmol) at room temperature. The reaction mixture was stirred for 3 hours. The reaction mixture was then concentrated under reduced pressure. The crude residue was washed with diethyl ether. The crude compound was then dissolved in tetrahydrofuran:dichloromethane:acetonitrile (1:1:1) mixture and stirred with Amberlyst A21 ion exchange resin for 1 hour. The mixture was filtered, the filter cake rinsed with the same solvent mixture, and the filtrate concentrated under reduced pressure to afford the title compound. LCMS: 425.4.

The following intermediates were made in a similar fashion to INT 12-1 and are shown below in Table 2J. To prepare the below intermediates, different reagents/starting materials were used than previously described in the steps toward INT 12-1 and are noted in the last column of Table 2J—“Changes to Procedure: Different Reagents/Starting Materials”. A person of ordinary skill in the art will readily recognize which reagents/starting materials used for the synthesis of INT 12-1 were replaced with the different reagents/starting materials noted below.

TABLE 2J
Intermediates

| Inter- | | | Changes to Procedure: Different |
|---|---|---|---|
| mediate | Structure | LCMS | Reagents/Starting Materials |
| INT 12-2 | 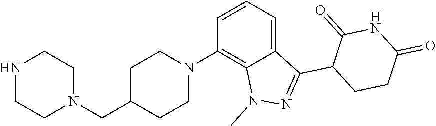 [SMILES: Cn1nc(C2CCC(=O)NC2=O)c2cccc(N3CCC(CN4CCNCC4)CC3)c21] | 425.3 | tert-butyl 4-(piperidin-4- ylmethyl)piperazine-1-carboxylate, and sodium tert-butoxide and palladium (II) acetate in step 2. |
| INT 12-3 | 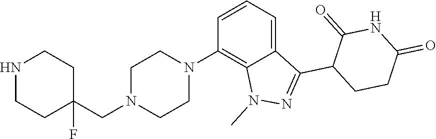 [SMILES: Cn1nc(C2CCC(=O)NC2=O)c2cccc(N3CCN(CC4(F)CCNCC4)CC3)c21] | 443.1 | INT 10-1.1, cesium carbonate, and Pd-PEPPSI-IHept-Cl (10 mol %) in step 2. Palladium on carbon (10 wt. %), H₂ (30 psi) at 30° C. for 12 hours in step 3. |
| INT 12-4 | 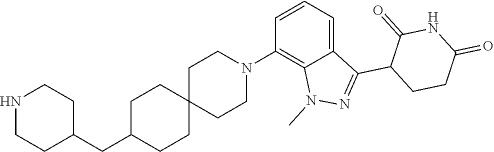 [SMILES: Cn1nc(C2CCC(=O)NC2=O)c2cccc(N3CCC4(CCC(CC5CCNCC5)CC4)CC3)c21] | 492.5 | INT 10-3, and Pd-PEPPSI-IHept-Cl (5 mol %) in step 2. H₂ (40 psi) for 16 hours in step 3. 4M HCl solution in 1,4-dioxane in step 4. |
| INT 12-5 | 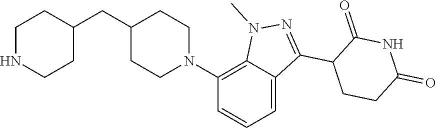 [SMILES: Cn1nc(C2CCC(=O)NC2=O)c2cccc(N3CCC(CC4CCNCC4)CC3)c21] | 424.5 | tert-butyl 4-(4- piperidylmethyl)piperidine-1- carboxylate, cesium carbonate, and Pd-PEPPSI-IHept-Cl (10 mol %) in step 2. palladium hydroxide on carbon (10 wt. %), H₂ (60 psi) at room temperature for 8 hours in step 3. |
| INT 12-6 | 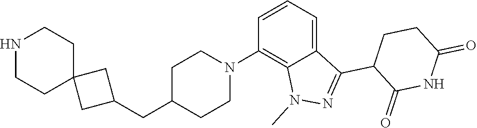 [SMILES: Cn1nc(C2CCC(=O)NC2=O)c2cccc(N3CCC(CC4CC5(CCNCC5)C4)CC3)c21] | 464.5 | INT 10-5a, cesium carbonate, and Pd-PEPPSI-IHept-Cl (10 mol %) in step 2. |
| INT 12-7 | 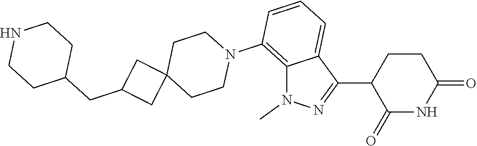 [SMILES: Cn1nc(C2CCC(=O)NC2=O)c2cccc(N3CCC4(CC3)CC(CC3CCNCC3)C4)c21] | 464.5 | INT 10-6a RuPhos Pd G3 (10 mol %) and lithium bis(trimethylsilyl)amide in step 2. Step 4 was omitted. |
| INT 12-8 | 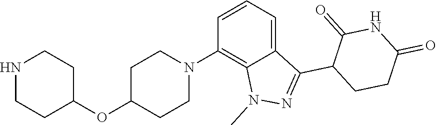 [SMILES: Cn1nc(C2CCC(=O)NC2=O)c2cccc(N3CCC(OC4CCNCC4)CC3)c21] | 426.3 | tert-butyl 4-(4- piperidyloxy)piperidine-1- carboxylate, cesium carbonate, and Pd-PEPPSI-IHept-Cl (5 mol %) in step 2. Palladium on carbon (10 wt. %), EtOAc/DMF (6:1), H₂ (15 psi) at room temperature for 1 hour in step 3. 4M HCl solution in |
| | | | 1,4-dioxane/EtOAc in step 4. |
| INT 12-9 | 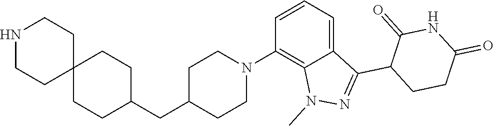 [SMILES: Cn1nc(C2CCC(=O)NC2=O)c2cccc(N3CCC(CC4CCC5(CCNCC5)CC4)CC3)c21] | 492.3 | INT 10-5b, and Pd-PEPPSI-IHept-Cl (5 mol %) in step 2. H₂ (40 psi) for 16 hours in step 3. 4M HCl solution in 1,4-dioxane in step 4. |
| INT 12-10 | 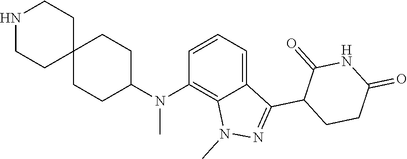 [SMILES: CN(c1cccc2c(C3CCC(=O)NC3=O)nn(C)c12)C1CCC2(CCNCC2)CC1] | 524.3 | tert-butyl 9-(methylamino)-3- azaspiro[5.5]undecane-3-carboxylate, Pd-PEPPSI-IHept-Cl (5 mol %), cesium carbonate (4.0 equiv) in step 2. |

### Intermediate 13-1.

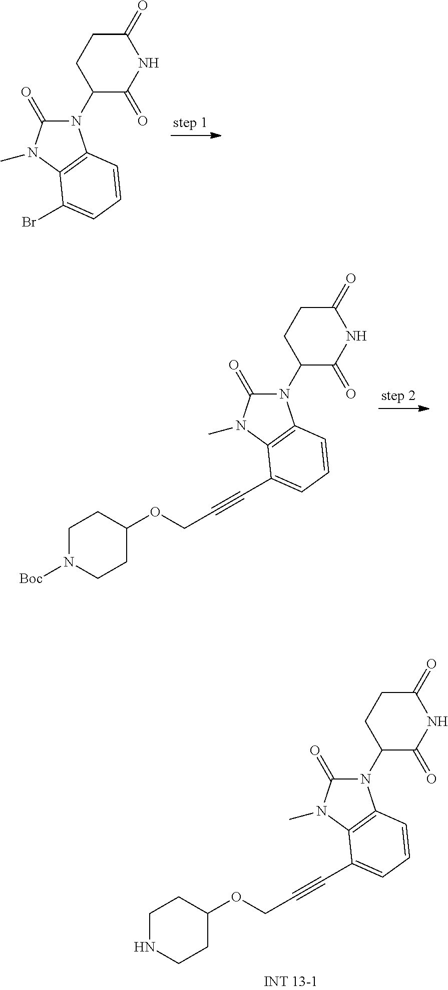 [SMILES: Cn1c(=O)n(C2CCC(=O)NC2=O)c2cccc(Br)c21 ; Cn1c(=O)n(C2CCC(=O)NC2=O)c2cccc(C#CCOC3CCN(C(=O)OC(C)(C)C)CC3)c21 ; Cn1c(=O)n(C2CCC(=O)NC2=O)c2cccc(C#CCOC3CCNCC3)c21]

Step 1: tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate. To a mixture of 3-(4-bromo-3-methyl-2-oxo-2,3-dihydro-1H-benzo[Id]imidazol-1-yl)piperidine-2,6-dione (3.0 mmol) and tert-butyl 4-prop-2-ynoxypiperidine-1I-carboxylate (4.4 mmol) in N,N-dimethylformamide (6 mL) was added copper(I) iodide (0.6 mmol), triethylamine (6.0 mmol) and tetrakis(triphenylphosphine)palladium(0) (0.15 mmol). The mixture was stirred at 80° C. for 12 hours under nitrogen. The reaction mixture was then cooled to room temperature, filtered and concentrated under reduced pressure to give a residue. The residue was purified by prep-HPLC (35% to 65% acetonitrile/water with 0.04% hydrochloric acid), and fractions containing the desired product were pooled and lyophilized to yield the title compound. LCMS: 441.2.

Step 2: 3-(3-methyl-2-oxo-4-(3-(piperidin-4-yloxy)prop-1-yn-1-yl)-2,3-dihydro-1H-benzo[d]imidazol-1-yl)piperidine-2,6-dione (INT 13-1). To the mixture of tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate (0.6 mmol) in dichloromethane (3 mL) was added trifluoroacetic (1 mL) and the mixture was stirred for 30 minutes at room temperature. Upon completion, the reaction mixture was concentrated and purified by prep-HPLC (5% to 45% acetonitrile/water with 0.1% trifluoroacetic acid), and fractions containing the desired product were pooled and lyophilized. The residue was then neutralized by treating it with a saturated aqueous sodium bicarbonate solution. The aqueous mixture was extracted with a dichloromethane/isopropanol mixture (4:1). The organic fractions were combined, dried over anhydrous sodium sulfate, filtered, and concentrated under reduced pressure to afford the title compound. LCMS: 397.1.

Intermediates 13-2a-13-2b.

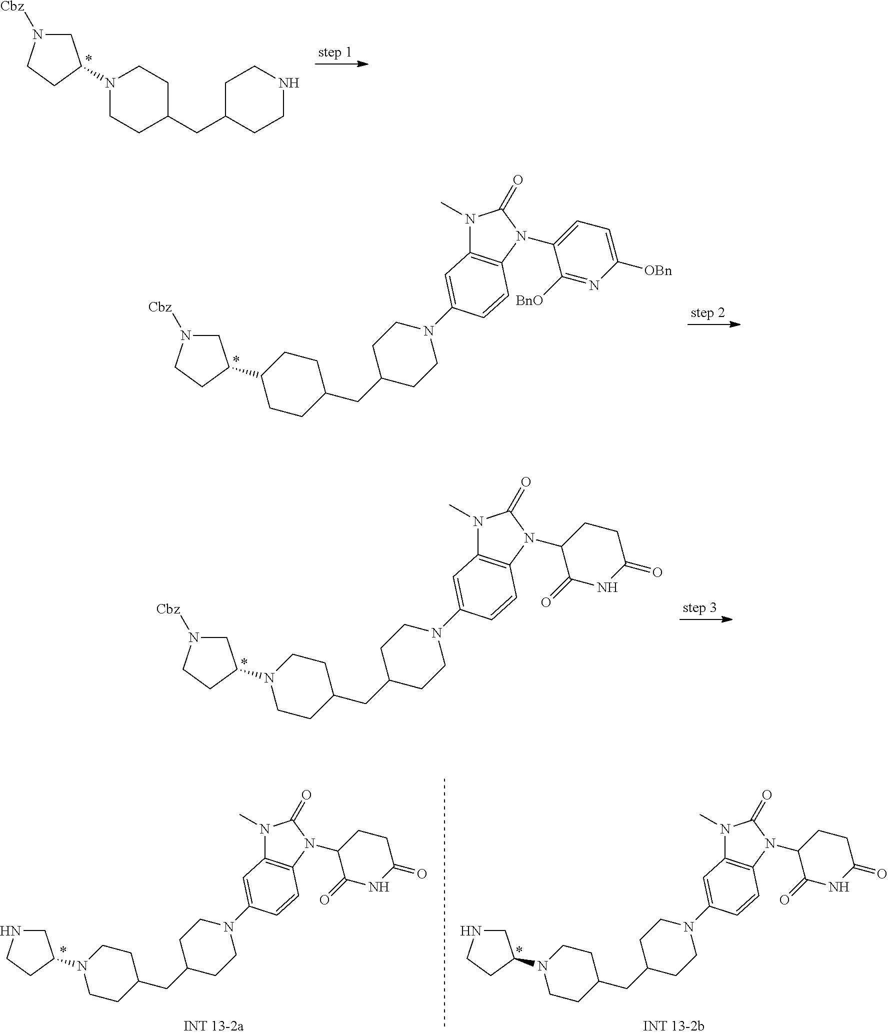 [SMILES: O=C(OCc1ccccc1)N1CC[C@@H](N2CCC(CC3CCNCC3)CC2)C1 ; Cn1c(=O)n(-c2ccc(OCc3ccccc3)nc2OCc2ccccc2)c2ccc(N3CCC(CC4CCC([C@@H]5CCN(C(=O)OCc6ccccc6)C5)CC4)CC3)cc21 ; Cn1c(=O)n(C2CCC(=O)NC2=O)c2ccc(N3CCC(CC4CCN([C@@H]5CCN(C(=O)OCc6ccccc6)C5)CC4)CC3)cc21 ; Cn1c(=O)n(C2CCC(=O)NC2=O)c2ccc(N3CCC(CC4CCN([C@@H]5CCNC5)CC4)CC3)cc21 ; Cn1c(=O)n(C2CCC(=O)NC2=O)c2ccc(N3CCC(CC4CCN([C@H]5CCNC5)CC4)CC3)cc21]

Step 1: Benzyl (3R)-3-(4-((1-(1-(2,6-dioxopiperidin-3-yl)-3-methyl-2-oxo-2,3-dihydro-1H-benzo[d]imidazol-5-yl)piperidin-4-yl)methyl)piperidin-1-yl)pyrrolidine-1-carboxylate. To a solution of benzyl (R)-3-(4-(piperidin-4-ylmethyl)piperidin-1-yl)pyrrolidine-1-carboxylate INT 10-7a (2.6 mmol) and 1-(2,6-bis(benzyloxy)pyridin-3-yl)-5-bromo-3-methyl-1,3-dihydro-2H-benzo[d]imidazol-2-one (2.6 mmol) in 1,4-dioxane (20 mL) under argon was added Ruphos (0.3 mmol), Ruphos Pd G3 (0.3 mmol), and molecular sieves (2 g, 4 Å) followed by LiHMDS (26 mmol, 1M in THF). The reaction mixture was degassed with argon, capped and stirred at 100° C. for 4 hours. Upon completion, the reaction mixture was cooled to room temperature and diluted with 1,4-dioxane. The pH of the reaction mixture was adjusted to ˜7.0 with formic acid. The mixture was filtered and the filtrate concentrated to dryness under reduced pressure. The remaining residue was purified by silica gel chromatography eluting with methanol in dichloromethane. Fractions containing the product were pooled and concentrated under reduced pressure to yield the title compound. LCMS: 821.6.

Step 2: tert-butyl (3R)-3-(4-((1-(1-(2,6-dioxopiperidin-3-yl)-3-methyl-2-oxo-2,3-dihydro-1H-benzo[d]imidazol-5-yl)piperidin-4-yl)methyl)piperidin-1-yl)pyrrolidine-1-carboxylate. To a stirred solution of benzyl (3R)-3-(4-((1-(1-(2,6-dioxopiperidin-3-yl)-3-methyl-2-oxo-2,3-dihydro-1H-benzo[d]imidazol-5-yl)piperidin-4-yl)methyl)piperidin-1-yl)pyrrolidine-1-carboxylate (1.1 mmol) in THE (20 mL) were added palladium hydroxide on carbon (4.4 mmol, 20 wt. % Pd; wetted with ca. 60% water), palladium on carbon (4.4 mmol, 10 wt. % Pd, wetted with ca. 55% water) and di-tert-butyl dicarbonate (3.3 mmol). The mixture was stirred at room temperature under a hydrogen atmosphere for 48 hours. Upon completion, the mixture was filtered over Celite and concentrated under reduced pressure. The residue was purified by flash chromatography eluting with a mixture of dichloromethane and methanol to obtain the title compound. LCMS: 609.4.

Step 3: 3-(3-methyl-2-oxo-5-(4-((1-((R)-pyrrolidin-3-yl)piperidin-4-yl)methyl)piperidin-1-yl)-2,3-dihydro-1H-benzo[d]imidazol-1-yl)piperidine-2,6-dione (INT 13-2a): To a stirred solution of tert-butyl (3R)-3-(4-((1-(1-(2,6-dioxopiperidin-3-yl)-3-methyl-2-oxo-2,3-dihydro-1H-benzo[d]imidazol-5-yl)piperidin-4-yl)methyl)piperidin-1-yl)pyrrolidine-1-carboxylate (0.4 mmol) in dichloromethane (5 mL) was added HCl (1.5 mL, 4 M in 1,4-dioxane) at 0° C. The resulting reaction mixture was stirred for 4 hours at room temperature. Upon completion, the solution was concentrated under reduced pressure, and washed with ether and ethyl acetate to obtain the title compound. LCMS: 509.4.

3-(3-methyl-2-oxo-5-(4-((1-((S)-pyrrolidin-3-yl)piperidin-4-yl)methyl)piperidin-1-yl)-2,3-dihydro-1H-benzo[d]imidazol-1-yl)piperidine-2,6-dione (INT 13-2b) was prepared analogously to INT 13-2a, substituting INT 10-7a in step 1 with INT 10-7b. LCMS: 509.4.

### Intermediate 13-3.

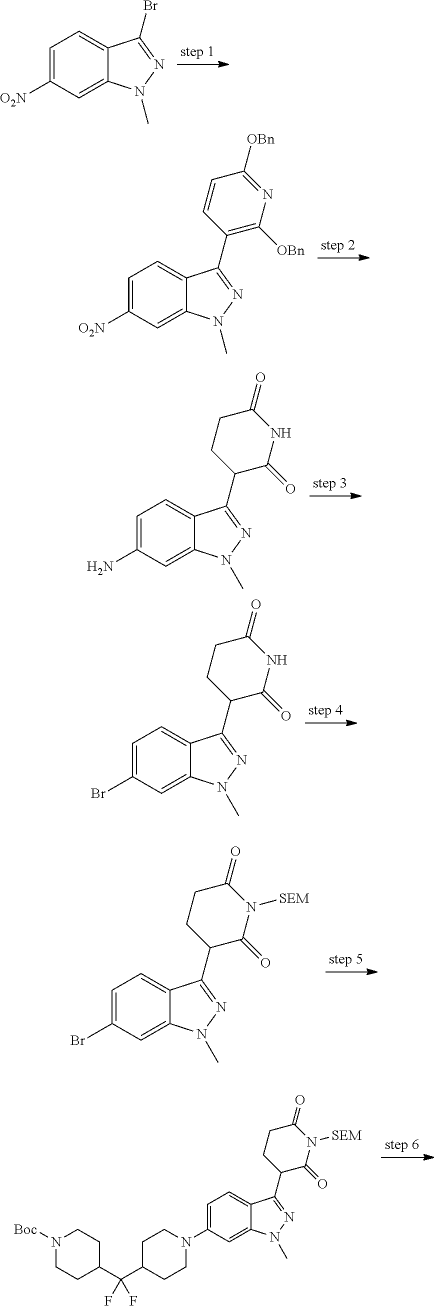

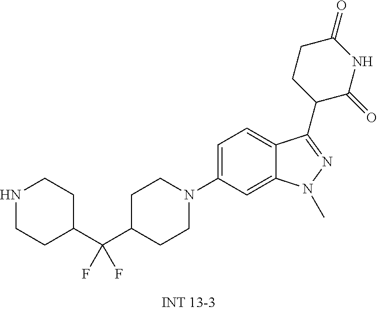 [SMILES: Cn1nc(C2CCC(=O)NC2=O)c2ccc(N3CCC(C(F)(F)C4CCNCC4)CC3)cc21]

Step 1: 3-(2,6-bis(benzyloxy)pyridin-3-yl)-1-methyl-6-nitro-1H-indazole. To a mixture of 3-iodo-1-methyl-6-nitro-1H-indazole (82.5 mmol) and 2,6-bis(benzyloxy)-3-(4,4,5,5-tetramethyl-1,3,2-dioxaborolan-2-yl)pyridine (165 mmol) was added 1,4-dioxane (250 mL) and water (75 mL). The reaction mixture was degassed with nitrogen for 5 minutes. Cesium carbonate (248 mmol) and [1,1′-bis(diphenylphosphino)ferrocene]dichloropalladium(II) (4.1 mmol) were added under nitrogen. The reaction mixture was then stirred at 100° C. for 6 hours. Water was then added, and the mixture was extracted with ethyl acetate. The organic layers were combined, washed with brine, dried over anhydrous sodium sulfate, filtered, and concentrated under reduced pressure. The residue was purified by silica gel chromatography eluting with 0 to 40% ethyl acetate in petroleum ether. Fractions containing the product were pooled and concentrated under reduced pressure to yield the title compound. LCMS: 467.2.

Step 2: 3-(6-amino-1-methyl-1H-indazol-3-yl)piperidine-2,6-dione. To a mixture of 3-(2,6-bis(benzyloxy)pyridin-3-yl)-1-methyl-6-nitro-1H-indazole (64 mmol) in tetrahydrofuran (150 mL) and EtOH (300 mL) was added palladium on carbon, 50% Pd, moisture 50% (15 g). The mixture was stirred under a hydrogen atmosphere (70 psi) at room temperature for 30 hours. Upon completion, the mixture was filtered over Celite, rinsed with THF/EtOH (1:1), then the filtrate was concentrated under reduced pressure and the residue was triturated with MTBE to afford the title compound. LCMS: 259.2.

Step 3: 3-(6-bromo-1-methyl-1H-indazol-3-yl)piperidine-2,6-dione. To a mixture of 3-(6-amino-1-methyl-1H-indazol-3-yl)piperidine-2,6-dione (19.3 mmol) in methanol (50 mL) was added HBr (20 mL, 48% in water). The mixture was cooled to −5° C. and a solution of sodium nitrite (23 mmol) in water (2 mL) was added dropwise. After 1 hour, CuBr (29 mmol) was added portion wise and the reaction mixture was warmed to room temperature and stirred for 1 hour. Upon completion, the reaction mixture was poured into 100 mL of cold water and extracted with ethyl acetate. The combined organic layers were washed with brine solution, dried over anhydrous sodium sulfate, filtered, and concentrated under reduced pressure and purified by RP-HPLC (0 to 50%, 0.1% trifluoroacetic acid in acetonitrile/0.1% trifluoroacetic acid in water). Fractions containing the product were pooled and lyophilized to afford the title compound. LCMS: 322.1.

Step 4: 3-(6-bromo-1-methyl-1H-indazol-3-yl)-1-((2-(trimethylsilyl)ethoxy)methyl)piperidine-2,6-dione. To a mixture of 3-(6-bromo-1-methyl-1H-indazol-3-yl)piperidine-2,6-dione (12.4 mmol) and 1,8-Diazabicyclo[5.4.0]undec-7-ene (37.3 mmol) in THE (50 mL) was added 2-(trimethylsilyl) ethoxy methyl chloride (37.3 mmol) at 0° C. The resulting reaction mixture was then warmed to room temperature and stirred for 30 minutes. The reaction mixture was then quenched with saturated aqueous sodium bicarbonate solution (30 mL) and the aqueous phase was extracted with ethyl acetate. The combined organic layers were washed with brine solution, dried over anhydrous sodium sulfate, filtered, and concentrated under reduced pressure. The residue was purified by silica gel chromatography eluting with ethyl acetate/petroleum ether. Fractions containing the product were pooled and concentrated under reduced pressure to yield the title compound. LCMS: 452.3.

Step 5: tert-butyl 4-((1-(3-(2,6-dioxo-1-((2-(trimethylsilyl)ethoxy)methyl)piperidin-3-yl)-1-methyl-1H-indazol-6-yl)piperidin-4-yl)difluoromethyl)piperidine-1-carboxylate. To a solution of tert-butyl 4-(difluoro(piperidin-4-yl)methyl)piperidine-1-carboxylate (0.6 mmol) in 1,4-dioxane (8 mL) was added 3-(6-bromo-1-methyl-indazol-3-yl)-1-(2-trimethylsilylethoxymethyl)piperidine-2,6-dione (0.6 mmol), X-Phos (0.2 mmol), cesium carbonate (1.7 mmol), and palladium acetate (0.1 mmol). The mixture was degassed with argon for 30 seconds then stirred at 100° C., under an argon atmosphere, for 16 hours. The reaction mixture was then filtered through Celite, concentrated under reduced pressure, and purified by flash column chromatography on silica gel eluting with ethyl acetate and hexanes to afford the title compound. LCMS: 690.0.

Step 6: 3-[6-[4-[difluoro(4-piperidyl)methyl]-1-piperidyl]-1-methyl-indazol-3-yl]piperidine-2,6-dione (INT 13-3): To a solution of tert-butyl 4-((1-(3-(2,6-dioxo-1-((2-(trimethylsilyl)ethoxy)methyl)piperidin-3-yl)-1-methyl-1H-indazol-6-yl)piperidin-4-yl)difluoromethyl)piperidine-1-carboxylate (0.5 mmol) in dichloromethane (8 mL) was added trifluoroacetic acid (10 mmol) at room temperature. The reaction was stirred for 1 hour then concentrated under reduced pressure. The residue was then taken up in dichloromethane (4 mL) and cooled to 0° C. To the cooled solution was added triethylamine (2.9 mmol) and N,N′-dimethylethylenediamine (1.1 mmol). The reaction mixture was then stirred at room temperature for 1 hour. The reaction mixture was concentrated under reduced pressure and purified by RP-HPLC (0 to 90%, 0.1% trifluoroacetic acid in acetonitrile/0.1% trifluoroacetic acid in water). Fractions containing the product were pooled and lyophilized to yield the title compound. The residue was then dissolved in tetrahydrofuran and treated with Amberlyst A21 ion exchange resin for 1 hour. The mixture was filtered, the filter cake rinsed with the same solvent mixture, and the filtrate concentrated under reduced pressure to afford the title compound. LCMS: 460.0.

### Intermediate 13-4.

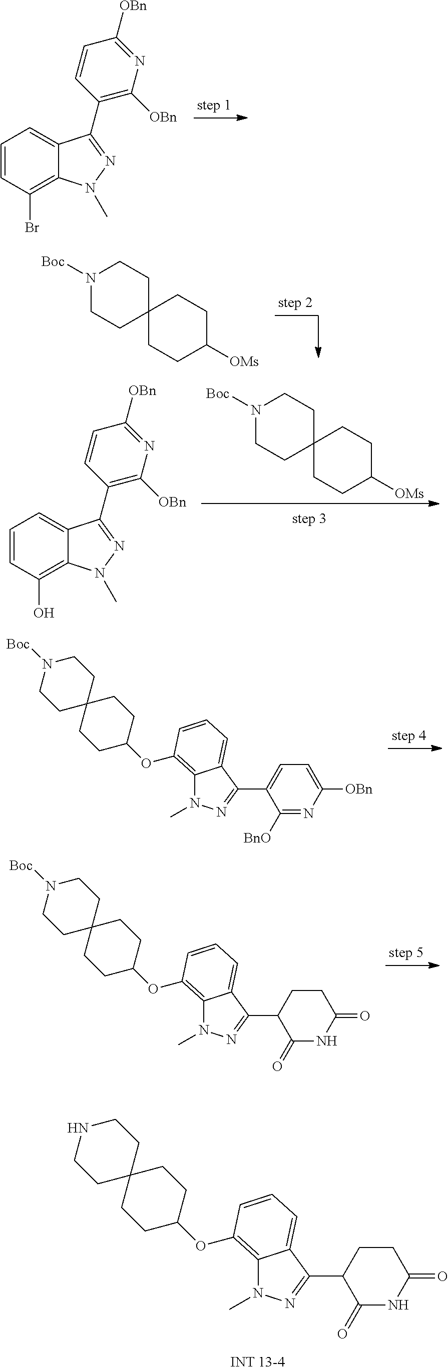

Step 1: 3-(2,6-dibenzyloxy-3-pyridyl)-1-methyl-indazol-7-ol. To a solution of 7-bromo-3-(2,6-dibenzyloxy-3-pyridyl)-1-methyl-indazole (2.0 mmol) dissolved in THE (20 mL) were added sodium tert-butoxide (6.0 mmol), tBuBrettPhos Pd G3 (0.2 mmol) and water (4.0 mmol). The reaction mixture was heated at 60° C. for 4 hours. Upon completion, the mixture was cooled to room temperature, filtered through Celite and concentrated under reduced pressure. The residue was then purified by flash column chromatography on silica gel eluting with ethyl acetate and hexanes to afford the title compound. LCMS: 438.1.

Step 2: tert-butyl 9-((methylsulfonyl)oxy)-3-azaspiro[5.5]undecane-3-carboxylate. To a solution of tert-butyl 9-hydroxy-3-azaspiro[5.5]undecane-3-carboxylate (3.7 mmol) and triethylamine (7.4 mmol) in dichloromethane (50 mL) at 0° C. was added methanesulfonyl chloride (5.6 mmol). The resulting solution was stirred at 0° C. for 1 hour. Upon completion, the reaction mixture was partitioned between sat. sodium bicarbonate solution and dichloromethane. The aqueous layer was extracted with dichloromethane (3×20 mL). The combined organic layers were washed with brine solution, dried over anhydrous sodium sulfate, filtered, and concentrated under reduced pressure to yield the title compound which was used without further purification. LCMS: 292.1.

Step 3: tert-butyl 9-((3-(2,6-bis(benzyloxy)pyridin-3-yl)-1-methyl-1H-indazol-7-yl)oxy)-3-azaspiro[5.5]undecane-3-carboxylate. To a stirred solution of 3-(2,6-dibenzyloxy-3-pyridyl)-1-methyl-indazol-7-ol (1.4 mmol) in DMF (7 mL) were added t-butyl 9-((methylsulfonyl)oxy)-3-azaspiro[5.5]undecane-3-carboxylate (8.2 mmol) and cesium carbonate (8.2 mmol). The resulting reaction mixture was stirred at 100° C. for 2 hours. Upon completion, the reaction mixture was quenched with water and extracted with ethyl acetate. The combined organic layers were dried over sodium sulfate, filtered and concentrated under reduced pressure. The residue was then purified by flash column chromatography on silica gel eluting with ethyl acetate and hexanes to afford the title compound. LCMS: 689.3.

Step 4: tert-butyl 9-[3-(2,6-dioxo-3-piperidyl)-1-methyl-indazol-7-yl]oxy-3-azaspiro[5.5]undecane-3-carboxylate. To a stirred mixture of tert-butyl 9-((3-(2,6-bis(benzyloxy)pyridin-3-yl)-1-methyl-1H-indazol-7-yl)oxy)-3-azaspiro[5.5]undecane-3-carboxylate (2.4 mmol) in tetrahydrofuran (10 mL) was added palladium hydroxide on carbon (1.1 mmol, 20 wt. % Pd). The reaction vessel was evacuated and backfilled with hydrogen (3×), and the mixture was stirred under a hydrogen atmosphere at room temperature for 16 hours. Upon completion, the mixture was filtered through a pad of Celite, rinsed with dichloromethane, and the filtrate was concentrated under reduced pressure to afford the title compound which was used without further purification. LCMS: 511.2.

Step 5: 3-[7-(3-azaspiro[5.5]undecan-9-yloxy)-1-methyl-indazol-3-yl]piperidine-2,6-dione (INT 13-4): To a solution of tert-butyl 9-[3-(2,6-dioxo-3-piperidyl)-1-methyl-indazol-7-yl]oxy-3-azaspiro[5.5]undecane-3-carboxylate (0.7 mmol) in dichloromethane (5 mL) was added TFA (1 mL). The resulting solution was stirred at room temperature for 1 hour. Upon completion, the solution was concentrated under reduced pressure, washed with diethyl ether, dissolved in 20% IPA in DCM and subsequently treated with Amberlyst A21 ion exchange resin. The mixture was then filtered and concentrated under reduced pressure to afford the title compound. LCMS: 411.2.

### Intermediate 13-5.

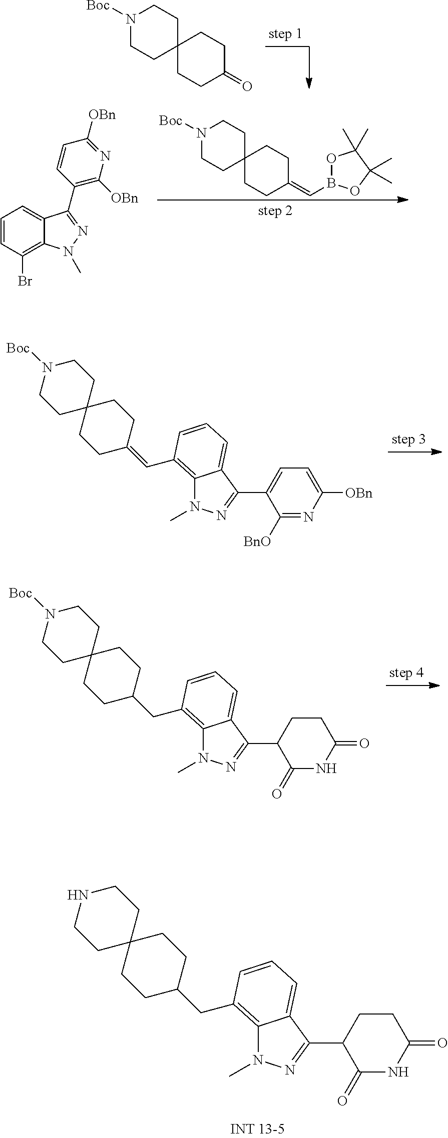 [SMILES: CC(C)(C)OC(=O)N1CCC2(CCC(=O)CC2)CC1 ; Cn1nc(-c2ccc(OCc3ccccc3)nc2OCc2ccccc2)c2cccc(Br)c21 ; CC(C)(C)OC(=O)N1CCC2(CCC(=CB3OC(C)(C)C(C)(C)O3)CC2)CC1 ; Cn1nc(-c2ccc(OCc3ccccc3)nc2OCc2ccccc2)c2cccc(C=C3CCC4(CC3)CCN(C(=O)OC(C)(C)C)CC4)c21 ; Cn1nc(C2CCC(=O)NC2=O)c2cccc(CC3CCC4(CC3)CCN(C(=O)OC(C)(C)C)CC4)c21 ; Cn1nc(C2CCC(=O)NC2=O)c2cccc(CC3CCC4(CCNCC4)CC3)c21]

Step 1: tert-butyl 9-[(4,4,5,5-tetramethyl-1,3,2-dioxaborolan-2-yl)methylene]-3-azaspiro[5.5]undecane-3-carboxylate: To a stirred solution of 4,4,5,5-tetramethyl-2-[(4,4,5,5-tetramethyl-1,3,2-dioxaborolan-2-yl)methyl]-1,3,2-dioxaborolane (3.9 mmol) in THE (12 mL) at −78° C. was added LDA (3.9 mmol, 2 M in THF/heptane/ethylbenzene) dropwise while maintaining the temperature below −70° C. After stirring the resulting reaction mixture for 15 minutes, a solution of tert-butyl 9-oxo-3-azaspiro[5.5]undecane-3-carboxylate (3.9 mmol) in THE (5 mL) was added while maintaining the temperature below −70° C. The combined solution was allowed to warm up to room temperature and stirred for 16 hours. Upon completion, the reaction mixture was poured into a solution of saturated aqueous ammonium chloride and extracted with ethyl acetate (3×50 mL). The combined organic layers were washed successively with water (50 mL) and brine (30 mL), dried over anhydrous sodium sulfate, filtered, and then concentrated under reduced pressure. The residue was purified by column chromatography eluting with a mixture of ethyl acetate and hexanes to provide the title compound. LCMS: 336.1.

Step 2: tert-butyl 9-[[3-(2,6-dibenzyloxy-3-pyridyl)-1-methyl-indazol-7-yl]methylene]-3-azaspiro[5.5]undecane-3-carboxylate: A mixture of 7-bromo-3-(2,6-dibenzyloxy-3-pyridyl)-1-methyl-indazole (2.0 mmol), tert-butyl 9-[(4,4,5,5-tetramethyl-1,3,2-dioxaborolan-2-yl)methylene]-3-azaspiro[5.5]undecane-3-carboxylate (2.4 mmol), Pd(dppf)Cl₂—CH₂Cl₂ (0.4 mmol), and K₃PO₄ (5.0 mmol) in DMF (8 mL) was degassed with argon for 5 minutes and stirred at 90° C. for 16 hours. The reaction mixture was filtered through a pad of Celite, washed with dichloromethane (3×30 mL), and then concentrated under reduced pressure. The residue was taken up in dichloromethane (30 mL) and washed successively with water (50 mL) and brine (50 mL). The organic fractions were dried over sodium sulfate, filtered and concentrated under reduced pressure. The residue was purified by flash column chromatography on silica gel eluting with a mixture of ethyl acetate and hexanes to provide the title compound. LCMS: 685.2.

3-[7-(3-azaspiro[5.5]undecan-9-ylmethyl)-1-methyl-indazol-3-yl]piperidine-2,6-dione (INT 13-5) was prepared analogously to INT 13-4, substituting tert-butyl 9-((3-(2,6-bis(benzyloxy)pyridin-3-yl)-1-methyl-1H-indazol-7-yl)oxy)-3-azaspiro[5.5]undecane-3-carboxylate in step 3 with tert-butyl 9-[[3-(2,6-dibenzyloxy-3-pyridyl)-1-methyl-indazol-7-yl]methylene]-3-azaspiro[5.5]undecane-3-carboxylate. LCMS: 409.2.

### Intermediate 14-1-14-7.

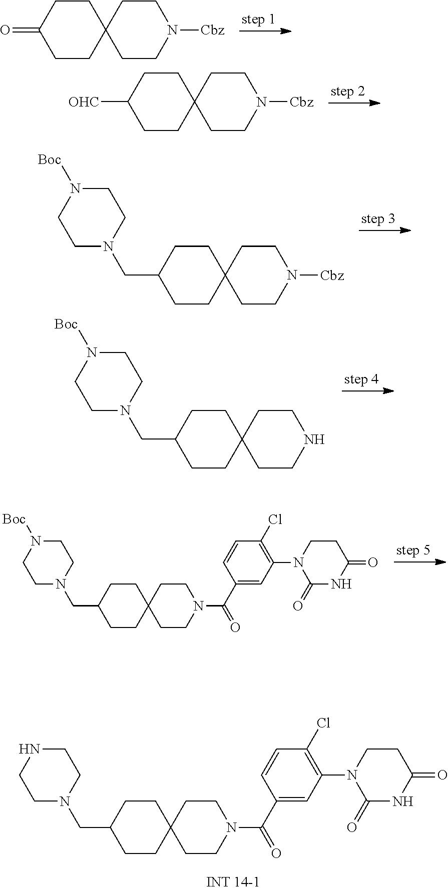

Step 1: Benzyl 9-formyl-3-azaspiro[5.5]undecane-3-carboxylate. To a stirred solution of (methoxymethyl)triphenylphosphonium chloride (21.5 mmol) in tetrahydrofuran (100 mL) was added potassium tert-butoxide (24.8 mmol) at room temperature. The reaction mixture was stirred at room temperature for 30 minutes, then benzyl 9-oxo-3-azaspiro[5.5]undecane-3-carboxylate (16.5 mmol) was added. The reaction mixture was stirred at room temperature for 12 hours. After completion, the reaction mixture was diluted with water and the aqueous layer was extracted with ethyl acetate. The organic layer was separated, washed with brine solution, dried over sodium sulfate, filtered, and concentrated under reduced pressure. The residue was then dissolved in tetrahydrofuran (53 mL) and treated with hydrochloric acid solution in water (39 mL, 1 M). The reaction mixture was stirred at room temperature for 16 hours. After completion, the reaction mixture was carefully diluted with aqueous sodium bicarbonate solution and extracted with ethyl acetate (100 mL×2). The combined organic layers were washed with brine solution, dried over anhydrous sodium sulfate, filtered, and concentrated under reduced pressure. The residue was purified by silica gel chromatography eluting with ethyl acetate/petroleum ether. Fractions containing the product were pooled and concentrated under reduced pressure to yield the title compound. LCMS: 316.2.

Step 2: Benzyl 9-((4-(tert-butoxycarbonyl)piperazin-1-yl)methyl)-3-azaspiro[5.5]undecane-3-carboxylate. To a mixture of benzyl 9-formyl-3-azaspiro[5.5]undecane-3-carboxylate (5.0 mmol), and tert-butyl piperazine-1-carboxylate (5.5 mmol) in tetrahydrofuran (16 mL) was added sodium triacetoxyborohydride (15.2 mmol) at 0° C. The reaction mixture was stirred at room temperature for 16 hours. It was then diluted with water (30 mL) and extracted with ethyl acetate. The combined organic layers were washed with brine solution, dried over anhydrous sodium sulfate, filtered, and concentrated under reduced pressure. The residue was purified by silica gel chromatography eluting with ethyl acetate/petroleum ether. Fractions containing the product were pooled and concentrated under reduced pressure to yield the title compound. LCMS: 486.4.

Step 3: tert-butyl 4-((3-azaspiro[5.5]undecan-9-yl)methyl)piperazine-1-carboxylate. To a stirred solution of benzyl 9-((4-(tert-butoxycarbonyl)piperazin-1-yl)methyl)-3-azaspiro [5.5]undecane-3-carboxylate (3.2 mmol) in tetrahydrofuran (16.0 mL) was added palladium, 10% on activated carbon (1.75 g). The reaction mixture was stirred at room temperature under 40 PSI hydrogen gas for 12 hours. The reaction mixture was filtered through Celite, rinsed with ethyl acetate, and concentrated under reduced pressure. The residue was purified by silica gel chromatography eluting with ethyl acetate/petroleum ether. Fractions containing the product were pooled and concentrated under reduced pressure to yield the title compound. LCMS: 352.3.

Step 4: tert-butyl 4-((3-(4-chloro-3-(2,4-dioxotetrahydropyrimidin-1(2H)-yl)benzoyl)-3-azaspiro[5.5]undecan-9-yl)methyl)piperazine-1-carboxylate. To a stirred solution of tert-butyl 4-((3-azaspiro[5.5]undecan-9-yl)methyl)piperazine-1-carboxylate (3.7 mmol) in dimethylacetamide (13.0 mL) was added 4-chloro-3-(2,4-dioxo tetrahydropyrimidin-1(2H)-yl)benzoic acid (3.3 mmol), N,N-diisopropylethylamine (11.1 mmol), and propanephosphonic acid anhydride (9.2 mmol) at 0° C. The reaction mixture was stirred at room temperature for 16 hours. The reaction mixture was diluted with ice-water and extracted with ethyl acetate (50 mL×3). The organic layer was washed with brine solution, dried over anhydrous sodium sulfate, filtered, and concentrated under reduced pressure. The residue was purified by silica gel chromatography eluting with 3-5% methanol in dichloromethane. Fractions containing the product were pooled and concentrated under reduced pressure to yield the title compound. LCMS: 602.6.

Step 5: 1-(2-chloro-5-(9-(piperazin-1-ylmethyl)-3-azaspiro[5.5]undecane-3-carbonyl)phenyl)dihydropyrimidine-2,4(1H,3H)-dione (INT 14-1). To a stirred solution of tert-butyl 4-((3-(4-chloro-3-(2,4-dioxotetrahydropyrimidin-1(2H)-yl) benzoyl)-3-azaspiro[5.5]undecan-9-yl)methyl)piperazine-1-carboxylate (1.1 mmol) in dichloromethane (6.7 mL) was added hydrogen chloride (4 M solution in 1,4-dioxane 0.55 mL) at 0° C., then the reaction mixture was stirred at room temperature for 2 hours. The reaction mixture was concentrated under reduced pressure. The crude compound was dissolved in dichloromethane, washed with a solution of NaHCO₃, dried over sodium sulfate, filtered and concentrated under reduced pressure to yield the title compound. LCMS: 502.4.

The following intermediates were made in a similar fashion to INT 14-1 and are shown below in Table 2K. To prepare the below intermediates, different reagents/starting materials were used than previously described in the steps toward INT 14-1 and are noted in the last column of Table 2K—“Changes to Procedure: Different Reagents/Starting Materials”. A person of ordinary skill in the art will readily recognize which reagents/starting materials used for the synthesis of INT 14-1 were replaced with the different reagents/starting materials noted below.

TABLE 2K
Intermediates

| Inter- | | | Changes to Procedure: Different |
|---|---|---|---|
| mediate | Structure | LCMS | Reagents/Starting Materials |
| INT 14-2 | 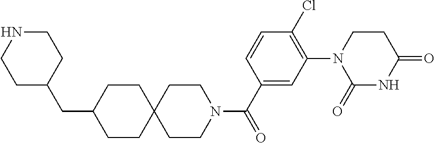 [SMILES: O=C1CCN(c2cc(C(=O)N3CCC4(CCC(CC5CCNCC5)CC4)CC3)ccc2Cl)C(=O)N1] | 501.4 | INT 10-3 in step 4. |
| INT 14-3 | 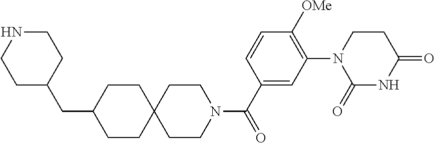 [SMILES: COc1ccc(C(=O)N2CCC3(CCC(CC4CCNCC4)CC3)CC2)cc1N1CCC(=O)NC1=O] | 497.5 | INT 10-3 and 3-(2,4- dioxotetrahydropyrimidin-1(2H)-yl)- 4-methoxybenzoic acid in step 4. |
| INT 14-4 | 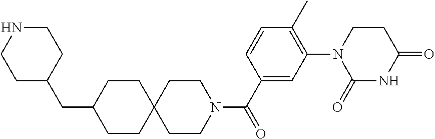 [SMILES: Cc1ccc(C(=O)N2CCC3(CCC(CC4CCNCC4)CC3)CC2)cc1N1CCC(=O)NC1=O] | 481.4 | INT-10-6b and 3-(2,4- dioxohexahydropyrimidin-1-yl)-4- methyl-benzoic acid, HATU (1.1 equiv) in step 4. H₂ (15 psi), palladium hydroxide on carbon, powder, unreduced, 20 wt. % Pd, moisture 60%, EtOH/THF in step 5. |
| INT 14-5 | 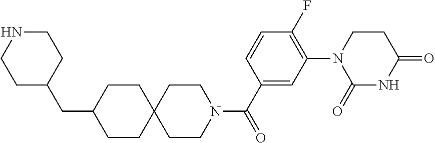 [SMILES: O=C1CCN(c2cc(C(=O)N3CCC4(CCC(CC5CCNCC5)CC4)CC3)ccc2F)C(=O)N1] | 485.4 | INT-10-6b and 3-(2,4- dioxotetrahydropyrimidin-1(2H)-yl)- 4-fluorobenzoic acid, HATU (1.1 equiv) in step 4. H₂ (15 psi), palladium hydroxide on carbon, powder, unreduced, 20 wt. % Pd, moisture 60%, EtOH/THF in step 5. |
| INT 14-6 | 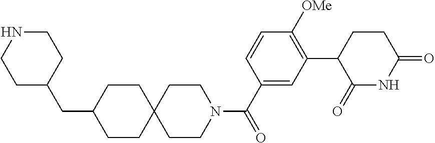 [SMILES: COc1ccc(C(=O)N2CCC3(CCC(CC4CCNCC4)CC3)CC2)cc1C1CCC(=O)NC1=O] | 496.3 | INT-10-6b and 3-(2,6- dioxopiperidin-3-yl)-4- methoxybenzoic acid, HATU (1.1 equiv) in step 4. H₂ (15 psi), palladium hydroxide on carbon, powder, unreduced, 20 wt. % Pd, moisture 60%, EtOH/THF in step 5. |
| INT 14-7 | 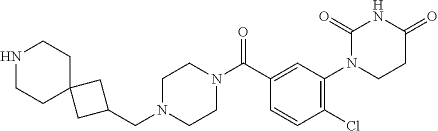 [SMILES: O=C1CCN(c2cc(C(=O)N3CCN(CC4CC5(CCNCC5)C4)CC3)ccc2Cl)C(=O)N1] | 1014.2 | Steps 1-3 were omitted. tert-butyl 2- (piperazin-1-ylmethyl)-7- azaspiro[3.5]nonane-7-carboxylate was used in step 4. |

### Intermediate 15-1.

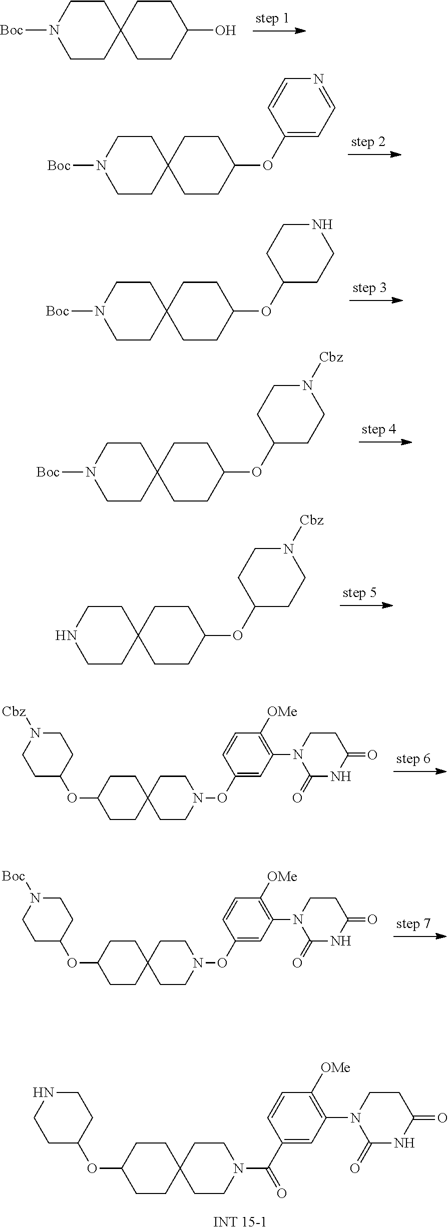

Step 1: tert-butyl 9-(pyridin-4-yloxy)-3-azaspiro[5.5]undecane-3-carboxylate. To a stirred solution of tert-butyl-9-hydroxy-3-azaspiro[5.5]undecane-3-carboxylate (11.1 mmol) in N,N-dimethylformamide (30 mL) at 0° C. was added NaH (60%, dispersion in paraffin liquid, 16.7 mmol). The mixture was stirred at room temperature for 10 minutes, then 4-nitropyridine (14.5 mmol) was added. The reaction mixture was stirred at room temperature for 6 hours. The reaction mixture was cooled to 0° C., and the product was precipitated and subsequently isolated through filtration and washing with cold water to yield the title compound. LCMS: 347.3.

Step 2: tert-butyl 9-(piperidin-4-yloxy)-3-azaspiro[5.5]undecane-3-carboxylate. To a stirred solution of tert-butyl 9-(pyridin-4-yloxy)-3-azaspiro[5.5]undecane-3-carboxylate (4.3 mmol) in acetic acid (15 mL) was added platinum on activated carbon, 10 wt. % Pt, ca. 50% moisture (0.49 g). The reaction mixture was stirred at room temperature under a hydrogen atmosphere (50 psi) for 16 hours. The reaction mixture was filtered over Celite, washed multiple times with ethanol and the filtrate was concentrated to yield the crude product which was used without further purification. LCMS: 353.3.

Step 3: tert-butyl 9-((1-((benzyloxy)carbonyl)piperidin-4-yl)oxy)-3-azaspiro [5.5]undecane-3-carboxylate. To a stirred solution of tert-butyl 9-(piperidin-4-yloxy)-3-azaspiro[5.5]undecane-3-carboxylate (3.4 mmol) in tetrahydrofuran (10 mL) and water (2 mL), sodium bicarbonate (10.2 mmol) was added. Subsequently, benzyl chloroformate (3.4 mmol) was added at 0° C. Then the reaction mixture was stirred at room temperature for 6 hours. Water was added to the reaction mixture, and the aqueous layer was extracted with ethyl acetate. The organic layers were separated and dried over anhydrous sodium sulfate, filtered, and concentrated under reduced pressure to yield the title compound which was used in the next step without further purification. LCMS: 487.6.

Step 4: Benzyl 4-((3-azaspiro[5.5]undecan-9-yl)oxy)piperidine-1-carboxylate. To a solution of tert-butyl 9-((1-((benzyloxy)carbonyl)piperidin-4-yl)oxy)-3-azaspiro [5.5]undecane-3-carboxylate (2.3 mmol) in dichloromethane (10 mL) was added hydrogen chloride (4M in 1,4-dioxane, 4 mL) at 0° C. The reaction mixture was stirred at room temperature for 12 hours then concentrated under reduced pressure. The residue was washed with diethyl ether to yield the title compound. LCMS: 387.6.

Step 5: Benzyl 4-((3-(3-(2,4-dioxotetrahydropyrimidin-1(2H)-yl)-4-methoxybenzoyl)-3-azaspiro[5.5]undecan-9-yl)oxy)piperidine-1-carboxylate. To a stirred solution of 3-(2,4-dioxotetrahydropyrimidin-1(2H)-yl)-4-methoxybenzoic acid (1.3 mmol) in acetonitrile (5 mL), chloro-N,N,N′,N′-tetramethylformamidinium hexafluorophosphate (1.5 mmol), 1-methylimidazole (5.0 mmol) and benzyl 4-((3-azaspiro[5.5]undecan-9-yl)oxy)piperidine-1-carboxylate (1.8 mmol) were added at room temperature. The reaction mixture was stirred at room temperature for 3 hours then concentrated under reduced pressure. The residue was purified by silica gel chromatography eluting with 1-3% methanol in dichloromethane. Fractions containing the product were pooled and concentrated to yield the title compound. LCMS: 633.7.

Step 6: tert-butyl 4-((3-(3-(2,4-dioxotetrahydropyrimidin-1(2H)-yl)-4-methoxy benzoyl)-3-azaspiro[5.5]undecan-9-yl)oxy)piperidine-1-carboxylate. To a stirred solution of benzyl 4-((3-(3-(2,4-dioxotetrahydropyrimidin-1(2H)-yl)-4-methoxy benzoyl)-3-azaspiro[5.5]undecan-9-yl)oxy)piperidine-1-carboxylate (1 mmol) in tetrahydrofuran (6 mL) were added palladium on carbon (0.3 g, 10 wt. %, wetted with ca. 55% water), palladium hydroxide (0.3 g, 20 wt. %, wetted with ca. 55% water) and di-tert-butyl dicarbonate (2 mmol). The reaction mixture was stirred at room temperature under a H₂ balloon for 6 hours. The reaction mixture was filtered through Celite and concentrated to yield the title compound which was used without further purification. LCMS: 599.5.

Step 7: 1-(2-methoxy-5-(9-(piperidin-4-yloxy)-3-azaspiro[5.5]undecane-3-carbonyl) phenyl)dihydropyrimidine-2,4(1H,3H)-dione (INT 15-1). To a stirred solution of tert-butyl 4-((3-(3-(2,4-dioxotetrahydropyrimidin-1(2H)-yl)-4-methoxybenzoyl)-3-azaspiro[5.5]undecan-9-yl)oxy)piperidine-1-carboxylate (0.9 mmol) in dichloromethane (5 mL) was added trifluoroacetic acid (0.2 mL) at 0° C. The reaction mixture was stirred at room temperature for 6 hours then concentrated under reduced pressure. The residue was washed with diethyl ether then dissolved in tetrahydrofuran and treated with Amberlyst A21 ion exchange resin for 1 hour. The mixture was filtered, the filter cake rinsed with the same solvent mixture, and the filtrate concentrated under reduced pressure to afford the title compound. LCMS: 499.6.

### Intermediate 16-1-16-5.

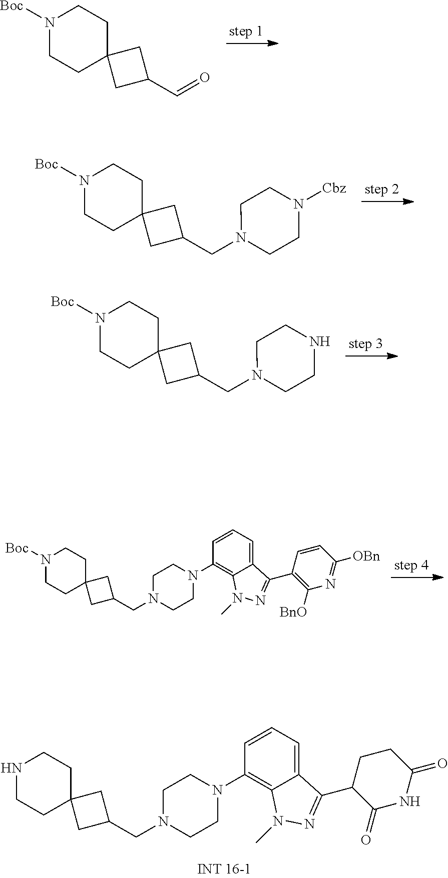 [SMILES: CC(C)(C)OC(=O)N1CCC2(CC1)CC(C=O)C2 ; CC(C)(C)OC(=O)N1CCC2(CC1)CC(CN1CCN(C(=O)OCc3ccccc3)CC1)C2 ; CC(C)(C)OC(=O)N1CCC2(CC1)CC(CN1CCNCC1)C2 ; Cn1nc(-c2ccc(OCc3ccccc3)nc2OCc2ccccc2)c2cccc(N3CCN(CC4CC5(CCN(C(=O)OC(C)(C)C)CC5)C4)CC3)c21 ; Cn1nc(C2CCC(=O)NC2=O)c2cccc(N3CCN(CC4CC5(CCNCC5)C4)CC3)c21]

Step 1: tert-butyl 2-[(4-benzyloxycarbonylpiperazin-1-yl)methyl]-7-azaspiro[3.5]nonane-7-carboxylate. To a solution of benzyl piperazine-1-carboxylate (13 mmol) in dichloromethane and methanol (58 mL, 1:1) was added diisopropylethylamine (7.9 mmol) and the mixture was stirred at room temperature for 10 minutes. Tert-butyl 2-formyl-7-azaspiro[3.5]nonane-7-carboxylate (7.9 mmol) and acetic acid (2.4 mmol) were then added and the mixture was stirred at room temperature for 30 minutes. Sodium cyanoborohydride (9.5 mmol) was then added and the mixture was stirred for an additional 1 hour. Upon completion, the mixture was then diluted with ethyl acetate (50 mL), washed with saturated aqueous sodium bicarbonate solution (2×100 mL), dried over magnesium sulfate, and concentrated under reduced pressure to give a crude residue. The residue was then purified by flash column chromatography on silica gel eluting with ethyl acetate and hexanes to afford the title compound. LCMS: 458.2.

Step 2: tert-butyl 2-(piperazin-1-ylmethyl)-7-azaspiro[3.5]nonane-7-carboxylate. To a solution of tert-butyl 2-[(4-benzyloxycarbonylpiperazin-1-yl)methyl]-7-azaspiro[3.5]nonane-7-carboxylate (2.2 mmol) in tetrahydrofuran (31 mL) was added palladium hydroxide on carbon (2.3 mmol, 20 wt. % Pd). The mixture was then evacuated and backfilled with hydrogen three times. The mixture was then stirred at room temperature, under a hydrogen atmosphere, for 16 hours. The mixture was then filtered through Celite and concentrated under reduced pressure to afford the title compound which was used without further purification. LCMS: 324.2.

Step 3: tert-butyl 2-[[4-[3-(2,6-dibenzyloxy-3-pyridyl)-1-methyl-indazol-7-yl]piperazin-1-yl]methyl]-7-azaspiro[3.5]nonane-7-carboxylate. To a solution of 7-bromo-3-(2,6-dibenzyloxy-3-pyridyl)-1-methyl-indazole (0.8 mmol) in 1,4-dioxanes (3 mL) was added tert-butyl 2-(piperazin-1-ylmethyl)-7-azaspiro[3.5]nonane-7-carboxylate (1.0 mmol), sodium tert-butoxide (2.9 mmol), RuPhos (0.1 mmol), and RuPhos Pd G3 (0.05 mmol). The mixture was then degassed with argon for 1 minute and subsequently stirred at 100° C., under argon, for 16 hours. The reaction mixture was then cooled to room temperature, filtered, and concentrated under reduced pressure to give a crude residue. The residue was then purified by flash column chromatography on silica gel eluting with ethyl acetate and hexanes to give the title compound. LCMS: 743.3.

Step 4: 3-[7-[4-(7-azaspiro[3.5]nonan-2-ylmethyl)piperazin-1-yl]-1-methyl-indazol-3-yl]piperidine-2,6-dione (INT 16-1). To a solution of tert-butyl 2-[[4-[3-(2,6-dibenzyloxy-3-pyridyl)-1-methyl-indazol-7-yl]piperazin-1-yl]methyl]-7-azaspiro[3.5]nonane-7-carboxylate (0.3 mmol) in tetrahydrofuran (4 mL) was added palladium hydroxide on carbon (0.3 mmol, 20 wt. % Pd). The mixture was then evacuated and backfilled with hydrogen three times and then stirred at room temperature, under a hydrogen atmosphere, for 16 hours. The mixture was then filtered through Celite and concentrated under reduced pressure. The resulting residue was then dissolved in dichloromethane (5 mL) and trifluoroacetic acid (1.1 mL) was added. The mixture was stirred at room temperature for one hour, concentrated under reduced pressure, then dissolved in acetonitrile (10 mL). The mixture was neutralized with Amberlyst A21 ion exchange resin, filtered, and concentrated under reduced pressure to afford the title compound without the need for further purification. LCMS: 465.2.

The following intermediates were made in a similar fashion to INT 16-1 and are shown below in Table 2L. To prepare the below intermediates, different reagents/starting materials were used than previously described in the steps toward INT 16-1 and are noted in the last column of Table 2L—“Changes to Procedure: Different Reagents/Starting Materials”. A person of ordinary skill in the art will readily recognize which reagents/starting materials used for the synthesis of INT 16-1 were replaced with the different reagents/starting materials noted below.

TABLE 2L
Intermediates

| | | | Changes to Procedure: |
|---|---|---|---|
| | | | Different Reagents/Starting |
| Intermediate | Structure | LCMS | Materials |
| INT 16-2 | 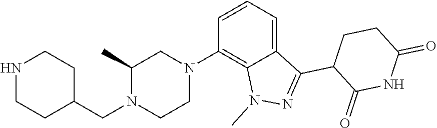 [SMILES: C[C@H]1CN(c2cccc3c(C4CCC(=O)NC4=O)nn(C)c23)CCN1CC1CCNCC1] | 439.2 | tert-butyl 4-formylpiperidine-1- carboxylate and benzyl (S)-3- methylpiperazine-1-carboxylate in step 1. |
| INT 16-3 | 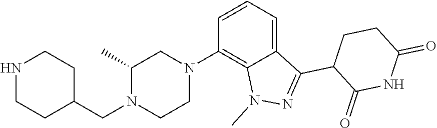 [SMILES: C[C@@H]1CN(c2cccc3c(C4CCC(=O)NC4=O)nn(C)c23)CCN1CC1CCNCC1] | 439.2 | tert-butyl 4-formylpiperidine-1- carboxylate and benzyl (R)-3- methylpiperazine-1-carboxylate in step 1. |
| INT 16-4 | 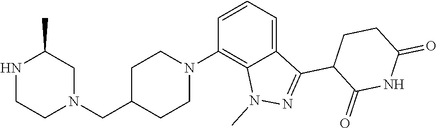 [SMILES: C[C@H]1CN(CC2CCN(c3cccc4c(C5CCC(=O)NC5=O)nn(C)c34)CC2)CCN1] | 439.2 | tert-butyl (S)-2- methylpiperazine-1-carboxylate and benzyl 4-formylpiperidine- 1-carboxylate in step 1. |
| INT 16-5 | 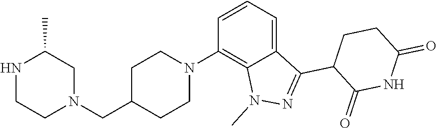 [SMILES: C[C@@H]1CN(CC2CCN(c3cccc4c(C5CCC(=O)NC5=O)nn(C)c34)CC2)CCN1] | 439.2 | tert-butyl (R)-2- methylpiperazine-1-carboxylate and benzyl 4-formylpiperidine- 1-carboxylate in step 1. |

Intermediates 17-1a-17-1b

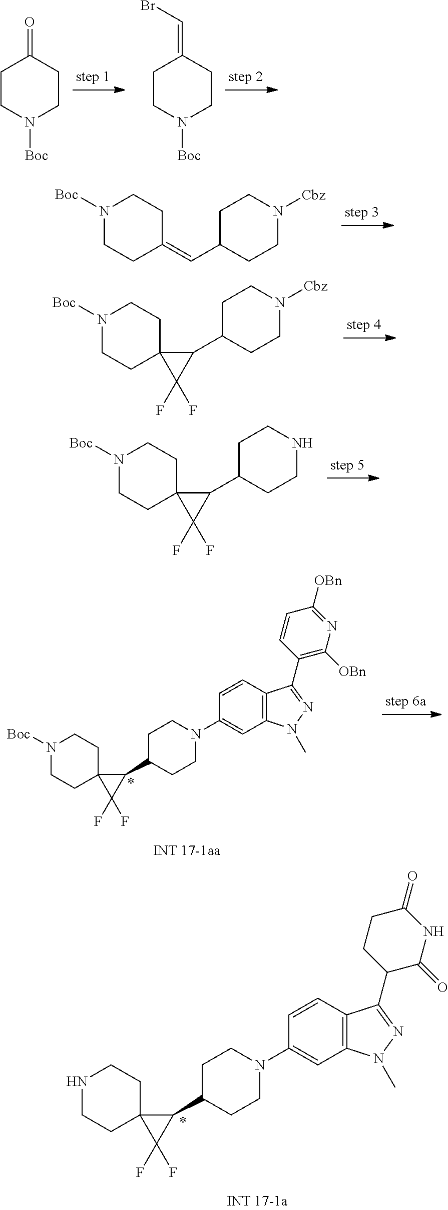

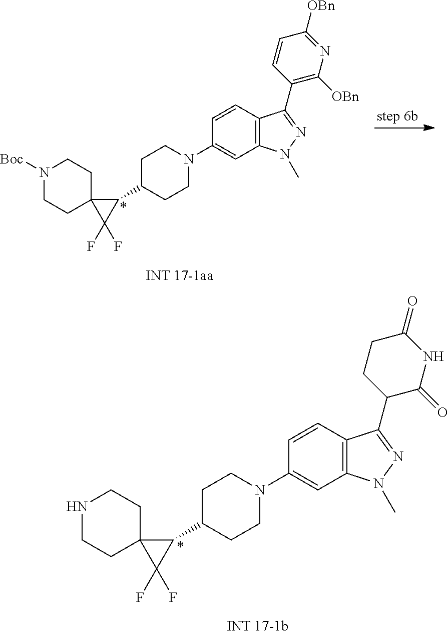 [SMILES: Cn1nc(-c2ccc(OCc3ccccc3)nc2OCc2ccccc2)c2ccc(N3CCC([C@@H]4C(F)(F)C45CCN(C(=O)OC(C)(C)C)CC5)CC3)cc21 ; Cn1nc(C2CCC(=O)NC2=O)c2ccc(N3CCC([C@@H]4C(F)(F)C45CCNCC5)CC3)cc21]

Step 1: tert-butyl 4-(bromomethylene)piperidine-1-carboxylate. To a solution of (bromomethyl)triphenylphosphonium bromide (18 mmol) in tetrahydrofuran (80 mL) at −78° C. was added potassium tert-butoxide (23 mmol) and the reaction mixture was stirred for one hour. Tert-butyl 4-oxopiperidine-1-carboxylate (23 mmol) was then added in one portion and the resulting mixture was then warmed to room temperature and stirred for 16 hours under an inert atmosphere. The reaction mixture was then diluted with saturated aqueous ammonium chloride solution (50 mL), extracted with ethyl acetate (2×30 mL), and washed with water (25 mL). The combined organic layers were then dried over anhydrous sodium sulfate, filtered and concentrated under reduced pressure. The residue was purified by flash column chromatography on silica gel eluting with ethyl acetate in hexanes to afford the title compound. ¹H NMR: (400 MHz, CDCl₃ (5.99 (s, 1H), 3.40-3.45 (m, 4H), 2.39 (t, J=5.6, 2H), 2.24 (t, J=5.6, 2H), 1.47 (s, 9H).

Step 2: Benzyl 4-((1-(tert-butoxycarbonyl)piperidin-4-ylidene)methyl)piperidine-1-carboxylate. To a solution of tert-butyl 4-(bromomethylene)piperidine-1-carboxylate (3.6 mmol) in dimethoxyethane (25 mL) was added benzyl 4-bromopiperidine-1-carboxylate (4.7 mmol), tris(trimethylsilyl)silane (3.6 mmol), and sodium carbonate (7.3 mmol). The resulting mixture was then degassed with argon for 15 minutes. Ir[dF(CF₃)ppy]₂(dtbbpy)PF₆ (0.04 mmol) and NiCl2.dtbbpy (0.04 mmol) were then added and the resulting mixture was stirred at room temperature in a photo reactor (equipped with a cooling fan) at 450 nM for 16 hours. The reaction mixture was then diluted with water (50 mL) and extracted with ethyl acetate (2×25 mL). The combined organic layers were then dried over sodium sulfate, filtered, and concentrated under reduced pressure. The residue was purified by flash column chromatography on silica gel eluting with ethyl acetate in hexanes to afford the title compound. LCMS: 415.3.

Step 3: tert-butyl 2-(1-((benzyloxy)carbonyl)piperidin-4-yl)-1,1-difluoro-6-azaspiro[2.5]octane-6-carboxylate. To a solution of benzyl 4-((1-(tert-butoxycarbonyl)piperidin-4-ylidene)methyl)piperidine-1-carboxylate (0.12 mmol) in tetrahydrofuran (10 mL) was added sodium iodide (0.04 mmol) and trimethyl(trifluoromethyl)silane (0.4 mmol). The reaction mixture was then stirred at room temperature for 16 hours under an inert atmosphere. The mixture was then diluted with water (20 mL), extracted with ethyl acetate (2×10 mL), and the combined organic layers were then dried over sodium sulfate, filtered, and concentrated under reduced pressure. The residue was purified by flash column chromatography on silica gel eluting with ethyl acetate in hexanes to afford the title compound. LCMS: 465.3.

Step 4: tert-butyl 1,1-difluoro-2-(piperidin-4-yl)-6-azaspiro[2.5]octane-6-carboxylate. To a solution of tert-butyl 2-(1-((benzyloxy)carbonyl)piperidin-4-yl)-1,1-difluoro-6-azaspiro[2.5]octane-6-carboxylate (3.0 mmol) in methanol (60 mL) was added palladium on carbon (3.0 mmol, 10 wt. % Pd). The reaction mixture was then evacuated and backfilled with hydrogen three times and stirred for 16 hours at room temperature. The mixture was then filtered through Celite, washed with methanol (100 mL), and concentrated under reduced pressure to afford the title compound which was taken forward without further purification. LCMS: 331.2.

Step 5: tert-butyl 2-(1-(3-(2,6-bis(benzyloxy)pyridin-3-yl)-1-methyl-1H-indazol-6-yl)piperidin-4-yl)-1,1-difluoro-6-azaspiro[2.5]octane-6-carboxylate. To a solution of tert-butyl 1,1-difluoro-2-(piperidin-4-yl)-6-azaspiro[2.5]octane-6-carboxylate (0.08 mmol) in 1,4-dioxane (0.3 mL) was added 3-(2,6-bis(benzyloxy)pyridin-3-yl)-6-bromo-1-methyl-1H-indazole (0.08 mmol), cesium carbonate (0.2 mmol), dicyclohexyl[2′,4′,6′-tris(propan-2-yl)[1,1′-biphenyl]-2-yl]phosphane (0.01 mmol) and tris(dibenzylideneacetone)dipalladium(0) (0.01 mmol). The resulting mixture was then degassed with argon for 5 minutes and subsequently stirred at 90° C. for 16 hours. The mixture was then diluted with water (5 mL), extracted with ethyl acetate (2×10 mL), and the combined organic layers were dried over sodium sulfate, filtered, and concentrated under reduced pressure. The residue was purified by flash column chromatography on silica gel eluting with ethyl acetate in hexanes to afford the title compound as a mixture of two enantiomers which were separated by chiral SFC (CHIRALPAK-IK 5 um-4.6×250 mm column with 70% CO₂ and 30% acetonitrile in methanol (1:1) to give two enantiomers, which were arbitrarily assigned as INT-17-1aa (Peak 1) and INT-17-1bb (Peak 2). INT-17-1aa: tert-butyl (R)-2-(1-(3-(2,6-bis(benzyloxy)pyridin-3-yl)-1-methyl-1H-indazol-6-yl)piperidin-4-yl)-1,1-difluoro-6-azaspiro[2.5]octane-6-carboxylate. LCMS: 750.5. INT-17-1bb: tert-butyl (S)-2-(1-(3-(2,6-bis(benzyloxy)pyridin-3-yl)-1-methyl-1H-indazol-6-yl)piperidin-4-yl)-1,1-difluoro-6-azaspiro[2.5]octane-6-carboxylate. LCMS: 750.3.

Step 6b: 3-(6-(4-((S)-2,2-difluoro-6-azaspiro[2.5]octan-1-yl)piperidin-1-yl)-1-methyl-1H-indazol-3-yl)piperidine-2,6-dione (INT 17-1b): To a solution of INT 17-1bb (0.5 mmol) in tetrahydrofuran (40 mL) was added palladium hydroxide on carbon (2.5 mmol, 20 wt. % Pd) and the reaction mixture was then evacuated and backfilled with hydrogen three times. The mixture was then stirred at room temperature, under a hydrogen atmosphere, for 16 hours. The mixture was then filtered through Celite then concentrated under reduced pressure to afford a crude residue. The residue was then dissolved in dichloromethane (5 mL) and hydrochloric acid (0.5 mL, 4 M in 1,4-dioxane) was added. The mixture was stirred for 4 hours at room temperature and then concentrated under reduced pressure. The crude residue was dissolved in tetrahydrofuran/dichloromethane/acetonitrile (1:1:1) and subsequently neutralized with Amberlyst A21 ion exchange resin. The mixture was then filtered and concentrated under reduced pressure to afford the title compound which was taken forward without further purification. LCMS: 472.4.

3-(6-(4-((R)-2,2-difluoro-6-azaspiro[2.5]octan-1-yl)piperidin-1-yl)-1-methyl-1H-indazol-3-yl)piperidine-2,6-dione (INT 17-1a) was prepared analogously to INT 17-1b, substituting INT 17-1bb in step 6b with INT 17-1aa. LCMS: 472.0.

### Intermediate 18-1-18-2

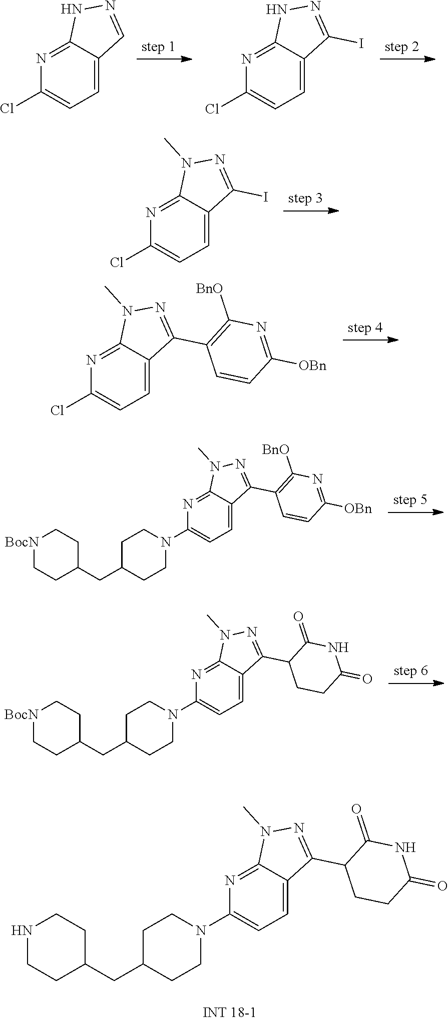

Step 1: 6-chloro-3-iodo-1H-pyrazolo[3,4-b]pyridine. To a solution of 6-chloro-1H-pyrazolo[3,4-b]pyridine (13 mmol) in DMF (20 mL) was added N-iodosuccinimide (16 mmol) at room temperature. The reaction mixture was stirred at 80° C. for 2 h. Upon completion, the solution was poured into 200 mL of water. The precipitate was isolated after filtration and used directly to obtain the title compound without further purification. LCMS: 280.0.

Step 2: 6-chloro-3-iodo-1-methyl-pyrazolo[3,4-b]pyridine. To a solution of 6-chloro-3-iodo-1H-pyrazolo[3,4-b]pyridine (7.2 mmol) and potassium carbonate (22 mmol) in DMF was added iodomethane (14 mmol). The reaction mixture was stirred at room temperature for 16 h. Upon completion, the solution was poured into 200 mL of water. The precipitate was isolated after filtration and used directly to obtain the title compound without further purification. LCMS: 294.1.

Step 3: 6-chloro-3-(2,6-dibenzyloxy-3-pyridyl)-1-methyl-pyrazolo[3,4-b]pyridine. A solution of 6-chloro-3-iodo-1-methyl-pyrazolo[3,4-b]pyridine (3.4 mmol), 2,6-dibenzyloxy-3-(4,4,5,5-tetramethyl-1,3,2-dioxaborolan-2-yl)pyridine (4.1 mmol), 1,1′-bis(diphenylphosphino)ferrocenedichloropalladium(II) (0.17 mmol) and cesium carbonate (10 mmol) in tetrahydrofuran (10 mL) and water (1 mL) was heated at 100° C. under argon for 16 h. The mixture was then cooled to room temperature, filtered through Celite and concentrated under reduced pressure. The residue was purified by flash column chromatography on silica gel (0% to 20% ethyl acetate in hexanes) to afford the title compound. LCMS: 457.3.

Step 4: tert-butyl 4-[[1-[3-(2,6-dibenzyloxy-3-pyridyl)-1-methyl-pyrazolo[3,4-b]pyridin-6-yl]-4-piperidyl]methyl]piperidine-1-carboxylate. A solution of 6-chloro-3-(2,6-dibenzyloxy-3-pyridyl)-1-methyl-pyrazolo[3,4-b]pyridine (1.6 mmol), tert-butyl 4-(4-piperidylmethyl)piperidine-1-carboxylate (2.43 mmol), tris(dibenzylideneacetone)dipalladium(0) (0.049 mmol), RuPhos (0.081 mmol), sodium tert-butoxide (6.5 mmol) in 1,4-dioxane (8 mL) was heated under argon at 100° C. for 16 h. Upon completion, the reaction was cooled to room temperature, then saturated ammonium chloride solution was added. The mixture was extracted with ethyl acetate (3×25 mL), the combined organic layers were dried over anhydrous sodium sulfate, filtered and concentrated under reduced pressure. The residue was purified by flash column chromatography on silica gel (0% to 30% ethyl acetate in hexanes). Fractions containing the product were pooled and concentrated under reduced pressure to yield the title compound. LCMS: 703.5.

Step 5: tert-butyl 4-[[1-[3-(2,6-dioxo-3-piperidyl)-1-methyl-pyrazolo[3,4-b]pyridin-6-yl]-4-piperidyl]methyl]piperidine-1-carboxylate. A mixture of palladium hydroxide on carbon (4.6 mmol) and tert-butyl 4-[[1-[3-(2,6-dibenzyloxy-3-pyridyl)-1-methyl-pyrazolo[3,4-b]pyridin-6-yl]-4-piperidyl]methyl]piperidine-1-carboxylate (0.93 mmol) in tetrahydrofuran (7 mL) was evacuated under vacuum and backfilled with hydrogen for three times. The reaction mixture was vigorously stirred at room temperature for 12 h. Upon completion, the solution was filtered through a short pad of Celite, washed with dichloromethane (3×10 mL), concentrated under reduced pressure, and used for the next step without further purification. LCMS: 525.4.

Step 6: 3-[1-methyl-6-[4-(4-piperidylmethyl)-1-piperidyl]pyrazolo[3,4-b]pyridin-3-yl]piperidine-2,6-dione (INT 18-1). Trifluoroacetic acid (0.5 mL) was added into a solution of tert-butyl 4-[[1-[3-(2,6-dioxo-3-piperidyl)-1-methyl-pyrazolo[3,4-b]pyridin-6-yl]-4-piperidyl]methyl]piperidine-1-carboxylate (0.88 mmol) in dichloromethane (5 mL) at room temperature. The resulting solution was stirred for 2 h. Upon completion, the solution was concentrated under reduced pressure. The crude was dissolved in tetrahydrofuran/dichloromethane/acetonitrile (1:1:1) and subsequently neutralized with Amberlyst A21 ion exchange resin resin. The mixture was then filtered and concentrated under reduced pressure to afford the title compound which was taken forward without further purification. LCMS: 425.3.

The following intermediates were made in a similar fashion to INT 18-1 and are shown below in Table 2L. To prepare the below intermediates, different reagents/starting materials were used than previously described in the steps toward INT 18-1 and are noted in the last column of Table 2L—“Changes to Procedure: Different Reagents/Starting Materials”. A person of ordinary skill in the art will readily recognize which reagents/starting materials used for the synthesis of INT 18-1 were replaced with the different reagents/starting materials noted below.

TABLE 2M
Intermediates

| | | | Changes to Procedure: |
|---|---|---|---|
| | | | Different Reagents/Starting |
| Intermediate | Structure | LCMS | Materials |
| INT 18-2 | 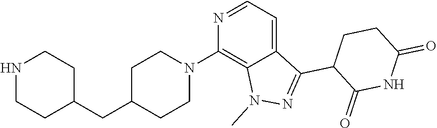 [SMILES: Cn1nc(C2CCC(=O)NC2=O)c2ccnc(N3CCC(CC4CCNCC4)CC3)c21] | 425.3 | 7-chloro-1H-pyrazolo[3,4- c]pyridine in step 1 |

### Intermediate 19-1

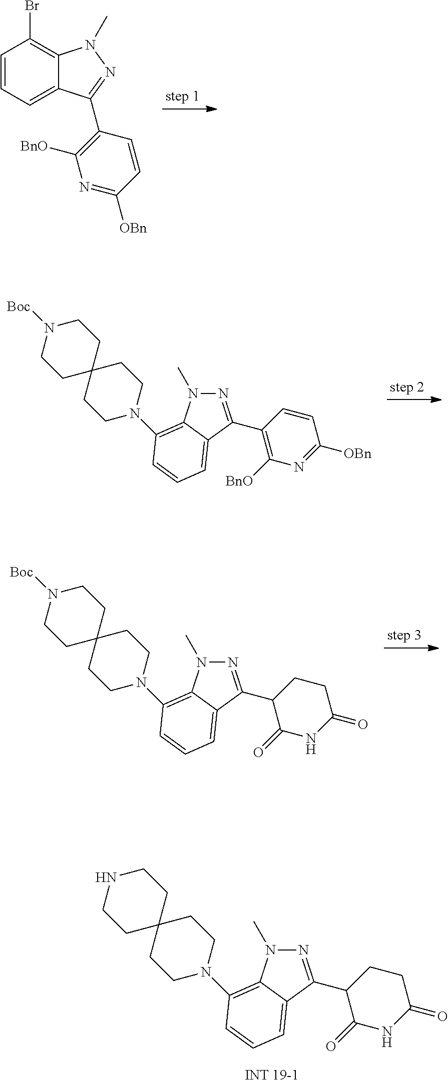 [SMILES: Cn1nc(-c2ccc(OCc3ccccc3)nc2OCc2ccccc2)c2cccc(Br)c21 ; Cn1nc(-c2ccc(OCc3ccccc3)nc2OCc2ccccc2)c2cccc(N3CCC4(CCN(C(=O)OC(C)(C)C)CC4)CC3)c21 ; Cn1nc(C2CCC(=O)NC2=O)c2cccc(N3CCC4(CCN(C(=O)OC(C)(C)C)CC4)CC3)c21 ; Cn1nc(C2CCC(=O)NC2=O)c2cccc(N3CCC4(CCNCC4)CC3)c21]

Step 1: tert-butyl 9-(3-(2,6-bis(benzyloxy)pyridin-3-yl)-1-methyl-1H-indazol-7-yl)-3,9-diazaspiro[5.5]undecane-3-carboxylate. To a solution of 3-(2,6-bis(benzyloxy)pyridin-3-yl)-7-bromo-1-methyl-1H-indazole (7.8 mmol) and tert-butyl 3,9-diazaspiro[5.5]undecane-3-carboxylate (9.8 mmol) in 1,4-dioxane (20 mL) was added cesium carbonate (29.5 mmol) and Pd-PEPPSI-IHept-Cl (0.5 mmol). The reaction mixture was degassed with nitrogen for 5 minutes, capped and stirred at 100° C. for 16 hours. Upon completion, the mixture was cooled to room temperature, filtered over Celite and partitioned between water and ethyl acetate. The organic layer was dried over sodium sulfate, filtered, and concentrated under reduced pressure. The residue was purified by silica gel chromatography eluting with 80% dichloromethane in petroleum ether. Fractions containing the product were pooled and concentrated under reduced pressure to yield the title compound LCMS: 674.3.

Step 2: tert-butyl 9-(3-(2,6-dioxopiperidin-3-yl)-1-methyl-1H-indazol-7-yl)-3,9-diazaspiro[5.5]undecane-3-carboxylate. To a stirred mixture of tert-butyl 9-(3-(2,6-bis(benzyloxy)pyridin-3-yl)-1-methyl-1H-indazol-7-yl)-3,9-diazaspiro[5.5]undecane-3-carboxylate (3.7 mmol) in tetrahydrofuran (25 mL) was added palladium hydroxide on carbon, powder, unreduced, 20% Pd, moisture 60% (2.5 g). The reaction vessel was evacuated and backfilled with hydrogen (3×), and the mixture was stirred under a hydrogen atmosphere at room temperature for 12 hours. Upon completion, the mixture was filtered over Celite, rinsed with ethyl acetate, and the filtrate was concentrated under reduced pressure. The residue was purified by silica gel chromatography eluting with 30% ethyl acetate in petroleum ether. Fractions containing the product were pooled and concentrated under reduced pressure to yield the title compound to afford the title compound which was used without further purification. LCMS: 496.3.

Step 3: 3-(1-methyl-7-(3,9-diazaspiro[5.5]undecan-3-yl)-1H-indazol-3-yl)piperidine-2,6-dione (INT-19-1). To a stirred solution of tert-butyl 9-(3-(2,6-dioxopiperidin-3-yl)-1-methyl-1H-indazol-7-yl)-3,9-diazaspiro[5.5]undecane-3-carboxylate (1.2 mmol) in dichloromethane (5 mL) was added 4 M HCl in 1,4-dioxane (4 ml) at room temperature and stirred for 3 hours. Upon completion, the reaction mixture was then concentrated under reduced pressure. The residue was dissolved in 20% IPA in dichloromethane and stirred with Amberlyst A21 ion exchange resin for 1 hour. The mixture was filtered and concentrated under reduced pressure to afford the title compound. LCMS: 396.3.

### Intermediate 20-1

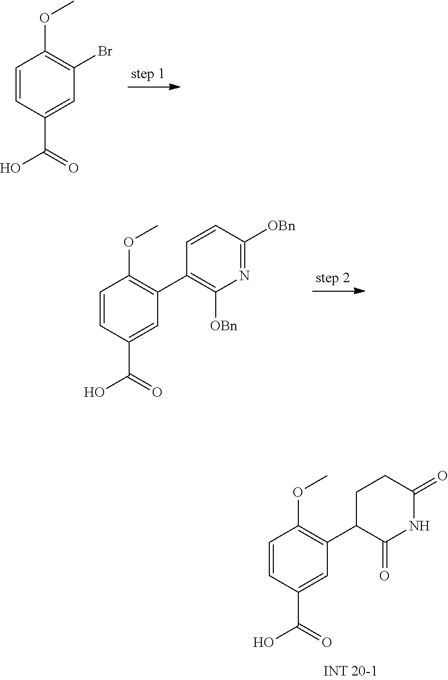 [SMILES: COc1ccc(C(=O)O)cc1Br ; COc1ccc(C(=O)O)cc1-c1ccc(OCc2ccccc2)nc1OCc1ccccc1 ; COc1ccc(C(=O)O)cc1C1CCC(=O)NC1=O]

Step 1: 3-(2,6-bis(benzyloxy)pyridin-3-yl)-4-methoxybenzoic acid. To a mixture of 3-bromo-4-methoxy-benzoic acid (39 mmol) and 2,6-bis(benzyloxy)-3-(4,4,5,5-tetramethyl-1,3,2-dioxaborolan-2-yl)pyridine (46.7 mmol) was added 1,4-dioxane (160 mL) and water (40 mL). The reaction mixture was degassed with nitrogen for 5 minutes. Sodium carbonate (78 mmol) and [1,1′-bis(diphenylphosphino)ferrocene]dichloropalladium(II) (3.90 mmol) were added under nitrogen. The reaction mixture was then stirred at 90° C. for 1 hour. Water was then added, and the mixture was extracted with ethyl acetate. The organic layers were combined, washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 442.2.

Step 2: 3-(2,6-dioxopiperidin-3-yl)-4-methoxybenzoic acid (INT 20-1). To a solution of 3-(2,6-bis(benzyloxy)pyridin-3-yl)-4-methoxybenzoic acid (14.7 mmol) in ethanol/ethyl acetate/DMF (120 mL, 1:1:0.16) was added Pd on carbon (3.0 g, 10 wt. % Pd). The mixture was then evacuated and backfilled with hydrogen three times and then stirred at 30° C. under a hydrogen atmosphere (15 psi) for 12 hours. The mixture was then filtered through Celite and concentrated under reduced pressure. To this remaining residue in DMF was added water and the precipitate was isolated after filtration, which was used without further purification. LCMS: 264.1.

Intermediate 21-1a-21-1b.

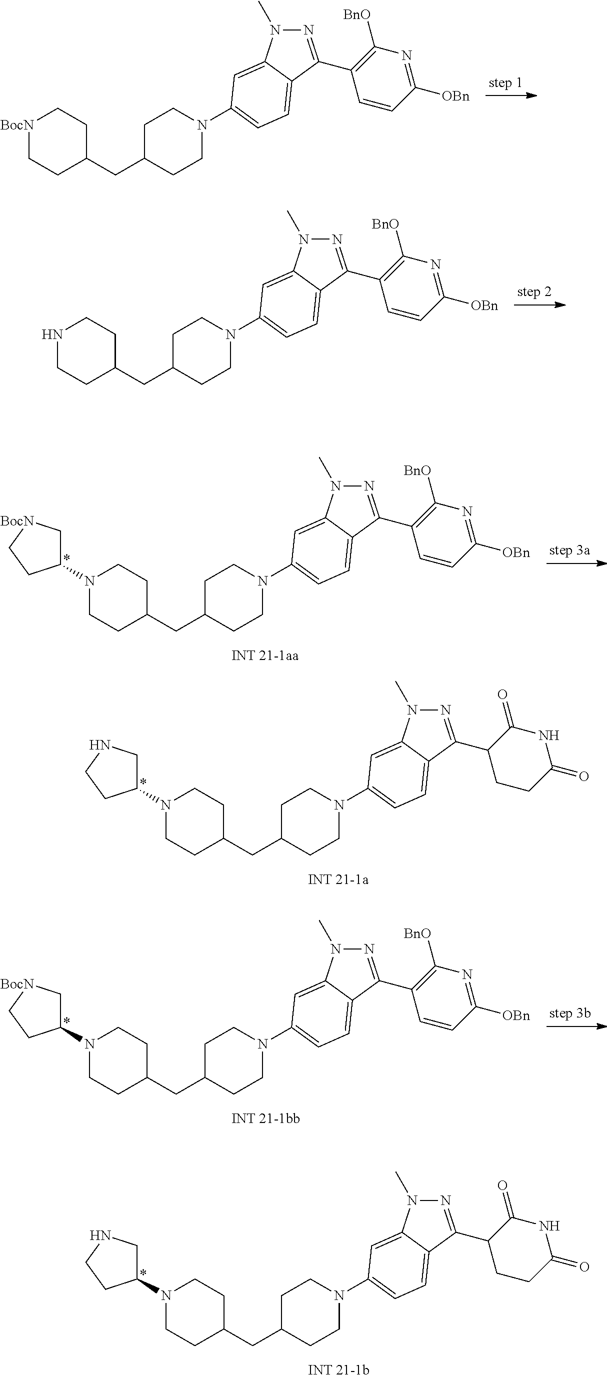 [SMILES: Cn1nc(-c2ccc(OCc3ccccc3)nc2OCc2ccccc2)c2ccc(N3CCC(CC4CCN(C(=O)OC(C)(C)C)CC4)CC3)cc21 ; Cn1nc(-c2ccc(OCc3ccccc3)nc2OCc2ccccc2)c2ccc(N3CCC(CC4CCNCC4)CC3)cc21 ; Cn1nc(-c2ccc(OCc3ccccc3)nc2OCc2ccccc2)c2ccc(N3CCC(CC4CCN([C@@H]5CCN(C(=O)OC(C)(C)C)C5)CC4)CC3)cc21 ; Cn1nc(C2CCC(=O)NC2=O)c2ccc(N3CCC(CC4CCN([C@@H]5CCNC5)CC4)CC3)cc21 ; Cn1nc(-c2ccc(OCc3ccccc3)nc2OCc2ccccc2)c2ccc(N3CCC(CC4CCN([C@H]5CCN(C(=O)OC(C)(C)C)C5)CC4)CC3)cc21 ; Cn1nc(C2CCC(=O)NC2=O)c2ccc(N3CCC(CC4CCN([C@H]5CCNC5)CC4)CC3)cc21]

Step 1: 3-(2,6-bis(benzyloxy)pyridin-3-yl)-1-methyl-6-(4-(piperidin-4-ylmethyl)piperidin-1-yl)-1H-indazole. Trifluoroacetic acid (1 mL) was added into a solution of tert-butyl 4-((1-(3-(2,6-bis(benzyloxy)pyridin-3-yl)-1-methyl-1H-indazol-6-yl)piperidin-4-yl)methyl)piperazine-1-carboxylate (2.9 mmol) in dichloromethane (10 mL) at room temperature. The resulting solution was stirred at room temperature for 2 hours. Upon completion, the reaction mixture was concentrated and purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 602.3.

Step 2: Tert-butyl 3-(4-((1-(3-(2,6-bis(benzyloxy)pyridin-3-yl)-1-methyl-1H-indazol-6-yl)piperidin-4-yl)methyl)piperidin-1-yl)pyrrolidine-1-carboxylate. To a stirred solution of 3-(2,6-bis(benzyloxy)pyridin-3-yl)-1-methyl-6-(4-(piperidin-4-ylmethyl)piperidin-1-yl)-1H-indazole (2.8 mmol) in ethanol (10 mL) was added tert-butyl 3-oxopyrrolidine-1-carboxylate (11.0 mmol), acetic acid (5.6 mmol) and 2-methylpyridine borane (18.2 mmol). The resulting mixture was stirred at 50° C. for 16 hours under an argon atmosphere. Upon completion, the reaction was cooled to room temperature, concentrated under reduced pressure and the residue product was purified by flash column chromatography on silica gel (0% to 30% 10% ammonium hydroxide in ethanol/dichloromethane). Fractions containing the product were pooled and concentrated under reduced pressure to afford the title compound as a mixture of two enantiomers which were separated by chiral SFC (CHIRALPAK-IK 5 um-4.6×100 mm column with ETOH-TFA to give two enantiomers, which were arbitrarily assigned as INT-21-1aa (Peak 1) and INT-21-1bb (Peak 2). INT-21-1aa: tert-butyl (R)-3-(4-((1-(3-(2,6-bis(benzyloxy)pyridin-3-yl)-1-methyl-1H-indazol-6-yl)piperidin-4-yl)methyl)piperidin-1-yl)pyrrolidine-1-carboxylate. LCMS: 772.3. INT-21-1bb: tert-butyl (S)-3-(4-((1-(3-(2,6-bis(benzyloxy)pyridin-3-yl)-1-methyl-1H-indazol-6-yl)piperidin-4-yl)methyl)piperidin-1-yl)pyrrolidine-1-carboxylate. LCMS: 772.3.

Step 3a: 3-(1-methyl-6-(4-((1-((R)-pyrrolidin-3-yl)piperidin-4-yl)methyl)piperidin-1-yl)-1H-indazol-3-yl)piperidine-2,6-dione (INT 21-1a). To a solution of INT-21-1aa (1.3 mmol) in tetrahydrofuran (20 mL) was added palladium hydroxide on carbon (6.5 mmol, 20 wt. % Pd) and the reaction mixture was then evacuated and backfilled with hydrogen three times. The mixture was then stirred at room temperature, under a hydrogen atmosphere, for 16 hours. The mixture was then filtered through Celite then concentrated under reduced pressure to afford a crude residue. The residue was then dissolved in dichloromethane (5 mL) and hydrochloric acid (0.5 mL, 4 M in 1,4-dioxane) was added and the mixture was stirred for 4 hours at room temperature. Upon completion, the reaction mixture was concentrated and purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 493.3.

3-(1-methyl-6-(4-((1-((s)-pyrrolidin-3-yl)piperidin-4-yl)methyl)piperidin-1-yl)-1H-indazol-3-yl)piperidine-2,6-dione (INT 21-1b) was prepared analogously to INT 21-1a, substituting INT 21-1aa in step 6b with INT 21-1bb. LCMS: 493.3.

### Intermediate 22-1-22-2.

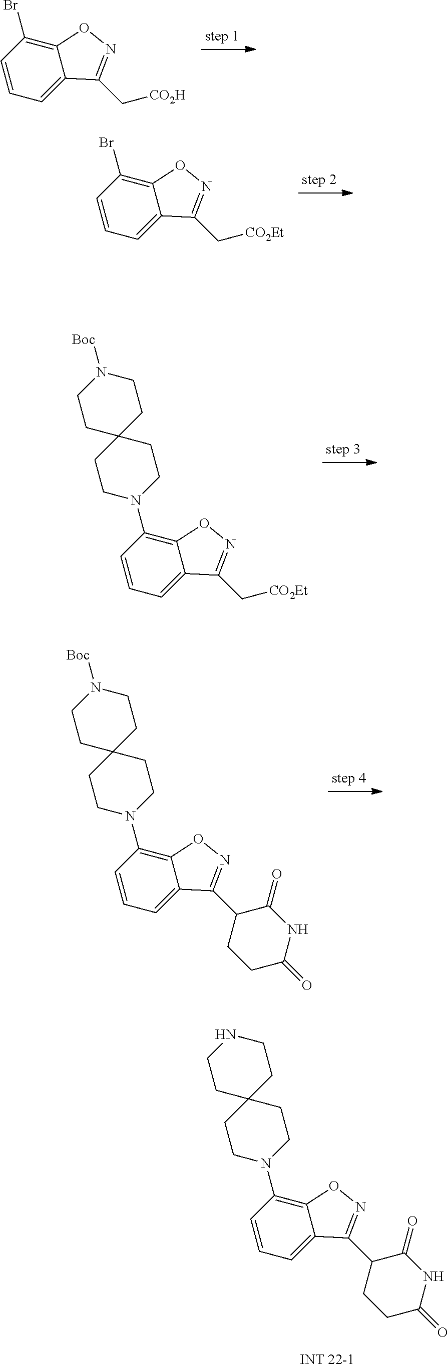

Step 1: Ethyl 2-(7-bromobenzo[d]isoxazol-3-yl)acetate. To a solution of 2-(7-bromobenzo[d]isoxazol-3-yl)acetic acid (70 mmol) in EtOH (180 mL) was added H₂SO₄ (9.4 mL) dropwise at 0° C. Upon addition, the reaction was heated at 80° C. for 6 hours. After completion, the reaction mixture was concentrated and the residue was partitioned between sat. sodium bicarbonate solution and ethyl acetate. The organic layers were combined, washed with brine, dried over anhydrous sodium sulfate, filtered, and concentrated under reduced pressure to yield the title compound. LCMS: 285.05.

Step 2: tert-butyl 9-(3-(2-ethoxy-2-oxoethyl)benzo[d]isoxazol-7-yl)-3,9-diazaspiro[5.5]undecane-3-carboxylate. To a solution of ethyl 2-(7-bromobenzo[d]isoxazol-3-yl)acetate (15.8 mmol) and tert-butyl 3,9-diazaspiro[5.5]undecane-3-carboxylate (15.8 mmol) in 1,4-dioxane (40 mL) was added potassium carbonate (60 mmol) and Pd-PEPPSI-IHept-Cl (1.1 mmol). The reaction mixture was degassed with nitrogen for 5 minutes, capped and stirred at 105° C. for 16 hours. Upon completion, the mixture was cooled to room temperature, filtered over Celite and partitioned between water and ethyl acetate. The organic layer was dried over sodium sulfate, filtered, and concentrated under reduced pressure. The residue was purified by silica gel chromatography eluting with 10-20% ethyl acetate in petroleum ether. Fractions containing the product were pooled and concentrated under reduced pressure to yield the title compound LCMS: 458.4.

Step 3: tert-butyl 9-(3-(2,6-dioxopiperidin-3-yl)benzo[d]isoxazol-7-yl)-3,9-diazaspiro[5.5]undecane-3-carboxylate. To a solution of tert-butyl 9-(3-(2-ethoxy-2-oxoethyl)benzo[d]isoxazol-7-yl)-3,9-diazaspiro[5.5]undecane-3-carboxylate (4.3 mmol) in THE (40 mL) were added acrylamide (8.7 mmol) and potassium t-butoxide (4.3 mmol) at 0° C. and the resulting reaction mixture was stirred for 1 h 0° C. Upon completion, water and ethyl acetate were added. The organic layer was separated, dried over anhydrous magnesium sulfate, filtered, and concentrated under reduced pressure. The residue was purified by flash column chromatography on silica gel (40% to 50% ethyl acetate in hexanes) to afford the title compound. LCMS: 483.4.

Step 4: 3-(7-(3,9-diazaspiro[5.5]undecan-3-yl)benzo[d]isoxazol-3-yl)piperidine-2,6-dione (INT 22-1). To a solution of tert-butyl 9-(3-(2,6-dioxopiperidin-3-yl)benzo[d]isoxazol-7-yl)-3,9-diazaspiro[5.5]undecane-3-carboxylate (2.9 mmol) in dichloromethane (6 mL) was added trifluoroacetic acid (2 mL) at 0° C. and the mixture was stirred for 2 hours at room temperature. After completion, the reaction mixture was concentrated and washed with ethyl ether. The residue was dissolved in 20% isopropyl alcohol and subsequently neutralized with Amberlyst A21 ion exchange resin resin to provide the title compound. LCMS: 383.4.

The following intermediates were made in a similar fashion to INT 22-1 and are shown below in Table 2M. To prepare the below intermediates, different reagents/starting materials were used than previously described in the steps toward INT 22-1 and are noted in the last column of Table 2M—“Changes to Procedure: Different Reagents/Starting Materials”. A person of ordinary skill in the art will readily recognize which reagents/starting materials used for the synthesis of INT 22-1 were replaced with the different reagents/starting materials noted below.

TABLE 2N
Intermediates

| | | | Changes to Procedure: Different |
|---|---|---|---|
| Intermediate | Structure | LCMS | Reagents/Starting Materials |
| INT 22-2 | 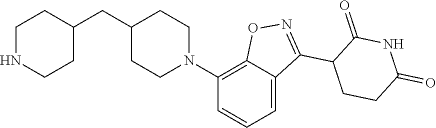 [SMILES: O=C1CCC(c2noc3c(N4CCC(CC5CCNCC5)CC4)cccc23)C(=O)N1] | 411.4 | tert-butyl 4-(piperidin-4- ylmethyl)piperidine-1-carboxylate in step 2 |

### III. Compounds

### Examples 1-1-1-10

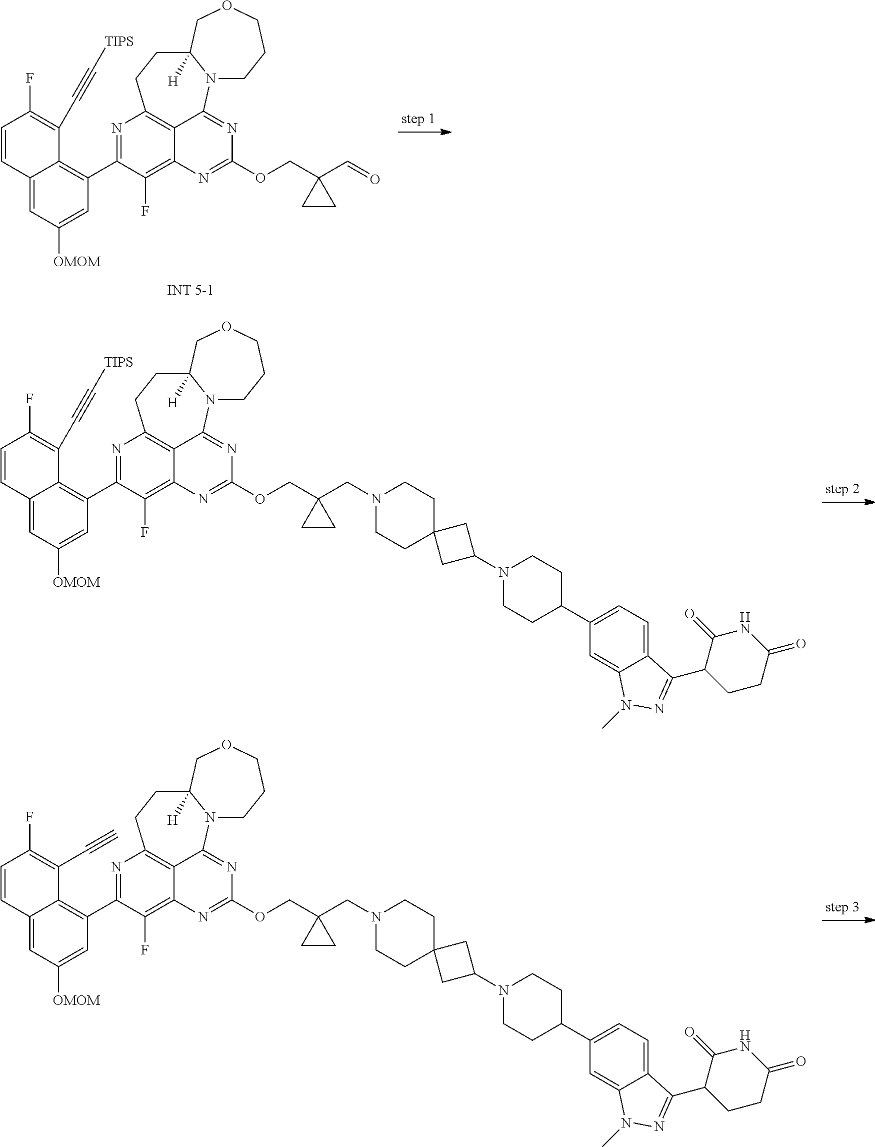

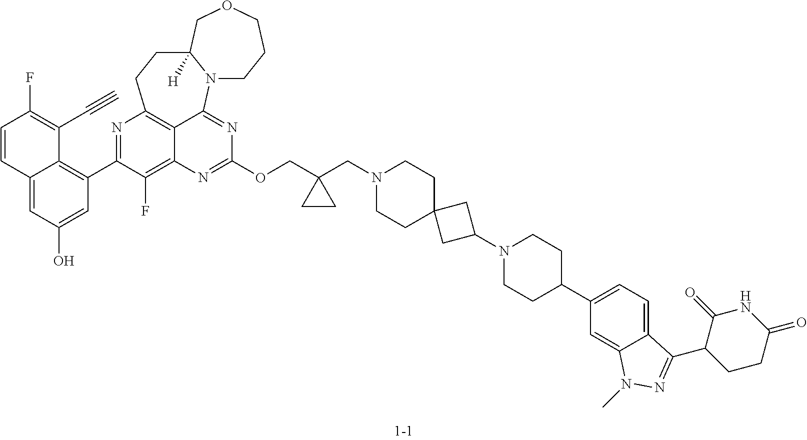

Step 1: 3-(6-(1-(7-((1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione. To a stirred solution of (S)-1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropane-1-carbaldehyde INT 5-1 (0.11 mmol) in ethanol (3.2 mL) at room temperature was added 3-(6-(1-(7-azaspiro[3.5]nonan-2-yl)piperidin-4-yl)-1-methyl-1H-indazol-3-yl)piperidine-2,6-dione INT 6-2 (0.32 mmol), acetic acid (1.8 mmol) and 2-methylpyridine borane (0.7 mmol). The resulting mixture was stirred at 50° C. for 2 hours under an argon atmosphere. Upon completion, the reaction was cooled to room temperature, then saturated sodium bicarbonate solution was added. The mixture was extracted with ethyl acetate (3×25 mL), the combined organic layers were dried over anhydrous sodium sulfate, filtered and concentrated under reduced pressure. The residue was purified by flash column chromatography on silica gel (0% to 30% 10% ammonium hydroxide in ethanol/dichloromethane). Fractions containing the product were pooled and concentrated under reduced pressure to yield the title compound. LCMS: 1191.7.

Step 2: 3-(6-(1-(7-((1-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione. To a stirred solution of 3-(6-(1-(7-((1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione (0.09 mmol) in N,N-dimethylformamide (2 mL) was added cesium fluoride (1.3 mmol) in one portion. The resulting mixture was heated to 45° C. for 1 hour. The mixture was then cooled to room temperature, filtered through Celite and concentrated under reduced pressure to yield the title compound which was used without further purification. LCMS: 1034.6.

Step 3: 3-(6-(1-(7-((1-((((S)-2-(8-ethynyl-7-fluoro-3-hydroxynaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione (Example 1-1). 3-(6-(1-(7-((1-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione was dissolved in dichloromethane (1 mL) and cooled to 0° C. A solution of hydrochloric acid in 1,4-dioxane (4M, 0.3 mL) was added dropwise and the mixture was stirred for 30 minutes. Upon completion, the crude mixture was isolated by filtration and purified by RP-HPLC (0 to 50%, 0.1% trifluoroacetic acid in acetonitrile/0.1% trifluoroacetic acid in water). Fractions containing the product were pooled and lyophilized to yield the title compound. ¹H NMR (400 MHz, Methanol-d₄) δ 7.90-7.82 (m, 1H), 7.71 (d, J=8.5 Hz, 1H), 7.39-7.20 (m, 4H), 7.08 (d, J=8.3 Hz, 1H), 4.66 (d, J=12.0 Hz, 1H), 4.58-4.45 (m, 1H), 4.44-4.31 (m, 2H), 4.15-3.90 (m, 7H), 3.72 (ddt, J=54.1, 27.1, 15.6 Hz, 9H), 3.44-3.36 (m, 2H), 3.12-2.91 (m, 6H), 2.85-2.66 (m, 2H), 2.65-2.52 (m, 11H), 2.52-2.41 (m, 11H), 2.41-2.27 (m, 4H), 2.25-2.11 (m, 5H), 2.09-1.90 (m, 7H), 1.01 (s, 2H), 0.89 (s, 2H). LCMS: 990.5.

The following Examples were made in a similar fashion to Example 1-1 and are shown below in Table 3A1. To prepare the below Examples, different reagents/starting materials were used than some of those described in the steps toward Example 1-1 and are noted in the last column of Table 3A2—“Changes to Procedure: Different Reagents/Starting Materials”. A person of ordinary skill in the art will readily recognize which reagents/starting materials used for the synthesis of Example 1-1 were replaced with the different reagents/starting materials noted below.

TABLE 3A1
Compounds

| Example | Structure |
|---|---|
| 1-2 | 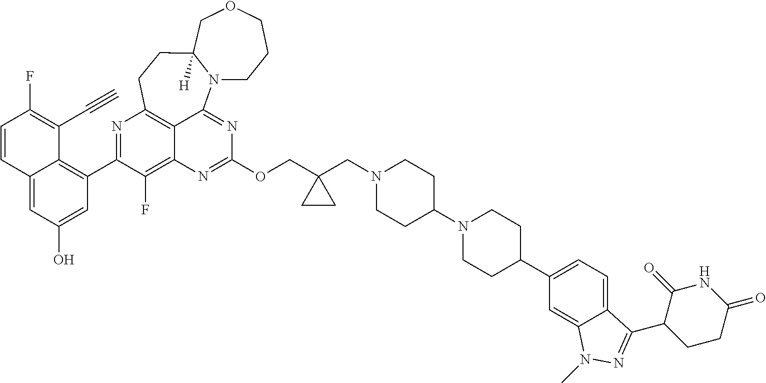 |
| 1-3 | 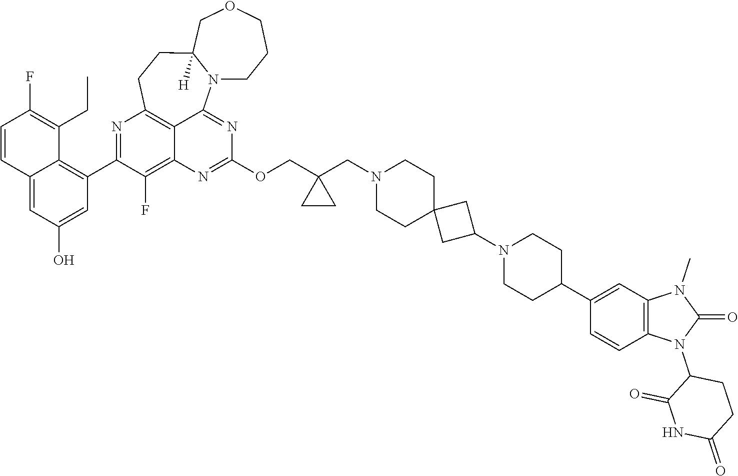 |
| 1-4 | 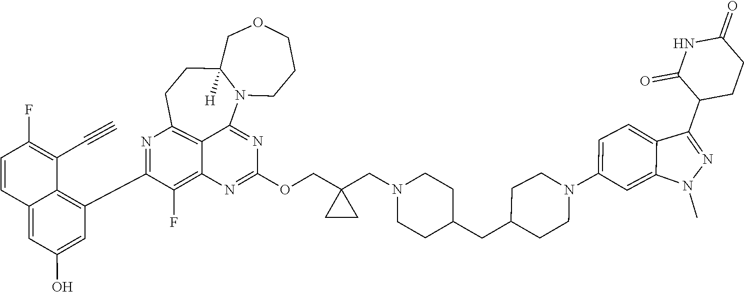 |
| 1-5 | 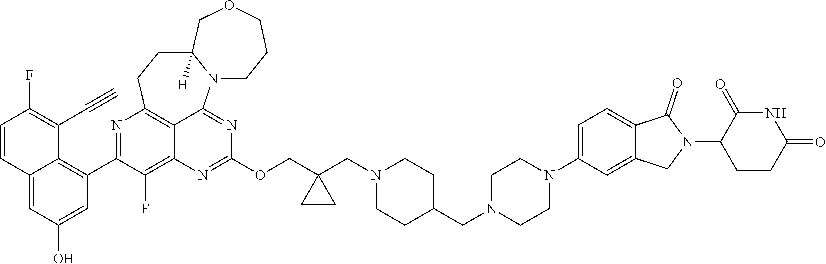 |
| 1-6 | 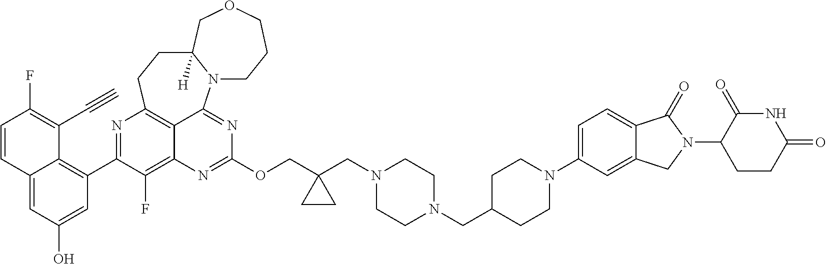 |
| 1-7 | 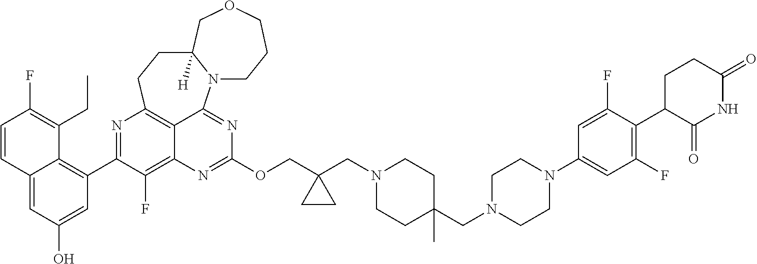 |
| 1-8 | 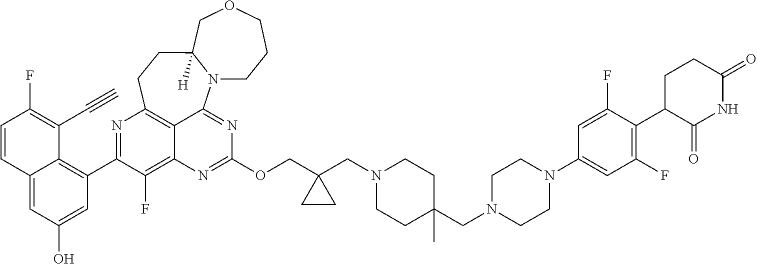 |
| 1-9 | 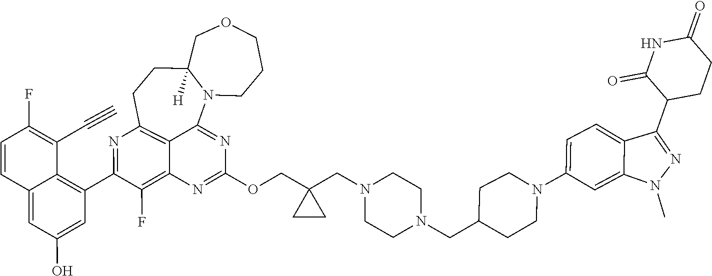 |
| 1-10 | 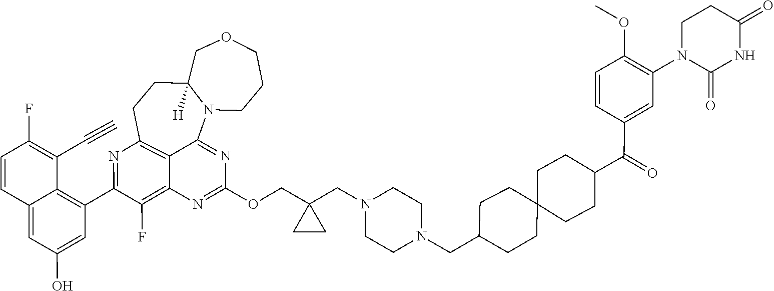 |

TABLE 3A2
Compounds

| | | | Changes to |
|---|---|---|---|
| | | | Procedure: |
| | | | Different |
| | | | Reagents/ |
| | | | Starting |
| Example | LCMS | ¹H NMR (400 MHz, Methanol-d₄) | Materials |
| 1-2 | 950.5 | δ 7.83-7.65 (m, 2H), 7.38-7.05 (m, 4H), 6.97 (dd, | INT 6-1 |
| | | J = 38.4, 8.5 Hz, 1H), 5.03-4.88 (m, 1H), 4.66 (d, J = | in step 1 |
| | | 11.9 Hz, 1H), 4.57-4.34 (m, 3H), 4.20-3.78 (m, | |
| | | 10H), 3.79-3.53 (m, 6H), 3.47-3.38 (m, 0H), 3.28- | |
| | | 2.66 (m, 10H), 2.56-2.25 (m, 9H), 2.25-1.90 (m, | |
| | | 6H), 1.18-0.81 (m, 4H). | |
| 1-3 | 1010.6 | δ 7.69 (dd, J = 9.0, 5.8 Hz, 1H), 7.34-7.31 (m, 1H), | INT 5-2 and |
| | | 7.27 (td, J = 9.3, 3.0 Hz, 1H), 7.18-6.99 (m, 4H), | INT 7-1 |
| | | 5.35 (dd, J = 12.5, 5.4 Hz, 1H), 4.67-4.48 (m, 2H), | in step 1. Step 2 |
| | | 4.47-4.37 (m, 1H), 4.17-3.66 (m, 10H), 3.61 (d, J = | was omitted. |
| | | 11.9 Hz, 2H), 3.45 (s, 3H), 3.25-2.89 (m, 8H), | |
| | | 2.88-2.74 (m, 2H), 2.64-2.52 (m, 1H), 2.52-1.89 | |
| | | (m, 19H), 1.03 (s, 2H), 0.96-0.80 (m, 5H). | |
| 1-4 | 964.5 | δ 7.93-7.83 (m, 2H), 7.57 (s, 1H), 7.43-7.20 (m, | INT 8-1 in step |
| | | 4H), 4.77 (dd, J = 12.0, 5.5 Hz, 1H), 4.60-4.49 (m, | 1. Step 2 was |
| | | 1H), 4.45-4.38 (m, 1H), 4.37-4.23 (m, 1H), 4.14 | performed at |
| | | (dd, J = 14.1, 3.6 Hz, 1H), 4.07 (s, 3H), 4.04-3.80 | room |
| | | (m, 6H), 3.79-3.67 (m, 4H), 3.48-3.39 (m, 3H), | temperature in |
| | | 3.25-3.07 (m, 3H), 3.06-2.91 (m, 2H), 2.89-2.71 | THF for 30 |
| | | (m, 2H), 2.58-2.46 (m, 1H), 2.46-2.30 (m, 3H), | minutes using a |
| | | 2.27-2.17 (m, 1H), 2.10-1.94 (m, 5H), 1.86-1.70 | TBAF solution |
| | | (m, 2H), 1.70-1.52 (m, 4H), 1.35 (t, J = 6.9 Hz, | (3.0 equiv., 1M |
| | | 1H), 1.30-1.22 (m, 1H), 1.11-0.98 (m, 2H), 0.97- | in THF). |
| | | 0.84 (m, 2H). | |
| 1-5 | 966.5 | δ 7.89 (dt, J = 9.2, 5.6 Hz, 1H), 7.70 (d, J = 9.2 Hz, | INT 9-1 |
| | | 1H), 7.42-7.26 (m, 3H), 7.20-7.09 (m, 2H), 5.12 | in step 1 |
| | | (dd, J = 13.3, 5.2 Hz, 1H), 4.68-4.49 (m, 2H), 4.49- | |
| | | 4.37 (m, 2H), 4.29-3.77 (m, 9H), 3.77-3.36 (m, | |
| | | 11H), 3.26-3.01 (m, 6H), 2.97-2.87 (m, 1H), 2.84- | |
| | | 2.72 (m, 1H), 2.52-2.24 (m, 4H), 2.24-2.07 (m, | |
| | | 4H), 1.99-1.92 (m, 1H), 1.86-1.70 (m, 2H), 1.11- | |
| | | 0.83 (m, 4H). | |
| 1-6 | 966.6 | δ 7.92-7.84 (m, 1H), 7.63 (d, J = 8.7 Hz, 1H), 7.42- | INT 9-2 |
| | | 7.23 (m, 3H), 7.10-7.01 (m, 2H), 5.11 (dd, J = | in step 1 |
| | | 13.4, 5.1 Hz, 1H), 4.65-4.33 (m, 5H), 4.16-3.79 | |
| | | (m, 8H), 3.77-3.67 (m, 2H), 3.48 (m, 1H), 3.25- | |
| | | 2.98 (m, 8H), 2.97-2.66 (m, 8H), 2.52-2.27 (m, | |
| | | 3H), 2.26-2.09 (m, 2H), 2.02-1.78 (m, 4H), 1.46- | |
| | | 1.28 (m, 2H), 0.90 (s, 2H), 0.74 (s, 2H). | |
| 1-7 | 965.6 | δ 7.72-7.60 (m, 1H), 7.29 (dd, J = 10.0, 2.6 Hz, | INT 5-2 and |
| | | 1H), 7.21 (t, J = 9.4 Hz, 1H), 7.14-6.99 (m, 1H), | INT 10-2 |
| | | 6.53-6.39 (m, 2H), 4.71-4.27 (m, 4H), 4.14 (dd, J = | in step 1. Step 2 |
| | | 12.5, 5.2 Hz, 1H), 4.11-3.81 (m, 6H), 3.80-3.65 | was omitted. |
| | | (m, 2H), 3.60-3.34 (m, 8H), 3.28 (m, 1H), 3.26- | |
| | | 3.01 (m, 6H), 2.88-2.74 (m, 1H), 2.74-2.65 (m, | |
| | | 1H), 2.47-2.06 (m, 9H), 2.00-1.89 (m, 1H), 1.89- | |
| | | 1.73 (m, 2H), 1.35-1.20 (m, 3H), 1.07-0.97 (m, | |
| | | 2H), 0.95-0.78 (m, 5H). | |
| 1-8 | 961.5 | δ 7.93-7.73 (m, 1H), 7.40-7.13 (m, 3H), 6.47 (d, J = | INT 10-2 |
| | | 12.4 Hz, 1H), 6.36-6.24 (m, 1H), 4.98-4.90 (m, | in step 1 |
| | | 1H), 4.68-4.46 (m, 2H), 4.38 (d, J = 11.8 Hz, 1H), | |
| | | 4.28-3.78 (m, 8H), 3.78-3.58 (m, 3H), 3.55-3.37 | |
| | | (m, 4H), 3.25-2.91 (m, 8H), 2.90-2.60 (m, 3H), | |
| | | 2.47-2.05 (m, 7H), 2.00-1.89 (m, 2H), 1.76 (dd, J = | |
| | | 37.0, 14.2 Hz, 2H), 1.34-1.21 (m, 3H), 1.17- | |
| | | 1.06 (m, 1H), 1.06-0.76 (m, 4H). | |
| 1-9 | 965.5 | δ 7.91-7.84 (m, 1H), 7.74 (d, J = 9.0 Hz, 1H), 7.41- | INT 8-2 |
| | | 7.29 (m, 3H), 7.27-7.18 (m, 1H), 7.15-7.08 (m, | in step 1. Step 2 |
| | | 1H), 4.70-4.34 (m, 5H), 4.18-4.05 (m, 2H), 4.01 | was performed |
| | | (s, 3H), 3.98-3.74 (m, 5H), 3.73-3.69 (m, 1H), | at room |
| | | 3.47 (s, 1H), 3.24-2.99 (m, 10H), 2.87-2.59 (m, | temperature in |
| | | 5H), 2.55-2.29 (m, 5H), 2.27-2.17 (m, 1H), 2.10- | THF for 30 |
| | | 1.89 (m, 5H), 1.62-1.45 (m, 2H), 1.00-0.88 (m, | minutes using a |
| | | 2H), 0.85-0.74 (m, 2H). | TBAF solution |
| | | | (3.0 equiv., 1M |
| | | | in THF). |
| 1-10 | 1038.6 | δ 7.90 (dd, J = 9.1, 5.7 Hz, 1H), 7.44 (dd, J = 8.5, 2.2 | INT 11-1 |
| | | Hz, 1H), 7.41-7.33 (m, 3H), 7.28 (dd, J = 11.4, 2.6 | in step 1 |
| | | Hz, 1H), 7.19 (d, J = 8.5 Hz, 1H), 4.62-4.41 (m, | |
| | | 3H), 4.19-3.79 (m, 8H), 3.79-3.38 (m, 7H), 3.27- | |
| | | 2.91 (m, 9H), 2.91-2.68 (m, 6H), 2.50-2.26 (m, | |
| | | 2H), 2.26-2.09 (m, 1H), 2.03-1.93 (m, 1H), 1.84- | |
| | | 1.66 (m, 3H), 1.60 (s, 3H), 1.54-1.10 (m, 9H), 0.88 | |
| | | (s, 2H), 0.68 (s, 2H) | |

### Examples 2-1-2-5

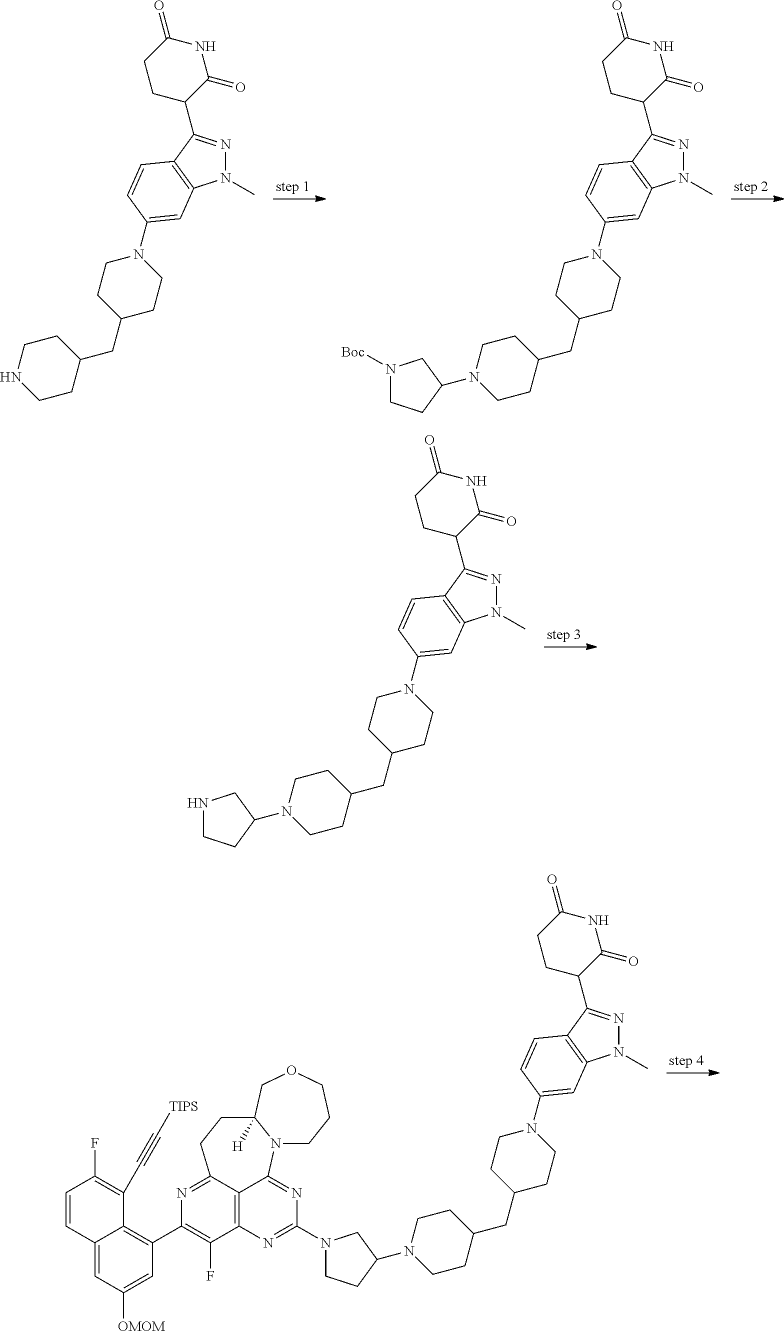

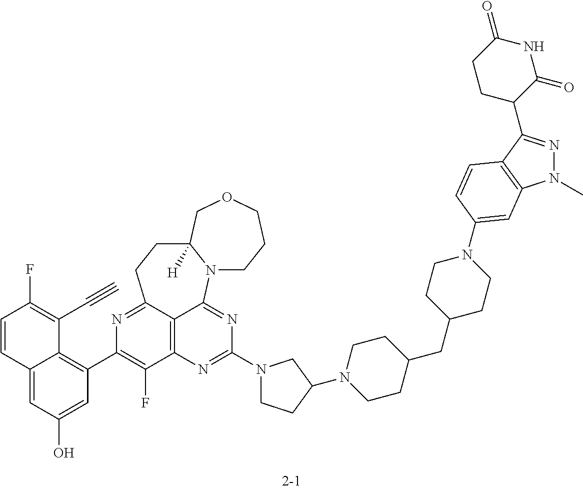

Step 1: tert-butyl 3-(4-((1-(3-(2,6-dioxopiperidin-3-yl)-1-methyl-1H-indazol-6-yl)piperidin-4-yl)methyl)piperidin-1-yl)pyrrolidine-1-carboxylate. To a solution of 3-(1-methyl-6-(4-(piperidin-4-ylmethyl)piperidin-1-yl)-1H-indazol-3-yl)piperidine-2,6-dione (100 mg, 0.2 mmol) in ethanol (1 mL) was added tert-butyl 3-oxopyrrolidine-1-carboxylate (175 mg, 0.9 mmol) and the mixture was stirred at room temperature for 20 minutes. 2-methylpyridine borane complex (164 mg, 1.5 mmol) was added and the resulting reaction mixture was stirred for 16 hours at 60° C. Upon completion, the resulting mixture was cooled to room temperature and concentrated under reduced pressure to afford the title compound which was used without further purification. LCMS: 593.3.

Step 2: 3-(1-methyl-6-(4-((1-(pyrrolidin-3-yl)piperidin-4-yl)methyl)piperidin-1-yl)-1H-indazol-3-yl)piperidine-2,6-dione. To a solution of tert-butyl 3-(4-((1-(3-(2,6-dioxopiperidin-3-yl)-1-methyl-1H-indazol-6-yl)piperidin-4-yl)methyl)piperidin-1-yl)pyrrolidine-1-carboxylate (420 mg, 0.7 mmol) in dichloromethane (5 mL) was added trifluoracetic acid (0.7 mL) and the reaction mixture was stirred for 16 hours at room temperature. After completion, the reaction mixture was concentrated and purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 493.3.

Step 3: 3-(6-(4-((1-(1-((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)pyrrolidin-3-yl)piperidin-4-yl)methyl)piperidin-1-yl)-1-methyl-1H-indazol-3-yl)piperidine-2,6-dione. A mixture of (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (30 mg, 0.04 mmol), 3-[1-methyl-6-[4-[(1-pyrrolidin-3-yl-4-piperidyl)methyl]-1-piperidyl]indazol-3-yl]piperidine-2,6-dione (115 mg, 0.2 mmol) and N,N-diisopropylethylamine (0.2 mL, 1.2 mmol) in dimethylacetamide (1 mL) was stirred for 3 hours at 130° C. The reaction mixture was then allowed to cool to room temperature, concentrated and purified by RP-HPLC (0 to 90% 0.1% TFA in MeCN/0.1% TFA in H₂O). Fractions containing the product were pooled and lyophilized to afford the title compound. LCMS: 1149.4.

Step 4: 3-(6-(4-((1-(1-((S)-2-(8-ethynyl-7-fluoro-3-hydroxynaphthalen-1-yl)-1-fluoro-4,5,5a,6,9,10-hexahydro-8H-7-oxa-3,10a,11,13-tetraazanaphtho[1,8-ab]heptalen-12-yl)pyrrolidin-3-yl)piperidin-4-yl)methyl)piperidin-1-yl)-1-methyl-1H-indazol-3-yl)piperidine-2,6-dione (Example 2-1). To a solution of 3-(6-(4-((1-(1-((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)pyrrolidin-3-yl)piperidin-4-yl)methyl)piperidin-1-yl)-1-methyl-1H-indazol-3-yl)piperidine-2,6-dione (35 mg, 0.03 mmol) in N,N-dimethylformamide (1 mL) was added cesium fluoride (69 mg, 0.5 mmol). After stirring for 4 hours at 45° C., the reaction mixture was cooled to room temperature, filtered and concentrated under reduced pressure. The crude product was then dissolved in dichloromethane (5 mL) and treated with trifluoracetic acid (0.5 mL). The resulting reaction mixture was stirred for 6 hours at room temperature. Upon completion, the reaction mixture was concentrated and purified by RP-HPLC (0 to 90% 0.1% TFA in MeCN/0.1% TFA in H₂O). Fractions containing the product were pooled and lyophilized to afford the title compound. ¹H NMR (400 MHz, Methanol-d₄) δ 7.97 (dd, J=9.2, 5.7 Hz, 1H), 7.82 (d, J=8.9 Hz, 1H), 7.56-7.32 (m, 4H), 7.23 (d, J=8.9 Hz, 1H), 4.63-4.45 (m, 1H), 4.39 (dd, J=9.5, 5.1 Hz, 1H), 4.22-3.60 (m, 19H), 3.20-3.03 (m, 3H), 2.92-2.60 (m, 4H), 2.55-2.21 (m, 7H), 2.16-1.74 (m, 8H), 1.68-1.26 (m, 7H). LCMS: 949.3.

The following Examples were made in a similar fashion to Example 2-1 and are shown below in Table 3A3. To prepare the below Examples, different reagents/starting materials were used than some of those described in the steps toward Example 2-1 and are noted in the last column of Table 3A4—“Changes to Procedure: Different Reagents/Starting Materials”. A person of ordinary skill in the art will readily recognize which reagents/starting materials used for the synthesis of Example 2-1 were replaced with the different reagents/starting materials noted below.

TABLE 4A3
Compounds

| Example | Structure |
|---|---|
| 2-2 | 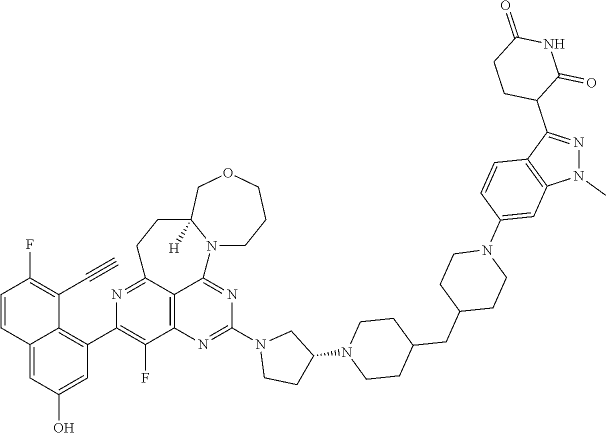 |
| 2-3 | 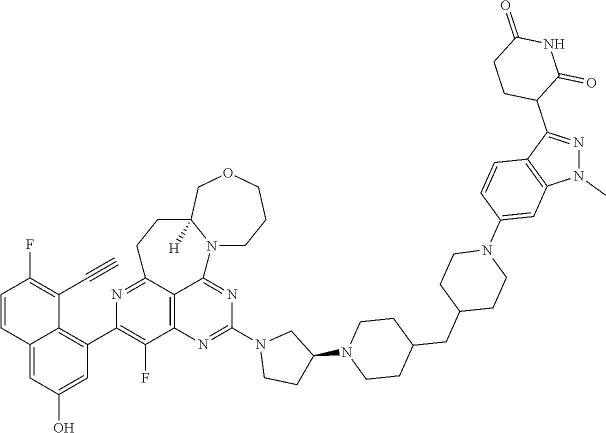 |
| 2-4 | 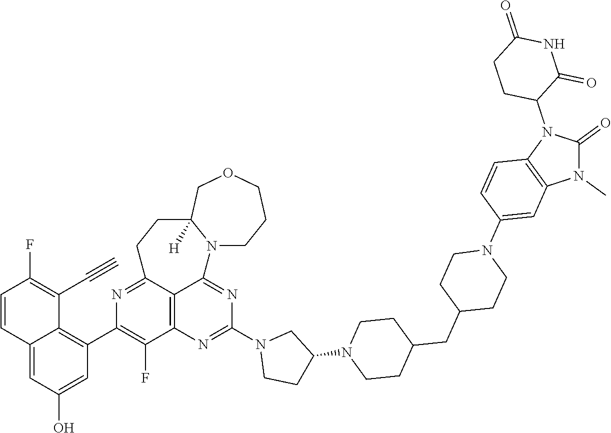 |
| 2-5 | 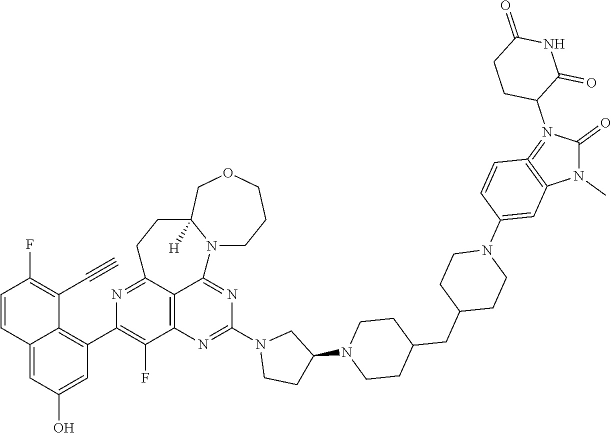 |

Notes: The stereochemistry at the chiral center of pyrrolidinyl of Examples 2-2, 2-3, 2-4, and 2-5 is assigned arbitrarily, and the specific configuration (R or S) is not known.

TABLE 3A4
Compounds

| | | | Changes to |
|---|---|---|---|
| | | | Procedure: |
| | | | Different |
| | | | Reagents/ |
| | | | Starting |
| Example | LCMS | ¹H NMR (400 MHz, Methanol-d₄) | Materials |
| 2-2 | 949.3 | δ 7.97 (dd, J = 9.2, 5.7 Hz, 1H), 7.82 (d, J = 8.9 | Step 1 and 2 were |
| | | Hz, 1H), 7.56-7.32 (m, 4H), 7.23 (d, J = 8.9 Hz, | omitted. INT 21-1a |
| | | 1H), 4.63-4.45 (m, 1H), 4.39 (dd, J = 9.5, 5.1 | in step 3. Step 4 |
| | | Hz, 1H), 4.22-3.60 (m, 19H), 3.20-3.03 (m, | was performed at |
| | | 3H), 2.92-2.60 (m, 4H), 2.55-2.21 (m, 7H), | room temperature |
| | | 2.16-1.74 (m, 8H), 1.68-1.26 (m, 7H). | in THF for 30 |
| | | | minutes using a |
| | | | TBAF solution (3.0 |
| | | | equiv., 1M in |
| | | | THF). |
| 2-3 | 949.3 | δ 7.97 (dd, J = 9.2, 5.7 Hz, 1H), 7.82 (d, J = 8.9 | Step 1 and 2 were |
| | | Hz, 1H), 7.56-7.32 (m, 4H), 7.23 (d, J = 8.9 Hz, | omitted. INT 21-1b |
| | | 1H), 4.63-4.45 (m, 1H), 4.39 (dd, J = 9.5, 5.1 | in step 3. Step 4 |
| | | Hz, 1H), 4.22-3.60 (m, 19H), 3.20-3.03 (m, | was performed at |
| | | 3H), 2.92-2.60 (m, 4H), 2.55-2.21 (m, 7H), | room temperature |
| | | 2.16-1.74 (m, 8H), 1.68-1.26 (m, 7H). | in THF for 30 |
| | | | minutes using a |
| | | | TBAF solution (3.0 |
| | | | equiv., 1M in |
| | | | THF). |
| 2-4 | 965.3 | δ 8.00-7.91 (m, 1H), 7.50-7.23 (m, 6H), 5.41 | Step 1 and 2 were |
| | | (dd, J = 12.4, 5.4 Hz, 1H), 4.57-4.45 (m, 1H), | omitted. INT 13-2a |
| | | 4.42-3.58 (m, 18H), 3.49 (s, 3H), 3.27-3.04 (m, | in step 3. Step 4 |
| | | 4H), 3.00-2.80 (m, 3H), 2.72-2.60 (m, 1H), | was performed at |
| | | 2.53-1.34 (m, 18H). | room temperature |
| | | | in THF for 30 |
| | | | minutes using a |
| | | | TBAF solution (3.0 |
| | | | equiv., 1M in |
| | | | THF). |
| 2-5 | 965.3 | δ 8.00-7.92 (m, 1H), 7.52-7.22 (m, 6H), 5.41 | Step 1 and 2 were |
| | | (dd, J = 12.4, 5.4 Hz, 1H), 4.61-4.38 (m, 2H), | omitted. INT 13-2b |
| | | 4.18-3.61 (m, 17H), 3.49 (s, 3H), 3.31-3.18 (m, | in step 3. Step 4 |
| | | 3H), 3.15-3.06 (m, 1H), 2.99-2.78 (m, 3H), | was performed at |
| | | 2.73-1.36 (m, 19H). | room temperature |
| | | | in THF for 30 |
| | | | minutes using a |
| | | | TBAF solution (3.0 |
| | | | equiv., 1M in |
| | | | THF). |

### Examples 3-1-3-48

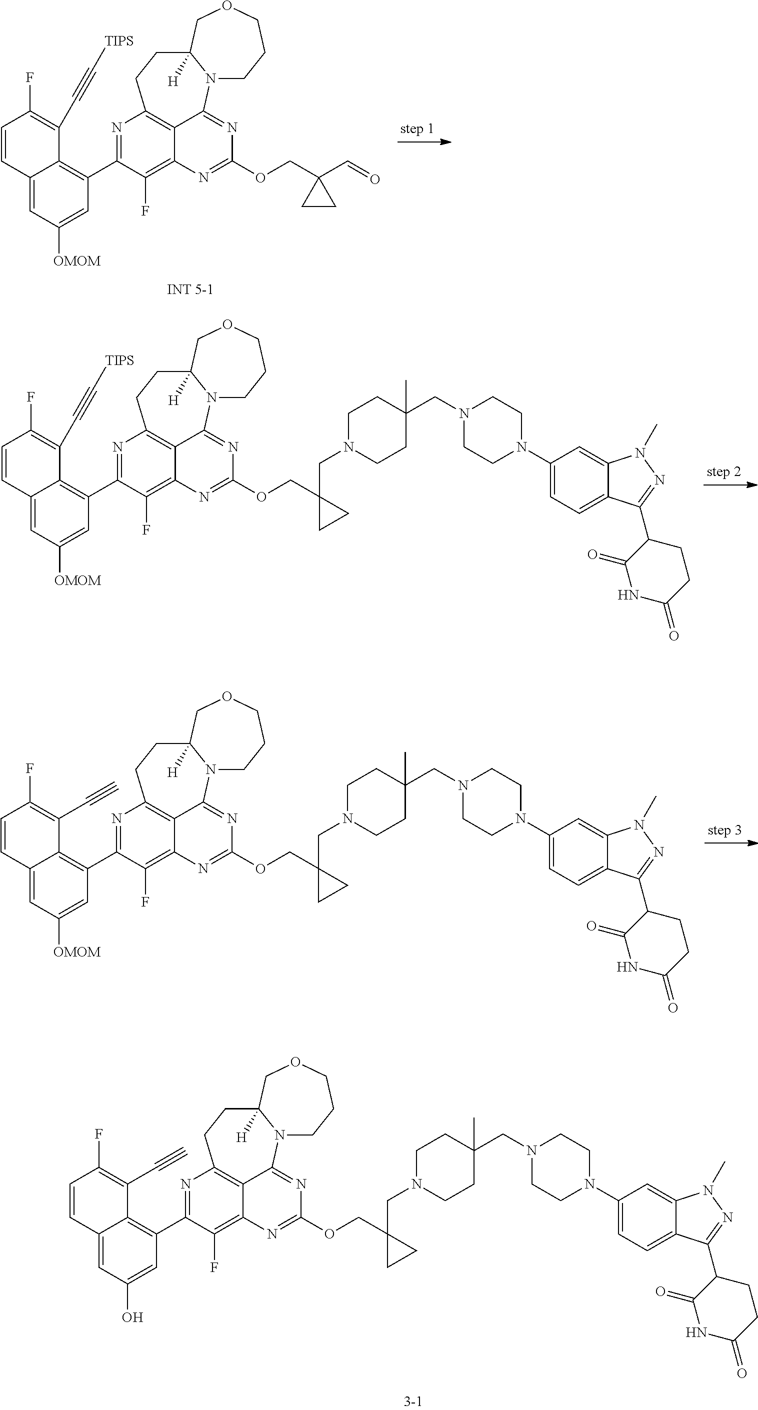

Step 1: 3-(6-(1-(7-((1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione. To a stirred solution of (S)-1-(((1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropane-1-carbaldehyde INT 5-1 (0.09 mmol) in ethanol (2.5 mL) at room temperature was added 3-(1-methyl-6-(4-((4-methylpiperidin-4-yl)methyl)piperazin-1-yl)-1H-indazol-3-yl)piperidine-2,6-dione INT 8-3 (0.26 mmol) as free base, acetic acid (1.5 mmol) and 2-methylpyridine borane (0.6 mmol). The resulting mixture was stirred at 50° C. for 2 hours under an argon atmosphere. Upon completion, the reaction was cooled to room temperature, then concentrated under reduced pressure. The residue was purified by flash column chromatography on silica gel (0% to 30% 10% ammonium hydroxide in ethanol/dichloromethane). Fractions containing the product were pooled and concentrated under reduced pressure to yield the title compound. LCMS: 1179.7.

Step 2: 3-(6-(4-((1-((1-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)cyclopropyl)methyl)-4-methylpiperidin-4-yl)methyl)piperazin-1-yl)-1-methyl-1H-indazol-3-yl)piperidine-2,6-dione. To a stirred solution of 3-(6-(1-(7-((1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-1-methyl-1H-indazol-3-yl)piperidine-2,6-dione (0.09 mmol) in THE (3 mL) was added tetrabutylammonium fluoride (0.1 mL, 1M solution in THF) dropwise. The resulting mixture was stirred at room temperature for 15 minutes. The reaction mixture was then concentrated under reduced pressure. The residue was purified by flash column chromatography on silica gel (0% to 30% 10% ammonium hydroxide in ethanol/dichloromethane). Fractions containing the product were pooled and concentrated under reduced pressure to yield the title compound. LCMS: 1023.6.

Step 3: 3-(6-(4-((1-((1-((((S)-2-(8-ethynyl-7-fluoro-3-hydroxynaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)cyclopropyl)methyl)-4-methylpiperidin-4-yl)methyl)piperazin-1-yl)-1-methyl-1H-indazol-3-yl)piperidine-2,6-dione (Example 3-1). 3-(6-(4-((1-((1-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)cyclopropyl)methyl)-4-methylpiperidin-4-yl)methyl)piperazin-1-yl)-1-methyl-1H-indazol-3-yl)piperidine-2,6-dione from step 2 was dissolved in dichloromethane (3 mL) and cooled to 0° C. A solution of hydrochloric acid in 1,4-dioxane (4M, 0.3 mL) was added dropwise and the mixture was stirred for 30 minutes. Upon completion, the reaction mixture was concentrated and purified by RP-HPLC (0 to 50%, 0.1% trifluoroacetic acid in acetonitrile/0.1% trifluoroacetic acid in water). Fractions containing the product were pooled and lyophilized to yield the title compound. ¹H NMR (400 MHz, Methanol-d₄) δ 7.84-7.74 (m, 1H), 7.64-7.51 (m, 2H), 7.32-7.17 (m, 3H), 6.90-6.73 (m, 1H), 4.67-4.50 (m, 2H), 4.47-4.32 (m, 2H), 4.22-4.12 (m, 1H), 4.12-3.78 (m, 12H), 3.78-3.61 (m, 5H), 3.25-3.19 (m, 3H), 2.90-2.70 (m, 3H), 2.57-2.09 (m, 9H), 2.03-1.71 (m, 4H), 1.39-1.25 (m, 4H), 1.17-1.09 (m, 1H), 1.08-0.80 (in, 5H). LCMS: 979.6.

The following Examples were made in a similar fashion to Example 3-1 and are shown below in Table 3A5. To prepare the below Examples, different reagents/starting materials were used than some of those described in the steps toward Example 3-1 and are noted in the last column of Table 3A6—“Changes to Procedure: Different Reagents/Starting Materials”. A person of ordinary skill in the art will readily recognize which reagents/starting materials used for the synthesis of Example 3-1 were replaced with the different reagents/starting materials noted below.

TABLE 3A5
Compounds

| Example | Structure |
|---|---|
| 3-2 | 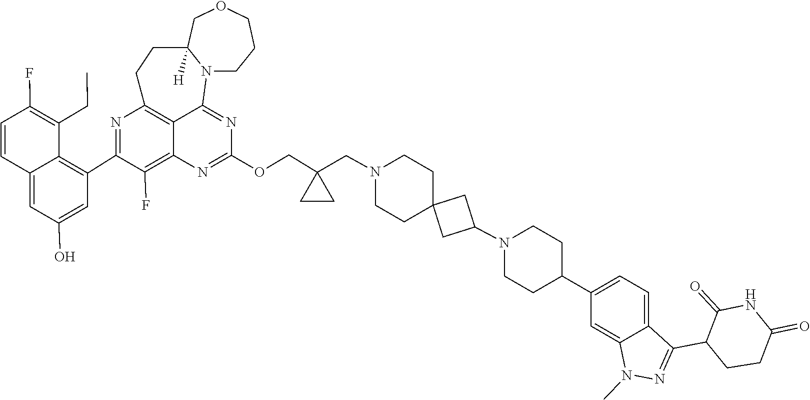 |
| 3-3 | 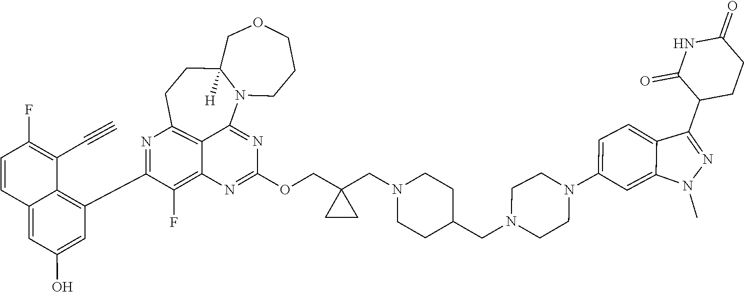 |
| 3-4 | 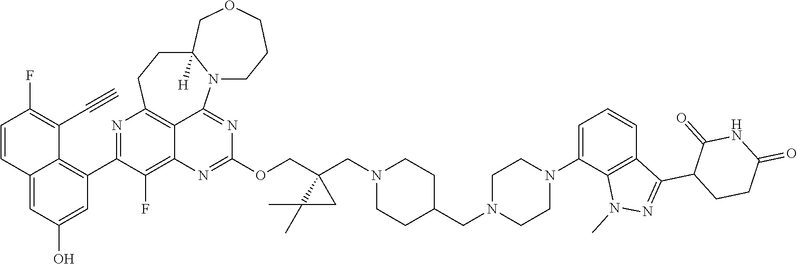 |
| 3-5 | 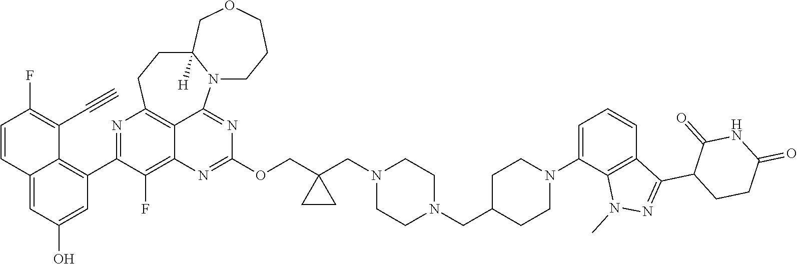 |
| 3-6 | 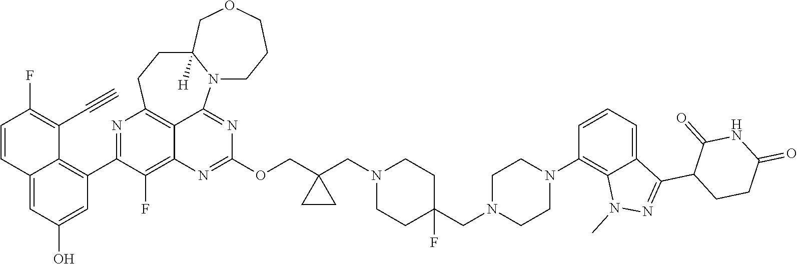 |
| 3-7 | 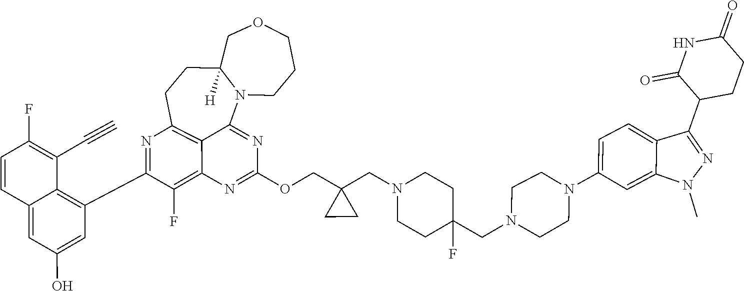 |
| 3-8 | 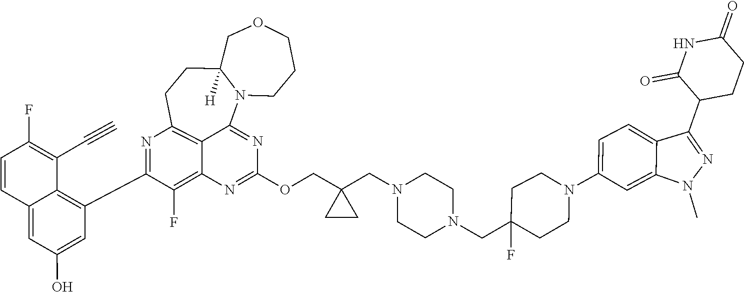 |
| 3-9 | 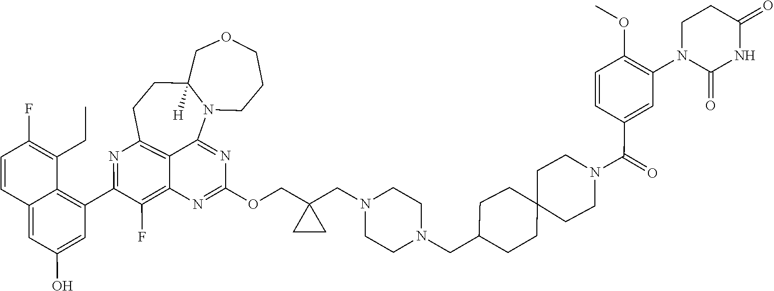 |
| 3-10 | 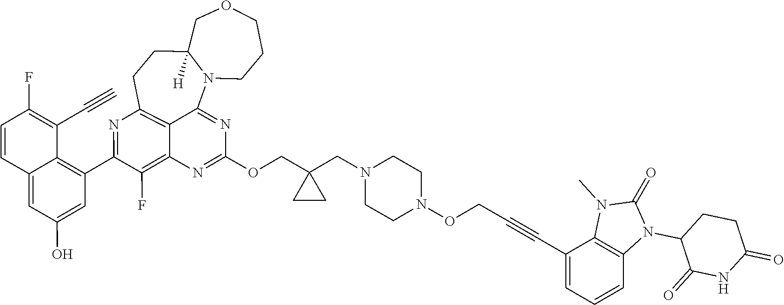 |
| 3-11 | 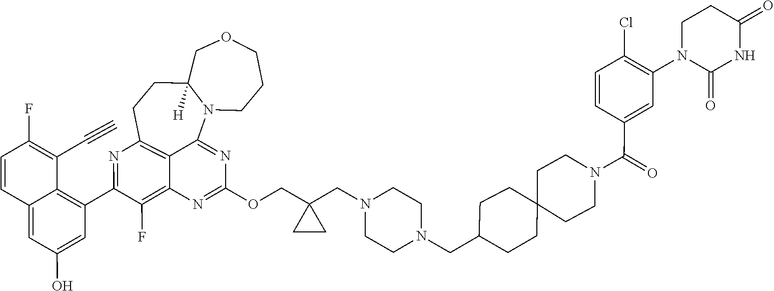 |
| 3-12 | 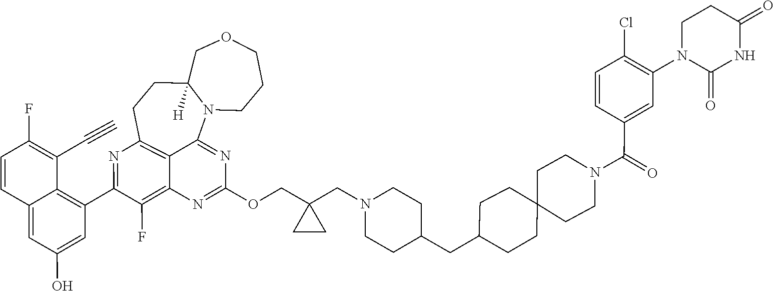 |
| 3-13 | 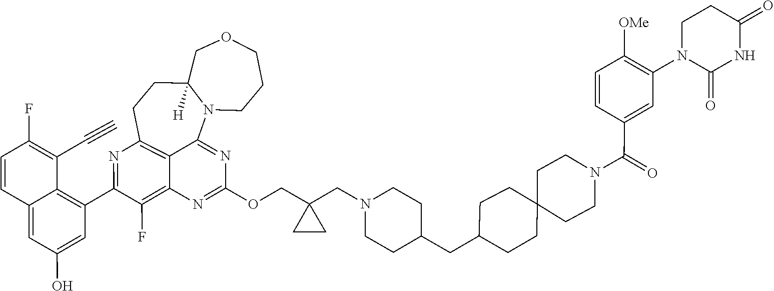 |
| 3-14 | 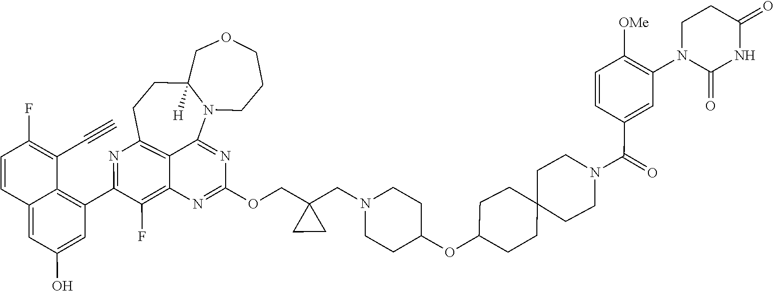 |
| 3-15 | 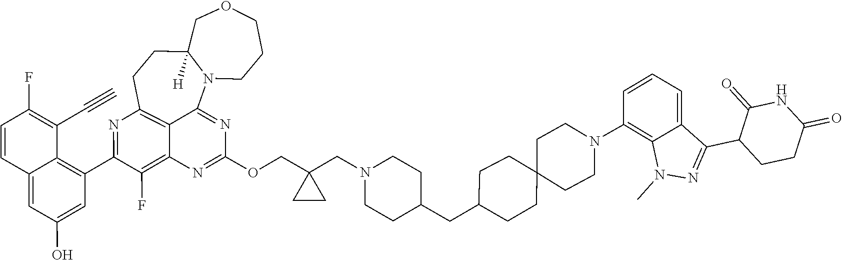 |
| 3-16 | 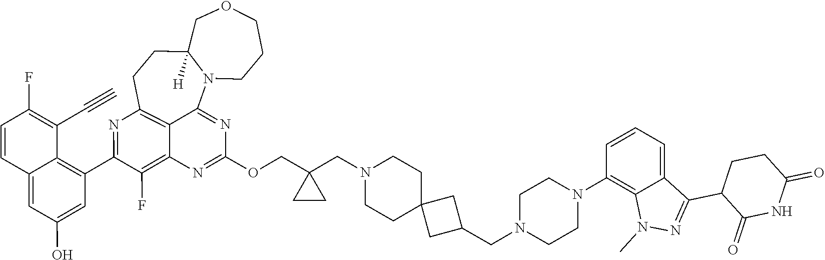 |
| 3-17 | 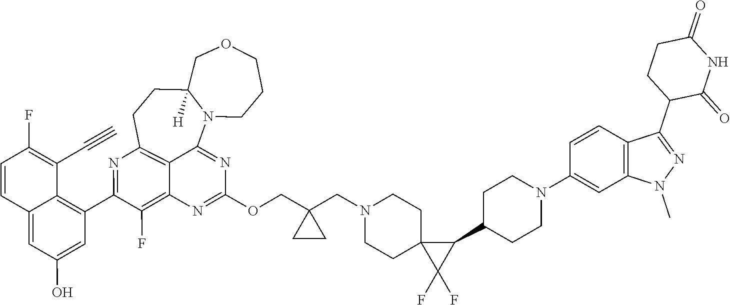 |
| 3-18 | 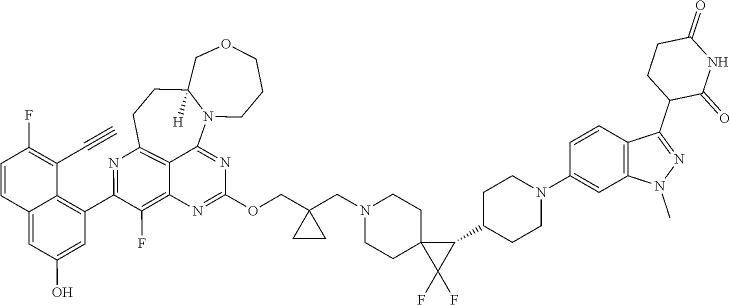 |
| 3-19 | 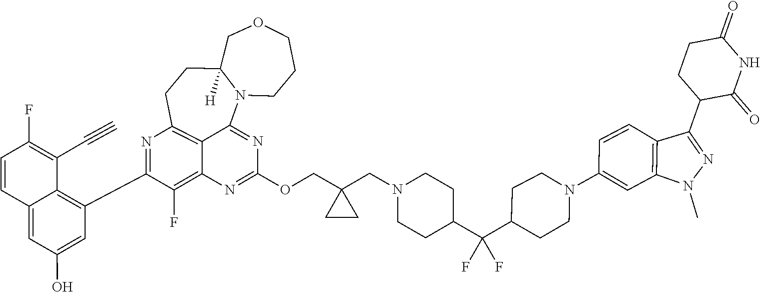 |
| 3-20 | 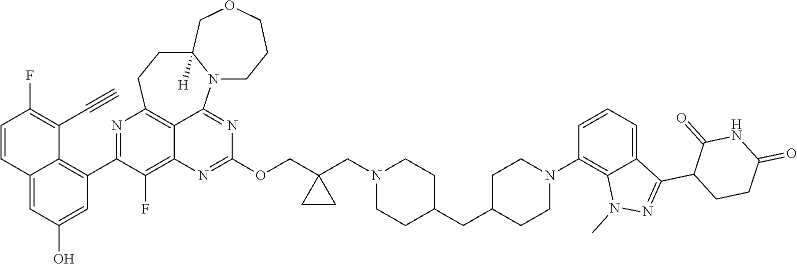 |
| 3-21 | 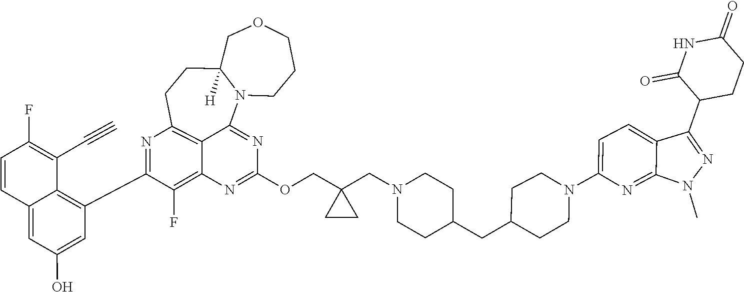 |
| 3-22 | 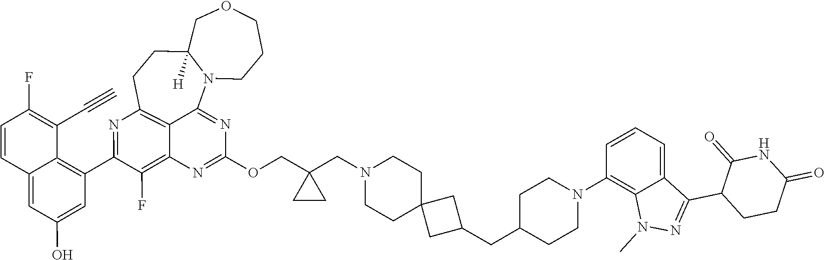 |
| 3-23 | 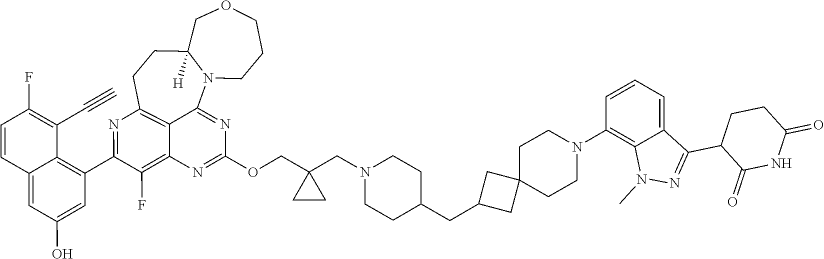 |
| 3-24 | 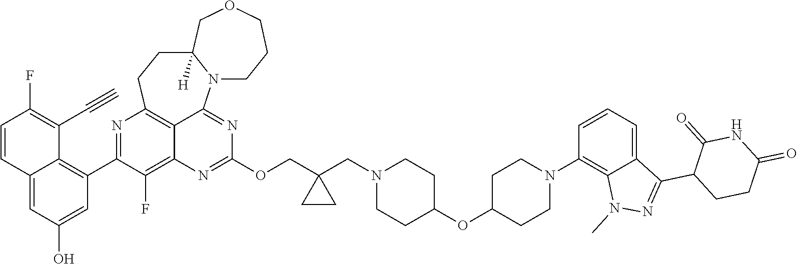 |
| 3-25 | 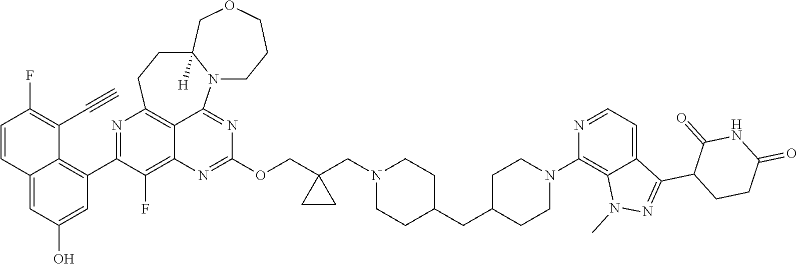 |
| 3-26 | 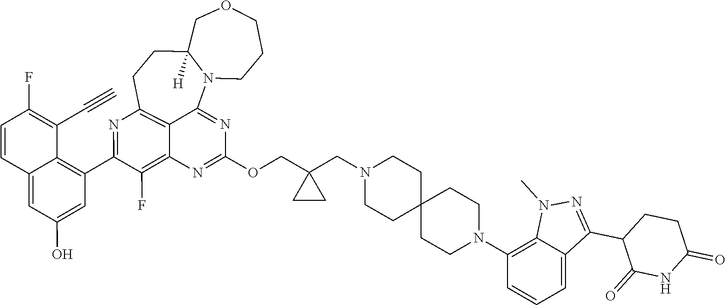 |
| 3-27 | 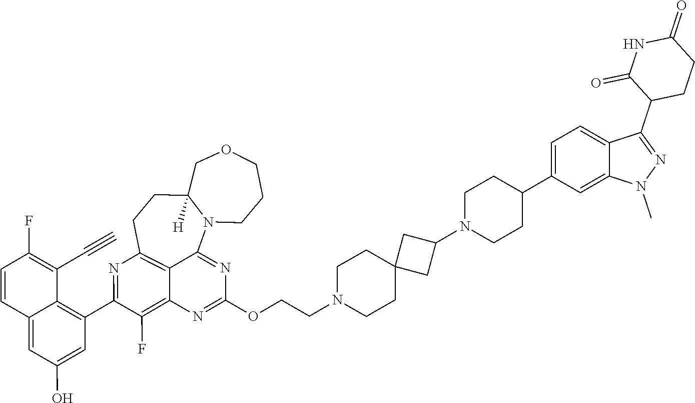 |
| 3-28 | 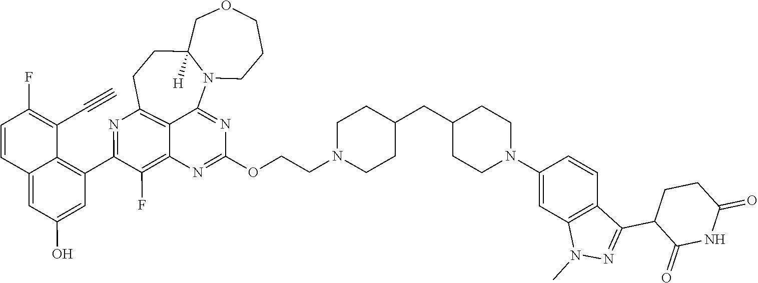 |
| 3-29 | 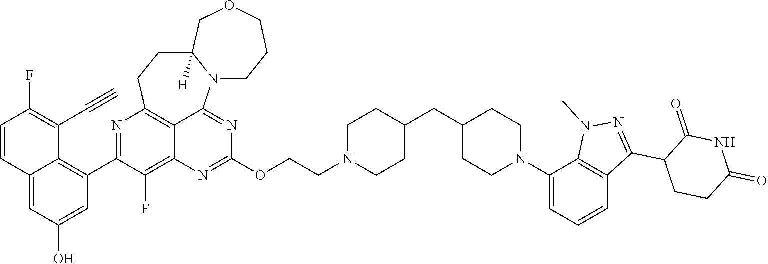 |
| 3-30 | 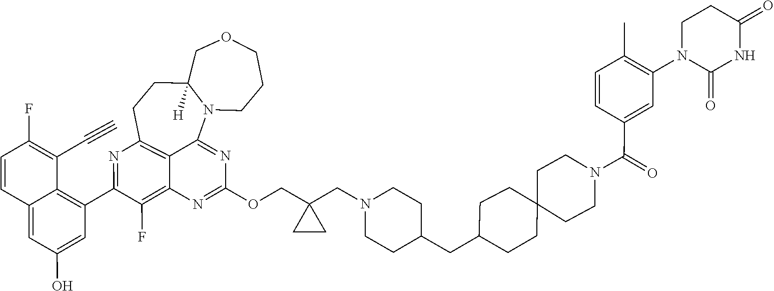 |
| 3-31 | 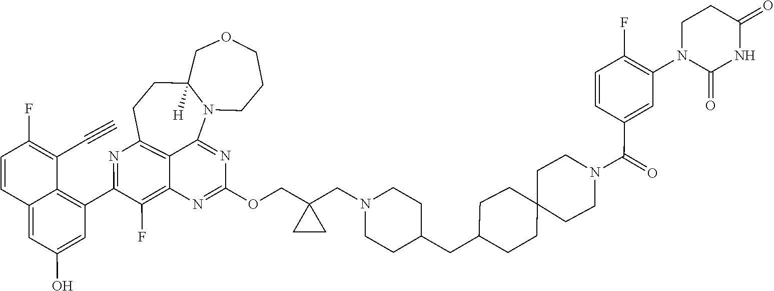 |
| 3-32 | 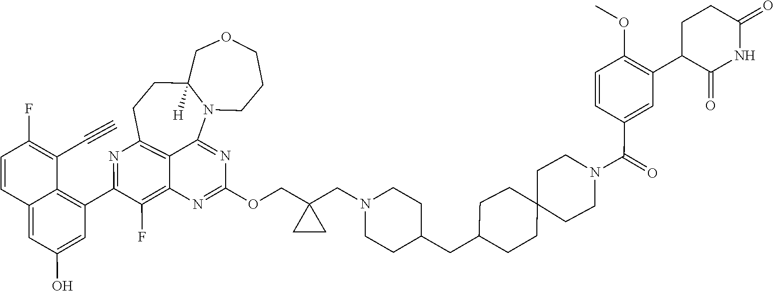 |
| 3-33 | 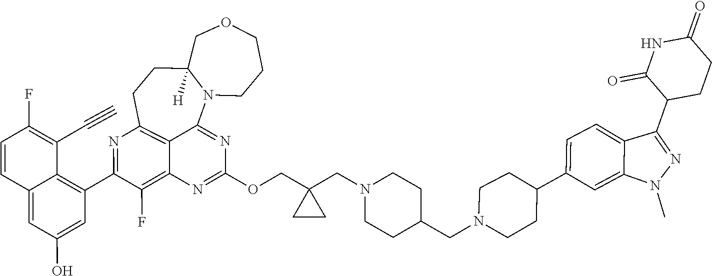 |
| 3-34 | 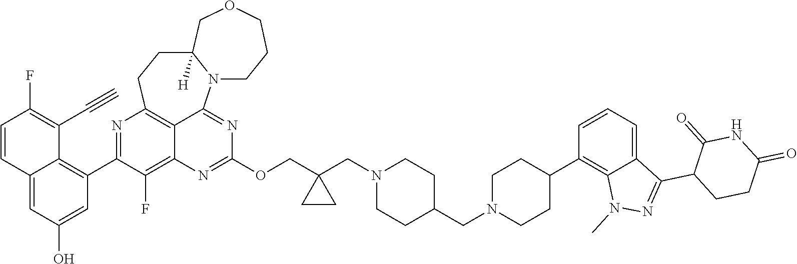 |
| 3-35 | 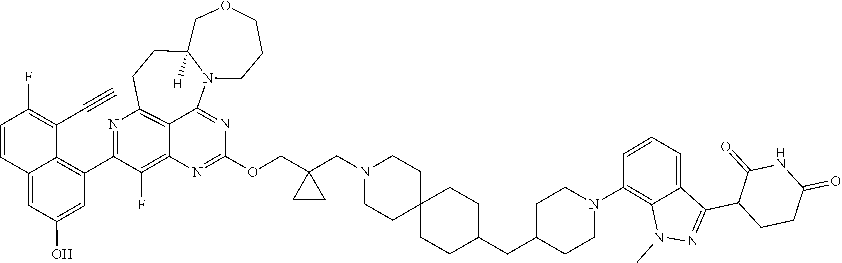 |
| 3-36 | 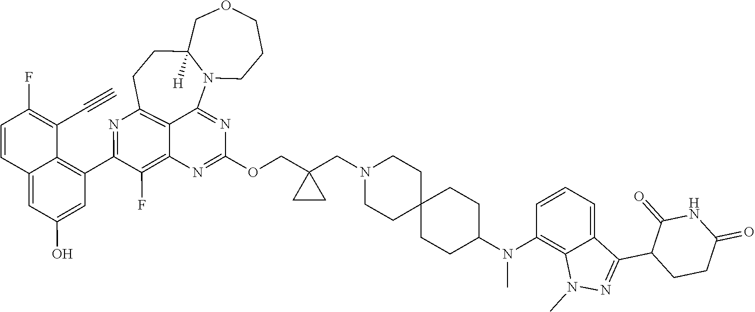 |
| 3-37 | 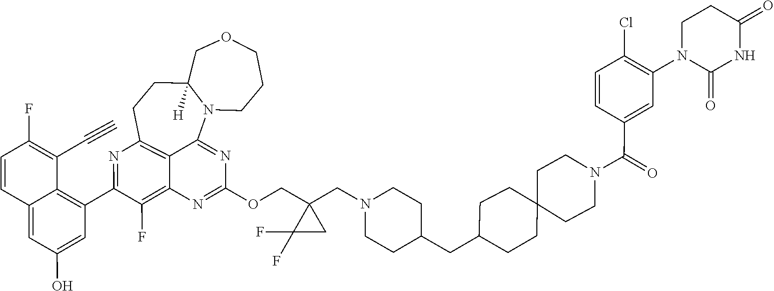 |
| 3-38 | 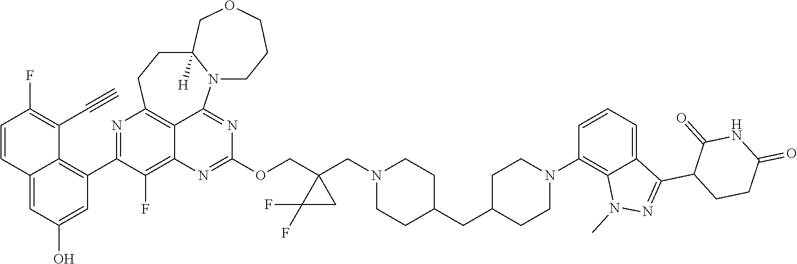 |
| 3-39 | 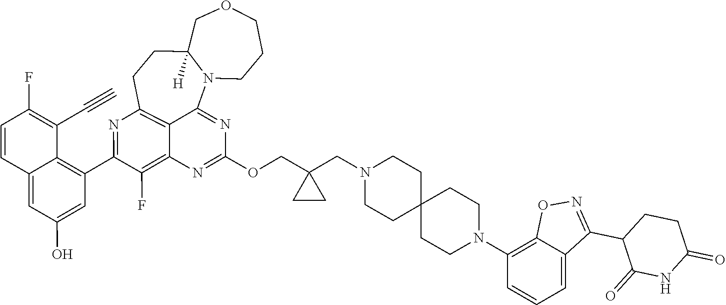 |
| 3-40 | 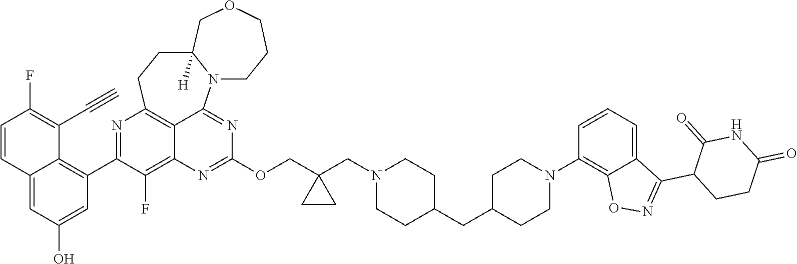 |
| 3-41 | 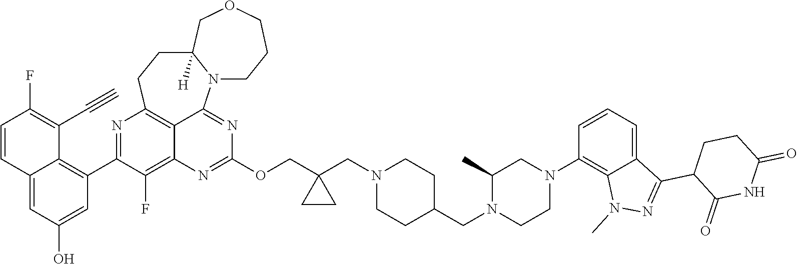 |
| 3-42 | 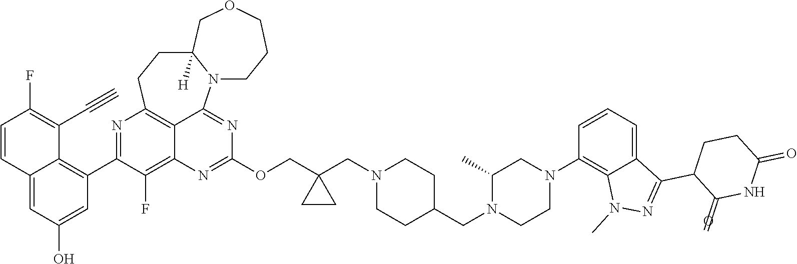 |
| 3-43 | 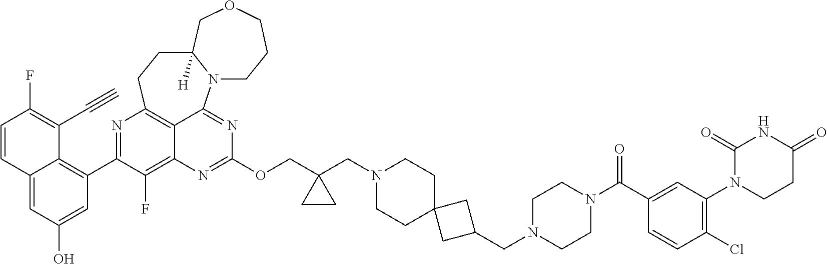 |
| 3-44 | 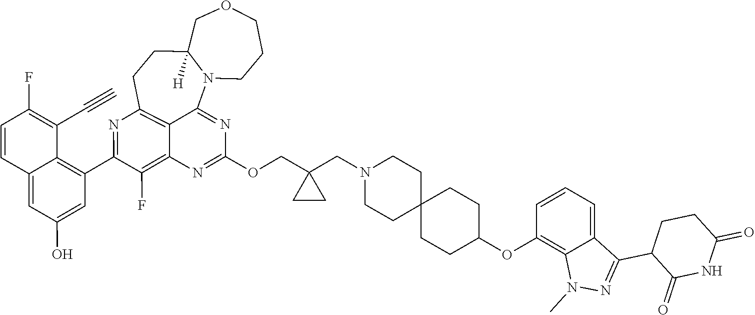 |
| 3-45 | 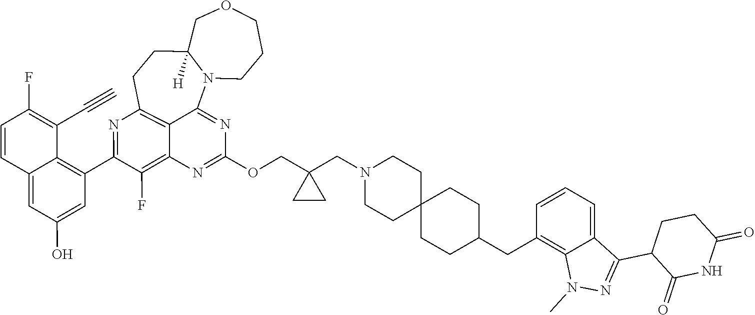 |
| 3-46 | 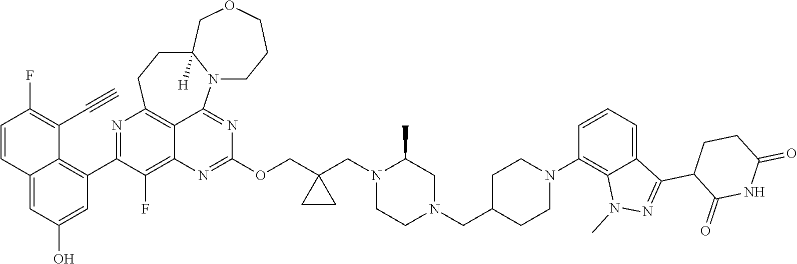 |
| 3-47 | 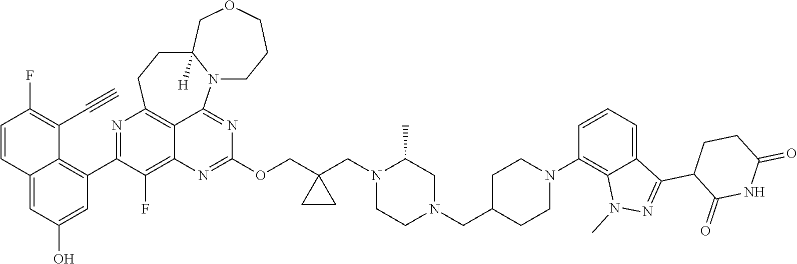 |
| 3-48 | 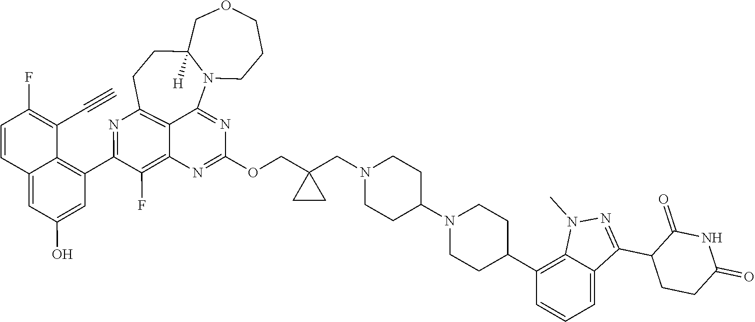 |

Notes: The sterochemistry at the chiral center of cyclopropyl of Examples 3-17 and 3-18 is assigned arbitrarily, and the specific configuration (R or S) is not known.

TABLE 3A6
Compounds

| | | | Changes to |
|---|---|---|---|
| | | | Procedure: |
| | | | Different |
| | | | Reagents/ |
| | | | Starting |
| Example | LCMS | ¹H NMR (400 MHz, Methanol-d₄) | Materials |
| 3-2 | 994.6 | δ 7.76-7.65 (m, 2H), 7.43-7.37 (m, 1H), 7.35- | INT 5-2 and |
| | | 7.23 (m, 2H), 7.17-7.03 (m, 2H), 4.70-4.48 (m, | INT 6-2 |
| | | 2H), 4.47-4.35 (m, 2H), 4.17-3.57 (m, 15H), 3.15- | in step 1. Step 2 |
| | | 2.93 (m, 6H), 2.88-2.69 (m, 2H), 2.64-2.54 (m, | was omitted. |
| | | 1H), 2.52-1.90 (m, 21H), 1.08-0.99 (m, 2H), 0.95- | |
| | | 0.83 (m, 5H). | |
| 3-3 | 965.6 | δ 7.93-7.83 (m, 1H), 7.64 (d, J = 8.9 Hz, 1H), 7.42- | INT 8-4 |
| | | 7.33 (m, 2H), 7.33-7.26 (m, 1H), 7.01-6.88 (m, | in step 1 |
| | | 2H), 4.71-4.63 (m, 1H), 4.60-4.50 (m, 1H), 4.44- | |
| | | 4.23 (m, 2H), 4.16-3.79 (m, 13H), 3.79-3.57 (m, | |
| | | 5H), 3.26-2.99 (m, 8H), 2.86-2.66 (m, 2H), 2.51- | |
| | | 2.26 (m, 6H), 2.25-2.09 (m, 3H), 2.02-1.92 (m, | |
| | | 1H), 1.89-1.71 (m, 2H), 1.16-0.81 (m, 5H). | |
| 3-4 | 965.6 | δ 7.90-7.78 (m, 1H), 7.60-7.54 (m, 1H), 7.40- | INT 12-1 in |
| | | 7.22 (m, 3H), 7.21-7.10 (m, 2H), 4.79-4.62 (m, | step 1 |
| | | 1H), 4.60-4.49 (m, 1H), 4.47-4.33 (m, 2H), 4.32- | |
| | | 4.27 (m, 3H), 4.16-4.08 (m, 1H), 4.08-3.80 (m, | |
| | | 6H), 3.79-3.63 (m, 4H), 3.49-3.37 (m, 5H), 3.28- | |
| | | 3.18 (m, 5H), 3.14-3.04 (m, 3H), 2.88-2.69 (m, | |
| | | 2H), 2.54-2.10 (m, 9H), 2.02-1.93 (m, 1H), 1.92- | |
| | | 1.72 (m, 2H), 1.15-0.98 (m, 2H), 0.98-0.84 (m, | |
| | | 2H). | |
| 3-5 | 965.5 | δ 7.89 (dd, J = 9.2, 5.6 Hz, 1H), 7.49-7.30 (m, 4H), | INT 12-2 in |
| | | 7.11-7.02 (m, 2H), 4.79-4.65 (m, 1H), 4.63-4.53 | step 1 |
| | | (m, 1H), 4.50-4.34 (m, 2H), 4.33-4.23 (m, 3H), | |
| | | 4.19-3.80 (m, 6H), 3.80-3.39 (m, 5H), 3.31-3.03 | |
| | | (m, 8H), 3.02-2.90 (m, 3H), 2.87-2.68 (m, 4H), | |
| | | 2.60-2.10 (m, 6H), 2.06-1.83 (m, 4H), 1.69-1.50 | |
| | | (m, 2H), 1.06-0.90 (m, 2H), 0.89-0.75 (m, 2H). | |
| 3-6 | 983.5 | 7.84-7.70 (m, 1H), 7.55-7.48 (m, 1H), 7.36-7.15 | INT 12-3 in |
| | | (m, 3H), 7.14-7.03 (m, 2H), 4.78-4.68 (m, 1H), | step 1 |
| | | 4.60-4.48 (m, 1H), 4.46-4.36 (m, 2H), 4.31-4.28 | |
| | | (m, 2H), 4.28-4.24 (m, 1H), 4.16-4.09 (m, 1H), | |
| | | 4.09-3.80 (m, 7H), 3.79-3.70 (m, 1H), 3.70-3.66 | |
| | | (m, 1H), 3.50-3.39 (m, 3H), 3.24-2.96 (m, 11H), | |
| | | 2.88-2.70 (m, 2H), 2.57-2.14 (m, 10H), 2.04- | |
| | | 1.91 (m, 1H), 1.12-0.98 (m, 2H), 0.98-0.84 (m, | |
| | | 2H). | |
| 3-7 | 983.5 | δ 7.92-7.77 (m, 1H), 7.65-7.59 (m, 1H), 7.41- | INT 8-5 in |
| | | 7.23 (m, 3H), 7.00-6.81 (m, 2H), 4.72-4.66 (m, | step 1 |
| | | 1H), 4.60-4.50 (m, 1H), 4.45-4.37 (m, 1H), 4.37- | |
| | | 4.27 (m, 2H), 4.16-3.66 (m, 13H), 3.65-3.53 (m, | |
| | | 1H), 3.49-3.38 (m, 6H), 3.27-3.07 (m, 5H), 2.87- | |
| | | 2.68 (m, 2H), 2.54-2.13 (m, 10H), 2.03-1.92 (m, | |
| | | 1H), 1.23-0.74 (m, 5H). | |
| 3-8 | 983.5 | δ 7.95-7.85 (m, 1H), 7.70-7.58 (m, 1H), 7.44- | INT 8-6 in |
| | | 7.24 (m, 3H), 7.17-6.99 (m, 2H), 4.76-4.66 (m, | step 1 |
| | | 1H), 4.64-4.45 (m, 2H), 4.41-4.27 (m, 2H), 4.17- | |
| | | 3.80 (m, 9H), 3.79-3.56 (m, 5H), 3.48-3.38 (m, | |
| | | 2H), 3.29-3.07 (m, 7H), 2.92-2.60 (m, 6H), 2.52- | |
| | | 2.28 (m, 4H), 2.26-2.08 (m, 3H), 2.04-1.80 (m, | |
| | | 3H), 1.08-0.96 (m, 2H), 0.96-0.82 (m, 2H). | |
| 3-9 | 1042.6 | δ 7.71 (dd, J = 9.1, 5.8 Hz, 1H), 7.46 (dd, J = 8.5, 2.2 | INT 5-2 and |
| | | Hz, 1H), 7.40 (d, J = 2.1 Hz, 1H), 7.36-7.32 (m, | INT 11-1 |
| | | 1H), 7.29 (td, J = 9.3, 3.2 Hz, 1H), 7.21 (d, J = 8.6 | in step 1. Step 2 |
| | | Hz, 1H), 7.17-7.04 (m, 1H), 4.64-4.45 (m, 3H), | was omitted. |
| | | 4.16-3.86 (m, 8H), 3.84-3.38 (m, 10H), 3.26- | |
| | | 3.02 (m, 6H), 2.97-2.85 (m, 4H), 2.83 (t, J = 6.7 | |
| | | Hz, 2H), 2.54-2.12 (m, 6H), 2.04-1.96 (m, 1H), | |
| | | 1.87-1.72 (m, 3H), 1.71-1.50 (m, 4H), 1.50-1.33 | |
| | | (m, 2H), 1.33-1.15 (m, 4H), 0.95-0.83 (m, 5H), | |
| | | 0.76-0.66 (m, 2H). | |
| 3-10 | 937.5 | δ 7.94-7.81 (m, 1H), 7.42-7.23 (m, 3H), 7.20- | INT 13-1 in |
| | | 7.14 (m, 2H), 7.12-7.03 (m, 1H), 5.42-5.33 (m, | step 1 |
| | | 1H), 4.77-4.65 (m, 1H), 4.63-4.46 (m, 4H), 4.40- | |
| | | 4.28 (m, 1H), 4.15-3.81 (m, 7H), 3.79-3.62 (m, | |
| | | 6H), 3.24-3.05 (m, 4H), 3.01-2.76 (m, 3H), 2.48- | |
| | | 2.04 (m, 8H), 2.03-1.86 (m, 2H), 1.06-0.96 (m, | |
| | | 2H), 0.95-0.83 (m, 2H). | |
| 3-11 | 1042.5 | δ 7.91 (dd, J = 9.2, 5.8 Hz, 1H), 7.67 (d, J = 8.3 Hz, | INT 14-1 in |
| | | 1H), 7.55 (d, J = 2.0 Hz, 1H), 7.46-7.34 (m, 3H), | step 1 |
| | | 7.33-7.26 (m, 1H), 4.62-4.53 (m, 2H), 4.53-4.44 | |
| | | (m, 1H), 4.17-3.61 (m, 12H), 3.47-3.38 (m, 2H), | |
| | | 3.28-3.17 (m, 5H), 3.14-3.07 (m, 2H), 2.99-2.67 | |
| | | (m, 8H), 2.51-2.28 (m, 2H), 2.29-2.12 (m, 1H), | |
| | | 2.04-1.93 (m, 1H), 1.87-1.69 (m, 3H), 1.69-1.55 | |
| | | (m, 3H), 1.55-1.41 (m, 2H), 1.40-1.30 (m, 1H), | |
| | | 1.30-1.10 (m, 4H), 0.96-0.80 (m, 2H), 0.75-0.64 | |
| | | (m, 2H). | |
| 3-12 | 1041.5 | δ 7.91 (dd, J = 9.2, 5.7 Hz, 1H), 7.67 (d, J = 8.3 Hz, | INT 14-2 in |
| | | 1H), 7.55 (d, J = 2.0 Hz, 1H), 7.44 (dd, J = 8.2, 2.0 | step 1 |
| | | Hz, 1H), 7.41-7.34 (m, 2H), 7.32-7.23 (m, 1H), | |
| | | 4.84-4.78 (m, 1H), 4.59-4.47 (m, 2H), 4.26-4.18 | |
| | | (m, 1H), 4.17-4.09 (m, 1H), 4.09-3.78 (m, 8H), | |
| | | 3.78-3.65 (m, 4H), 3.48-3.37 (m, 3H), 3.13-3.07 | |
| | | (m, 1H), 3.06-2.81 (m, 4H), 2.46-2.14 (m, 3H), | |
| | | 2.02-1.84 (m, 4H), 1.83-1.67 (m, 3H), 1.66-1.40 | |
| | | (m, 7H), 1.32 (s, 2H), 1.23-0.95 (m, 8H), 0.94- | |
| | | 0.81 (m, 2H). | |
| 3-13 | 1037.6 | δ 7.91 (dd, J = 9.2, 5.7 Hz, 1H), 7.49-7.43 (m, 1H), | INT 14-3 in |
| | | 7.43-7.34 (m, 3H), 7.33-7.26 (m, 1H), 7.24-7.18 | step 1 |
| | | (m, 1H), 4.84-4.75 (m, 1H), 4.66-4.45 (m, 2H), | |
| | | 4.29-4.19 (m, 1H), 4.17-4.10 (m, 1H), 4.10-3.81 | |
| | | (m, 9H), 3.80-3.62 (m, 6H), 3.25-3.07 (m, 4H), | |
| | | 3.06-2.92 (m, 2H), 2.83 (t, J = 6.7 Hz, 2H), 2.49- | |
| | | 2.29 (m, 2H), 2.29-2.13 (m, 1H), 2.04-1.88 (m, | |
| | | 3H), 1.82-1.24 (m, 13H), 1.23-0.95 (m, 8H), 0.95- | |
| | | 0.80 (m, 2H). | |
| 3-14 | 1039.5 | δ 7.95-7.88 (m, 1H), 7.48-7.33 (m, 4H), 7.32- | INT 15-1 in |
| | | 7.26 (m, 1H), 7.25-7.18 (m, 1H), 4.77-4.64 (m, | step 1 |
| | | 1H), 4.62-4.47 (m, 2H), 4.42-4.26 (m, 1H), 4.17- | |
| | | 3.80 (m, 10H), 3.79-3.60 (m, 7H), 3.56-3.40 (m, | |
| | | 4H), 3.22-3.05 (m, 3H), 2.83 (t, J = 6.7 Hz, 2H), | |
| | | 2.48-2.30 (m, 2H), 2.27-2.13 (m, 2H), 2.11-1.92 | |
| | | (m, 4H), 1.91-1.62 (m, 5H), 1.62-1.35 (m, 6H), | |
| | | 1.35-1.17 (m, 2H), 1.07-0.96 (m, 2H), 0.96-0.84 | |
| | | (m, 2H). | |
| 3-15 | 1032.6 | δ 7.95-7.86 (m, 1H), 7.45-7.23 (m, 4H), 7.13- | INT 12-4 in |
| | | 7.06 (m, 2H), 4.60-4.46 (m, 2H), 4.39-4.32 (m, | step 1 |
| | | 5H), 4.17-3.66 (m, 10H), 2.83-2.73 (m, 2H), 2.53- | |
| | | 2.16 (m, 7H), 2.08-1.91 (m, 5H), 1.90-1.68 (m, | |
| | | 3H), 1.68-1.24 (m, 14H), 1.23-0.96 (m, 8H), 0.96- | |
| | | 0.82 (m, 2H). | |
| 3-16 | 1005.2 | δ 7.90-7.84 (m, 1H), 7.55 (d, J = 7.9 Hz, 1H), 7.39- | INT 16-1 in |
| | | 7.30 (m, 2H), 7.29-7.22 (m, 1H), 7.20-7.09 | step 1 |
| | | (m, 2H), 4.69-4.61 (m, 1H), 4.57-4.48 (m, 1H), | |
| | | 4.41-4.34 (m, 1H), 4.31 (d, J = 3.7 Hz, 2H), 4.14- | |
| | | 3.57 (m, 11H), 3.50-3.35 (m, 5H), 3.22-3.03 | |
| | | (m, 5H), 3.01-2.91 (m, 2H), 2.87-2.70 (m, 3H), | |
| | | 2.52-2.26 (m, 5H), 2.24-2.10 (m, 3H), 2.03-1.89 | |
| | | (m, 3H), 1.85-1.74 (m, 2H), 1.73-1.63 (m, 2H), | |
| | | 1.43 (h, J = 7.4 Hz, 2H), 1.01 (t, J = 7.3 Hz, 3H), | |
| | | 0.88 (s, 2H). | |
| 3-17 | 1012.2 | δ 7.79-7.70 (m, 1H), 7.58-7.49 (m, 1H), 7.28- | INT 17-1a in |
| | | 6.85 (m, 5H), 4.60-4.37 (m, 3H), 4.16-3.64 (m, | step 1 |
| | | 11H), 3.53-3.44 (m, 2H), 3.21-2.96 (m, 7H), 2.87- | |
| | | 2.75 (m, 2H), 2.57-2.05 (m, 8H), 2.00-1.62 (m, | |
| | | 7H), 1.58-1.37 (m, 2H), 1.15-0.76 (m, 5H). | |
| 3-18 | 1012.2 | δ 7.72-7.56 (m, 2H), 7.29-7.05 (m, 3H), 6.98- | INT 17-1b in |
| | | 6.89 (m, 1H), 6.84-6.72 (m, 1H), 4.60-4.30 (m, | step 1 |
| | | 4H), 4.25-3.59 (m, 12H), 3.48 (q, J = 1.7 Hz, 2H), | |
| | | 3.20-2.93 (m, 5H), 2.86-2.65 (m, 3H), 2.54-2.14 | |
| | | (m, 7H), 2.00-1.55 (m, 6H), 1.49-1.27 (m, 3H), | |
| | | 1.09-0.75 (m, 5H), 0.10 (s, 1H). | |
| 3-19 | 1000.2 | δ 7.91-7.81 (m, 1H), 7.65 (d, J = 9.3 Hz, 1H), 7.38- | INT 13-3 in |
| | | 7.19 (m, 3H), 7.08-7.01 (m, 2H), 4.73-4.68 (m, | step 1 |
| | | 1H), 4.58-4.41 (m, 2H), 4.37-4.29 (m, 1H), 4.16- | |
| | | 3.79 (m, 11H), 3.77-3.65 (m, 2H), 3.48 (p, J = 1.7 | |
| | | Hz, 1H), 3.17-2.91 (m, 7H), 2.83-2.68 (m, 2H), | |
| | | 2.51-2.11 (m, 9H), 2.05-1.90 (m, 4H), 1.85-1.64 | |
| | | (m, 3H), 1.43 (h, J = 7.4 Hz, 1H), 1.01 (t, J = 7.4 Hz, | |
| | | 3H), 0.94-0.84 (m, 1H). | |
| 3-20 | 964.6 | δ 7.99-7.81 (m, 1H), 7.50-7.25 (m, 4H), 7.21- | INT 12-5 in |
| | | 6.91 (m, 2H), 4.87 (dd, J = 12.0 Hz, 11.7 Hz, 1H), | step 1 |
| | | 4.54 (dt, J = 18.3, 9.1 Hz, 1H), 4.34 (dd, J = 9.2, 5.2 | |
| | | Hz, 1H), 4.31-4.19 (m, 4H), 4.18-3.65 (m, 10H), | |
| | | 3.26-3.10 (m, 4H), 3.07-2.90 (m, 3H), 2.83-2.59 | |
| | | (m, 4H), 2.55-2.24 (m, 4H), 2.17 (t, J = 11.6 Hz, | |
| | | 1H), 2.04-1.91 (m, 3H), 1.83-1.69 (m, 3H), 1.66- | |
| | | 1.35 (m, 5H), 1.34-1.20 (m, 2H), 1.14-0.79 (m, | |
| | | 4H). | |
| 3-21 | 965.6 | δ 7.96-7.72 (m, 2H), 7.46-7.24 (m, 3H), 6.69 (dd, | INT 18-1 in |
| | | J = 13.3, 8.7 Hz, 1H), 4.85 (dd, J = 12.0 Hz, 11.5 | step 1 |
| | | Hz, 1H), 4.56-4.39 (m, 3H), 4.32-4.16 (m, 2H), | |
| | | 4.15-3.89 (m, 6H), 3.89-3.65 (m, 6H), 3.52 (d, J = | |
| | | 13.6 Hz, 1H), 3.27-3.08 (m, 2H), 3.07-2.83 (m, | |
| | | 4H), 2.83-2.60 (m, 2H), 2.56-2.24 (m, 4H), 2.17 | |
| | | (t, J = 11.5 Hz, 1H), 2.05-1.86 (m, 3H), 1.78-1.46 | |
| | | (m, 6H), 1.34-1.06 (m, 5H), 1.06-0.75 (m, 4H). | |
| 3-22 | 1004.3 | δ 7.94-7.86 (m, 1H), 7.45-7.24 (m, 4H), 7.09- | INT 12-6 in |
| | | 7.03 (m, 2H), 4.72 (d, J = 12.1 Hz, 1H), 4.61-4.49 | step 1 |
| | | (m, 2H), 4.41-4.29 (m, 4H), 4.18-3.82 (m, 5H), | |
| | | 3.79-3.67 (m, 3H), 3.38-2.91 (m, 7H), 2.87-2.68 | |
| | | (m, 4H), 2.53-2.39 (m, 3H), 2.38-2.26 (m, 2H), | |
| | | 2.23-2.18 (m, 2H), 2.15-2.07 (m, 1H), 2.02-1.77 | |
| | | (m, 8H), 1.54-1.49 (m, 8H), 1.03-0.98 (m, 2H), | |
| | | 0.89 (m, 2H). | |
| 3-23 | 1004.3 | δ 7.90-7.73 (m, 1H), 7.43-7.26 (m, 4H), 7.07- | INT 12-7 in |
| | | 6.83 (m, 2H), 4.84-4.66 (m, 1H), 4.58-4.50 (m, | step 1 |
| | | 1H), 4.38-4.24 (m, 5H), 4.13-3.65 (m, 9H), 3.59- | |
| | | 3.43 (m, 1H), 3.23-3.06 (m, 4H), 3.00-2.88 (m, | |
| | | 3H), 2.86-2.70 (m, 3H), 2.51-2.07 (m, 7H), 2.02- | |
| | | 1.27 (m, 16H), 1.01 (s, 2H), 0.95-0.84 (m, 2H). | |
| 3-24 | 967.5 | δ 7.86 (s, 1H), 7.44-7.17 (m, 4H), 7.10-6.98 (m, | INT 12-8 in |
| | | 2H), 4.99-4.87 (m, 2H), 4.72-4.44 (m, 2H), 4.40- | step 1 |
| | | 4.24 (m, 6H), 4.15-3.78 (m, 9H), 3.78-3.64 (m, | |
| | | 4H), 3.63-3.53 (m, 1H), 3.45-3.27 (m, 5H), 3.26- | |
| | | 3.04 (m, 3H), 2.83-2.68 (m, 4H), 2.51-2.18 (m, | |
| | | 3H), 2.10 (s, 3H), 2.02-1.86 (m, 2H), 1.04-0.95 | |
| | | (m, 2H), 0.95-0.85 (m, 2H). | |
| 3-25 | 965.5 | δ 7.87 (ddd, J = 13.7, 9.0, 5.6 Hz, 1H), 7.65 (dd, J = | INT 18-2 in |
| | | 6.6, 4.7 Hz, 1H), 7.60 (d, J = 6.6 Hz, 1H), 7.41- | step 1 |
| | | 7.26 (m, 3H), 4.85 (dd, J = 16.0, 12.3 Hz, 1H), 4.61- | |
| | | 4.36 (m, 2H), 4.60-4.36 (m, 2H), 4.31-4.18 (m, | |
| | | 4H), 4.16-3.64 (m, 10H), 3.52 (d, J = 13.7 Hz, 1H), | |
| | | 3.29-2.91 (m, 6H), 2.82 (dt, J = 9.8, 5.3 Hz, 2H), | |
| | | 2.61-2.27 (m, 4H), 2.25-2.10 (m, 1H), 2.07-1.86 | |
| | | (m, 5H), 1.75 (m, 2H), 1.67-1.42 (m, 4H), 1.40- | |
| | | 1.22 (m, 2H), 1.12-0.80 (m, 4H). | |
| 3-26 | 936.5 | δ 7.86 (dd, J = 9.0, 5.7 Hz, 1H), 7.45-7.39 (m, 1H), | INT 19-1 |
| | | 7.38-7.25 (m, 3H), 7.14-7.02 (m, 2H), 4.78-4.41 | in step 1 |
| | | (m, 2H), 4.36 (dd, J = 9.2, 5.1 Hz, 1H), 4.29 (s, 3H), | |
| | | 4.16-3.63 (m, 10H), 3.52-3.35 (m, 2H), 3.27- | |
| | | 2.84 (m, 8H), 2.84-2.67 (m, 2H), 2.51-2.26 (m, | |
| | | 5H), 2.26-2.12 (m, 2H), 2.00-1.90 (m, 2H), 1.84 | |
| | | (s, 1H), 1.78-1.59 (m, 3H), 1.29 (s, 1H), 1.08-0.83 | |
| | | (m, 4H). | |
| 3-27 | 950.5 | δ 7.88 (dd, J = 9.1, 5.7 Hz, 1H), 7.71 (d, J = 8.5 Hz, | INT 5-4 and |
| | | 1H), 7.40-7.32 (m, 3H), 7.29-7.19 (m, 1H), 7.10 | INT 6-2 |
| | | (dd, J = 8.6, 1.2 Hz, 1H), 4.94-4.88 (m, 1H), 4.54 | in step 1. Step 3 |
| | | (td, J = 13.9, 6.0 Hz, 1H), 4.37 (dd, J = 9.3, 5.1 Hz, | was omitted. |
| | | 1H), 4.15-4.06 (m, 1H), 4.05-3.89 (m, 6H), 3.89- | |
| | | 3.56 (m, 10H), 3.36-3.32 (m, 2H), 3.23-2.94 (m, | |
| | | 6H), 2.84-2.68 (m, 2H), 2.53-2.41 (m, 2H), 2.39- | |
| | | 2.14 (m, 9H), 2.13-1.91 (m, 7H). | |
| 3-28 | 924.6 | δ 7.96-7.84 (m, 2H), 7.71-7.57 (m, 1H), 7.42- | INT 5-4, INT 8- |
| | | 7.34 (m, 2H), 7.33-7.24 (m, 2H), 4.63-4.50 (m, | 1 used in Step 1 |
| | | 1H), 4.46-4.38 (m, 1H), 4.17-4.10 (m, 1H), 4.10- | and DMF was |
| | | 4.04 (m, 4H), 4.04-3.70 (m, 9H), 3.70-3.63 (m, | added as a co- |
| | | 3H), 3.60-3.47 (m, 3H), 3.24-3.06 (m, 4H), 2.88- | solvent (0.5 |
| | | 2.71 (m, 2H), 2.58-2.45 (m, 1H), 2.45-2.28 (m, | mL). Step 3 was |
| | | 3H), 2.27-2.17 (m, 1H), 2.14-2.02 (m, 4H), 2.01- | omitted. |
| | | 1.96 (m, 1H), 1.92-1.75 (m, 2H), 1.74-1.48 (m, | |
| | | 4H), 1.45-1.30 (m, 2H). | |
| 3-29 | 924.5 | δ 7.90 (dt, J = 9.2, 5.4 Hz, 1H), 7.45-7.31 (m, 4H), | INT 5-4 and |
| | | 7.07-6.99 (m, 2H), 5.06-4.78 (m, 2H), 4.63-4.48 | INT 12-5 |
| | | (m, 1H), 4.38-4.24 (m, 4H), 4.17-3.54 (m, 11H), | in step 1. Step 3 |
| | | 3.29-3.03 (m, 6H), 2.87-2.64 (m, 4H), 2.53-2.11 | was omitted. |
| | | (m, 5H), 2.08-1.96 (m, 3H), 1.89-1.71 (m, 3H), | |
| | | 1.61-1.40 (m, 5H), 1.36-1.24 (m, 2H). | |
| 3-30 | 1021.6 | δ 7.91 (dd, J = 9.1, 5.7 Hz, 1H), 7.47-7.31 (m, 5H), | INT 14-4 in |
| | | 7.31-7.24 (m, 1H), 4.84-4.78 (m, 1H), 4.63-4.44 | step 1 |
| | | (m, 2H), 4.27-4.17 (m, 1H), 4.17-3.80 (m, 8H), | |
| | | 3.80-3.63 (m, 6H), 3.49-3.37 (m, 3H), 3.06-2.78 | |
| | | (m, 4H), 2.47-2.27 (m, 5H), 2.26-2.15 (m, 1H), | |
| | | 2.03-1.88 (m, 3H), 1.83-1.65 (m, 3H), 1.64-1.39 | |
| | | (m, 7H), 1.38-1.25 (m, 3H), 1.24-0.95 (m, 8H), | |
| | | 0.95-0.81 (m, 2H). | |
| 3-31 | 1025.6 | δ 7.91 (dd, J = 9.2, 5.7 Hz, 1H), 7.58-7.50 (m, 1H), | INT 14-5 in |
| | | 7.49-7.43 (m, 1H), 7.42-7.32 (m, 3H), 7.32-7.23 | step 1 |
| | | (m, 1H), 4.84-4.78 (m, 1H), 4.61-4.46 (m, 2H), | |
| | | 4.25-3.78 (m, 9H), 3.78-3.64 (m, 4H), 3.49-3.38 | |
| | | (m, 3H), 3.06-2.92 (m, 2H), 2.86 (t, J = 6.7 Hz, | |
| | | 2H), 2.51-2.10 (m, 3H), 2.03-1.88 (m, 3H), 1.81- | |
| | | 1.24 (m, 14H), 1.23-0.95 (m, 9H), 0.95-0.81 (m, | |
| | | 2H). | |
| 3-32 | 1036.6 | δ 7.91 (dd, J = 9.2, 5.7 Hz, 1H), 7.44-7.33 (m, 3H), | INT 14-6 in |
| | | 7.29 (td, J = 8.7, 2.6 Hz, 2H), 7.10 (d, J = 8.4 Hz, | step 1 |
| | | 1H), 4.84-4.74 (m, 1H), 4.62-4.46 (m, 2H), 4.27- | |
| | | 3.78 (m, 11H), 3.79-3.61 (m, 4H), 3.47-3.36 (m, | |
| | | 2H), 3.26-3.14 (m, 2H), 3.14-3.07 (m, 1H), 3.06- | |
| | | 2.92 (m, 2H), 2.80-2.60 (m, 2H), 2.48-2.27 (m, | |
| | | 3H), 2.27-2.14 (m, 1H), 2.14-2.04 (m, 1H), 2.04- | |
| | | 1.88 (m, 3H), 1.81-1.25 (m, 13H), 1.24-0.95 (m, | |
| | | 8H), 0.95-0.81 (m, 2H). | |
| 3-33 | 964.5 | δ 7.90 (dd, J = 9.1, 5.7 Hz, 1H), 7.72 (d, J = 8.4 Hz, | INT 6-3 in |
| | | 1H), 7.42-7.32 (m, 3H), 7.29 (dd, J = 8.8, 2.4 Hz, | step 1 |
| | | 1H), 7.13-7.07 (m, 1H), 4.71-4.62 (m, 1H), 4.61- | |
| | | 4.50 (m, 1H), 4.44-4.35 (m, 2H), 4.08-3.92 (m, | |
| | | 8H), 3.92-3.81 (m, 2H), 3.78-3.69 (m, 4H), 3.28- | |
| | | 2.97 (m, 10H), 2.87-2.68 (m, 2H), 2.55-2.29 (m, | |
| | | 5H), 2.25-2.06 (m, 8H), 2.01-1.92 (m, 1H), 1.86- | |
| | | 1.70 (m, 2H), 1.07-0.83 (m, 4H). | |
| 3-34 | 964.5 | δ 7.90-7.79 (m, 1H), 7.66 (d, J = 8.1 Hz, 1H), 7.40- | INT 6-4 in |
| | | 7.23 (m, 4H), 7.16 (t, J = 7.6 Hz, 1H), 4.66 (d, J = | step 1 |
| | | 11.9 Hz, 1H), 4.60-4.49 (m, 1H), 4.47-4.35 (m, | |
| | | 2H), 4.35-4.20 (m, 4H), 4.19-3.91 (m, 6H), 3.91- | |
| | | 3.81 (m, 2H), 3.81-3.66 (m, 5H), 3.64-3.57 (m, | |
| | | 1H), 3.22-3.03 (m, 6H), 2.88-2.66 (m, 2H), 2.55- | |
| | | 2.07 (m, 13H), 2.03-1.92 (m, 1H), 1.89-1.72 (m, | |
| | | 2H), 1.16-0.80 (m, 4H). | |
| 3-35 | 1032.3 | δ 7.94-7.88 (m, 1H), 7.45-7.27 (m, 4H), 7.10- | INT 12-9 in |
| | | 7.02 (m, 2H), 4.82-4.63 (m, 2H), 4.58-4.20 (m, | step 1 |
| | | 7H), 4.17-3.82 (m, 5H), 3.79-3.57 (m, 4H), 3.55- | |
| | | 3.50 (m, 1H), 3.28-3.08 (m, 6H), 2.87-2.69 (m, | |
| | | 4H), 2.49-2.04 (m, 5H), 1.99-1.24 (m, 16H), 1.20- | |
| | | 0.83 (m, 8H). | |
| 3-36 | 964.3 | δ 7.92-7.04 (m, 7H), 4.70 (d, J = 12.0 Hz, 1H), 4.59- | INT 12-10 |
| | | 4.45 (m, 2H), 4.42-4.28 (m, 3H), 4.18-3.58 (m, | in step 1 |
| | | 9H), 3.52-3.43 (m, 1H), 3.36 (d, J = 5.9 Hz, 1H), | |
| | | 3.30-3.26 (m, 1H), 3.20-3.06 (m, 4H), 2.86-2.71 | |
| | | (m, 4H), 2.51-2.14 (m, 6H), 2.09-2.03 (m, 1H), | |
| | | 1.97-1.89 (m, 1H), 1.87-1.41 (m, 7H), 1.39-1.11 | |
| | | (m, 4H), 1.04-0.74 (m, 5H). | |
| 3-37 | 1077.2 | δ 7.94-7.86 (m, 1H), 7.67 (d, J = 8.2 Hz, 1H), 7.58- | INT 5-3 and |
| | | 7.54 (m, 1H), 7.44-7.21 (m, 4H), 4.73-4.43 (m, | INT 14-2 |
| | | 4H), 4.19-3.57 (m, 11H), 3.54-3.37 (m, 3H), 3.21- | in step 1. |
| | | 2.76 (m, 8H), 2.46-0.80 (m, 26H). | |
| 3-38 | 1000.2 | δ 7.92-7.84 (m, 1H), 7.45-7.23 (m, 4H), 7.12- | INT 5-3 and |
| | | 7.03 (m, 2H), 5.07-5.03 (m, 1H), 4.75-4.67 (m, | INT 12-5 |
| | | 1H), 4.59-4.51 (m, 2H), 4.38 (dd, J = 9.2, 5.1 Hz, | in step 1. |
| | | 1H), 4.34-4.26 (m, 2H), 4.16-3.63 (m, 9H), 3.55- | |
| | | 3.42 (m, 2H), 3.28-3.03 (m, 6H), 2.86-2.68 (m, | |
| | | 3H), 2.53-2.18 (m, 5H), 2.14-1.93 (m, 4H), 1.91- | |
| | | 1.70 (m, 4H), 1.68-1.19 (m, 8H). | |
| 3-39 | 923.5 | δ 7.95-7.86 (m, 1H), 7.42-7.24 (m, 5H), 7.07- | INT 22-1 |
| | | 6.98 (m, 1H), 4.82-4.76 (m, 1H), 4.60-4.42 (m, | in step 1 |
| | | 3H), 4.33-4.22 (m, 1H), 4.18-3.62 (m, 11H), 3.28- | |
| | | 3.05 (m, 6H), 2.90-2.75 (m, 2H), 2.64-2.51 (m, | |
| | | 1H), 2.50-2.29 (m, 3H), 2.28-2.17 (m, 1H), 2.13- | |
| | | 1.89 (m, 6H), 1.89-1.76 (m, 2H), 1.73-1.67 (m, | |
| | | 1H), 1.67-1.53 (m, 1H), 1.09-0.98 (m, 2H), 0.98- | |
| | | 0.84 (m, 2H). | |
| 3-40 | 951.5 | δ 7.96-7.84 (m, 1H), 7.44-7.22 (m, 5H), 7.08- | INT 22-2 |
| | | 6.98 (m, 1H), 4.86-4.74 (m, 1H), 4.60-4.47 (m, | in step 1 |
| | | 2H), 4.18-3.58 (m, 12H), 3.47-3.38 (m, 1H), 3.22- | |
| | | 3.11 (m, 2H), 3.08-2.93 (m, 2H), 2.86-2.71 (m, | |
| | | 4H), 2.65-2.51 (m, 1H), 2.51-2.29 (m, 3H), 2.29- | |
| | | 2.13 (m, 1H), 2.08-1.92 (m, 4H), 1.85-1.71 (m, | |
| | | 3H), 1.66-1.49 (m, 3H), 1.49-1.32 (m, 2H), 1.32- | |
| | | 1.26 (m, 1H), 1.26-1.16 (m, 1H), 1.11-0.81 (m, | |
| | | 4H). | |
| 3-41 | 979.3 | δ 7.81 (s, 1H), 7.55 (d, J = 7.4 Hz, 1H), 7.40-7.08 | INT 16-2 |
| | | (m, 5H), 4.69-4.61 (m, 1H), 4.58-4.48 (m, 2H), | in step 1 |
| | | 4.43-4.35 (m, 2H), 4.34-4.20 (m, 3H), 4.14-3.65 | |
| | | (m, 11H), 3.51-3.40 (m, 3H), 3.18-3.01 (m, 7H), | |
| | | 2.82-2.73 (m, 2H), 2.52-2.02 (m, 9H), 2.00-1.74 | |
| | | (m, 4H), 1.59 (s, 1H), 1.47-1.28 (m, 2H), 1.11- | |
| | | 0.97 (m, 2H), 0.96-0.84 (m, 2H). | |
| 3-42 | 979.3 | δ 7.86-7.77 (m, 1H), 7.59-7.52 (m, 1H), 7.38-7.09 | INT 16-3 |
| | | (m, 5H), 4.71-4.64 (m, 1H), 4.58-4.47 (m, 2H), 4.42- | in step 1 |
| | | 4.23 (m, 5H), 4.15-3.64 (m, 11H), 3.50-3.37 (m, | |
| | | 4H), 3.22-2.96 (m, 9H), 2.80-2.72 (m, 2H), 2.51- | |
| | | 2.04 (m, 10H), 2.00-1.91 (m, 2H), 1.88-1.75 (m, | |
| | | 2H), 1.73-1.64 (m, 2H), 1.62-1.55 (m, 1H), 1.49- | |
| | | 1.31 (m, 4H), 1.05-0.97 (m, 4H), 0.94-0.84 (m, 2H). | |
| 3-43 | 1014.2 | δ 7.89 (dd, J = 9.1, 5.7 Hz, 1H), 7.70 (d, J = 8.3 Hz, | INT 14-7 |
| | | 1H), 7.62 (d, J = 2.0 Hz, 1H), 7.51 (dd, J = 8.3, 2.0 | in step 1 |
| | | Hz, 1H), 7.39-7.31 (m, 2H), 7.24 (dd, J = 20.0, 2.5 | |
| | | Hz, 1H), 4.64 (t, J = 12.1 Hz, 2H), 4.57-4.46 (m, | |
| | | 2H), 4.41-4.27 (m, 2H), 4.14-3.63 (m, 13H), 3.50- | |
| | | 3.46 (m, 1H), 3.17-3.01 (m, 4H), 2.99-2.70 (m, | |
| | | 5H), 2.42-1.82 (m, 12H), 1.81-1.63 (m, 3H), 1.48- | |
| | | 1.38 (m, 1H), 1.05-0.95 (m, 2H), 0.87 (s, 2H). | |
| 3-44 | 951.2 | δ 7.94-7.72 (m, 1H), 7.48-7.13 (m, 4H), 7.10-6.97 | INT 13-4 |
| | | (m, 1H), 6.91-6.65 (m, 1H), 4.80-4.41 (m, 3H), 4.37- | in step 1 |
| | | 3.59 (m, 15H), 3.52-3.38 (m, 1H), 3.25-3.02 (m, | |
| | | 4H), 2.89-2.63 (m, 2H), 2.56-2.14 (m, 5H), 2.09- | |
| | | 1.54 (m, 12H), 1.51-1.23 (m, 1H), 1.09-0.79 (m, | |
| | | 4H). | |
| 3-45 | 949.2 | δ 7.95-7.83 (m, 1H), 7.66-7.55 (m, 1H), 7.47-7.22 | INT 13-5 |
| | | (m, 3H), 7.19-6.96 (m, 2H), 4.59-4.46 (m, 2H), 4.42- | in step 1 |
| | | 4.26 (m, 2H), 4.25-3.82 (m, 8H), 3.79-3.68 (m, | |
| | | 3H), 3.64-3.55 (m, 1H), 3.49 (s, 1H), 3.23-2.94 (m, | |
| | | 6H), 2.86-2.67 (m, 2H), 2.51-1.91 (m, 9H), 1.85- | |
| | | 1.76 (m, 1H), 1.70-0.78 (m, 14H). | |
| 3-46 | 979.3 | δ 7.89-7.81 (m, 1H), 7.47-7.24 (m, 4H), 7.12-7.01 | INT 16-4 |
| | | (m, 2H), 4.62-4.46 (m, 3H), 4.38 (dd, J = 9.3, 5.0 | in step 1 |
| | | Hz, 1H), 4.30 (d, J = 5.7 Hz, 3H), 4.21-3.61 (m, | |
| | | 11H), 3.54-3.48 (m, 2H), 3.27-2.97 (m, 5H), 2.89- | |
| | | 2.65 (m, 6H), 2.54-2.13 (m, 6H), 2.08-1.81 (m, | |
| | | 5H), 1.62-1.46 (m, 2H), 1.32 (d, J = 6.2 Hz, 3H), | |
| | | 1.08-0.95 (m, 1H), 0.92-0.81 (m, 2H), 0.78-0.63 | |
| | | (m, 1H). | |
| 3-47 | 979.3 | δ 7.91-7.83 (m, 1H), 7.48-7.24 (m, 4H), 7.12-7.04 | INT 16-5 |
| | | (m, 2H), 4.73 (d, J = 11.7 Hz, 1H), 4.64-4.45 (m, | in step 1 |
| | | 2H), 4.42-4.27 (m, 5H), 4.20-3.65 (m, 9H), 3.52- | |
| | | 2.99 (m, 6H), 2.92-2.60 (m, 8H), 2.51-2.17 (m, 6H), | |
| | | 2.09-1.82 (m, 5H), 1.66-1.43 (m, 2H), 1.39-1.23 | |
| | | (m, 3H), 1.05-0.93 (m, 1H), 0.88-0.84 (m, 2H), 0.71- | |
| | | 0.67 (m, 1H). | |
| 3-48 | 950.5 | δ 7.69-7.59 (m, 2H), 7.32-7.07 (m, 5H), 5.11 (t, J = | INT 6-5 |
| | | 12.1 Hz, 1H), 4.65-4.37 (m, 3H), 4.13-3.51 (m, 16H), | in step 1 |
| | | 3.19-3.08 (m, 5H), 2.91-2.76 (m, 3H), 2.53-2.27 (m, | |
| | | 9H), 2.18-1.95 (m, 7H), 1.24-0.71 (m, 4H). | |

### Examples 4-1-4-2

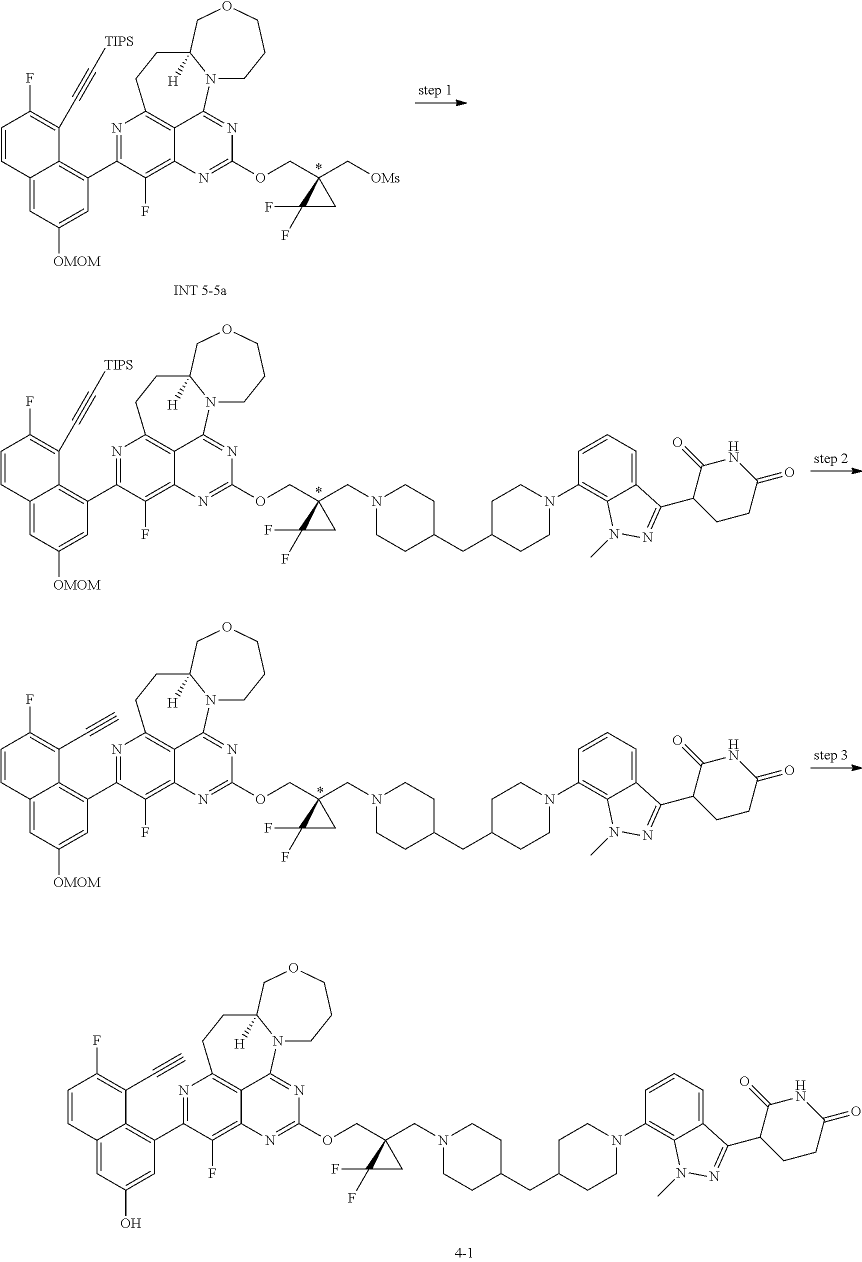

Step 1: 3-(7-(4-((1-(((R)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl)piperidin-4-yl)methyl)piperidin-1-yl)-1-methyl-1H-indazol-3-yl)piperidine-2,6-dione: A mixture of ((S)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl methanesulfonate INT 5-5a (0.07 mmol), 3-(1-methyl-7-(4-(piperidin-4-ylmethyl)piperidin-1-yl)-1H-indazol-3-yl)piperidine-2,6-dione (0.2 mmol) and K₂CO₃ (0.6 mmol) in THE (1 mL) was stirred at 60° C. for 2 days. The reaction mixture was concentrated under reduced pressure and the residue was purified by flash chromatography on silica gel eluting with 10% ammonium hydroxide in ethanol/dichloromethane to provide the title compound. LCMS: 1200.4.

Step 2: 3-(7-(4-((1-(((R)-1-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)-2,2-difluorocyclopropyl)methyl)piperidin-4-yl)methyl)piperidin-1-yl)-1-methyl-1H-indazol-3-yl)piperidine-2,6-dione: To a solution of 3-(7-(4-((1-(((R)-2,2-difluoro-1-((((S)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalen-12-yl)oxy)methyl)cyclopropyl)methyl)piperidin-4-yl)methyl)piperidin-1-yl)-1-methyl-1H-indazol-3-yl)piperidine-2,6-dione (0.03 mmol) in THE (2 mL) was added TBAF (0.1 mL, 1 M in THF) and the resulting reaction mixture was stirred at room temperature for 10 minutes. Upon completion, the solvents were evaporated under reduced pressure without applying heat. The residue was purified by flash chromatography on silica gel eluting with a mixture of ethyl acetate and hexanes followed by 10% ammonium hydroxide in ethanol/dichloromethane to obtain the title compound. LCMS: 1044.3.

Step 3: 3-(7-(4-((1-(((R)-1-((((S)-2-(8-ethynyl-7-fluoro-3-hydroxynaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)-2,2-difluorocyclopropyl)methyl)piperidin-4-yl)methyl)piperidin-1-yl)-1-methyl-1H-indazol-3-yl)piperidine-2,6-dione (Example 4-1): To a solution of 3-(7-(4-((1-(((R)-1-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)-2,2-difluorocyclopropyl)methyl)piperidin-4-yl)methyl)piperidin-1-yl)-1-methyl-1H-indazol-3-yl)piperidine-2,6-dione (0.02 mmol) in acetonitrile (2 mL) was added HCl (1 mL, 4N HCl in 1,4-dioxane) and the resulting solution was stirred at room temperature for 1 hour. The solution was then concentrated under reduced pressure and purified by RP-HPLC (0 to 90%, 0.1% trifluoroacetic acid in acetonitrile/0.1% trifluoroacetic acid in water). Fractions containing the product were pooled and lyophilized to afford the title compound. ¹H NMR (400 MHz, Methanol-d₄) δ 7.91-7.83 (m, 1H), 7.48-7.24 (m, 4H), 7.11-7.01 (m, 2H), 5.07 (d, J=12.6 Hz, 1H), 4.64-4.47 (m, 2H), 4.38 (dd, J=9.3, 5.2 Hz, 1H), 4.33-4.27 (m, 3H), 4.19-3.61 (m, 12H), 3.54-3.03 (m, 3H), 2.83-2.64 (m, 4H), 2.52-2.28 (m, 4H), 2.16-1.95 (m, 6H), 1.92-1.71 (m, 4H), 1.68-1.21 (m, 8H). LCMS: 1000.2. Notes: The stereochemistry at the chiral center of cyclopropyl of Example 4-1 is assigned arbitrarily, and the specific configuration (R or S) is not known.

The following Examples were made in a similar fashion to Example 4-1 and are shown below in Table 3A7. To prepare the below Examples, different reagents/starting materials were used than some of those described in the steps toward Example 4-1 and are noted in the last column of Table 3A8—“Changes to Procedure: Different Reagents/Starting Materials”. A person of ordinary skill in the art will readily recognize which reagents/starting materials used for the synthesis of Example 4-1 were replaced with the different reagents/starting materials noted below.

TABLE 3A7
Compounds

| Example | Structure |
|---|---|
| 4-2 | 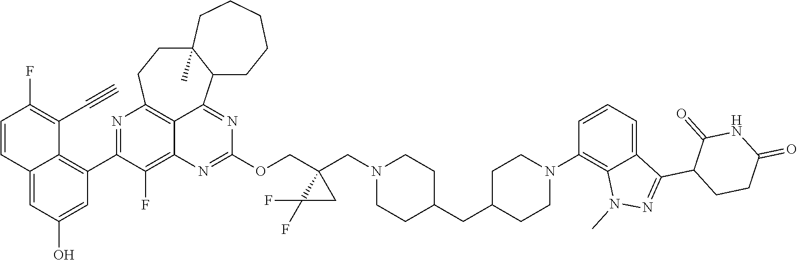 |

Notes: The sterochemistry at the chiral center of cyclopropyl of Example 4-2 is assigned arbitrarily, and the specific configuration (R or S) is not known.

TABLE 3A8
Compounds

| | | | Changes to |
|---|---|---|---|
| | | | Procedure: |
| | | | Different |
| | | | Reagents/ |
| | | | Starting |
| Example | LCMS | ¹H NMR (400 MHz, Methanol-d₄) | Materials |
| 4-2 | 1000.2 | δ 7.92-7.84 (m, 1H), 7.45-7.22 (m, 4H), 7.11- | INT 5-5b |
| | | 7.01 (m, 2H), 4.89 (s, 1H), 4.71 (d, J = 13.0 Hz, 1H), | in step 1 |
| | | 4.57-4.49 (m, 1H), 4.37 (dd, J = 9.2, 5.1 Hz, 1H), | |
| | | 4.34-4.28 (m, 3H), 4.21-3.64 (m, 12H), 3.45- | |
| | | 3.02 (m, 3H), 2.82-2.62 (m, 4H), 2.45-2.18 (m, | |
| | | 5H), 2.14-1.95 (m, 5H), 1.91-1.20 (m, 12H). | |

### Reference Compound 1 (RC-1)

| Example | Structure |
|---|---|
| RC-1 | 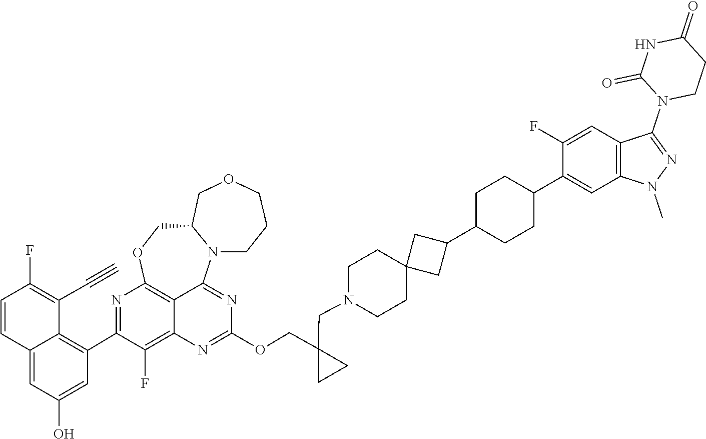 [SMILES: C#Cc1c(F)ccc2cc(O)cc(-c3nc4c5c(nc(OCC6(CN7CCC8(CC7)CC(C7CCC(c9cc%10c(cc9F)c(N9CCC(=O)NC9=O)nn%10C)CC7)C8)CC6)nc5c3F)N3CCCOC[C@H]3CO4)c12] |

Notes: Reference Compound RC-1 (Compound 88, WO 2025/168051) (S)-1-(6-(1-(7-((1-(((2-(8-ethynyl-7-fluoro-3-hydroxynaphthalen-1-yl)-1-fluoro-5a,6,9,10-tetrahydro-5H,8H-4,7-dioxa-3,10a,11,13-tetraazanaphtho[1,8-ab]heptalen-12-yl)oxy)methyl)cyclopropyl)methyl)-7-azaspiro[3.5]nonan-2-yl)piperidin-4-yl)-5-fluoro-1-methyl-1H-indazol-3-yl)dihydropyrimidine-2,4(1H,3H)-dione was prepared according to the protocol described in WO 2025/168051. The analytical characterization data were consistent with reported values.

### IV. Biological Examples

### AsPC-1 KRAS G12D HiBit Assay 1

AsPC-1 KRAS(G12D)-HiBit cells (Promega product #CS3023190) were maintained in growth medium containing RPMI 1640 medium (Gibco product #32404-014) supplemented with GlutaMAX (Gibco product #35050-061), penicillin/streptomycin (Gibco product #15140-122) and 10% fetal bovine serum (HyClone product #SH30396.03). Cells were passaged 2 times per week to maintain sub-confluent densities. Passages 5 to 20 were used for assays.

Test compounds were prepared in 100% DMSO in 384-well polypropylene plates (Greiner Bio-One, product #784201) with 8 compounds per plate in grouped replicates of 4 at 10 serially diluted concentrations (1:3). The serially diluted compounds were transferred to low dead volume Echo plates (Beckman Coulter, product #001-12782).

Serially diluted test compounds were transferred to black 384-well cell culture-treated plates (Greiner Bio-One, product #781086) at 200 nL per well using an Echo acoustic dispenser (Beckman Coulter). AsPC-1 KRAS-HiBit cells were harvested and suspended in growth medium (described above). The cells were seeded in the assay plates containing test compounds at 4,000 cells per well in 40 μL. The plates containing cells and test compounds were incubated at 37° C., 5% CO₂ for 24 hours. The cells were then equilibrated to room temperature for 30 minutes and Nano-Glo HiBiT Lytic reagent (Promega, product #N3050) was prepared according to manufacturer protocol and equilibrated to room temperature. Medium (20 μL per well) was aspirated from assay plates using a BioTek EL406 washer dispenser (Agilent), and 20 μL of Nano-Glo HiBiT Lytic reagent was added per well. The plates were placed on an orbital shaker at 1000 rpm for 3 minutes, and incubated at room temperature for an additional 7 minutes before reading luminescence on an EnVision multimode microplate reader (Revvity).

The luminescence signal was normalized to negative (DMSO, 100%) and positive (no cells, 0%) controls and plotted against log 10 concentration of test compounds. The data was fitted to a 4 variables non-linear regression analysis to determine DC₅₀ and Dₘₐₓ. DC₅₀ is defined as the concentration at which the mid-point between the top and bottom plateaus is reached. Dₘₐₓ is defined as 100% minus the lowest normalized data observed across the dose response (maximal signal loss). Unless otherwise noted, Dₘₐₓ standard deviation was less than 5.

TABLE 4
Biological data

| | DC₅₀ | Dₘₐₓ |
|---|---|---|
| Example | (nM) | (%) |
| 1-1 | 1.3 | 78 |
| 1-2 | 1.7 | 72 |
| 1-3 | 1.4 | 66 |
| 1-4 | 2.2 | 65 |
| 1-5 | 5.1 | 74 |
| 1-6 | 4.5 | 67 |
| 1-7 | 55 | 46 |
| 1-8 | 4.7 | 58 |
| 1-9 | 1.1 | 72 |
| 1-10 | 4.4 | 82 |
| 2-1 | 83 | 60 |

### AsPC-1 KRAS G12D HiBit Assay 2

AsPC-1 KRAS(G12D)-HiBit cells (Promega product #CS3023190) were maintained in growth medium containing RPMI 1640 medium, no phenol red (Gibco product #11835030) supplemented with 1% penicillin/streptomycin/Glutamine (Corning product #30-009-CI) and 10% fetal bovine serum (HyClone product #SH30396.03). Cells were passaged 2 times per week to maintain sub-confluent densities. Passages 5 to 12 were used for assays.

Test compounds were prepared in 100% DMSO in 384-well polypropylene plates (Greiner Bio-One, product #784201) with 8 compounds per plate in grouped replicates of 4 at 10 serially diluted concentrations (1:3). The serially diluted compounds were transferred to low dead volume Echo plates (Beckman Coulter, product #001-12782).

Serially diluted test compounds were transferred to black 384-well cell culture-treated plates (Greiner Bio-One, product #781086) at 200 nL per well using an Echo acoustic dispenser (Beckman Coulter). AsPC-1 KRAS-HiBit cells were harvested and suspended in growth medium (described above). The cells were seeded in the assay plates containing test compounds at 5,000 cells per well in 40 μL. The plates containing cells and test compounds were incubated at 37° C., 5% CO₂ for 24 hours. The cells were then equilibrated to room temperature for 30 minutes and Nano-Glo HiBiT Lytic reagent (Promega, product #N3050) was prepared according to manufacturer protocol and equilibrated to room temperature. Medium (20 μL per well) was aspirated from assay plates using a BioTek EL406 washer dispenser (Agilent), and 20 μL of Nano-Glo HiBiT Lytic reagent was added per well. The plates were placed on an orbital shaker at 1000 rpm for 3 minutes, and incubated at room temperature for an additional 7 minutes before reading luminescence on an EnVision multimode microplate reader (Revvity).

The luminescence signal was normalized to negative (DMSO, 100%) and positive (no cells, 0%) controls and plotted against log 10 concentration of test compounds. The data was fitted to a 4 variables non-linear regression analysis to determine DC₅₀ and Dₘₐₓ. DC₅₀ is defined as the concentration at which the mid-point between the top and bottom plateaus is reached. Dₘₐₓ is defined as 100% minus the lowest normalized data observed across the dose response (maximal signal loss). Unless otherwise noted, Dₘₐₓ standard deviation was less than 5.

TABLE 5
Biological data

| | DC₅₀ | Dₘₐₓ |
|---|---|---|
| Example | (nM) | (%) |
| 2-2 | 136 | 33 |
| 2-3 | 58.4 | 67 |
| 2-4 | 44.3 | 36 |
| 2-5 | 25.4 | 71 |
| 3-1* | 1.9 | 60 |
| 3-2 | 3.6 | 64 |
| 3-3 | 1.3 | 74 |
| 3-4 | 5.1 | 77 |
| 3-5 | 6.4 | 72 |
| 3-6 | 4.4 | 75 |
| 3-7 | 1.1 | 60 |
| 3-8 | 1.5 | 69 |
| 3-9* | 45.2 | 65 |
| 3-10 | 11.0 | 68 |
| 3-11 | 10.7 | 79 |
| 3-12 | 11.1 | 80 |
| 3-13 | 10.7 | 79 |
| 3-14 | 7.8 | 82 |
| 3-15 | 75.8 | 67 |
| 3-16 | 5.4 | 82 |
| 3-17 | 5.7 | 62 |
| 3-18 | 5.7 | 73 |
| 3-19* | 1.8 | 68 |
| 3-20 | 9.1 | 76 |
| 3-21* | 3.4 | 56 |
| 3-22 | 33.5 | 75 |
| 3-23 | 15.1 | 75 |
| 3-24 | 5.0 | 78 |
| 3-25 | 7.5 | 69 |
| 3-26 | 3.4 | 80 |
| 3-27 | 1.1 | 75 |
| 3-28 | 12.1 | 67 |
| 3-29 | 37.0 | 71 |
| 3-30 | 7.2 | 81 |
| 3-31 | 18.0 | 74 |
| 3-32 | 11.7 | 80 |
| 3-33 | 1.3 | 79 |
| 3-34 | 6.3 | 78 |
| 3-35 | 64.4 | 74 |
| 3-36 | 9.8 | 79 |
| 3-37 | 17.4 | 77 |
| 3-38 | 24.3 | 74 |
| 3-39 | 6.9 | 72 |
| 3-40 | 11.5 | 70 |
| 3-41 | 4.5 | 76 |
| 3-42 | 5.4 | 73 |
| 3-43 | 9.4 | 83 |
| 3-44 | 8.3 | 79 |
| 3-45 | 9.1 | 81 |
| 3-46* | 15.3 | 57 |
| 3-47 | 10.7 | 72 |
| 3-48 | 10.6 | 75 |
| 4-1 | 18.6 | 73 |
| 4-2 | 36.9 | 69 |
| RC-1 | 1.6 | 66 |

*Dₘₐₓ standard deviation of 5-10.

### AsPC-1 KRAS G12D Cell Viability Assay 1

AsPC-1 KRAS(G12D) cells (ACH-000222) were maintained in growth medium containing RPMI 1640 medium with GlutaMax (Gibco, product #61870-036) supplemented with 1× penicillin/streptomycin (Gibco, product 15140-122) and 10% fetal bovine serum (HyClone, product #SH30396.03). The cell culture was passaged 2 times per week to maintain sub-confluent densities and used for assays within 7 weeks of initial thaw. Cells were harvested and suspended in growth medium and seeded at 1000 cells per well in 40 μL in 384-well, ultra-low binding, U-shaped microplates (Corning, product #4516). The plates were incubated at 37° C., 5% CO2 for 48 hours to form spheroids prior to dosing with test compounds. Test compounds were prepared in 100% DMSO and applied directly to cells in plates using a Hewlitt Packard Digital Dispenser D300e in a 15 point logarithmically distributed dose range from 10 μM to 50 pM in grouped triplicates. Cells treated with compound were incubated at 37° C., 5% CO2 for another 96 hours. The plates were then equilibrated to room temperature for 15 minutes and CellTiter-Glo 3D reagent (Promega, product #G9683) was added at 40 μL per well using a Biotek MultiFLo FX dispenser (Agilent) for a final volume of 80 μL. Plates were placed on an orbital shaker at 600 rpm for 5 minutes and incubated at room temperature for an additional 20 minutes before reading luminescence on a Biotek Neo2 plate reader (Agilent). The luminescence signal was normalized to negative (DMSO, 100%) and positive (4 μM staurosporine, 0%) controls and plotted against log 10 concentration of test compounds. The data was fitted to a 4 variables non-linear regression analysis to determine EC₅₀ (Table 6). EC₅₀ is defined as the concentration at which the mid-point between the top and bottom plateaus is reached.

TABLE 6
Biological data

| Examples | EC₅₀ (nM) |
|---|---|
| 1-1 | 0.3 |
| 1-2 | 4.1 |
| 1-3 | 0.8 |
| 1-4 | 4.3 |
| 1-5 | 9.2 |
| 1-6 | 6.4 |
| 1-7 | 236 |
| 1-8 | 7.3 |
| 1-9 | 0.9 |
| 1-10 | 2.6 |
| 2-1 | 297 |
| 3-1 | 1.1 |
| 3-2 | 2.6 |
| 3-3 | 0.8 |
| 3-17 | 6.4 |
| 3-20 | 9.1 |
| 3-21 | 3.6 |
| 3-28 | 9.2 |

### AsPC-1 KRAS G12D Cell Viability Assay 2

AsPC-1 KRAS(G12D)-HiBit cells (Promega product #CS3023190) were maintained in growth medium containing RPMI 1640 medium, no phenol red (Gibco product #11835030) supplemented with 1× penicillin/streptomycin/Glutamine (Corning product #30-009-CI) and 10% fetal bovine serum (HyClone product #SH30396.03). Cells were passaged 2 times per week to maintain sub-confluent densities. Passages 5 to 12 were used for assays. Test compounds were prepared in 100% DMSO in 384-well polypropylene plates (Greiner Bio-One, product #784201) with 8 compounds per plate in grouped replicates of 4 at 10 serially diluted concentrations (1:3). AsPC-1 KRAS-HiBit cells were harvested and suspended in growth medium (described above). The cells were seeded in 384-well black clear round bottom ultra-low attachment spheroid microplate (Corning, product #3830) at 1,000 cells per well in 80 μL. The plates containing cells were incubated at 37° C. with 5% CO2 for 72 hours to allow spheroid formation, prior to dosing with 0.4 μL of test compounds using a Biomek 17. The cells treated with compounds were incubated at 37° C., 5% CO₂ for another 96 hours. The cells were then equilibrated to room temperature for 30 minutes and CellTiter-Glo® 3D Cell Viability reagent (Promega, product #G9683) was prepared according to manufacturer protocol and equilibrated to room temperature. Medium was aspirated from assay plates until 40 μL remained using a BioTek EL406 washer dispenser (Agilent), and 40 μL of CellTiter-Glo® 3D Cell Viability reagent was added per well. The plates were placed on a Biomek 17, where mixing was performed by aspirating and dispensing 50 μL 15 times across the entire plate to ensure thorough homogenization. The plates were then incubated at room temperature for an additional 5 minutes before luminescence was measured using an EnVision multimode microplate reader (Revvity). The luminescence signal was normalized to negative (DMSO, 100%) and positive (10 μM puromycin, 0%) controls and plotted against log 10 concentration of test compounds. The data was fitted to a 4 variables non-linear regression analysis to determine EC₅₀ (Table 7). EC₅₀ is defined as the concentration at which the mid-point between the top and bottom plateaus is reached.

TABLE 7
Biological data

| Examples | EC₅₀ (nM) |
|---|---|
| 2-4 | 132 |
| 2-5 | 23 |
| 3-4 | 1.5 |
| 3-5 | 5.5 |
| 3-6 | 2.7 |
| 3-7 | 0.9 |
| 3-11 | 5.5 |
| 3-12 | 6.2 |
| 3-13 | 3.1 |
| 3-16 | 4.5 |
| 3-18 | 3.4 |
| 3-23 | 18 |
| 3-24 | 2.9 |
| 3-26 | 2.9 |
| 3-27 | 0.5 |
| 3-29 | 26 |
| 3-30 | 3.8 |
| 3-31 | 9.9 |
| 3-32 | 6.6 |
| 3-33 | 0.6 |
| 3-34 | 2.2 |
| 3-35 | 63 |
| 3-36 | 5.8 |
| 3-37 | 27 |
| 3-38 | 29 |
| 3-39 | 22 |
| 3-40 | 15 |
| 3-41 | 2.2 |
| 3-42 | 3.2 |
| 3-43 | 8.8 |
| 3-44 | 6.1 |
| 3-45 | 7.2 |
| 3-46 | 12 |
| 3-47 | 6.6 |
| 3-48 | 5.7 |
| 4-1 | 27 |
| 4-2 | 33 |

### V. Pharmacokinetic Studies

The purpose of the studies is to assess plasma pharmacokinetics (PK) following oral administration (PO) to athymic nude mice. The in-life phase of the studies was conducted at Charles River Laboratories (Worcester, MA) and Meadowhawk Biolabs (Hayward, CA). Animals were housed and handled in accordance with the Guide for the Care and Use of Laboratory Animals, Institute of Laboratory Animal Resources. The protocols were reviewed and approved by the Institutional Animal Care and Use Committees (IACUC). Female athymic nude mice weighing approximately 0.018 to 0.025 kg were used for the in-life portion of the studies. The animals were not fasted overnight prior to test article administration. Test compounds were formulated in 10% dimethyl sulfoxide, 30% polyethylene glycol 300, 12% 2-hydroxypropyl-β-cyclodextrin and 48% water as a solution and administered via oral gavage at 30-100 mg/kg to the animals (n=4/group). Approximately 30 μL blood samples were collected at the designated timepoints from each animal over a 24-hour period following PO administration. Plasma samples were harvested and analyzed to determine test compound concentrations by LC-MS/MS. The plasma samples were prepared through protein precipitation extraction and analyzed on a Sciex API 6500+Qtrap LC-MS/MS instrument (Framingham, MA). Analytes were eluted on a 1.7 μm 50×2.1 mm Waters Acquity UPLC BEH C18 column (Milford, MA) using mobile phases containing 0.1% formic acid and a linear gradient from 30% to 90% acetonitrile at a flow rate of 1 mL/min. Analyst® 1.7.2 software (Framingham, MA) were used for data acquisition and chromatographic integration. Noncompartmental pharmacokinetic analysis was performed on plasma concentration data using Dotmatics (Boston, MA).

TABLE 8
Mouse PK data

| Compounds | Dose (mg/kg) | C_{Max} (nM) | AUC_{Last} (nM · h) |
|---|---|---|---|
| 3-18 | 50 | 2500 | 12100 |
| 1-4 | 100 | 1224 | 12077 |
| 3-13 | 100 | 456 | 2250 |
| 3-12 | 50 | 187 | 1150 |
| 3-6 | 50 | 232 | 966 |
| 3-7 | 30 | 174 | 456 |
| RC-1 | 50 | 22 | 118 |

1. A compound of Formula (I): 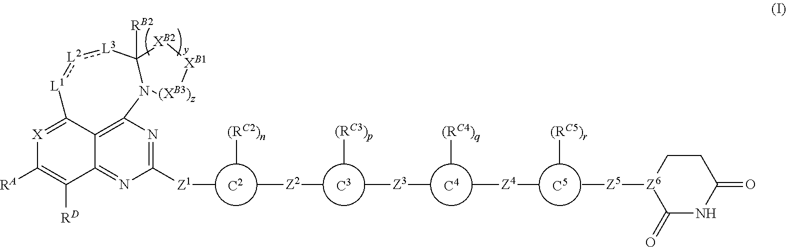
or a pharmaceutically acceptable salt thereof,
wherein
Z¹ is O, 4- to 16-membered heterocyclyl having at least one ring nitrogen, or 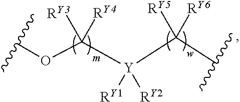
wherein the heterocyclyl is substituted with 0, 1, 2, 3, or 4 Z^{1A};
each Z^{1A} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
R^{Z1A} and R^{Z1B} are each independently C₁-C₆ alkyl, C₁-C₆ haloalkyl, or C₃-C₈ cycloalkyl;
Y is C or Si;
m is 0, 1, 2, or 3;
w is 0, 1, 2, or 3;
R^{Y1} is H, C₁-C₆ alkyl, C₁-C₆ haloalkyl or C₁-C₆ haloalkoxy;
R^{Y2} is H, C₁-C₆ alkyl, C₁-C₆ haloalkyl or C₁-C₆ haloalkoxy;
alternatively, R^{Y1} and R^{Y2} can combine to form ═CH₂, ═CHF, or ═CF₂;
alternatively, R^{Y1} and R^{Y2} can combine with the atom to which they are attached to form C₃-C₆ cycloalkyl, or a 3- to 10-membered heterocyclyl, wherein the cycloalkyl or heterocyclyl is substituted with 0, 1, 2, 3, or 4 R^{Y1C};
each R^{Y1C} is independently halo, C₁-C₆ alkyl, or C₁-C₆ haloalkyl;
each R^{Y3}, R^{Y4}, R^{Y5}, and R^{Y6} is independently H, halo, C₁-C₃ alkyl, C₁-C₃ haloalkyl or C₁-C₃ haloalkoxy;
ring C2 is 4- to 16-membered heterocyclyl having at least one ring nitrogen;
each R^{C2} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
n is 0, 1, 2, 3, or 4;
Z² is C₁-C₄ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z2a}—, —C(O)—C₁-C₃ alkyl, —C(O)NR^{Z2a}—, —C(O)—, —S(O)—, S(O)₂—, S(O)(═NR^{Z2})—, 4- to 10-membered heterocyclyl, or a bond, wherein the alkyl, cycloalkyl, and heterocyclyl is substituted with 0, 1, 2, 3, or 4 Z^{2a}, wherein the alkenyl is substituted with 0, 1, or 2 Z^{2c};
each Z^{2a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
each Z^{2c} is independently C₁-C₆ alkyl, halo, C₁-C₆ haloalkyl, or C₃-C₆ cycloalkyl;
R^{Z2} is H, C₁-C₆ alkyl, C₁-C₆ haloalkyl, or C₃-C₆ cycloalkyl;
R^{Z2a} is H, C₁-C₆ alkyl, C₁-C₆ haloalkyl, or C₃-C₆ cycloalkyl;
ring C3 is C₃-C₈ cycloalkyl, 4- to 16-membered heterocyclyl, C₆-C₁₀ aryl, 5- to 10-membered heteroaryl, or absent;
each R^{C3} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
p is 0, 1, 2, 3, or 4;
Z³ is C₁-C₄ alkyl, C₂-C₃ alkenyl, C₂-C₃ alkynyl, —O—C₁-C₃ alkyl, —O—C₂-C₃ alkenyl, —O—C₂-C₃ alkynyl, —O—, —S—, —NR^{Z3a}—, —C(O)—C₁-C₃ alkyl, —C(O)NR^{Z3a}—, —C(O)—, —S(O)—, —S(O)₂—, —S(O)(═NR^{Z3})—C₃-C₄ cycloalkyl, 4- to 10-membered heterocyclyl, or a bond, wherein the alkyl and cycloalkyl is substituted with 0, 1, 2, 3, or 4 Z^{3a};
each Z^{3a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z3} is H, C₁-C₆ alkyl, C₁-C₆ haloalkyl or C₃-C₆ cycloalkyl;
R^{Z3a} is H, C₁-C₆ alkyl, C₁-C₆ haloalkyl, or C₃-C₆ cycloalkyl;
ring C4 is C₃-C₈ cycloalkyl, 4- to 16-membered heterocyclyl, C₆-C₁₀ aryl, 5- to 10-membered heteroaryl, or absent;
each R^{C4} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₆ cyanoalkyl, ═CH₂, ═CHF, ═CF₂, or C₃-C₆-cycloalkyl;
q is 0, 1, 2, 3, or 4;
Z⁴ is C₁-C₄ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z4a}—, —C(O)—C₁-C₃ alkyl, —C(O)NR^{Z4a}—, —C(O)—, —S(O)—, S(O)₂—, S(O)(═NR^{Z4})—, 4- to 10-membered heterocyclyl, or a bond, wherein the alkyl and cycloalkyl is substituted with 0, 1, 2, 3, or 4 Z^{4a};
each Z^{4a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z4} is H, C₁-C₆ alkyl, C₁-C₆ haloalkyl or C₃-C₆ cycloalkyl;
R^{Z4a} is H, C₁-C₆ alkyl, C₁-C₆ haloalkyl, or C₃-C₆ cycloalkyl;
ring C5 is 5 to 14-membered heterocyclyl, C₆-C₁₀ aryl, or 5- to 10-membered heteroaryl;
each R^{C5} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, NR^{Z1A}R^{Z1B}, —CN, C₁-C₃ cyanoalkyl, C₃-C₆ cycloalkyl, or 4- to 10-membered heterocyclyl;
r is 0, 1, 2, 3, or 4;
Z⁵ is —NH—, —C(O)NH—, or a bond, and Z⁶ is —CH—;
alternatively, Z⁵ is a bond and Z⁶ is N;
X is N, CH, or CR^{x};
R^{x} is halo, C₁-C₃ haloalkyl, C₁-C₃ cyanoalkyl, or C₃-C₆ cycloalkyl;
L¹ is CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6-membered heterocyclyl having 1 heteroatom that is O, wherein each cycloalkyl and heterocyclyl is substituted with 0, 1, 2, or 3 Rix;
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
such that when L² is ═C(R^{2e})— then L¹ is —C(R^{1e})═ or L³ is ═C(R^{3e})—, and when L³ is ═C(R^{3e})— then L² is ═C(R^{2e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SF₅, C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, —SR^{A3a}, —N(R^{A3a})(R^{A3b}), C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ thiohaloalkyl, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently C₁-C₁₀ alkyl, C₂-C₁₀ alkenyl, C2-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} attached to two different atoms can combine to form a C₃-C₁₀ cycloalkyl or 4- to 10-membered heterocyclyl, wherein the cycloalkyl and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{D} is halo;
wherein, unless otherwise indicated, each heterocyclyl has 1, 2, 3, or 4 heteroatoms selected from N, O, S, and Si; and
each heteroaryl has 1, 2, 3, or 4 heteroatoms selected from N, O, and S.

2. (canceled)

3. (canceled)

4. The compound of claim 1, or pharmaceutically acceptable salt thereof, having the structure of Formula (Ic):

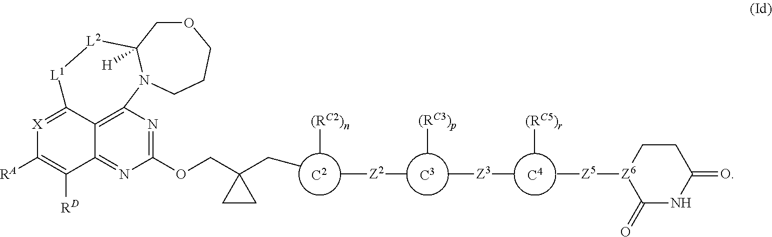

5. The compound of claim 1, or pharmaceutically acceptable salt thereof, wherein X is N; and R^{D} is F.

6. (canceled)

7. The compound of claim 1, or pharmaceutically acceptable salt thereof, wherein L¹ is CH₂; and L² is CHR^{2b}; R^{2b} is H or C₁-C₃ alkyl.

8-13. (canceled)

14. The compound of claim 1, or a pharmaceutically acceptable salt thereof, wherein R^{A} is naphthyl substituted with 2, 3, or 4 R^{A2}, each R^{A2} is independently Et, OH, —C≡CH, F, Cl, CF₃, —CN, or —NH₂.

15-19. (canceled)

20. The compound of claim 1, or a pharmaceutically acceptable salt thereof, wherein R^{A} is

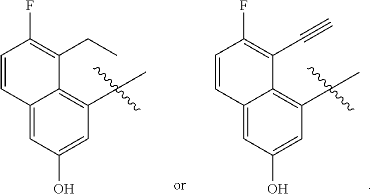 [SMILES: *c1cc(O)cc2ccc(F)c(CC)c12 ; *c1cc(O)cc2ccc(F)c(C#C)c12]

21-23. (canceled)

24. The compound of claim 1, or pharmaceutically acceptable salt thereof, having the structure of Formula (Id):

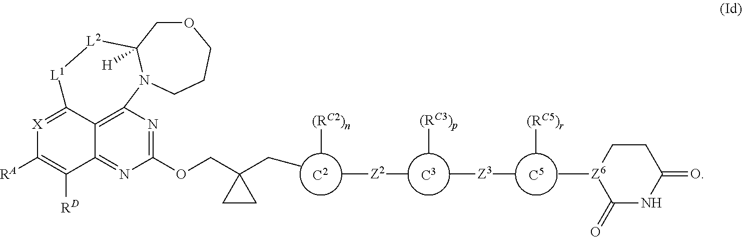

25. The compound of claim 1, or a pharmaceutically acceptable salt thereof, wherein Z¹ is

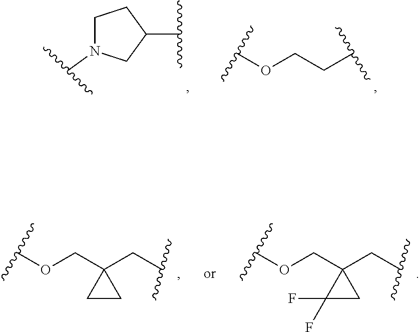 [SMILES: *C1CCN(*)C1 ; *CCO* ; *CC1(CO*)CC1 ; *CC1(CO*)CC1(F)F]

26. The compound of claim 1, or a pharmaceutically acceptable salt thereof, wherein
ring C2 is 6- to 14-membered heterocyclyl having at least one ring nitrogen, wherein the heterocyclyl has 1, 2, or 3 additional heteroatoms selected from N, O, and S;
each R^{C2} is independently C₁-C₆ alkyl, halo, C₁-C₆ haloalkyl, or —OH; and
n is 0 or 1.

27. (canceled)

28. The compound of claim 1, or pharmaceutically acceptable salt thereof, wherein
ring C2 is

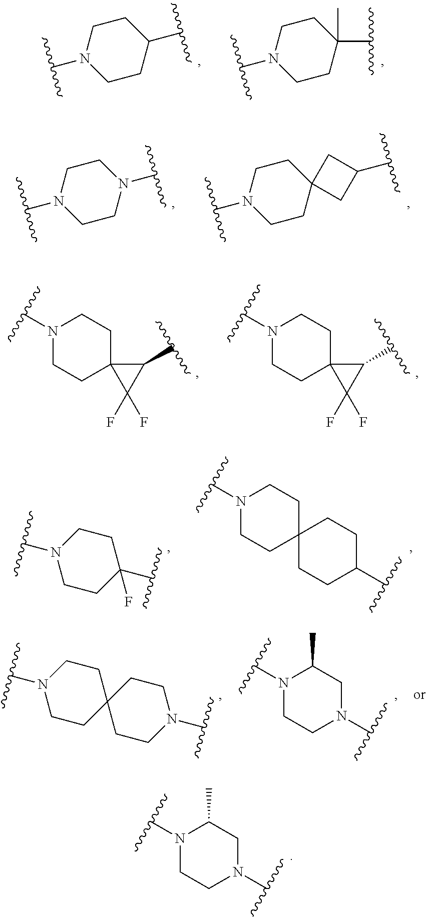

29. (canceled)

30. (canceled)

31. The compound of claim 1, or pharmaceutically acceptable salt thereof, wherein Z² is CH₂, CF₂, —OCH₂C≡C—, —O—, —N(CH₃)—, or a bond.

32. The compound of claim 1, or pharmaceutically acceptable salt thereof, wherein
ring C3 is 4- to 16-membered heterocyclyl or absent, wherein the heterocyclyl has 1, 2, 3, or 4 heteroatoms selected from N, O, and S;
each R^{C3} is independently C₁-C₆ alkyl or halo; and
p is 0 or 1.

33. (canceled)

34. The compound of claim 1, or pharmaceutically acceptable salt thereof, wherein
ring C3 is: 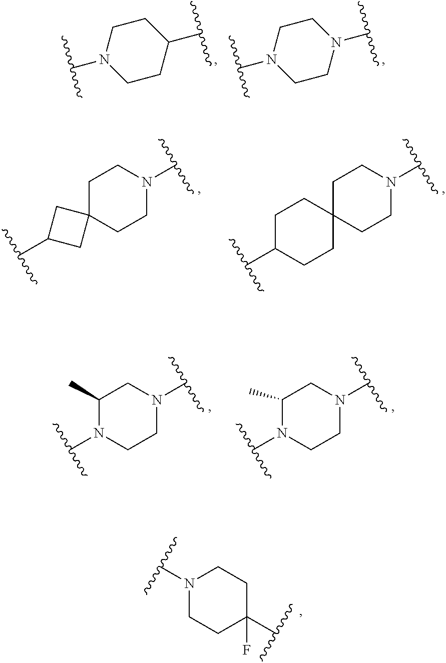
or absent.

35. The compound of claim 1, or pharmaceutically acceptable salt thereof, wherein
Z³ is —C(O)— or a bond,
ring C4 is absent;
Z⁴ is a bond; and
Z⁶ is —CH— or N.

36-38. (canceled)

39. The compound of claim 1, or pharmaceutically acceptable salt thereof, wherein
ring C5 is C₆-C₁₀ aryl or 5- to 10-membered heteroaryl, wherein the heteroaryl has 1 or 2 heteroatoms selected from N and O;
each R^{C5} is independently C₁-C₃ alkyl, C₁-C₃ alkoxy, halo, or oxo; and
r is 0, 1 or 2.

40. The compound of claim 1, or pharmaceutically acceptable salt thereof, wherein
ring C5 is

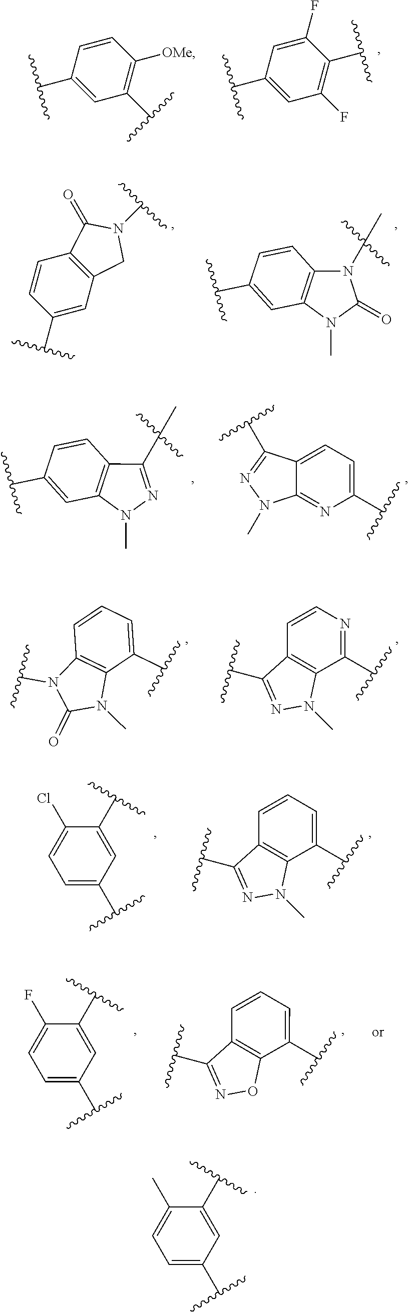

41-43. (canceled)

44. The compound of claim 1, or pharmaceutically acceptable salt thereof, wherein the moiety

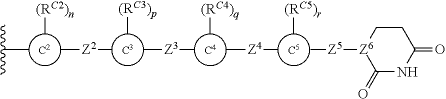

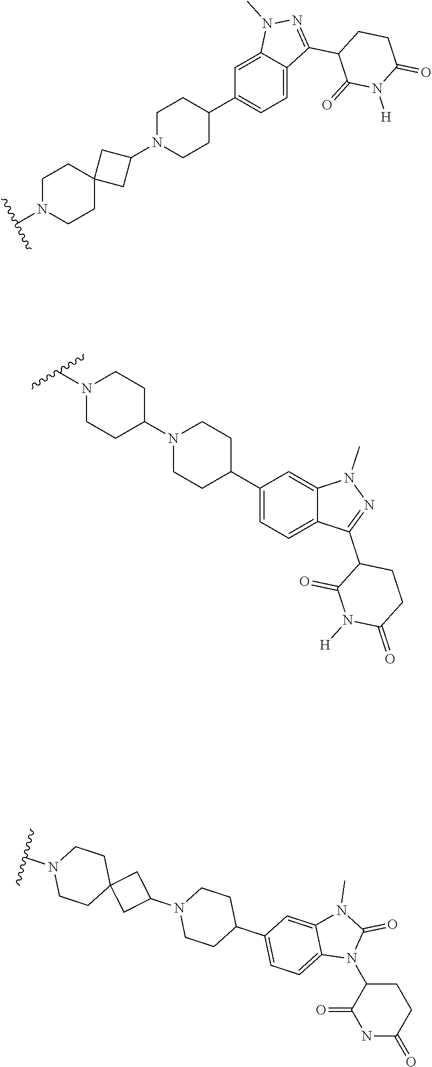 [SMILES: *N1CCC2(CC1)CC(N1CCC(c3ccc4c(C5CCC(=O)NC5=O)nn(C)c4c3)CC1)C2 ; *N1CCC(N2CCC(c3ccc4c(C5CCC(=O)NC5=O)nn(C)c4c3)CC2)CC1 ; *N1CCC2(CC1)CC(N1CCC(c3ccc4c(c3)n(C)c(=O)n4C3CCC(=O)NC3=O)CC1)C2]

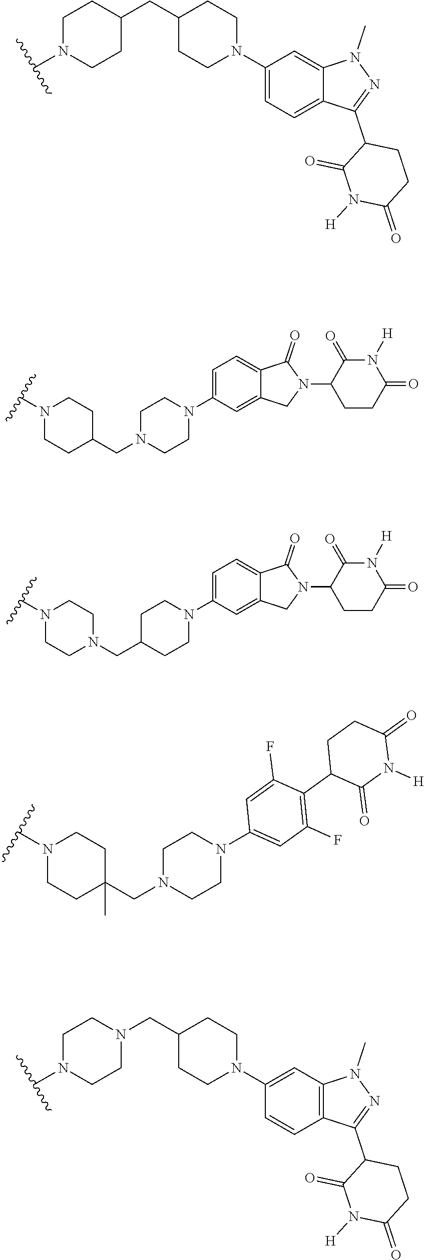 [SMILES: *N1CCC(CC2CCN(c3ccc4c(C5CCC(=O)NC5=O)nn(C)c4c3)CC2)CC1 ; *N1CCC(CN2CCN(c3ccc4c(c3)CN(C3CCC(=O)NC3=O)C4=O)CC2)CC1 ; *N1CCN(CC2CCN(c3ccc4c(c3)CN(C3CCC(=O)NC3=O)C4=O)CC2)CC1 ; *N1CCC(C)(CN2CCN(c3cc(F)c(C4CCC(=O)NC4=O)c(F)c3)CC2)CC1 ; *N1CCN(CC2CCN(c3ccc4c(C5CCC(=O)NC5=O)nn(C)c4c3)CC2)CC1]

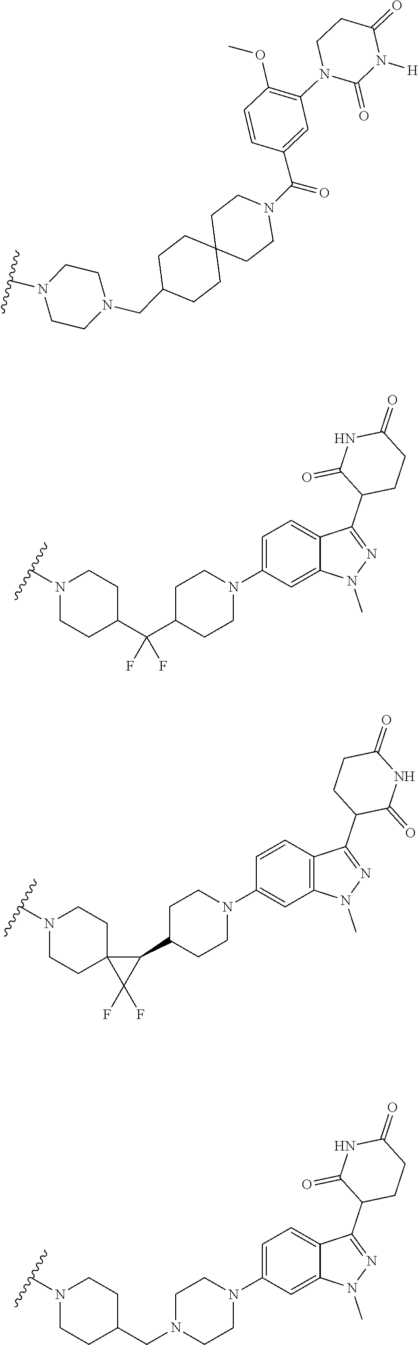 [SMILES: *N1CCN(CC2CCC3(CC2)CCN(C(=O)c2ccc(OC)c(N4CCC(=O)NC4=O)c2)CC3)CC1 ; *N1CCC(C(F)(F)C2CCN(c3ccc4c(C5CCC(=O)NC5=O)nn(C)c4c3)CC2)CC1 ; *N1CCC2(CC1)[C@@H](C1CCN(c3ccc4c(C5CCC(=O)NC5=O)nn(C)c4c3)CC1)C2(F)F ; *N1CCC(CN2CCN(c3ccc4c(C5CCC(=O)NC5=O)nn(C)c4c3)CC2)CC1]

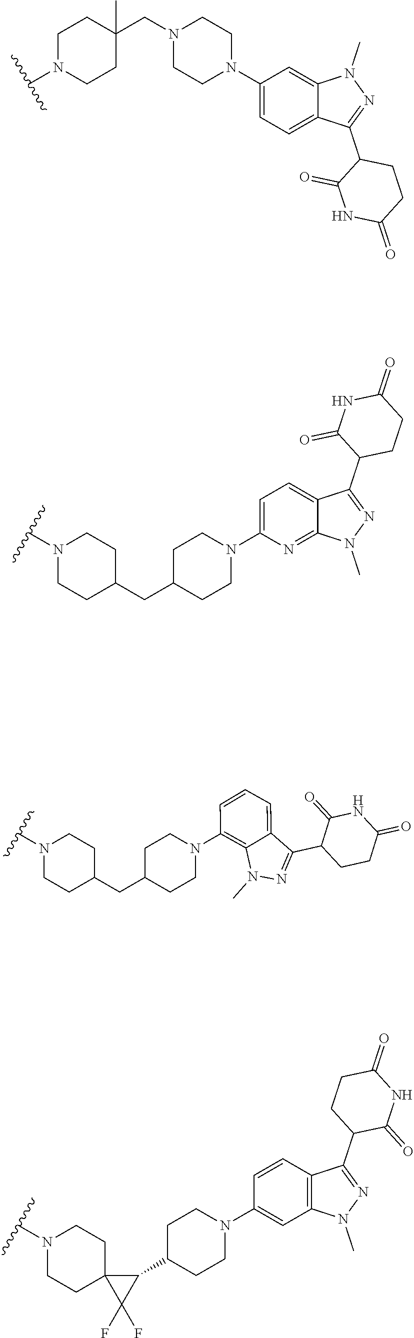 [SMILES: *N1CCC(C)(CN2CCN(c3ccc4c(C5CCC(=O)NC5=O)nn(C)c4c3)CC2)CC1 ; *N1CCC(CC2CCN(c3ccc4c(C5CCC(=O)NC5=O)nn(C)c4n3)CC2)CC1 ; *N1CCC(CC2CCN(c3cccc4c(C5CCC(=O)NC5=O)nn(C)c34)CC2)CC1 ; *N1CCC2(CC1)[C@H](C1CCN(c3ccc4c(C5CCC(=O)NC5=O)nn(C)c4c3)CC1)C2(F)F]

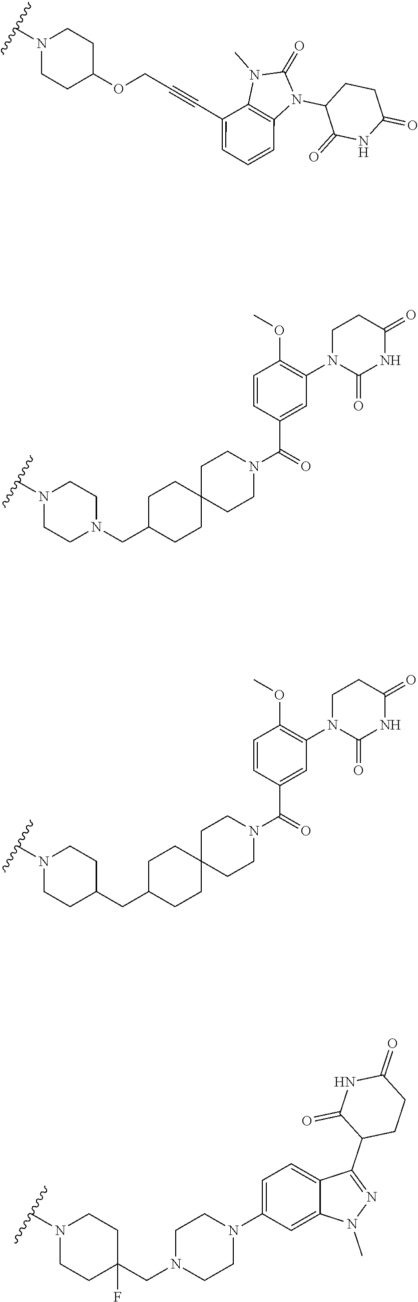 [SMILES: *N1CCC(OCC#Cc2cccc3c2n(C)c(=O)n3C2CCC(=O)NC2=O)CC1 ; *N1CCN(CC2CCC3(CC2)CCN(C(=O)c2ccc(OC)c(N4CCC(=O)NC4=O)c2)CC3)CC1 ; *N1CCC(CC2CCC3(CC2)CCN(C(=O)c2ccc(OC)c(N4CCC(=O)NC4=O)c2)CC3)CC1 ; *N1CCC(F)(CN2CCN(c3ccc4c(C5CCC(=O)NC5=O)nn(C)c4c3)CC2)CC1]

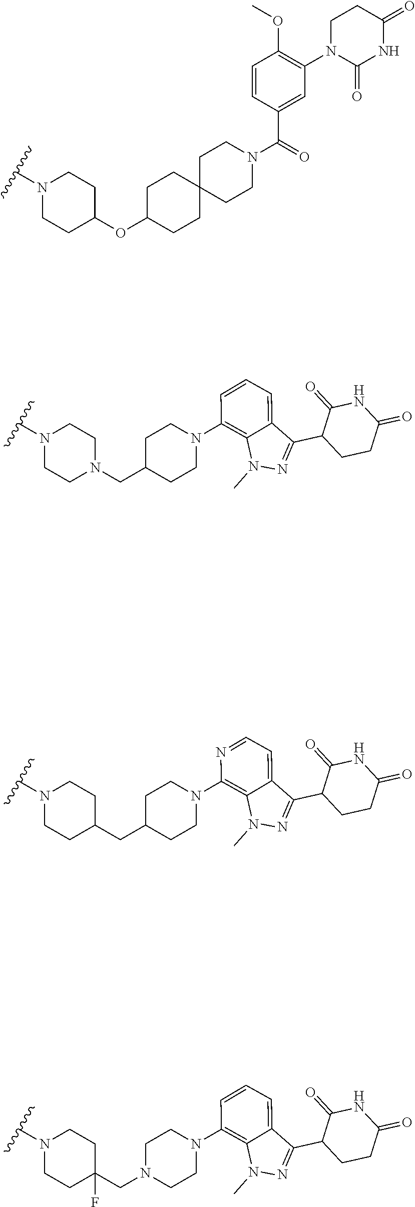 [SMILES: *N1CCC(OC2CCC3(CC2)CCN(C(=O)c2ccc(OC)c(N4CCC(=O)NC4=O)c2)CC3)CC1 ; *N1CCN(CC2CCN(c3cccc4c(C5CCC(=O)NC5=O)nn(C)c34)CC2)CC1 ; *N1CCC(CC2CCN(c3nccc4c(C5CCC(=O)NC5=O)nn(C)c34)CC2)CC1 ; *N1CCC(F)(CN2CCN(c3cccc4c(C5CCC(=O)NC5=O)nn(C)c34)CC2)CC1]

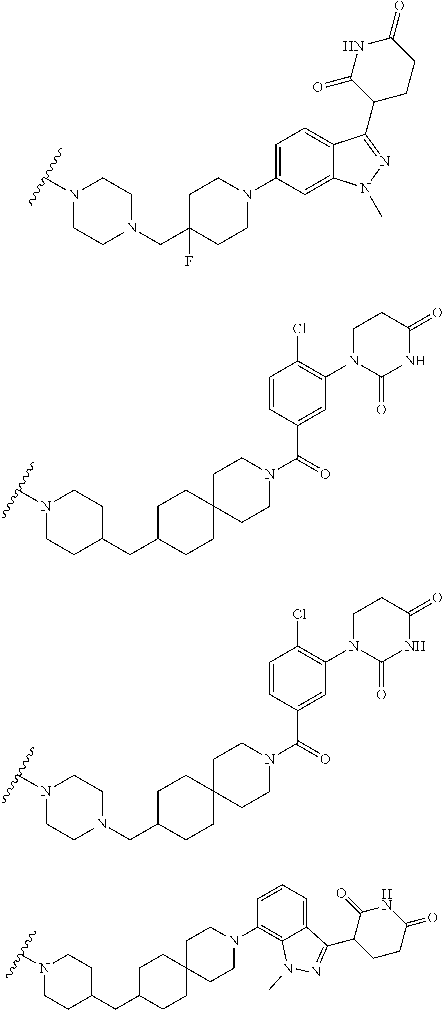 [SMILES: *N1CCN(CC2(F)CCN(c3ccc4c(C5CCC(=O)NC5=O)nn(C)c4c3)CC2)CC1 ; *N1CCC(CC2CCC3(CC2)CCN(C(=O)c2ccc(Cl)c(N4CCC(=O)NC4=O)c2)CC3)CC1 ; *N1CCN(CC2CCC3(CC2)CCN(C(=O)c2ccc(Cl)c(N4CCC(=O)NC4=O)c2)CC3)CC1 ; *N1CCC(CC2CCC3(CC2)CCN(c2cccc4c(C5CCC(=O)NC5=O)nn(C)c24)CC3)CC1]

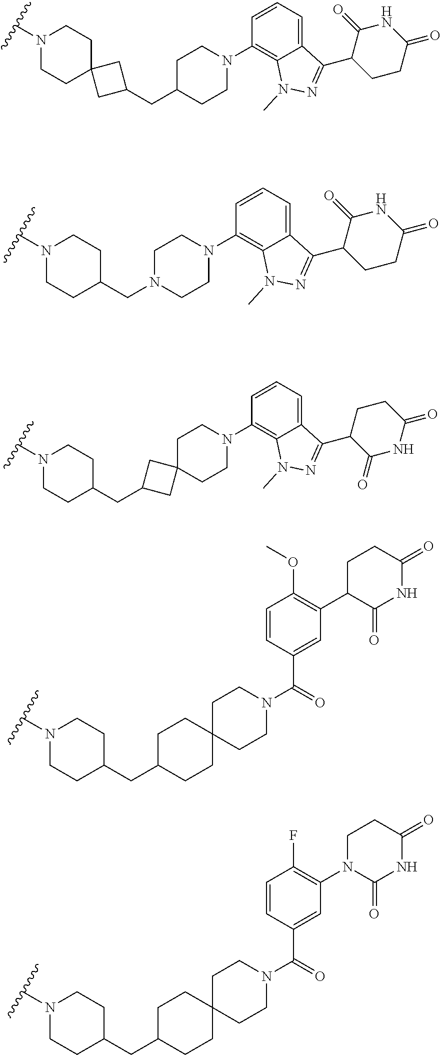 [SMILES: *N1CCC2(CC1)CC(CC1CCN(c3cccc4c(C5CCC(=O)NC5=O)nn(C)c34)CC1)C2 ; *N1CCC(CN2CCN(c3cccc4c(C5CCC(=O)NC5=O)nn(C)c34)CC2)CC1 ; *N1CCC(CC2CC3(CCN(c4cccc5c(C6CCC(=O)NC6=O)nn(C)c45)CC3)C2)CC1 ; *N1CCC(CC2CCC3(CC2)CCN(C(=O)c2ccc(OC)c(C4CCC(=O)NC4=O)c2)CC3)CC1 ; *N1CCC(CC2CCC3(CC2)CCN(C(=O)c2ccc(F)c(N4CCC(=O)NC4=O)c2)CC3)CC1]

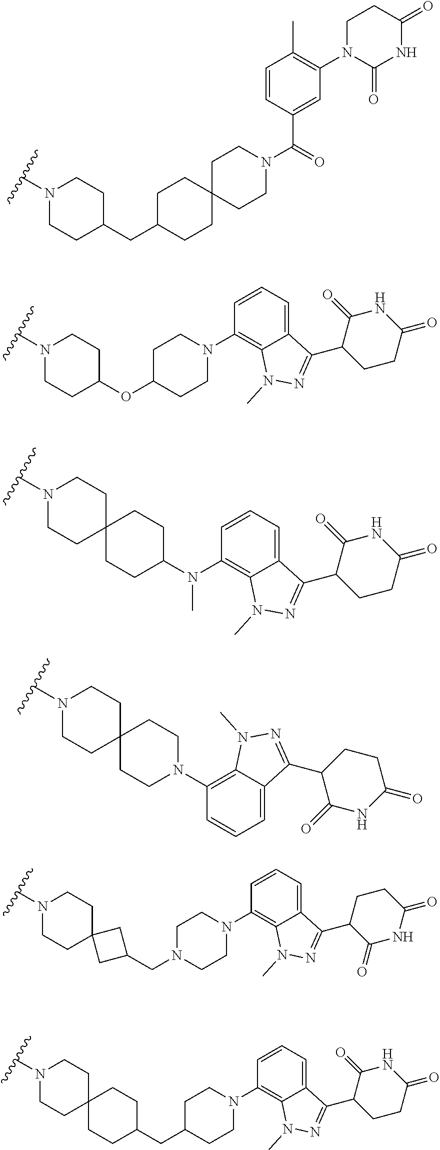 [SMILES: *N1CCC(CC2CCC3(CC2)CCN(C(=O)c2ccc(C)c(N4CCC(=O)NC4=O)c2)CC3)CC1 ; *N1CCC(OC2CCN(c3cccc4c(C5CCC(=O)NC5=O)nn(C)c34)CC2)CC1 ; *N1CCC2(CCC(N(C)c3cccc4c(C5CCC(=O)NC5=O)nn(C)c34)CC2)CC1 ; *N1CCC2(CC1)CCN(c1cccc3c(C4CCC(=O)NC4=O)nn(C)c13)CC2 ; *N1CCC2(CC1)CC(CN1CCN(c3cccc4c(C5CCC(=O)NC5=O)nn(C)c34)CC1)C2 ; *N1CCC2(CCC(CC3CCN(c4cccc5c(C6CCC(=O)NC6=O)nn(C)c45)CC3)CC2)CC1]

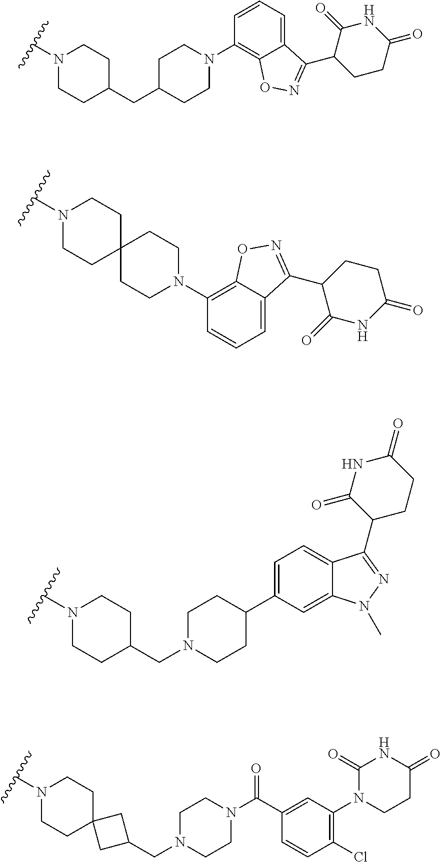 [SMILES: *N1CCC(CC2CCN(c3cccc4c(C5CCC(=O)NC5=O)noc34)CC2)CC1 ; *N1CCC2(CC1)CCN(c1cccc3c(C4CCC(=O)NC4=O)noc13)CC2 ; *N1CCC(CN2CCC(c3ccc4c(C5CCC(=O)NC5=O)nn(C)c4c3)CC2)CC1 ; *N1CCC2(CC1)CC(CN1CCN(C(=O)c3ccc(Cl)c(N4CCC(=O)NC4=O)c3)CC1)C2]

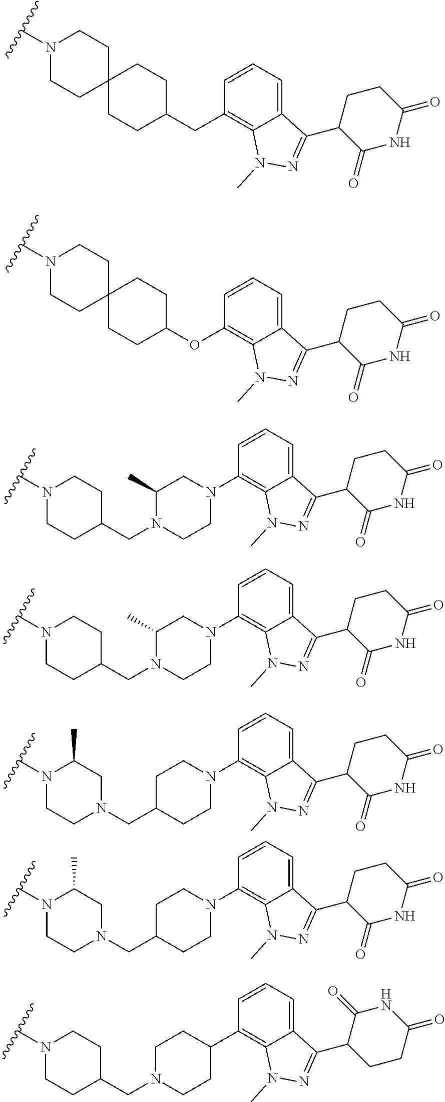

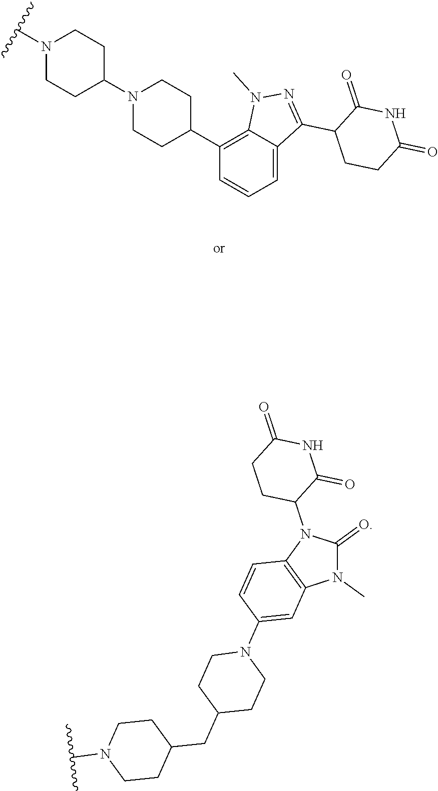 [SMILES: *N1CCC(N2CCC(c3cccc4c(C5CCC(=O)NC5=O)nn(C)c34)CC2)CC1 ; *N1CCC(CC2CCN(c3ccc4c(c3)n(C)c(=O)n4C3CCC(=O)NC3=O)CC2)CC1]

45. A compound having the structure of a compound of Example 1-1 to Example 1-10, Example 2-1 to Example 2-5, Example 3-1 to 3-40, Example 4-1, or Example 4-2, or pharmaceutically acceptable salt thereof.

46. A pharmaceutical composition comprising a compound of claim 1, or pharmaceutically acceptable salt thereof, and a pharmaceutically acceptable excipient.

47. (canceled)

48. (canceled)

49. A method of treating cancer in a subject in need thereof, the method comprising administering to the subject a therapeutically effective amount of a compound of claim 1, or a pharmaceutically acceptable salt thereof.

50-89. (canceled)
